(19) United States
(12) Patent Application Publication
(10) Pub. No.: US 2024/0196741 A1
(43) Pub. Date: Jun. 13, 2024

(54) ORGANIC COMPOUND, OPTO-ELECTRONIC DEVICE INCLUDING THE SAME, ELECTRONIC APPARATUS INCLUDING THE OPTO-ELECTRONIC DEVICE, AND ELECTRONIC DEVICE INCLUDING THE ELECTRONIC APPARATUS

(71) Applicant: Samsung Display Co., Ltd., Yongin-si (KR)

(72) Inventors: Hwasook Ryu, Yongin-si (KR); Hyejin Jung, Yongin-si (KR); Seokgyu Yoon, Yongin-si (KR)

(21) Appl. No.: 18/320,870

(22) Filed: May 19, 2023

(30) Foreign Application Priority Data
Oct. 25, 2022 (KR) 10-2022-0138624

(51) Int. Cl. H10K 85/60

(52) U.S. Cl. CPC H10K 85/6572; H10K 85/655

(57) ABSTRACT

An opto-electronic device includes a first electrode, a second electrode facing the first electrode, a photoactive layer arranged between the first electrode and the second electrode, and an organic compound represented by Formula 1, wherein Ar₁₁ in Formula 1 is a group represented by Formula 2:

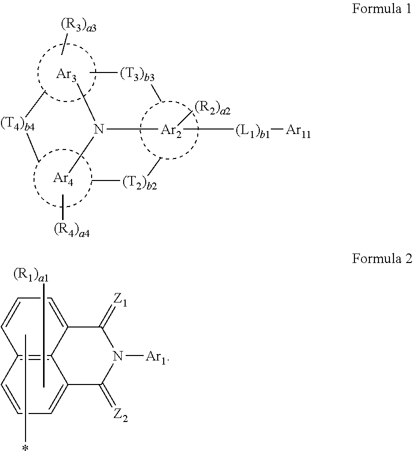

More detailed descriptions of Formulae 1 and 2 are as described herein.

### CROSS-REFERENCE TO RELATED APPLICATION

This application claims priority to and the benefit of Korean Patent Application No. 10-2022-0138624, filed on Oct. 25, 2022, in the Korean Intellectual Property Office, the content of which is incorporated by reference herein in its entirety.

### BACKGROUND

### 1. Field

One or more embodiments relate to an organic compound, an opto-electronic device including the same, an electronic apparatus including the opto-electronic device, and an electronic device including the electronic apparatus.

### 2. Description of the Related Art

Opto-electronic devices are devices that convert light energy or a light signal into electrical energy or an electrical signal. Examples of opto-electronic devices include a photovoltaic cell or a solar cell that converts light energy into electrical energy, a photodetector or a light sensor that detects light energy and converts the detected light energy into an electrical signal, and/or the like.

Electronic apparatuses including opto-electronic devices and organic light-emitting devices have been developed. As an example, light emitted from an organic light-emitting device may be reflected by an object (e.g., a finger of a user) in contact with an electronic apparatus, and then incident on an opto-electronic device. As the opto-electronic device detects incident light energy and converts the detected incident light energy into an electrical signal, it may be utilized to recognize that the object is in contact with the electronic apparatus. The opto-electronic device may be utilized as a fingerprint recognition sensor and/or the like.

### SUMMARY

Aspects according to one or more embodiments are directed toward an organic compound having a relatively small molecular weight and improved light absorption efficiency for green light, and an opto-electronic device having relatively high heat resistance characteristics and high photoelectric characteristics with respect to green light by including the organic compound. In addition, aspects according to one or more embodiments are directed toward a high-quality electronic apparatus and a high-quality electronic device, each including the opto-electronic device.

Additional aspects will be set forth in part in the description which follows and, in part, will be apparent from the description, or may be learned by practice of the presented embodiments of the disclosure.

According to one or more embodiments, an opto-electronic device includes a first electrode, a second electrode facing the first electrode, a photoactive layer between the first electrode and the second electrode, and an organic compound represented by Formula 1:

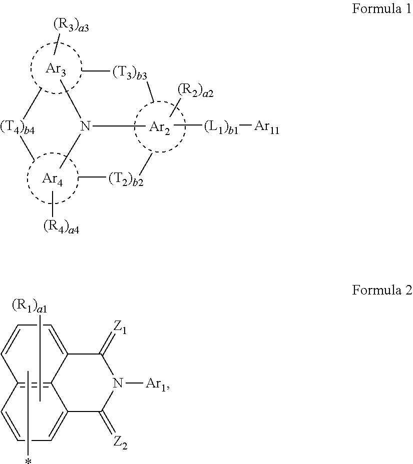

wherein, in Formula 1, Ar₁₁ may be a group represented by Formula 2,
in Formulae 1 and 2,
Ar₁ may be a C₁-C₆₀ alkyl group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
Z₁ and Z₂ may each independently be O, S, or N, wherein, i) when Z₁ is N, Z₁ and Ar₁ may be bonded to each other, and ii) when Z₂ is N, Z₂ and Ar₁ may be bonded to each other,
L₁ may be a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b1 may be an integer from 0 to 2,
when b1 is 2, two L₁(s) may be identical to or different from each other,
Ar₂ to Ar₄ may each independently be a C₃-C₆₀ carbocyclic group or a C₁-C₆₀ heterocyclic group,
T₂ may be a single bond, *—C(T₂₁)(T₂₂)-*′, *—Si(T₂₁)(T₂₂)-*′, *—O—*′, *—S—*′, or —N(T₂₁)-*′,
T₃ may be a single bond, *—C(T₃₁)(T₃₂)-*′, *—Si(T₃₁)(T₃₂)-*′, *—O—*′, *—S—*′, or —N(T₃₁)-*′,
T₄ may be a single bond, *—C(T₄₁)(T₄₂)-*′, *—Si(T₄₁)(T₄₂)-*′, *—O—*′, *—S—*′, or —N(T₄₁)-*′,
b2 to b4 may each independently be an integer from 0 to 2, wherein, i) when b2 is 0, *-(T₂)_{b2}-*′ may not be present, ii) when b3 is 0, *-(T₃)_{b3}-*′ may not be present, iii) when b4 is 0, *-(T₄)_{b4}-*′ may not be present, iv) when b2 is 2, two T₂(s) may be identical to or different from each other, v) when b3 is 2, two T₃(s) may be identical to or different from each other, and vi) when b4 is 2, two T₄(s) may be identical to or different from each other,
at least one of b2 to b4 may be 1 or 2,
no group other than Ar₁₁ in Formula 1 is represented by Formula 2,
R₁ to R₅, T₂₁, T₂₂, T₃₁, T₃₂, T₄₁, and T₄₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1 to a5 may each independently be an integer from 0 to 5,
R₁₀ₐ may be:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ may each independently be:

hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group, or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ may each indicate a binding site to a neighboring atom.

According to one or more embodiments, an electronic apparatus includes the opto-electronic device.

According to one or more embodiments, an electronic device includes the electronic apparatus.

According to one or more embodiments, an organic compound is represented by Formula 1.

### BRIEF DESCRIPTION OF THE DRAWINGS

The above and other aspects, features, and enhancements of certain embodiments of the disclosure will be more apparent from the following description taken in conjunction with the accompanying drawings, in which:

FIG. 1 is a schematic view of an opto-electronic device according to an embodiment;

FIG. 2 is a schematic view of a light-emitting device included in an electronic apparatus according to an embodiment;

FIG. 3 is a schematic view of an opto-electronic device according to another embodiment;

FIG. 4 is a schematic view of an opto-electronic device according to another embodiment;

FIG. 5 is a schematic view of an electronic apparatus according to an embodiment;

FIG. 6 is a schematic view of an electronic apparatus according to another embodiment;

FIG. 7 is a schematic perspective view of an electronic device including an opto-electronic device according to an embodiment;

FIG. 8 is a schematic view of the exterior of a vehicle as an electronic device including an opto-electronic device according to an embodiment; and

FIGS. 9A-9C are schematic views each being of the interior of a vehicle according to a respective embodiment.

### DETAILED DESCRIPTION

Reference will now be made in more detail to embodiments, examples of which are illustrated in the accompanying drawings, wherein like reference numerals refer to like elements throughout, and duplicative descriptions thereof may not be provided. In this regard, the present embodiments may have different forms and should not be construed as being limited to the descriptions set forth herein. Accordingly, the embodiments are merely described below, by referring to the drawings, to explain aspects of the present description. As used herein, the term “and/or” includes any and all combinations of one or more of the associated listed items. Throughout the disclosure, the expression “at least one of a, b or c”, “at least one selected from a, b, and c”, “at least one selected from the group consisting of a, b, and c”, “at least one from among a, b, and c”, “at least one of a to c”, etc., indicates only a, only b, only c, both (e.g., simultaneously) a and b, both (e.g., simultaneously) a and c, both (e.g., simultaneously) b and c, all of a, b, and c, or variations thereof.

According to one or more embodiments of the present disclosure, an opto-electronic device includes a first electrode, a second electrode facing the first electrode, a photoactive layer arranged between the first electrode and the second electrode, and an organic compound represented by Formula 1. Formula 1 will be described in more detail below.

In an embodiment, the first electrode may be an anode. The second electrode may be a cathode. The photoactive layer may be to absorb light to generate excitons. The excitons may be separated into electrons and holes. As the electrons and holes are generated, current may flow (e.g., an electrical current may be generated).

In an embodiment, the photoactive layer may include the organic compound.

The organic compound may be to absorb light to generate electrons and holes, and may serve as an electron donor. The organic compound may be referred to as an electron donating compound.

In an embodiment, the opto-electronic device may further include an electron accepting compound. The electron accepting compound may serve as an electron acceptor. For example, the electron accepting compound may be fullerene, or may be represented by one of Formulae 4-1 to 4-4:

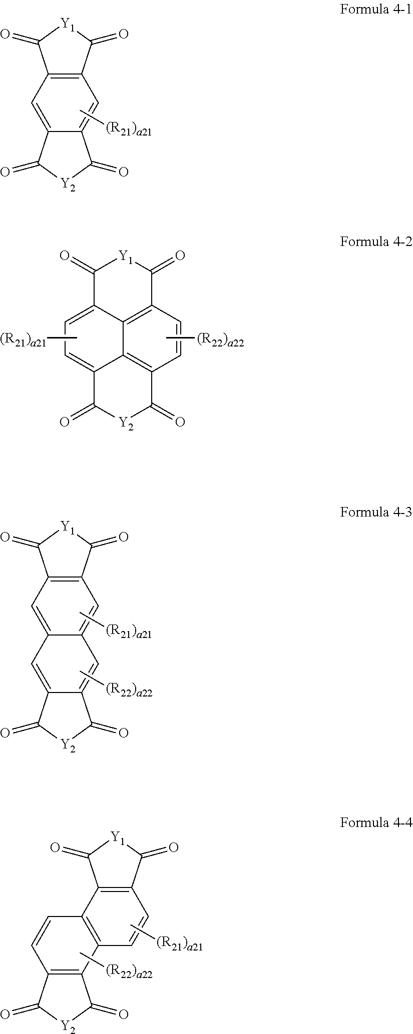

wherein, in Formulae 4-1 to 4-4,
Y₁ may be O or N(Ar₂₁),
Y₂ may be O or N(Ar₂₂),
Ar₂₁ and Ar₂₂ may each independently be hydrogen, deuterium, a C₁-C₆₀ alkyl group unsubstituted or substituted with at least one R_{10b}, a C₃-C₆₀ carbocyclic group unsubstituted or substituted with at least one R_{10b}, or a C₁-C₆₀ heterocyclic group unsubstituted or substituted with at least one R_{10b},
a21 and a22 may each independently be 0, 1, or 2,
R₂₁ and R₂₂ may each independently be hydrogen, deuterium, —F, —Cl, —Br, —I, a hydroxyl group, a cyano group, a nitro group, a C₁-C₆₀ alkyl group unsubstituted or substituted with at least one R_{10b}, a C₂-C₆₀ alkenyl group unsubstituted or substituted with at least one R_{10b}, a C₂-C₆₀ alkynyl group unsubstituted or substituted with at least one R_{10b}, a C₁-C₆₀ alkoxy group unsubstituted or substituted with at least one R_{10b}, a C₃-C₆₀ carbocyclic group unsubstituted or substituted with at least one R_{10b}, a C₁-C₆₀ heterocyclic group unsubstituted or substituted with at least one R_{10b}, a C₁-C₆₀ aryloxy group unsubstituted or substituted with at least one R_{10b}, a C₆-C₆₀ arylthio group unsubstituted or substituted with at least one R_{10b}, a C₇-C₆₀ arylalkyl group unsubstituted or substituted with at least one R_{10b}, a C₂-C₆₀ heteroarylalkyl group unsubstituted or substituted with at least one R_{10b}, —C(Q₅₁)(Q₅₂)(Q₅₃), —Si(Q₅₁)(Q₅₂)(Q₅₃), —N(Q₅₁)(Q₅₂), —B(Q₅₁)(Q₅₂), —C(═O)(Q₅₁), —S(═O)₂(Q₅₁), or —P(═O)(Q₅₁)(Q₅₂),
R_{10b} may be the same as defined herein in connection with R₁₀ₐ, and
Q₅₁ to Q₅₃ may each independently be the same as defined herein in connection with Q₁.

In an embodiment, the electron accepting compound may be fullerene. For example, the electron accepting compound may be fullerene 60 or fullerene 70:

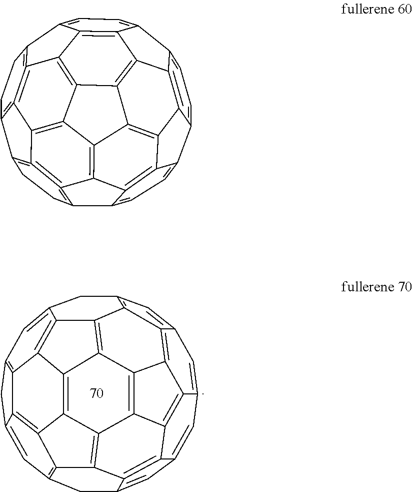

In an embodiment, the electron accepting compound may be one of Compounds N1 to N36:

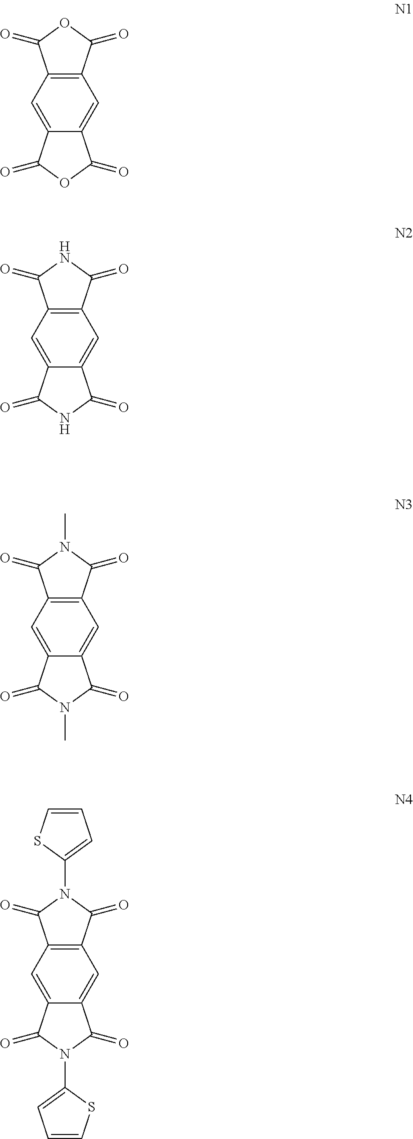 [SMILES: O=c1oc(=O)c2cc3c(=O)oc(=O)c3cc12 ; O=c1[nH]c(=O)c2cc3c(=O)[nH]c(=O)c3cc12 ; Cn1c(=O)c2cc3c(=O)n(C)c(=O)c3cc2c1=O ; O=c1c2cc3c(=O)n(-c4cccs4)c(=O)c3cc2c(=O)n1-c1cccs1]

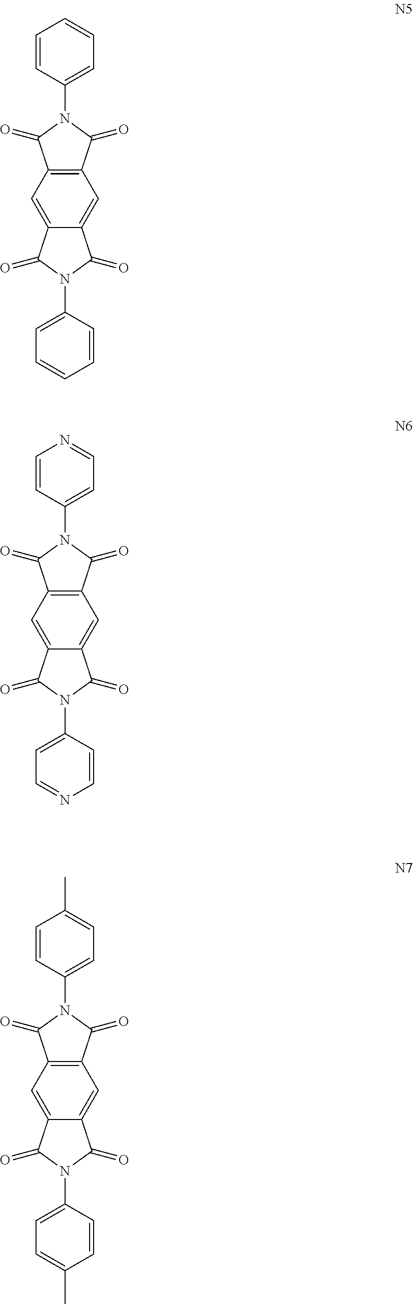 [SMILES: O=c1c2cc3c(=O)n(-c4ccccc4)c(=O)c3cc2c(=O)n1-c1ccccc1 ; O=c1c2cc3c(=O)n(-c4ccncc4)c(=O)c3cc2c(=O)n1-c1ccncc1 ; Cc1ccc(-n2c(=O)c3cc4c(=O)n(-c5ccc(C)cc5)c(=O)c4cc3c2=O)cc1]

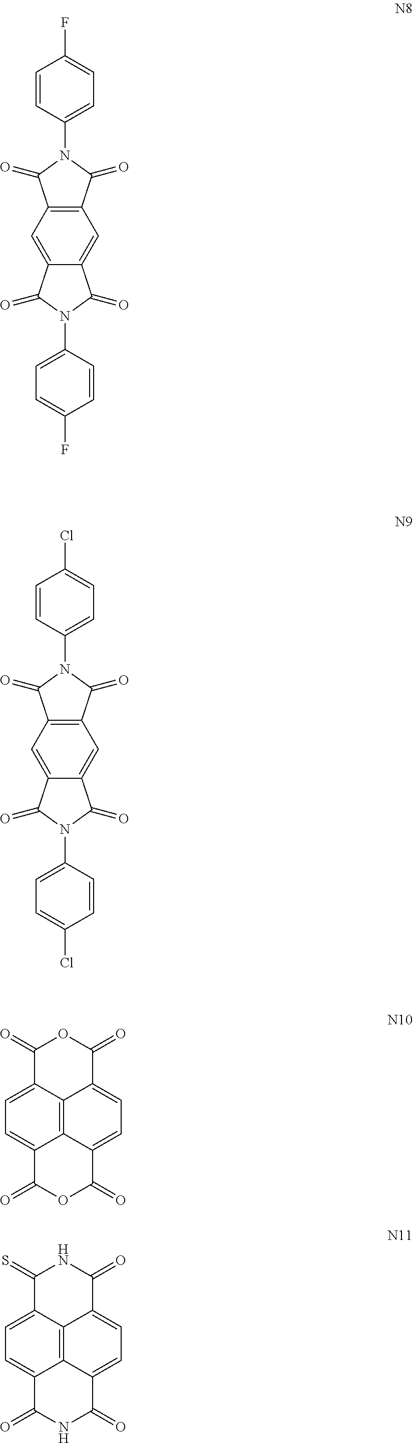 [SMILES: O=c1c2cc3c(=O)n(-c4ccc(F)cc4)c(=O)c3cc2c(=O)n1-c1ccc(F)cc1 ; O=c1c2cc3c(=O)n(-c4ccc(Cl)cc4)c(=O)c3cc2c(=O)n1-c1ccc(Cl)cc1 ; O=C1OC(=O)c2ccc3c4c(ccc1c24)C(=O)OC3=O ; O=C1NC(=O)c2ccc3c4c(ccc1c24)C(=O)NC3=S]

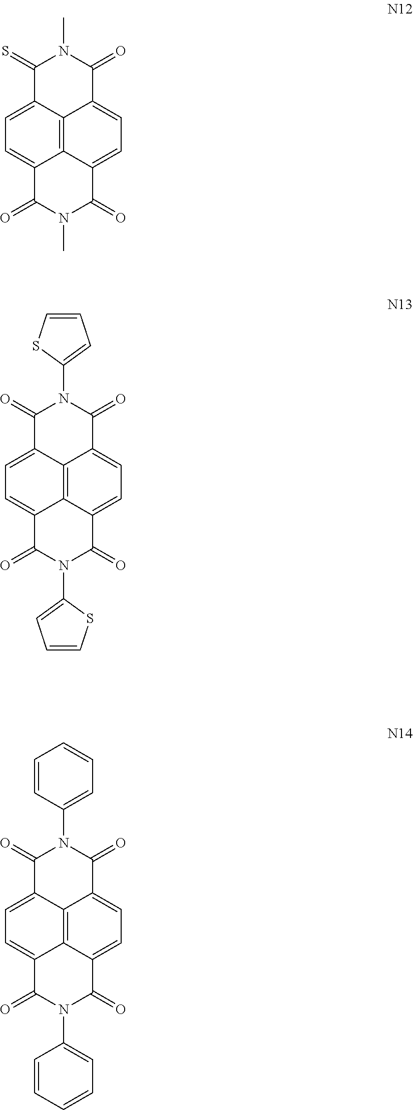 [SMILES: CN1C(=O)c2ccc3c4c(ccc(c24)C1=O)C(=S)N(C)C3=O ; O=C1c2ccc3c4c(ccc(c24)C(=O)N1c1cccs1)C(=O)N(c1cccs1)C3=O ; O=C1c2ccc3c4c(ccc(c24)C(=O)N1c1ccccc1)C(=O)N(c1ccccc1)C3=O]

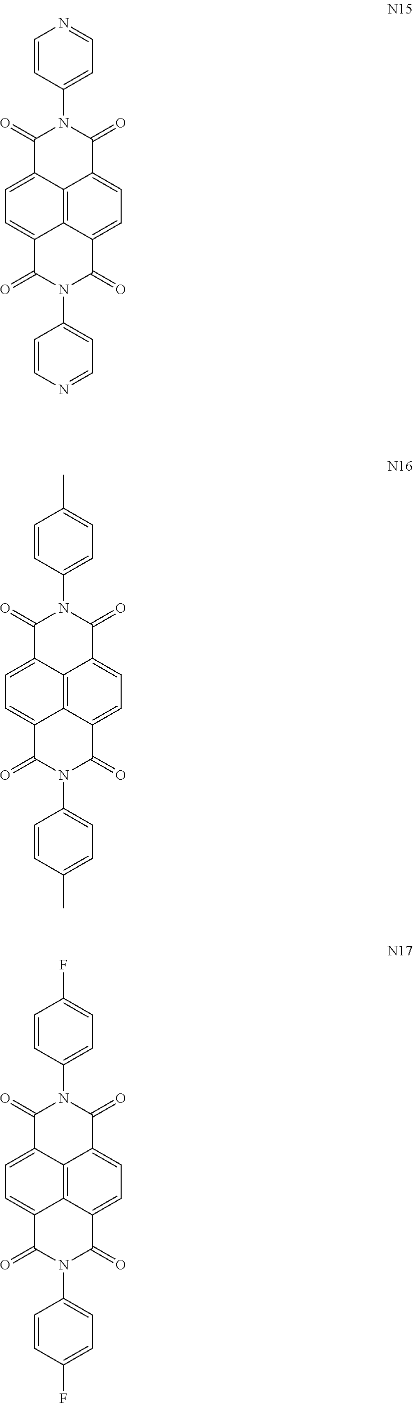 [SMILES: O=C1c2ccc3c4c(ccc(c24)C(=O)N1c1ccncc1)C(=O)N(c1ccncc1)C3=O ; Cc1ccc(N2C(=O)c3ccc4c5c(ccc(c35)C2=O)C(=O)N(c2ccc(C)cc2)C4=O)cc1 ; O=C1c2ccc3c4c(ccc(c24)C(=O)N1c1ccc(F)cc1)C(=O)N(c1ccc(F)cc1)C3=O]

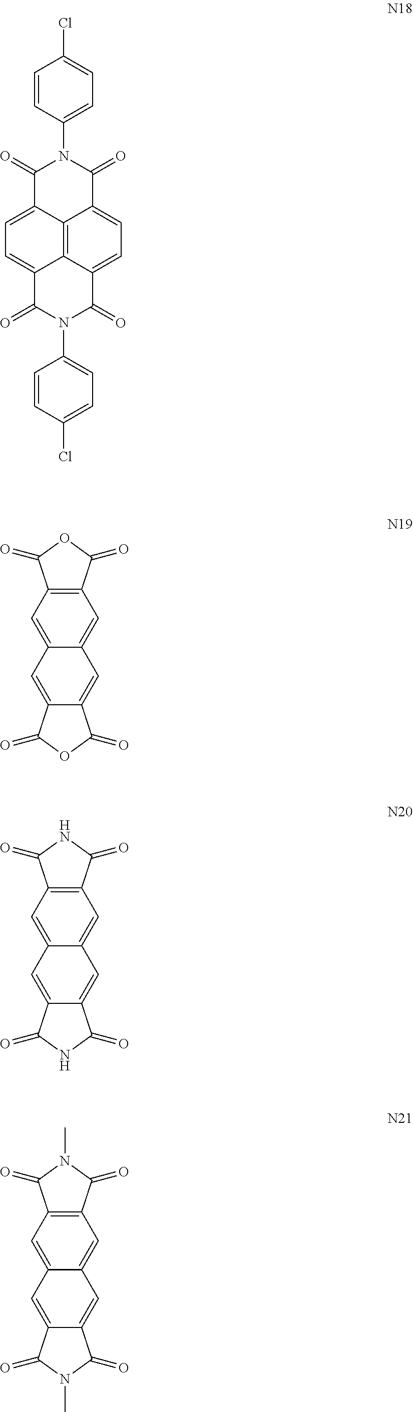 [SMILES: O=C1c2ccc3c4c(ccc(c24)C(=O)N1c1ccc(Cl)cc1)C(=O)N(c1ccc(Cl)cc1)C3=O ; O=c1oc(=O)c2cc3cc4c(=O)oc(=O)c4cc3cc12 ; O=c1[nH]c(=O)c2cc3cc4c(=O)[nH]c(=O)c4cc3cc12 ; Cn1c(=O)c2cc3cc4c(=O)n(C)c(=O)c4cc3cc2c1=O]

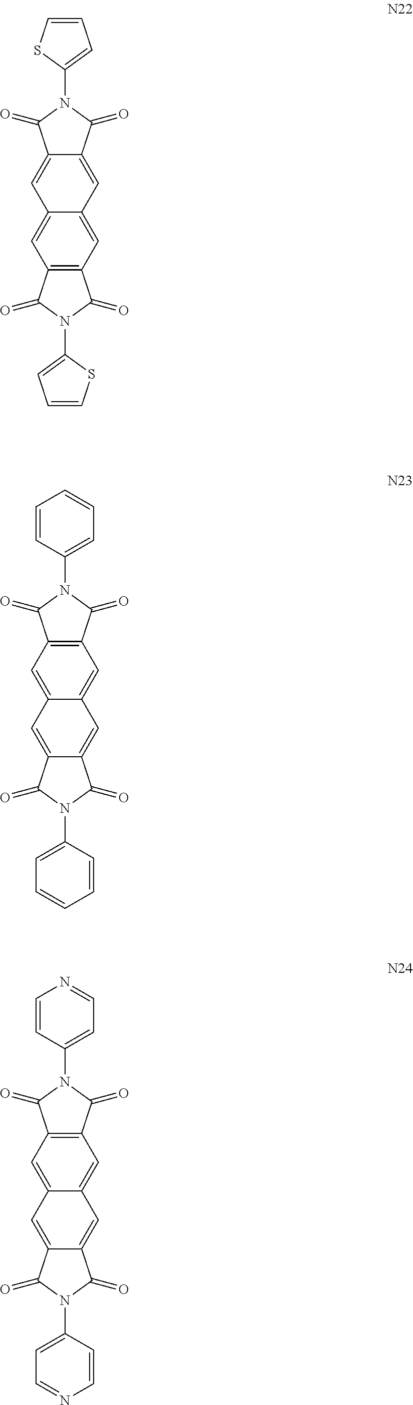 [SMILES: O=c1c2cc3cc4c(=O)n(-c5cccs5)c(=O)c4cc3cc2c(=O)n1-c1cccs1 ; O=c1c2cc3cc4c(=O)n(-c5ccccc5)c(=O)c4cc3cc2c(=O)n1-c1ccccc1 ; O=c1c2cc3cc4c(=O)n(-c5ccncc5)c(=O)c4cc3cc2c(=O)n1-c1ccncc1]

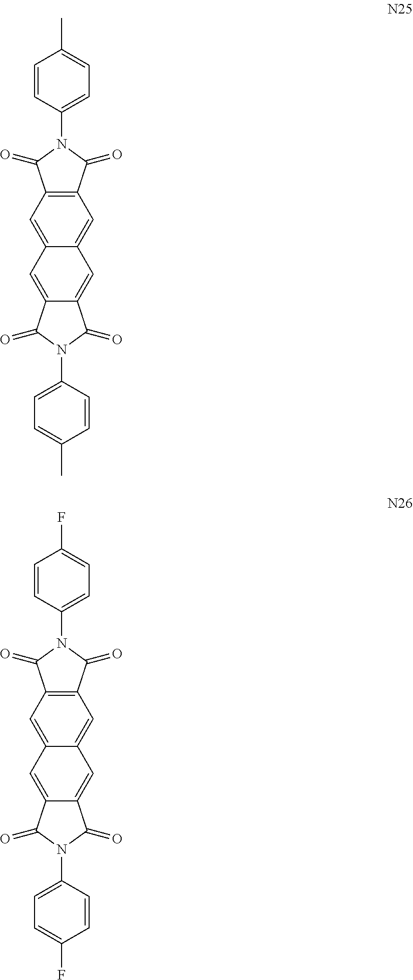

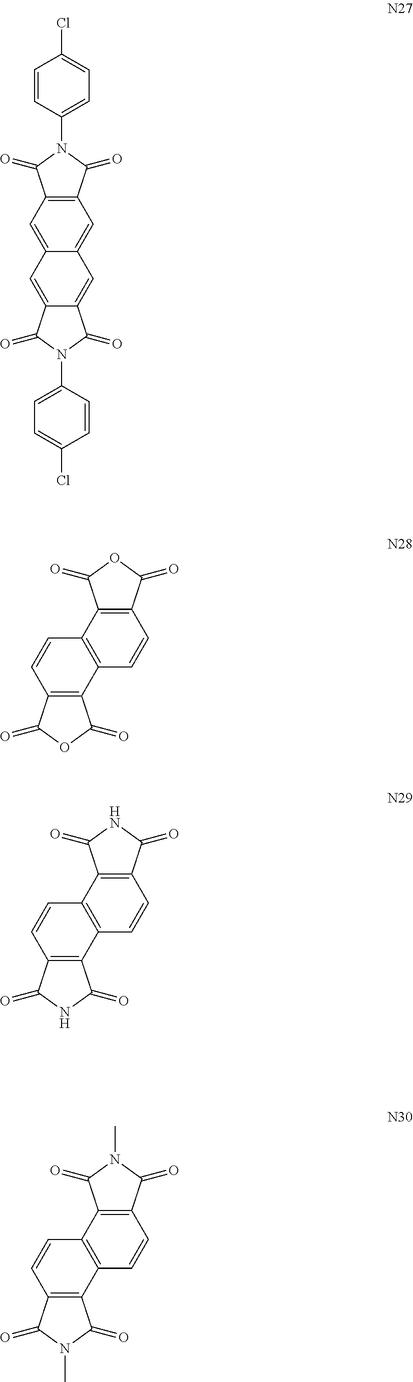 [SMILES: O=c1c2cc3cc4c(=O)n(-c5ccc(Cl)cc5)c(=O)c4cc3cc2c(=O)n1-c1ccc(Cl)cc1 ; CC1=C(/C=C\c2cccc3c2C(=O)OC3=O)C(=O)OC1=O ; CC1=C(/C=C\c2cccc3c2C(=O)NC3=O)C(=O)NC1=O ; CC1=C(/C=C\c2cccc3c2C(=O)N(C)C3=O)C(=O)N(C)C1=O]

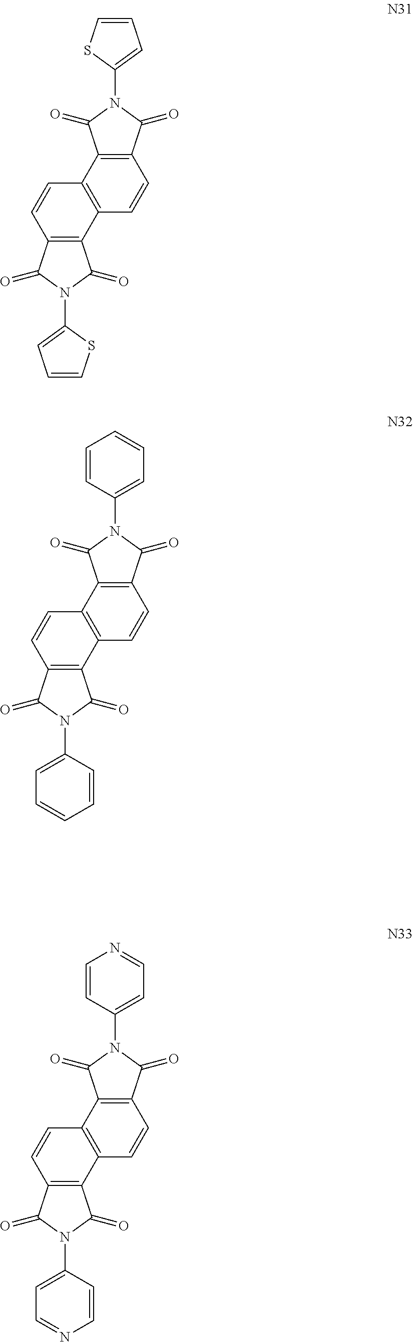 [SMILES: CC1=C(/C=C\c2cccc3c2C(=O)N(c2cccs2)C3=O)C(=O)N(c2cccs2)C1=O ; CC1=C(/C=C\c2cccc3c2C(=O)N(c2ccccc2)C3=O)C(=O)N(c2ccccc2)C1=O ; CC1=C(/C=C\c2cccc3c2C(=O)N(c2ccncc2)C3=O)C(=O)N(c2ccncc2)C1=O]

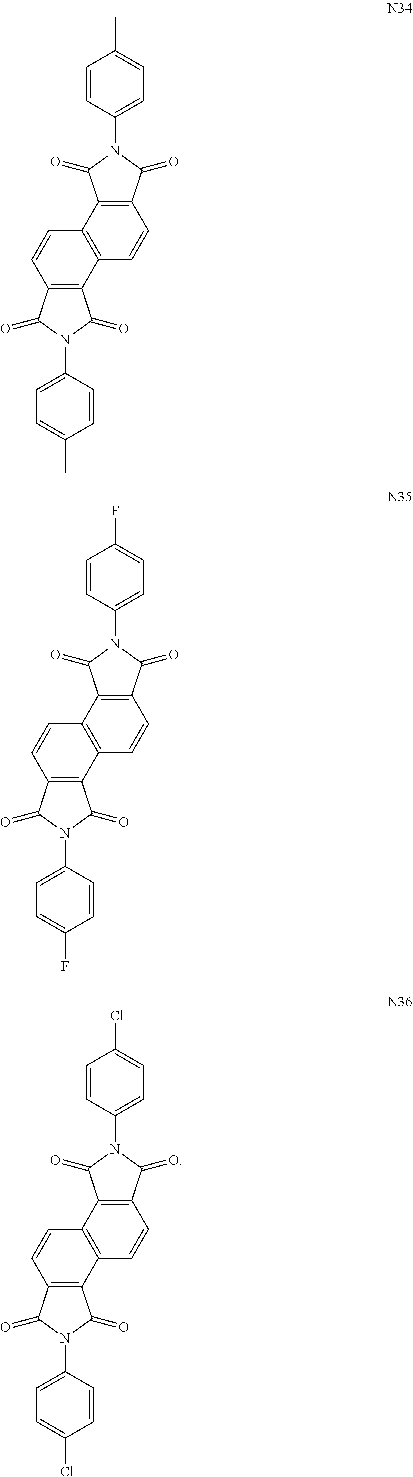 [SMILES: CC1=C(/C=C\c2cccc3c2C(=O)N(c2ccc(C)cc2)C3=O)C(=O)N(c2ccc(C)cc2)C1=O ; CC1=C(/C=C\c2cccc3c2C(=O)N(c2ccc(F)cc2)C3=O)C(=O)N(c2ccc(F)cc2)C1=O ; CC1=C(/C=C\c2cccc3c2C(=O)N(c2ccc(Cl)cc2)C3=O)C(=O)N(c2ccc(Cl)cc2)C1=O]

In an embodiment, the photoactive layer may include the organic compound and the electron accepting compound.

In an embodiment, the opto-electronic device may further include a hole transport region arranged between the first electrode and the photoactive layer and an electron transport region arranged between the photoactive layer and the second electrode. Holes generated by the photoactive layer may move to the first electrode through the hole transport region. Electrons generated by the photoactive layer may move to the second electrode through the electron transport region.

In an embodiment, the photoactive layer may include a first layer adjacent to the hole transport region and a second layer adjacent to the electron transport region.

In an embodiment, the first layer may include the organic compound. For example, the first layer may not include (e.g., may exclude) the electron accepting compound.

In an embodiment, the second layer may include the electron accepting compound. The electron accepting compound may be fullerene. For example, the second layer may not include (e.g., may exclude) the organic compound.

For example, the first layer may be referred to as a p-type or kind photoactive layer or an electron donor layer. The second layer may be referred to as an n-type or kind photoactive layer or an electron acceptor layer.

In an embodiment, the photoactive layer may further include a third layer arranged between the first layer and the second layer. The third layer may include (e.g., both) the organic compound and the electron accepting compound.

According to one or more embodiments of the present disclosure, an electronic apparatus includes the opto-electronic device.

In an embodiment, the electronic apparatus may further include a light-emitting device adjacent to the opto-electronic device. The light-emitting device may not overlap the opto-electronic device.

For example, light emitted by the light-emitting device may be extracted (e.g., emitted) to the outside of the electronic apparatus. The light may be reflected by an external object to be incident into the electronic apparatus. The opto-electronic device may be to absorb the incident light. For example, the opto-electronic device may be utilized as a sensor that recognizes an object outside the electronic apparatus.

In an embodiment, the light-emitting device may include a first electrode, a second electrode facing the first electrode, and an emission layer arranged between the first electrode and the second electrode. In an embodiment, the first electrode of the light-emitting device may be a portion of the first electrode of the opto-electronic device. In one or more embodiments, the first electrode of the light-emitting device may be apart from the first electrode of the opto-electronic device, but may include the same material as the first electrode of the opto-electronic device. In an embodiment, the second electrode of the light-emitting device may be a portion of the second electrode of the opto-electronic device. In one or more embodiments, the second electrode of the light-emitting device may be apart from the second electrode of the opto-electronic device, but may include the same material as the second electrode of the opto-electronic device. The emission layer may include a dopant and a host, and may be to emit light.

In an embodiment, the opto-electronic device may further include a hole transport region arranged between the first electrode and the emission layer and an electron transport region arranged between the emission layer and the second electrode. The hole transport region may include a hole injection layer, a hole transport layer, an emission auxiliary layer, an electron blocking layer, or any combination thereof. The electron transport region may include a buffer layer, a hole blocking layer, an electron control layer, an electron transport layer, an electron injection layer, or any combination thereof. For example, the emission auxiliary layer may generate a resonance phenomenon by light interference, and the buffer layer may control injection of electrons and prevent or reduce loss of holes.

For example, the first electrode, the hole transport region, the electron transport region, and the second electrode included in the opto-electronic device may be substantially identical to or different from the first electrode, the hole transport region, the electron transport region, and the second electrode included in the light-emitting device, respectively. For example, a portion of the opto-electronic device may extend (e.g., laterally or in a plan view) to constitute a portion of the light-emitting device.

In an embodiment, the electronic apparatus may further include a thin-film transistor, a color filter, a color conversion layer, a touch screen layer, a polarizing layer, or any combination thereof, and the thin-film transistor may be electrically connected to the first electrode.

According to one or more embodiments of the present disclosure, an electronic device includes the electronic apparatus. The electronic device may be a flat panel display, a curved display, a computer monitor, a medical monitor, a television, a billboard, an indoor or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual or augmented-reality display, a vehicle, a video wall including multiple displays tiled together, a theater or stadium screen, a phototherapy device, and/or a signboard.

According to one or more embodiments of the present disclosure, an organic compound is represented by Formula 1:

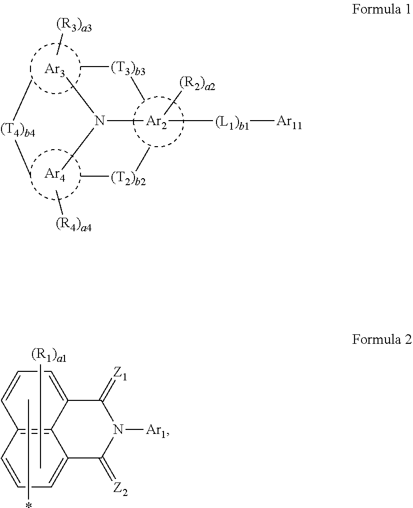

wherein, in Formula 1, Ar₁₁ may be a group represented by Formula 2,
in Formulae 1 and 2,
Ar₁ may be a C₁-C₆₀ alkyl group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
Z₁ and Z₂ may each independently be O, S, or N, wherein, i) when Z₁ is N, Z₁ and Ar₁ may be linked to each other, and ii) when Z₂ is N, Z₂ and Ar₁ may be linked to each other,
L₁ may be a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b1 may be an integer from 0 to 2,
when b1 is 2, two L₁(s) may be identical to or different from each other,
Ar₂ to Ar₄ may each independently be a C₃-C₆₀ carbocyclic group or a C₁-C₆₀ heterocyclic group,
T₂ may be a single bond, *—C(T₂₁)(T₂₂)-*′, *—Si(T₂₁)(T₂₂)-*′, *—O—*′, *—S—*′, or —N(T₂₁)-*′,
T₂₁ and T₂₂ may optionally be linked to each other via a single bond, a double bond, or *—O—*′,
T₃ may be a single bond, *—C(T₃₁)(T₃₂)-*′, *—Si(T₃₁)(T₃₂)-*′, *—O—*′, *—S—*′, or —N(T₃₁)-*′,
T₃₁ and T₃₂ may optionally be linked to each other via a single bond, a double bond, or *—O—*′,
T₄ may be a single bond, *—C(T₄₁)(T₄₂)-*′, *—Si(T₄₁)(T₄₂)-*′, *—O—*′, *—S—*′, or —N(T₄₁)-*′,
T₄₁ and T₄₂ may optionally be linked to each other via a single bond, a double bond, or *—O—*′,
b2 to b4 may each independently be an integer from 0 to 2, wherein, i) when b2 is 0, *-(T₂)_{b2}-*′ may not be present, ii) when b3 is 0, *-(T₃)_{b3}-*′ may not be present, iii) when b4 is 0, *-(T₄)_{b4}-*′ may not be present, iv) when b2 is 2, two T₂(s) may be identical to or different from each other, v) when b3 is 2, two T₃(s) may be identical to or different from each other, and vi) when b4 is 2, two T₄(s) may be identical to or different from each other,
at least one of b2 to b4 may be 1 or 2,
the number of groups represented by Formula 2 in Formula 1 may be one, that is, Formula 1 may only have one group that is represented by Formula 2, or no group other than Ar₁₁ in Formula 1 is represented by Formula 2,
R₁ to R₅, T₂₁, T₂₂, T₃₁, T₃₂, T₄₁, and T₄₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1 to a5 may each independently be an integer from 0 to 5,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P (═O) (Q₃₁) (Q₃₂),
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ may each indicate a binding site to a neighboring atom.

For example, when Z₁ is N, Z₁ and Ar₁ may be bonded to each other via a single bond. When Z₂ is N, Z₂ and Ar₁ may be bonded to each other via a single bond. When Z₁ is N, Ar₁ may be a C₃-C₂₀ carbocyclic group unsubstituted or substituted with at least one R₁₀ₐ or a C₁-C₂₀ heterocyclic group unsubstituted or substituted with at least one R₁₀ₐ. For example, when Z₁ is N, Ar₁ may not be a C₁-C₆₀ alkyl group unsubstituted or substituted with at least one R₁₀ₐ, and Z₁ and Ar₁ may be bonded to each other via a single bond.

In an embodiment, when Z₁ is N, a ring formed by bonding between Z₁ and Ar₁ may be a 5-membered ring.

In an embodiment, when Z₁ is N, Formula 2 may be represented by Formula 2-1:

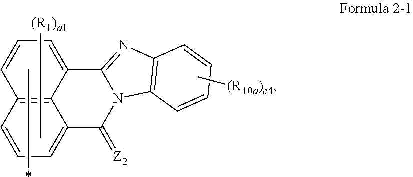

wherein, in Formula 2-1,
Z₂ may be O or S,
a1 may be an integer from 0 to 5,
c4 may be an integer from 0 to 4, and
R₁ and R₁₀ₐ may be the same as respectively defined herein in connection with Formulae 1 and 2.

In an embodiment, Formula 2 may be represented by one of Formulae 2a, 2b, or 2c:

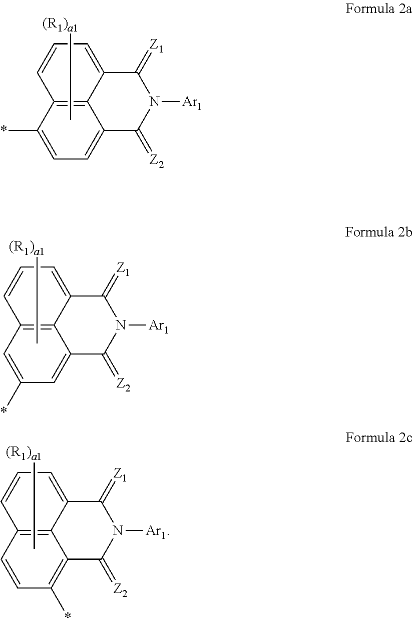

In an embodiment, i) b2 may be 1, b3 may be 0, and b4 may be 0; ii) b2 may be 0, b3 may be 1, and b4 may be 0; or iii) b2 may be 1, b3 may be 0, and b4 may be 1.

In an embodiment, Ar₁ may be: a methyl group, an ethyl group, an n-propyl group, an isopropyl group, a cyclohexyl group, a phenyl group, a naphthyl group, or a thiophenyl group; or a cyclohexyl group, a phenyl group, a naphthyl group, or a thiophenyl group, each substituted with at least one selected from the group consisting of —F, —Cl, and a cyano group.

In an embodiment, Formula 1 may be represented by one of Formulae 3-1 to 3-3:

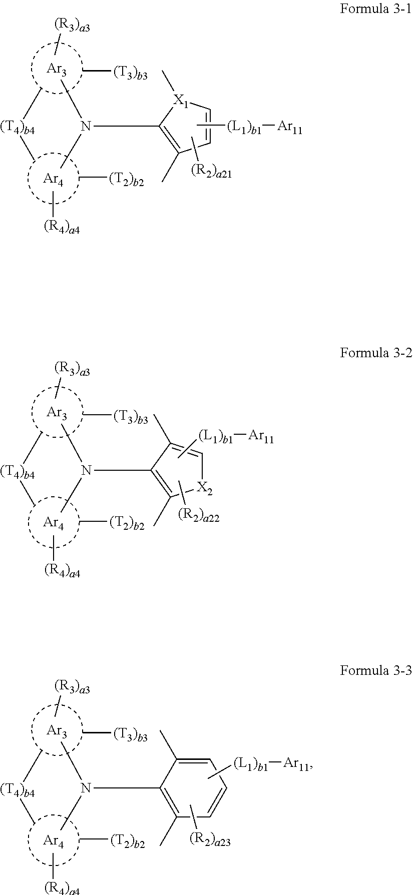

wherein, in Formulae 3-1 to 3-3,
X₁ and X₂ may each independently be O, S, Se, Te, C(Q₄₁)(Q₄₂), N(Q₄₁), B(Q₄₁), Si(Q₄₁)(Q₄₂), or Ge(Q₄₁)(Q₄₂),
when X₁ is O, S, Se, or Te, b3 may be 0,
a21 and a22 may each independently be an integer from 0 to 2,
a23 may be an integer from 0 to 4, and
Q₄₁ and Q₄₂ may each independently be the same as defined herein in connection with Q₁.

In an embodiment, L₁ may be a single bond.

In an embodiment, a molecular weight of the organic compound may be about 1,000 or less. For example, the molecular weight of the organic compound may be about 600 or less. In one embodiment, the molecular weight of the organic compound may be in a range of about 400 to about 560.

In an embodiment, at least one of Ar₂ to Ar₄ may include a thiophene group.

For example, the thiophene group may include thiophene, 2,3-dihydrothiophene, and/or 2,3-dihydrobenzothiophene.

In an embodiment, Ar₃ and Ar₄ may each independently be a benzene group, a naphthalene group, a thiophene group, a pyrrole group, a furan group, an indole group, a benzothiophene group, a benzofuran group, a quinoline group, or an isoquinoline group.

For example, the furan group may include furan, 2,3-dihydrofuran, benzofuran, and/or 2,3-dihyrobenzofuran.

In an embodiment, i) T₂ may be *—C(T₂₁)(T₂₂)-*′ or *—Si(T₂₁)(T₂₂)-*′, and T₂₁ and T₂₂ may be linked to each other via a single bond; ii) T₃ may be *—C(T₃₁)(T₃₂)-*′ or *—Si(T₃₁)(T₃₂)-*′, and T₃₁ and T₃₂ may be linked to each other via a single bond; or iii) T₄ may be *—C(T₄₁)(T₄₂)-*′ or *—Si(T₄₁)(T₄₂)-*′, and T₄₁ and T₄₂ may be linked to each other via a single bond.

In an embodiment, a highest occupied molecular orbital (HOMO) level of the organic compound may be in a range of about −6.0 eV to about −5.3 eV.

In an embodiment, a lowest unoccupied molecular orbital (LUMO) level of the organic compound may be in a range of about −3.0 eV to about −2.4 eV.

In an embodiment, a maximum absorption wavelength of the organic compound may be in a range of about 400 nm to about 550 nm. In one embodiment, the maximum absorption wavelength of the organic compound may be in a range of about 420 nm to about 535 nm.

In an embodiment, the organic compound may be one of Compounds 1 to 96:

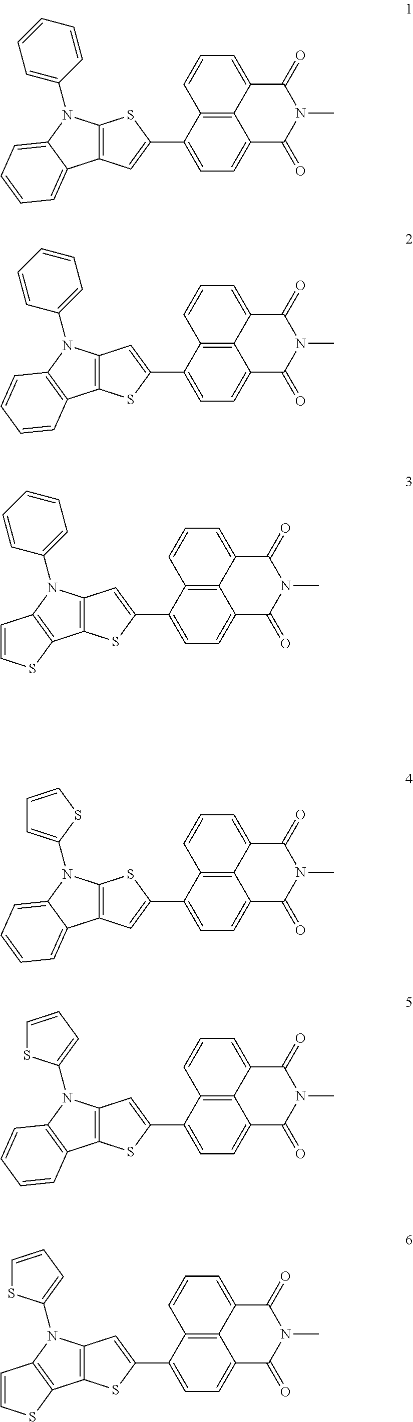 [SMILES: CN1C(=O)c2cccc3c(-c4cc5c6ccccc6n(-c6ccccc6)c5s4)ccc(c23)C1=O ; CN1C(=O)c2cccc3c(-c4cc5c(s4)c4ccccc4n5-c4ccccc4)ccc(c23)C1=O ; CN1C(=O)c2cccc3c(-c4cc5c(s4)c4sccc4n5-c4ccccc4)ccc(c23)C1=O ; CN1C(=O)c2cccc3c(-c4cc5c6ccccc6n(-c6cccs6)c5s4)ccc(c23)C1=O ; CN1C(=O)c2cccc3c(-c4cc5c(s4)c4ccccc4n5-c4cccs4)ccc(c23)C1=O ; CN1C(=O)c2cccc3c(-c4cc5c(s4)c4sccc4n5-c4cccs4)ccc(c23)C1=O]

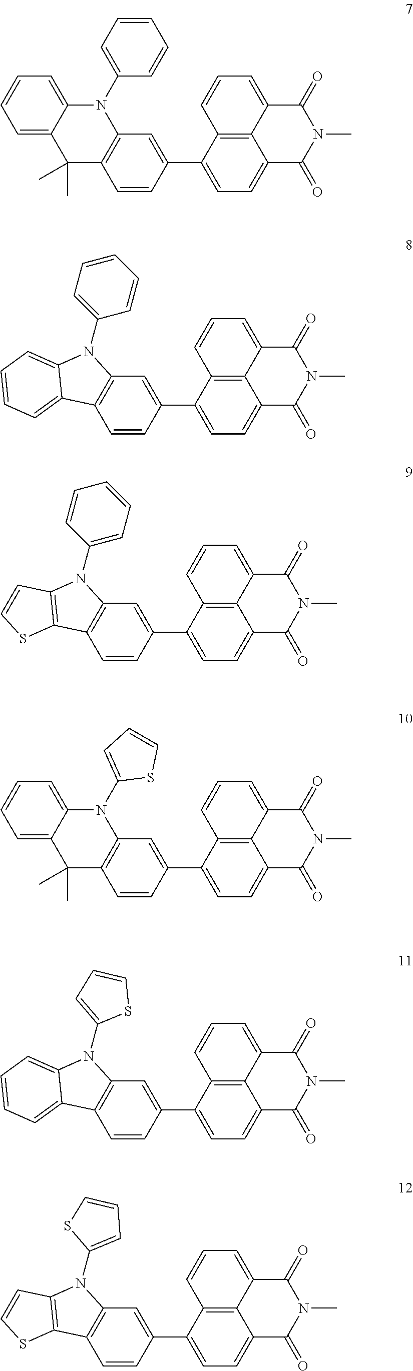 [SMILES: CN1C(=O)c2cccc3c(-c4ccc5c(c4)N(c4ccccc4)c4ccccc4C5(C)C)ccc(c23)C1=O ; CN1C(=O)c2cccc3c(-c4ccc5c6ccccc6n(-c6ccccc6)c5c4)ccc(c23)C1=O ; CN1C(=O)c2cccc3c(-c4ccc5c6sccc6n(-c6ccccc6)c5c4)ccc(c23)C1=O ; CN1C(=O)c2cccc3c(-c4ccc5c(c4)N(c4cccs4)c4ccccc4C5(C)C)ccc(c23)C1=O ; CN1C(=O)c2cccc3c(-c4ccc5c6ccccc6n(-c6cccs6)c5c4)ccc(c23)C1=O ; CN1C(=O)c2cccc3c(-c4ccc5c6sccc6n(-c6cccs6)c5c4)ccc(c23)C1=O]

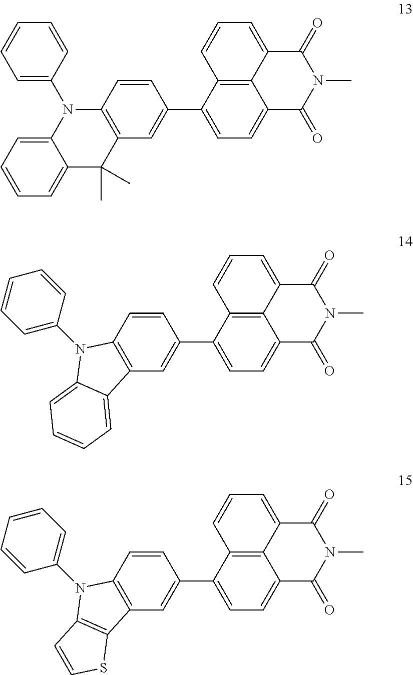 [SMILES: CN1C(=O)c2cccc3c(-c4ccc5c(c4)C(C)(C)c4ccccc4N5c4ccccc4)ccc(c23)C1=O ; CN1C(=O)c2cccc3c(-c4ccc5c(c4)c4ccccc4n5-c4ccccc4)ccc(c23)C1=O ; CN1C(=O)c2cccc3c(-c4ccc5c(c4)c4sccc4n5-c4ccccc4)ccc(c23)C1=O]

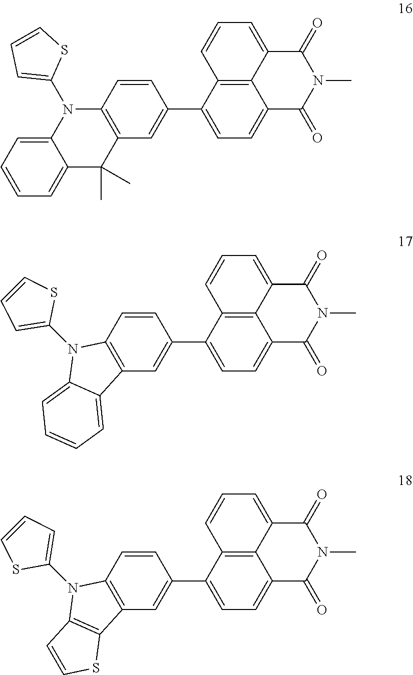 [SMILES: CN1C(=O)c2cccc3c(-c4ccc5c(c4)C(C)(C)c4ccccc4N5c4cccs4)ccc(c23)C1=O ; CN1C(=O)c2cccc3c(-c4ccc5c(c4)c4ccccc4n5-c4cccs4)ccc(c23)C1=O ; CN1C(=O)c2cccc3c(-c4ccc5c(c4)c4sccc4n5-c4cccs4)ccc(c23)C1=O]

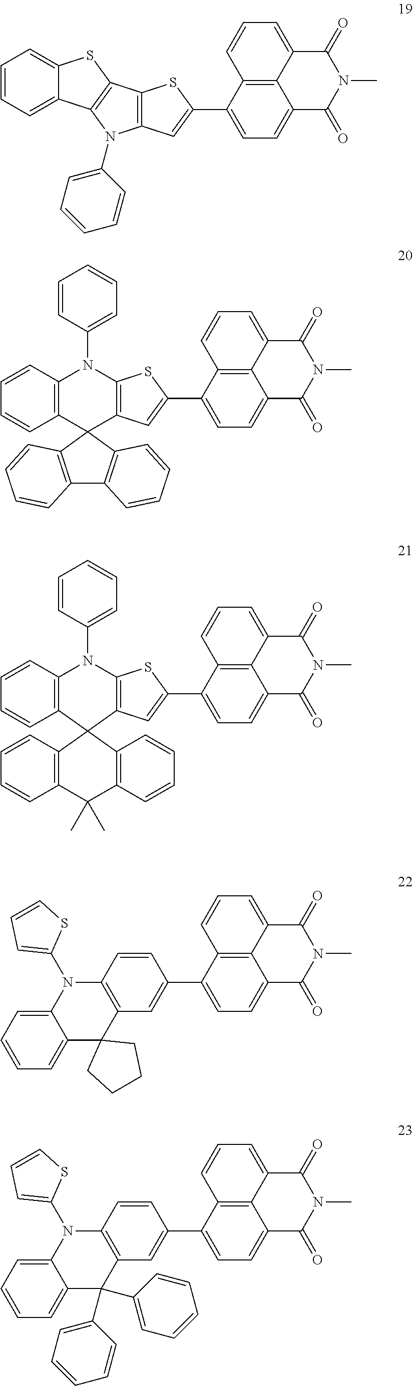

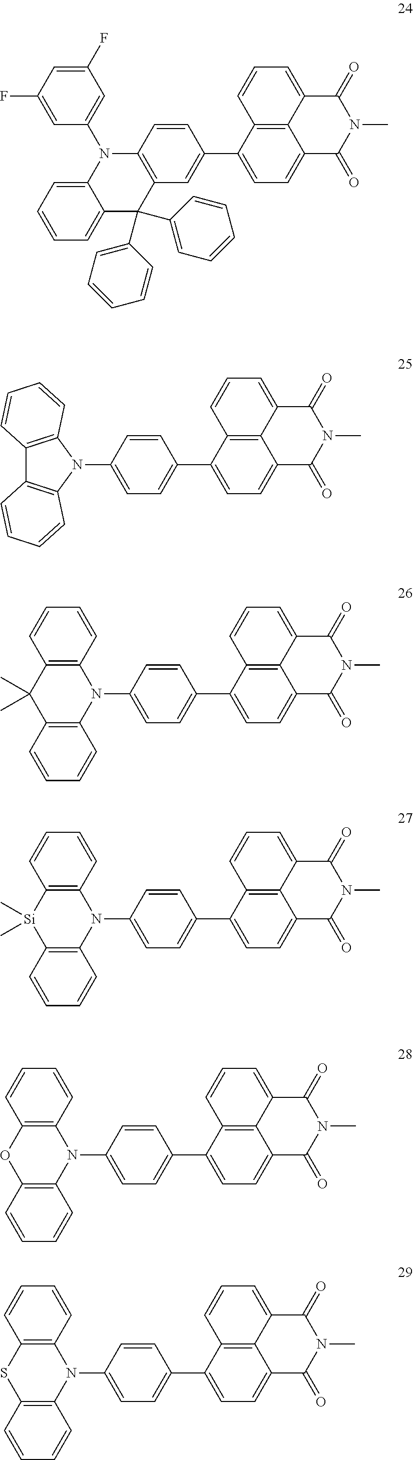

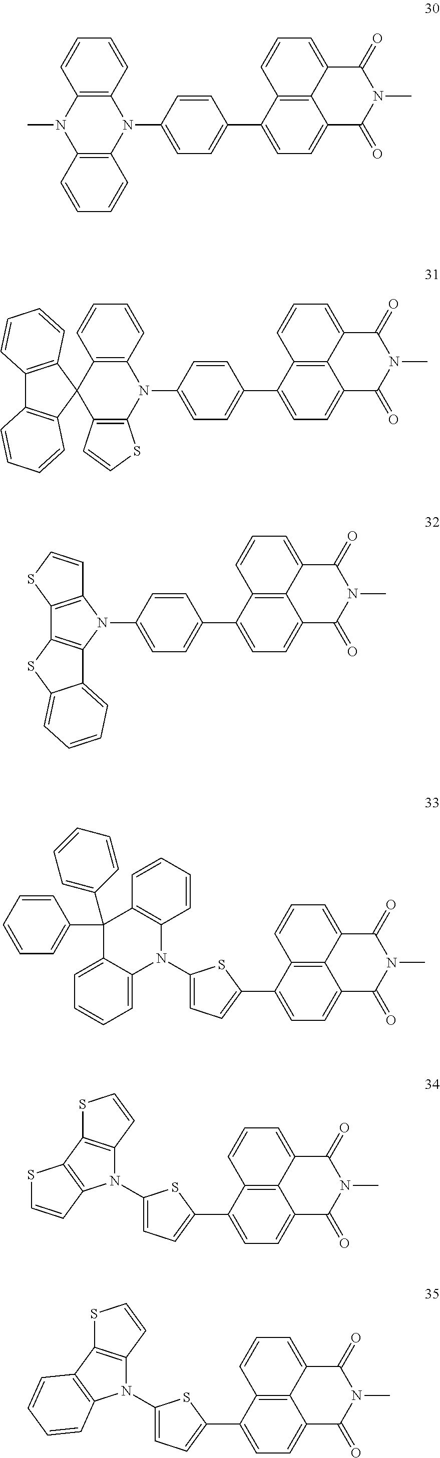

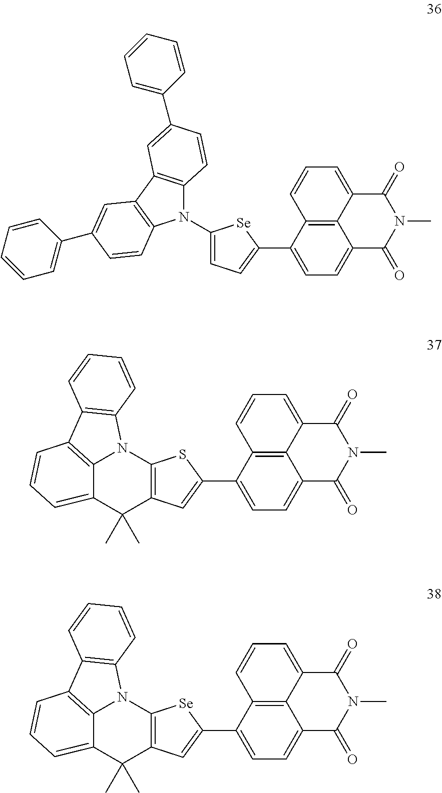 [SMILES: CN1C(=O)c2cccc3c(-c4ccc(-n5c6ccc(-c7ccccc7)cc6c6cc(-c7ccccc7)ccc65)[se]4)ccc(c23)C1=O ; CN1C(=O)c2cccc3c(-c4cc5c(s4)-n4c6ccccc6c6cccc(c64)C5(C)C)ccc(c23)C1=O ; CN1C(=O)c2cccc3c(-c4cc5c([se]4)-n4c6ccccc6c6cccc(c64)C5(C)C)ccc(c23)C1=O]

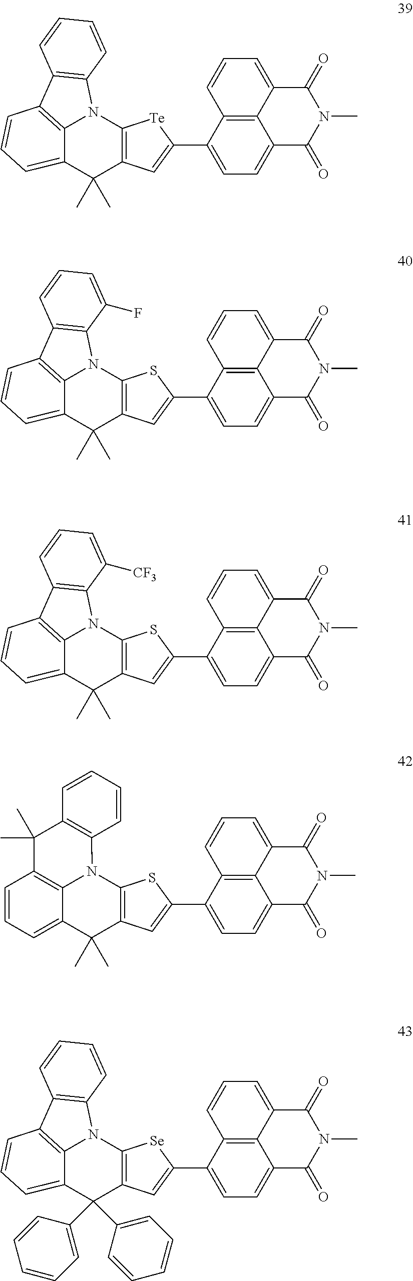

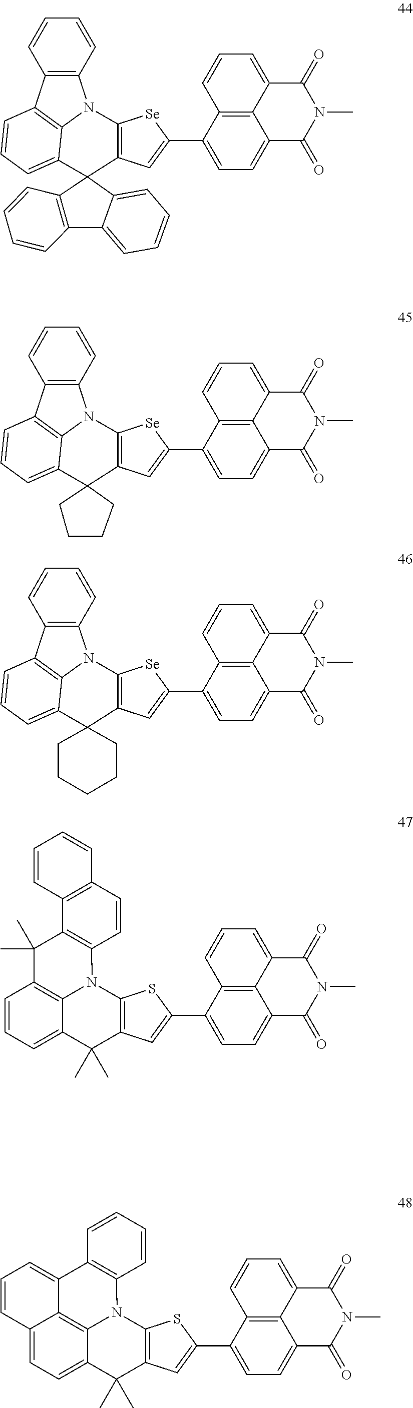

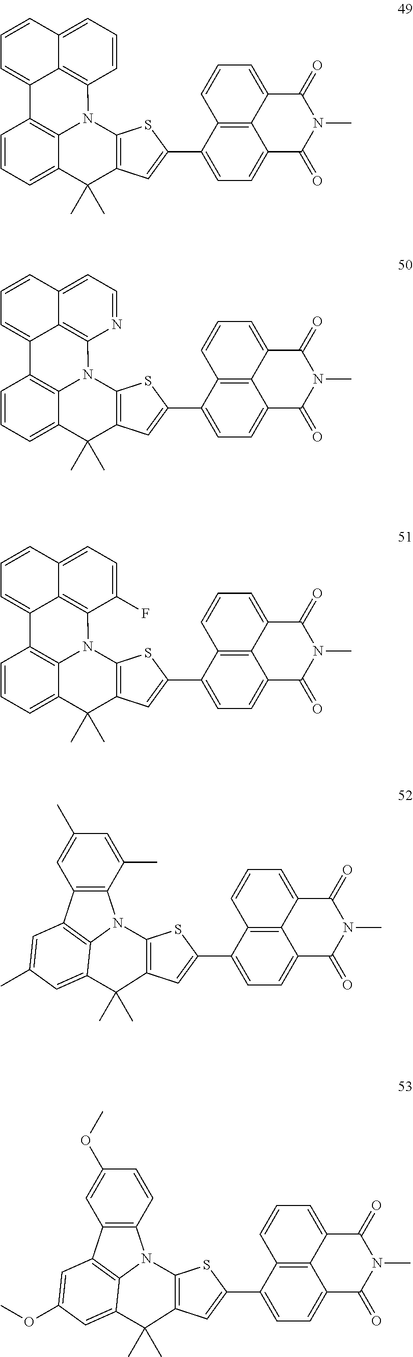

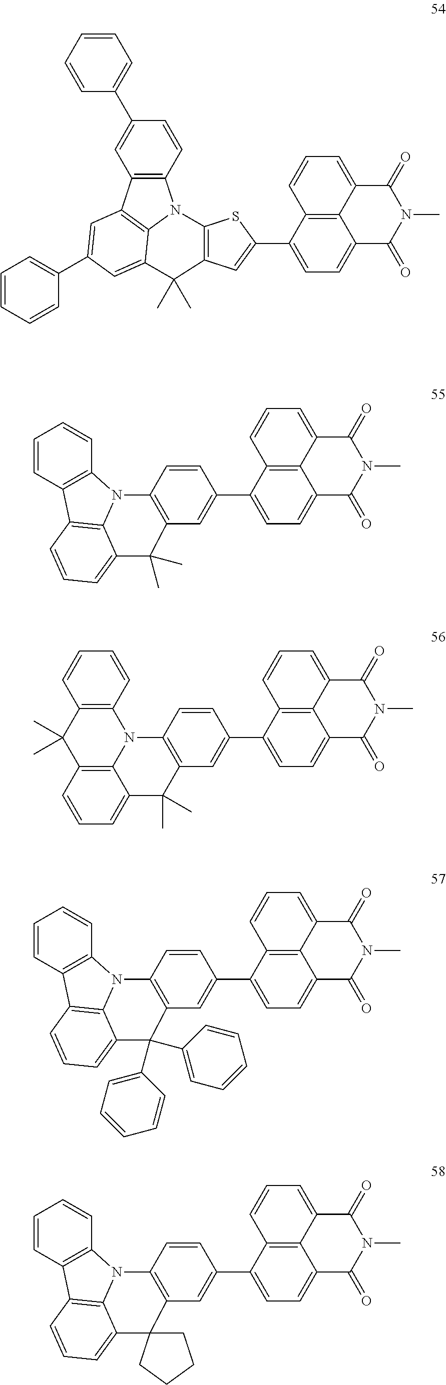

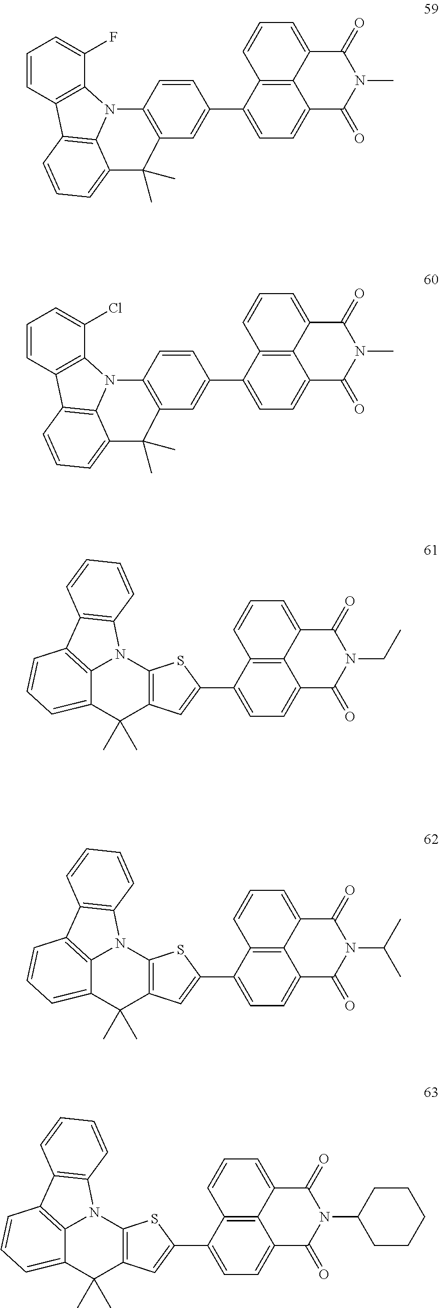 [SMILES: CN1C(=O)c2cccc3c(-c4ccc5c(c4)C(C)(C)c4cccc6c7cccc(F)c7n-5c46)ccc(c23)C1=O ; CN1C(=O)c2cccc3c(-c4ccc5c(c4)C(C)(C)c4cccc6c7cccc(Cl)c7n-5c46)ccc(c23)C1=O ; CCN1C(=O)c2cccc3c(-c4cc5c(s4)-n4c6ccccc6c6cccc(c64)C5(C)C)ccc(c23)C1=O ; CC(C)N1C(=O)c2cccc3c(-c4cc5c(s4)-n4c6ccccc6c6cccc(c64)C5(C)C)ccc(c23)C1=O ; CC1(C)c2cc(-c3ccc4c5c(cccc35)C(=O)N(C3CCCCC3)C4=O)sc2-n2c3ccccc3c3cccc1c32]

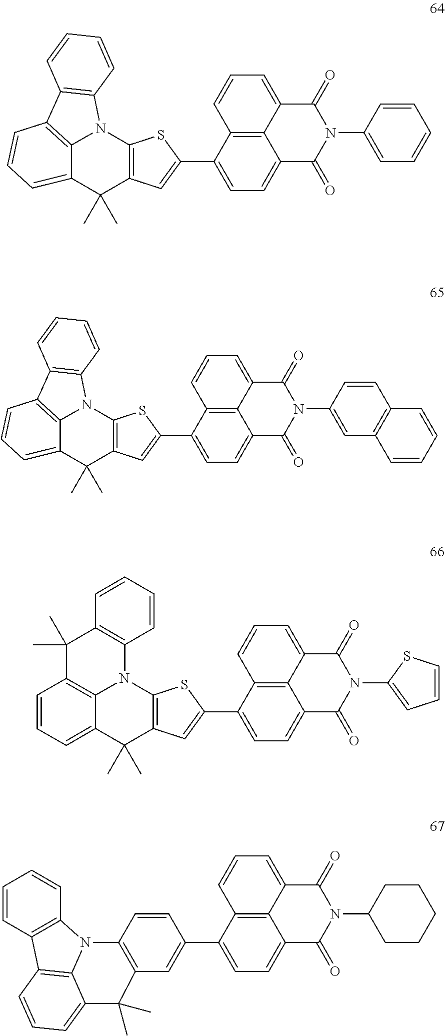 [SMILES: CC1(C)c2cc(-c3ccc4c5c(cccc35)C(=O)N(c3ccccc3)C4=O)sc2-n2c3ccccc3c3cccc1c32 ; CC1(C)c2cc(-c3ccc4c5c(cccc35)C(=O)N(c3ccc5ccccc5c3)C4=O)sc2-n2c3ccccc3c3cccc1c32 ; CC1(C)c2ccccc2N2c3sc(-c4ccc5c6c(cccc46)C(=O)N(c4cccs4)C5=O)cc3C(C)(C)c3cccc1c32 ; CC1(C)c2cc(-c3ccc4c5c(cccc35)C(=O)N(C3CCCCC3)C4=O)ccc2-n2c3ccccc3c3cccc1c32]

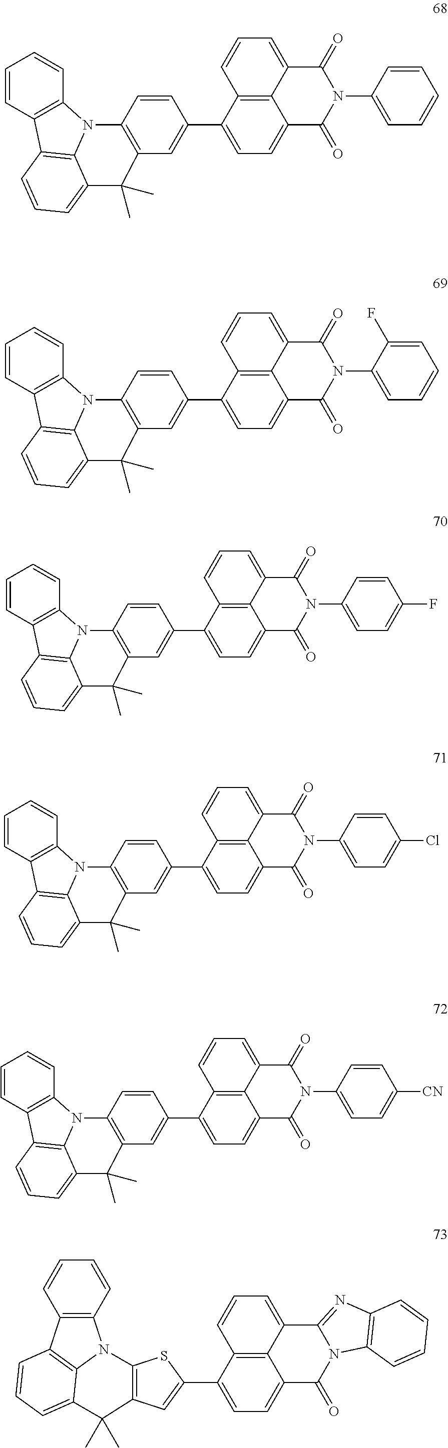 [SMILES: CC1(C)c2cc(-c3ccc4c5c(cccc35)C(=O)N(c3ccccc3)C4=O)ccc2-n2c3ccccc3c3cccc1c32 ; CC1(C)c2cc(-c3ccc4c5c(cccc35)C(=O)N(c3ccccc3F)C4=O)ccc2-n2c3ccccc3c3cccc1c32 ; CC1(C)c2cc(-c3ccc4c5c(cccc35)C(=O)N(c3ccc(F)cc3)C4=O)ccc2-n2c3ccccc3c3cccc1c32 ; CC1(C)c2cc(-c3ccc4c5c(cccc35)C(=O)N(c3ccc(Cl)cc3)C4=O)ccc2-n2c3ccccc3c3cccc1c32 ; CC1(C)c2cc(-c3ccc4c5c(cccc35)C(=O)N(c3ccc(C#N)cc3)C4=O)ccc2-n2c3ccccc3c3cccc1c32 ; CC1(C)c2cc(-c3ccc4c(=O)n5c6ccccc6nc5c5cccc3c45)sc2-n2c3ccccc3c3cccc1c32]

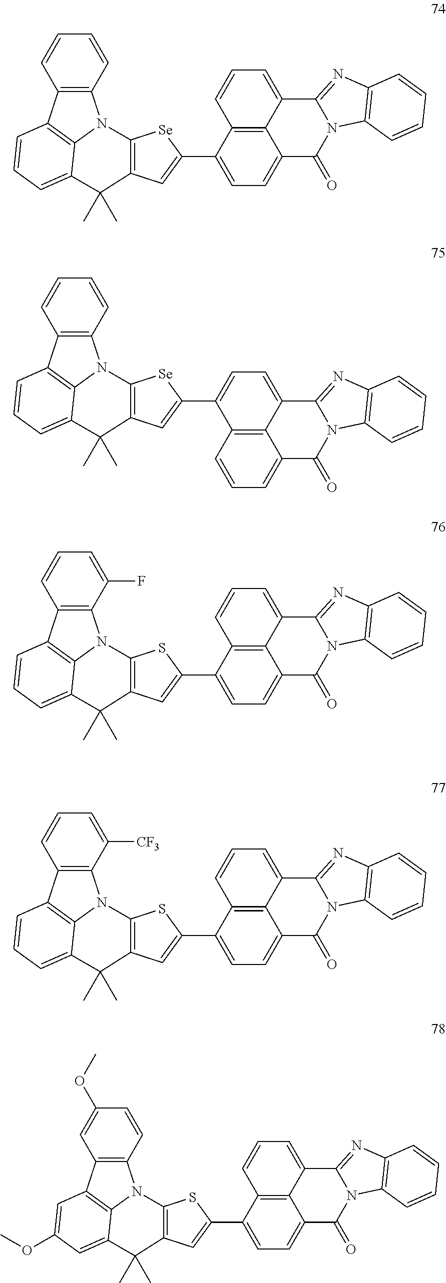 [SMILES: CC1(C)c2cc(-c3ccc4c(=O)n5c6ccccc6nc5c5cccc3c45)[se]c2-n2c3ccccc3c3cccc1c32 ; CC1(C)c2cc(-c3ccc4c5c3cccc5c(=O)n3c5ccccc5nc43)[se]c2-n2c3ccccc3c3cccc1c32 ; CC1(C)c2cc(-c3ccc4c(=O)n5c6ccccc6nc5c5cccc3c45)sc2-n2c3c(F)cccc3c3cccc1c32 ; CC1(C)c2cc(-c3ccc4c(=O)n5c6ccccc6nc5c5cccc3c45)sc2-n2c3c(C(F)(F)F)cccc3c3cccc1c32 ; COc1ccc2c(c1)c1cc(OC)cc3c1n2-c1sc(-c2ccc4c(=O)n5c6ccccc6nc5c5cccc2c45)cc1C3(C)C]

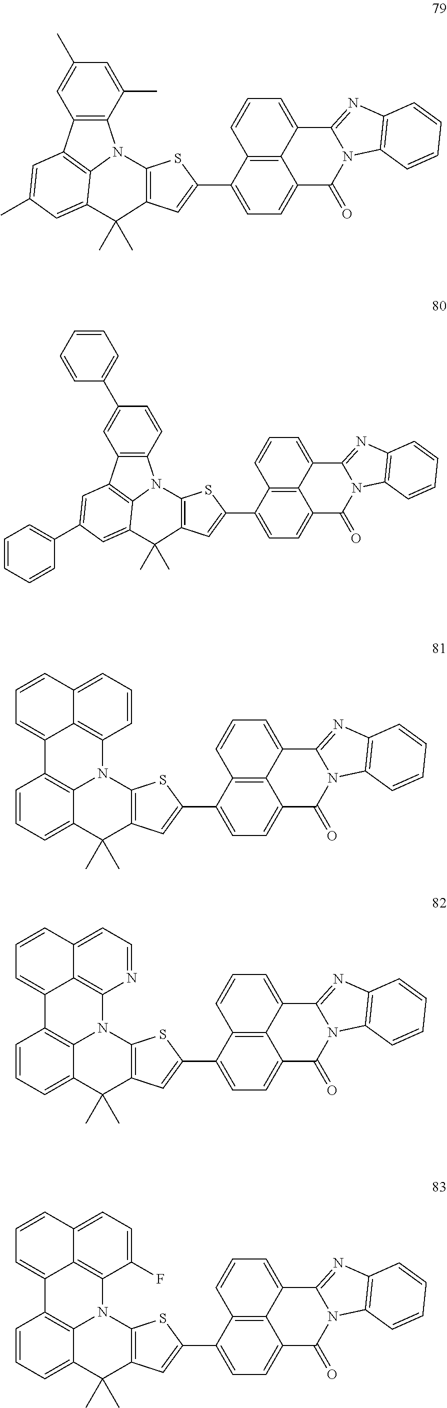 [SMILES: Cc1cc(C)c2c(c1)c1cc(C)cc3c1n2-c1sc(-c2ccc4c(=O)n5c6ccccc6nc5c5cccc2c45)cc1C3(C)C ; CC1(C)c2cc(-c3ccc4c(=O)n5c6ccccc6nc5c5cccc3c45)sc2-n2c3ccc(-c4ccccc4)cc3c3cc(-c4ccccc4)cc1c32 ; CC1(C)c2cc(-c3ccc4c(=O)n5c6ccccc6nc5c5cccc3c45)sc2N2c3c(cccc31)-c1cccc3cccc2c13 ; CC1(C)c2cc(-c3ccc4c(=O)n5c6ccccc6nc5c5cccc3c45)sc2N2c3c(cccc31)-c1cccc3ccnc2c13 ; CC1(C)c2cc(-c3ccc4c(=O)n5c6ccccc6nc5c5cccc3c45)sc2N2c3c(cccc31)-c1cccc3ccc(F)c2c13]

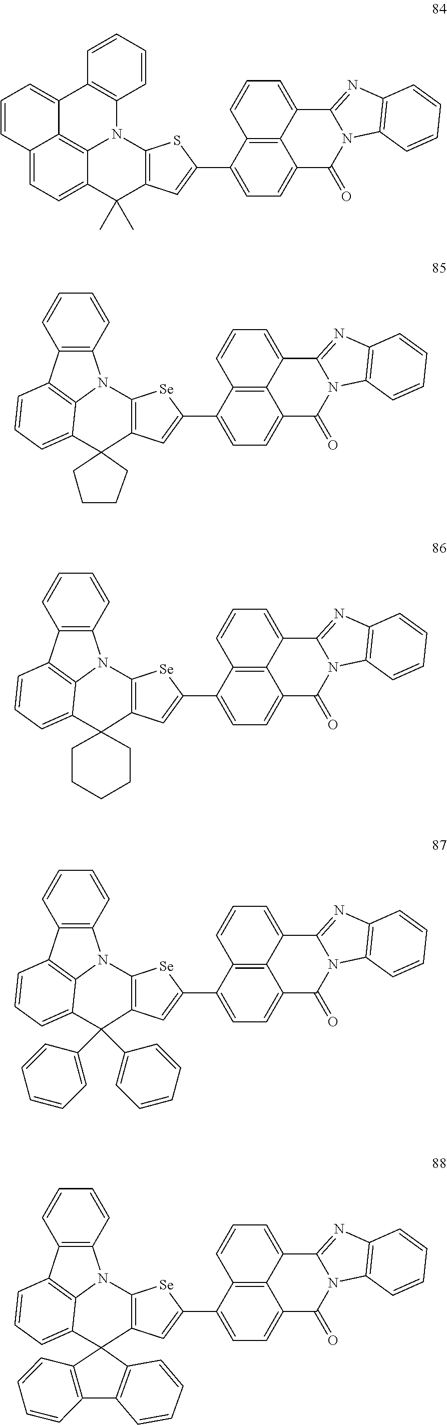 [SMILES: CC1(C)c2cc(-c3ccc4c(=O)n5c6ccccc6nc5c5cccc3c45)sc2N2c3ccccc3-c3cccc4ccc1c2c34 ; O=c1c2ccc(-c3cc4c([se]3)-n3c5ccccc5c5cccc(c53)C43CCCC3)c3cccc(c32)c2nc3ccccc3n12 ; O=c1c2ccc(-c3cc4c([se]3)-n3c5ccccc5c5cccc(c53)C43CCCCC3)c3cccc(c32)c2nc3ccccc3n12 ; O=c1c2ccc(-c3cc4c([se]3)-n3c5ccccc5c5cccc(c53)C4(c3ccccc3)c3ccccc3)c3cccc(c32)c2nc3ccccc3n12 ; O=c1c2ccc(-c3cc4c([se]3)-n3c5ccccc5c5cccc(c53)C43c4ccccc4-c4ccccc43)c3cccc(c32)c2nc3ccccc3n12]

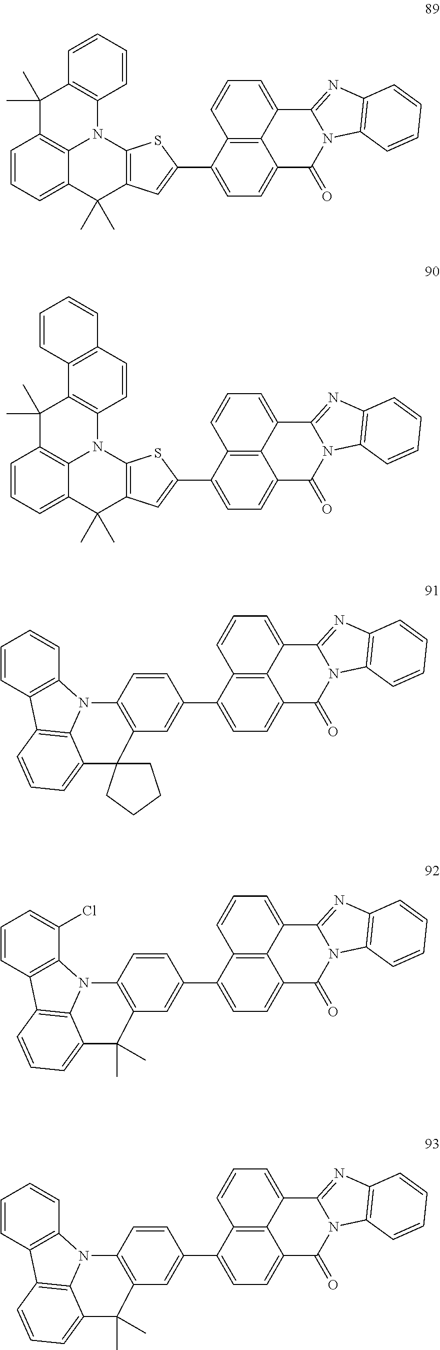 [SMILES: CC1(C)c2ccccc2N2c3sc(-c4ccc5c(=O)n6c7ccccc7nc6c6cccc4c56)cc3C(C)(C)c3cccc1c32 ; CC1(C)c2cc(-c3ccc4c(=O)n5c6ccccc6nc5c5cccc3c45)sc2N2c3ccc4ccccc4c3C(C)(C)c3cccc1c32 ; O=c1c2ccc(-c3ccc4c(c3)C3(CCCC3)c3cccc5c6ccccc6n-4c35)c3cccc(c32)c2nc3ccccc3n12 ; CC1(C)c2cc(-c3ccc4c(=O)n5c6ccccc6nc5c5cccc3c45)ccc2-n2c3c(Cl)cccc3c3cccc1c32 ; CC1(C)c2cc(-c3ccc4c(=O)n5c6ccccc6nc5c5cccc3c45)ccc2-n2c3ccccc3c3cccc1c32]

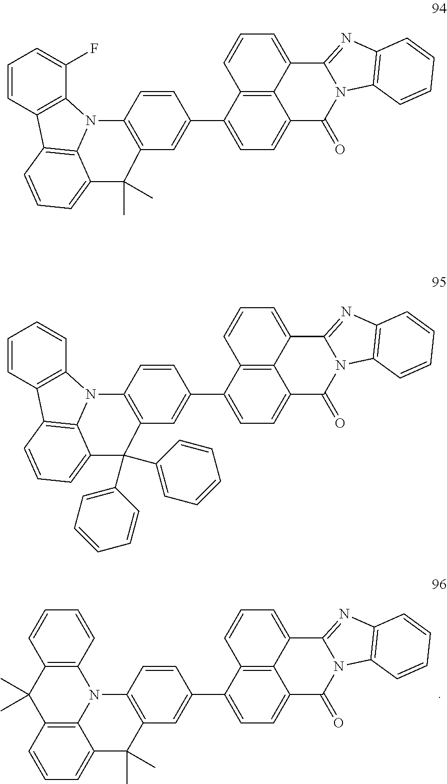 [SMILES: CC1(C)c2cc(-c3ccc4c(=O)n5c6ccccc6nc5c5cccc3c45)ccc2-n2c3c(F)cccc3c3cccc1c32 ; O=c1c2ccc(-c3ccc4c(c3)C(c3ccccc3)(c3ccccc3)c3cccc5c6ccccc6n-4c35)c3cccc(c32)c2nc3ccccc3n12 ; CC1(C)c2ccccc2N2c3ccc(-c4ccc5c(=O)n6c7ccccc7nc6c6cccc4c56)cc3C(C)(C)c3cccc1c32]

Because the organic compound has a molecular weight of about 1,000 or less, a deposition temperature for forming the organic compound may be relatively low. Thus, the organic compound may have excellent or suitable heat resistance characteristics.

The organic compound represented by Formula 1 may include only one Ar₁₁ (e.g., only one group) represented by Formula 2. Ar₁₁ represented by Formula 2 may not be directly bonded to N of Formula 1, and may be bonded to L₁ or Ar₂. In some embodiments, at least one of b2 to b4 in Formula 1 may be 1 or 2. In one embodiment, each of b2 to b4 in Formula 1 may not be 0. As a result, a twist angle within a molecule may be reduced so that relatively more (e.g., many) molecules may be included in a unit thickness, and thus, an opto-electronic device including the organic compound may have higher (e.g., high) light absorption efficiency for green wavelength light.

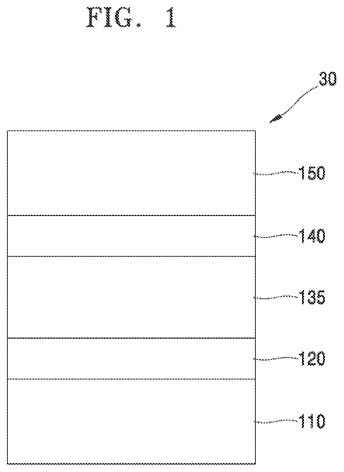

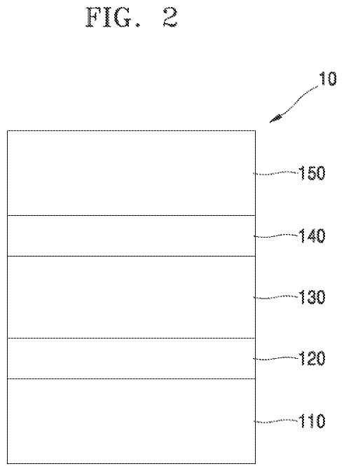

### Description of FIGS. 1 and 2

FIG. 1 is a schematic view of an opto-electronic device 30 according to an embodiment. The opto-electronic device 30 may include a first electrode 110, a hole transport region 120, a photoactive layer 135, an electron transport region 140, and a second electrode 150.

FIG. 2 is a schematic view of a light-emitting device 10. The light-emitting device 10 may include a first electrode 110, a hole transport region 120, an emission layer 130, an electron transport region 140, and a second electrode 150.

Hereinafter, the structures of the opto-electronic device 30 and the light-emitting device 10 according to embodiments and methods of manufacturing the opto-electronic device 30 and the light-emitting device 10 will be described in more detail with reference to FIGS. 1 and 2.

### First Electrode 110

In FIG. 1, a substrate may be additionally arranged under the first electrode 110 and/or on the second electrode 150. As the substrate, a glass substrate and/or a plastic substrate may be utilized. In an embodiment, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In an embodiment, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

### Hole Transport Region 120

The hole transport region 120 may have i) a single-layered structure consisting of a single layer consisting of a single material, ii) a single-layered structure consisting of a single layer including (e.g., consisting of) a plurality of different materials, or iii) a multi-layered structure including a plurality of layers including different materials.

The hole transport region 120 may include a hole injection layer, a hole transport layer, an emission auxiliary layer, an electron blocking layer, or any combination thereof.

For example, the hole transport region 120 may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein constituting layers of each structure are stacked sequentially from the first electrode 110 in the respective stated order.

The hole transport region 120 may include a compound represented by Formula 201, a compound represented by Formula 202, or any combination thereof:

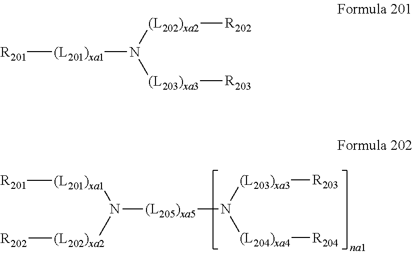

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group, etc.) unsubstituted or substituted with at least one R₁₀ₐ (e.g., Compound HT16, etc.),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and
na1 may be an integer from 1 to 4.

For example, each of Formulae 201 and 202 may include at least one of the groups represented by Formulae CY201 to CY217:

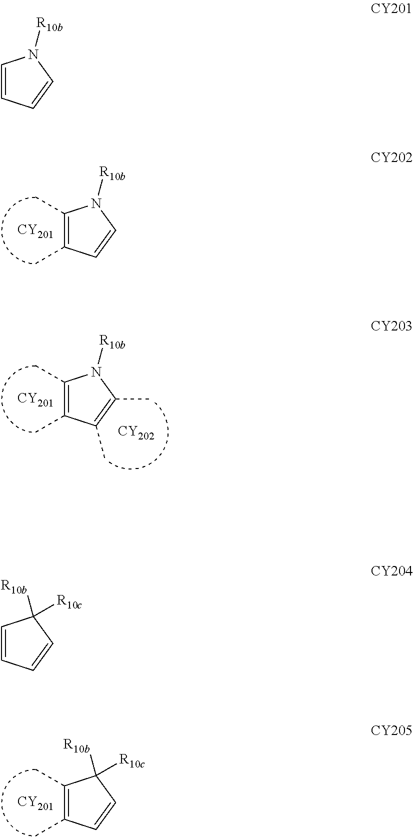

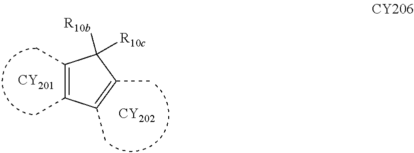

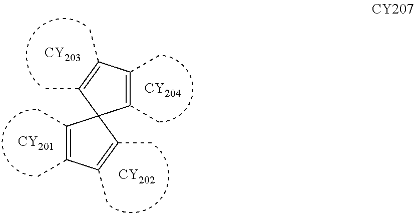

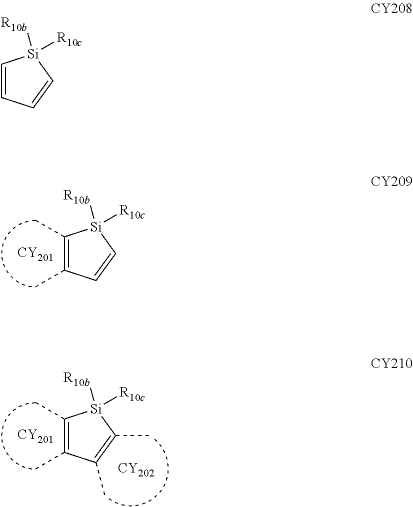

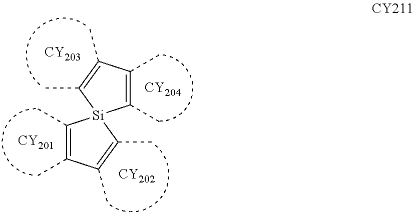

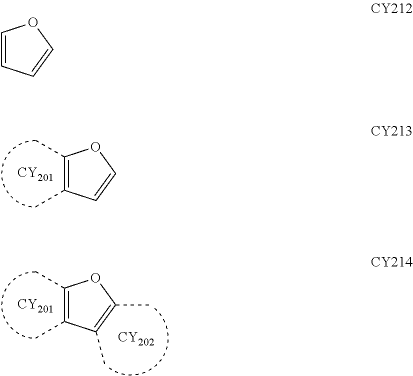

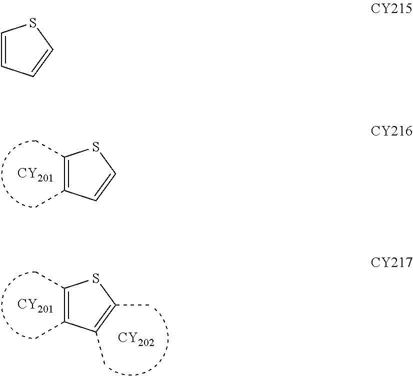

wherein, in Formulae CY201 to CY217, R_{10b} and R_{10c} may each independently be the same as defined herein in connection with R₁₀ₐ, ring CY201 to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In an embodiment, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one of the groups represented by Formulae CY201 to CY203.

In one or more embodiments, Formula 201 may include at least one of groups represented by Formulae CY201 to CY203 and at least one of the groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include (e.g., may exclude) any of the groups represented by Formulae CY201 to CY203.

In one or more embodiments, each of Formulae 201 and 202 may not include (e.g., may exclude) any of the groups represented by Formulae CY201 to CY203, and may include at least one of the groups represented by Formulae CY204 to CY217.

In one or more embodiments, each of Formulae 201 and 202 may not include (e.g., may exclude) any of the groups represented by Formulae CY201 to CY217.

For example, the hole transport region 120 may include at least one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

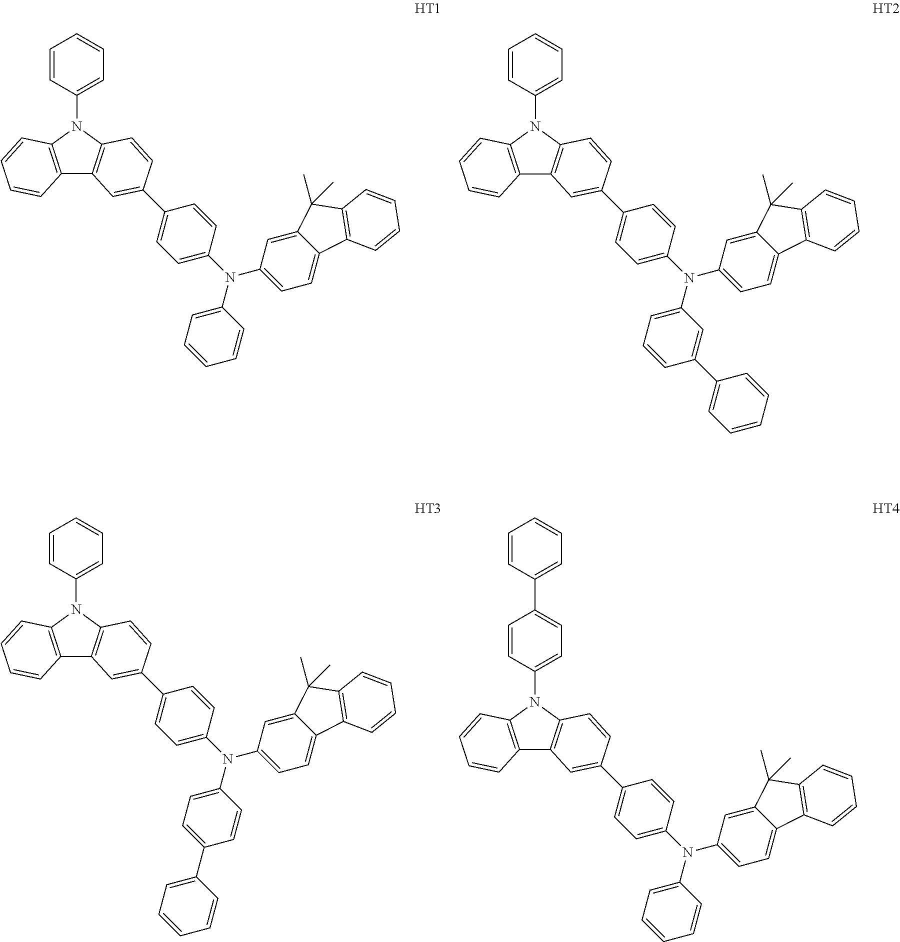 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

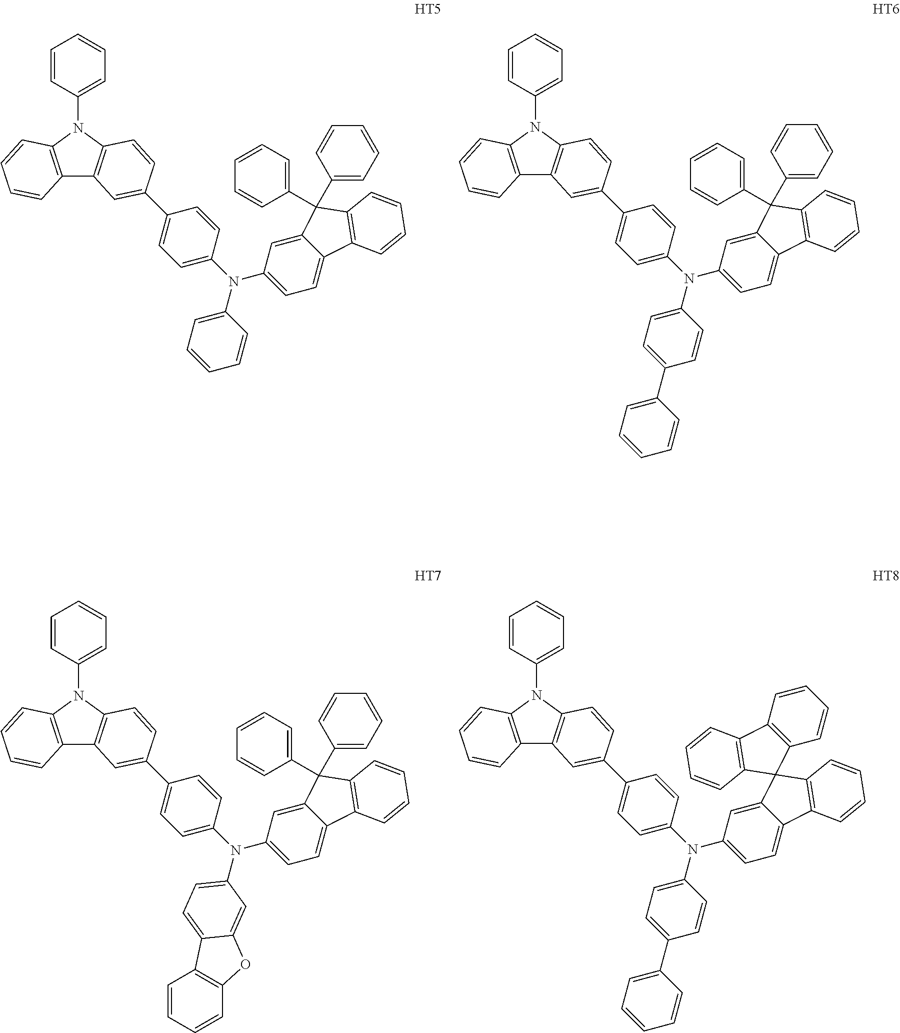 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

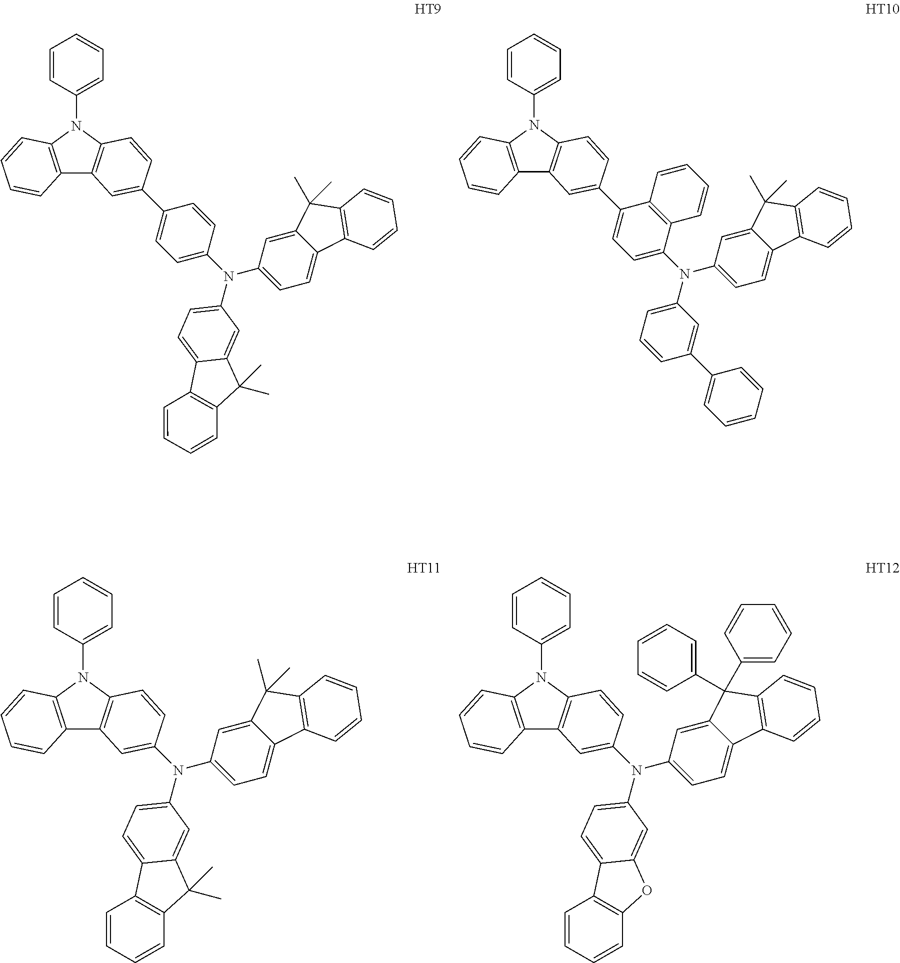 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

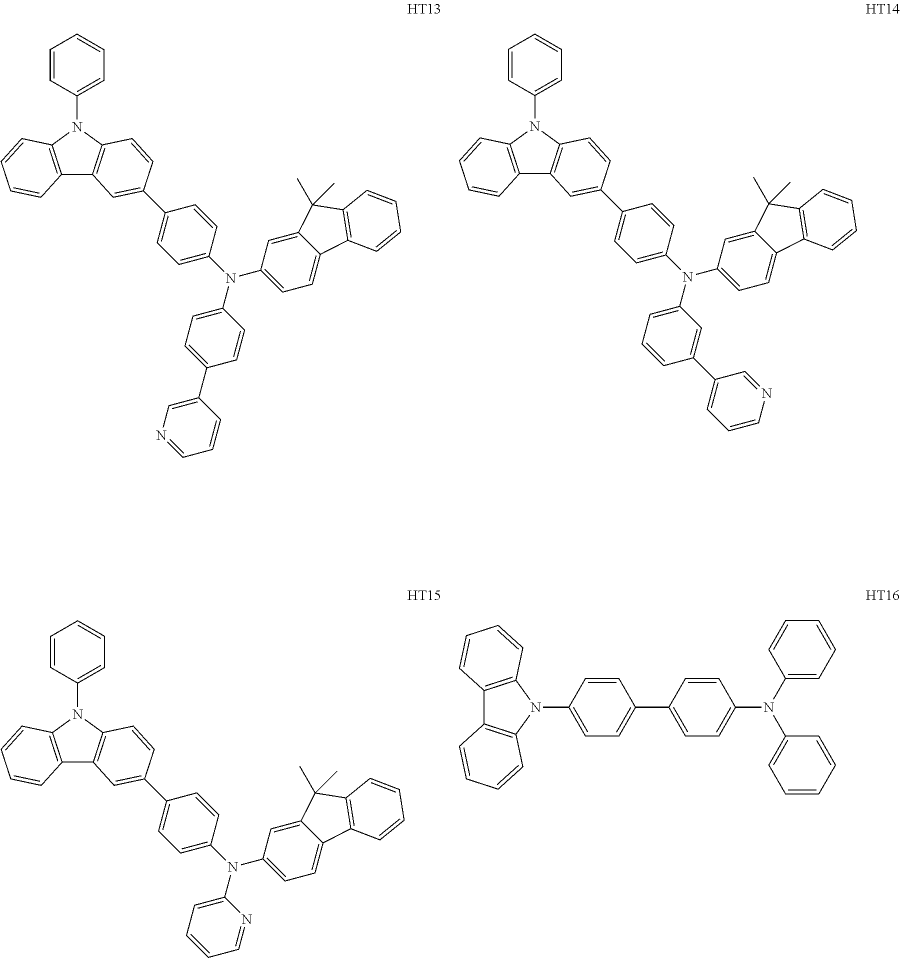 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

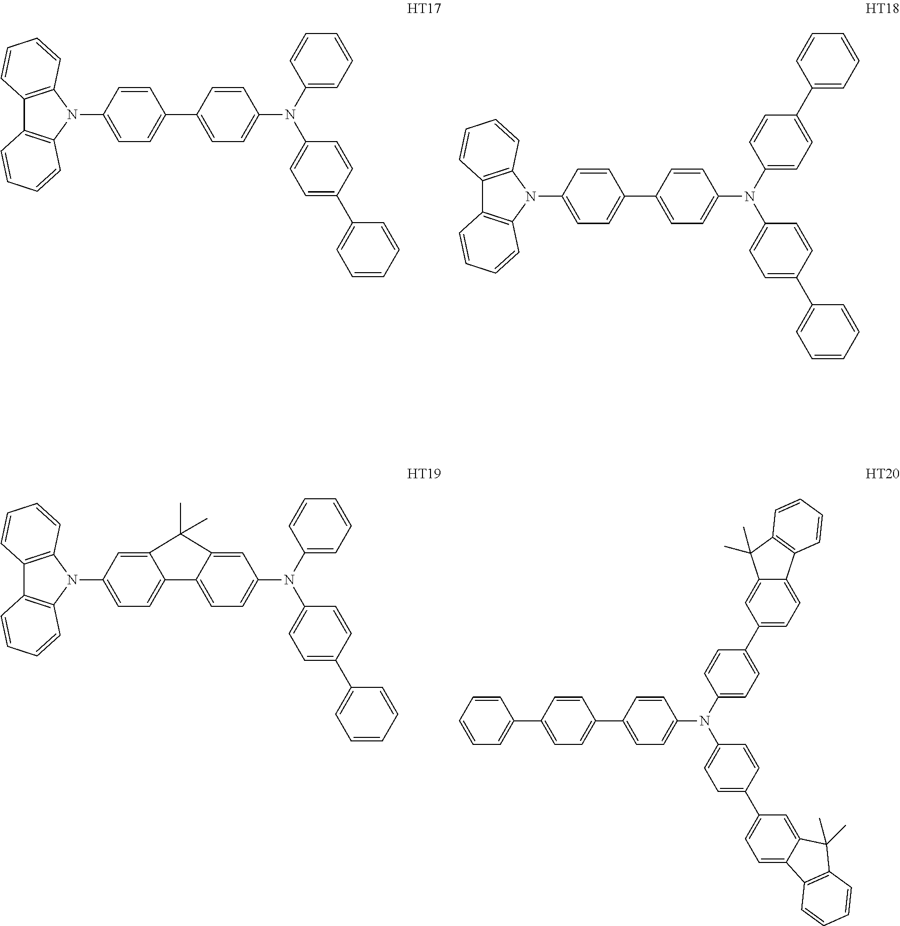 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

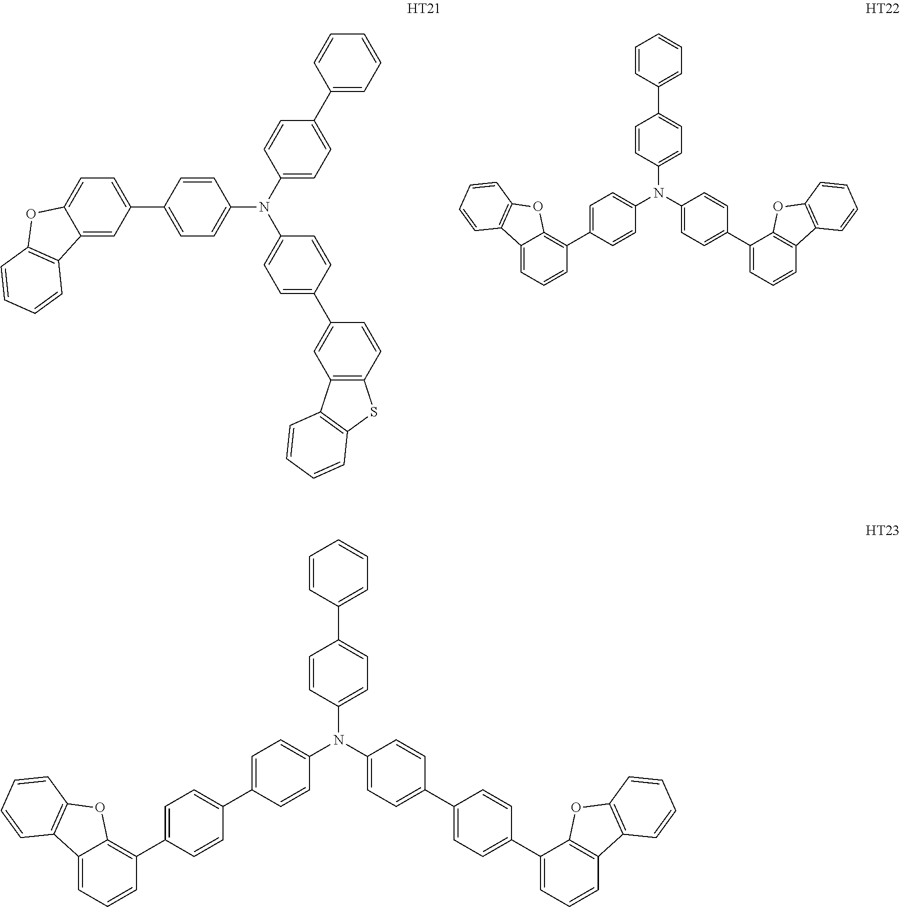 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

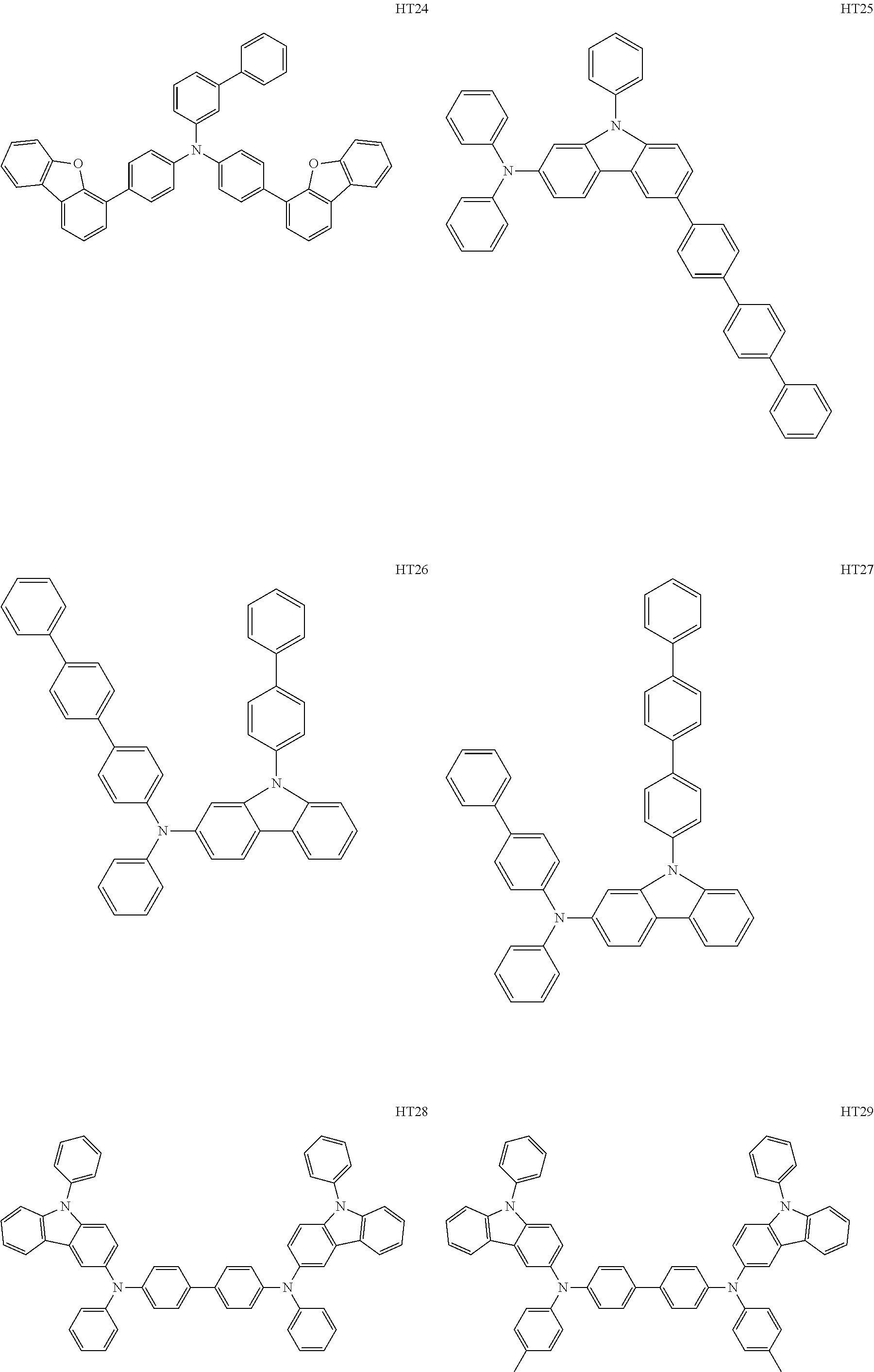

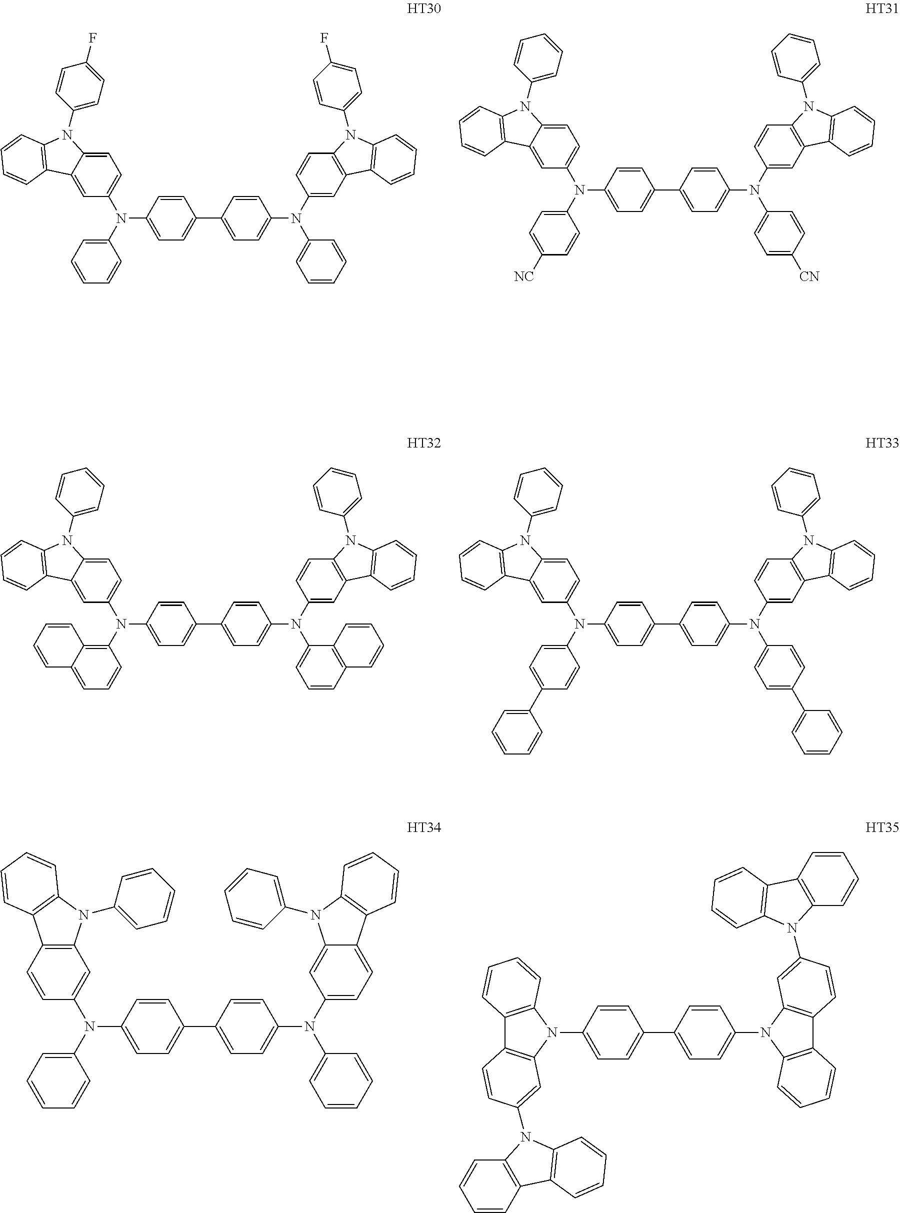

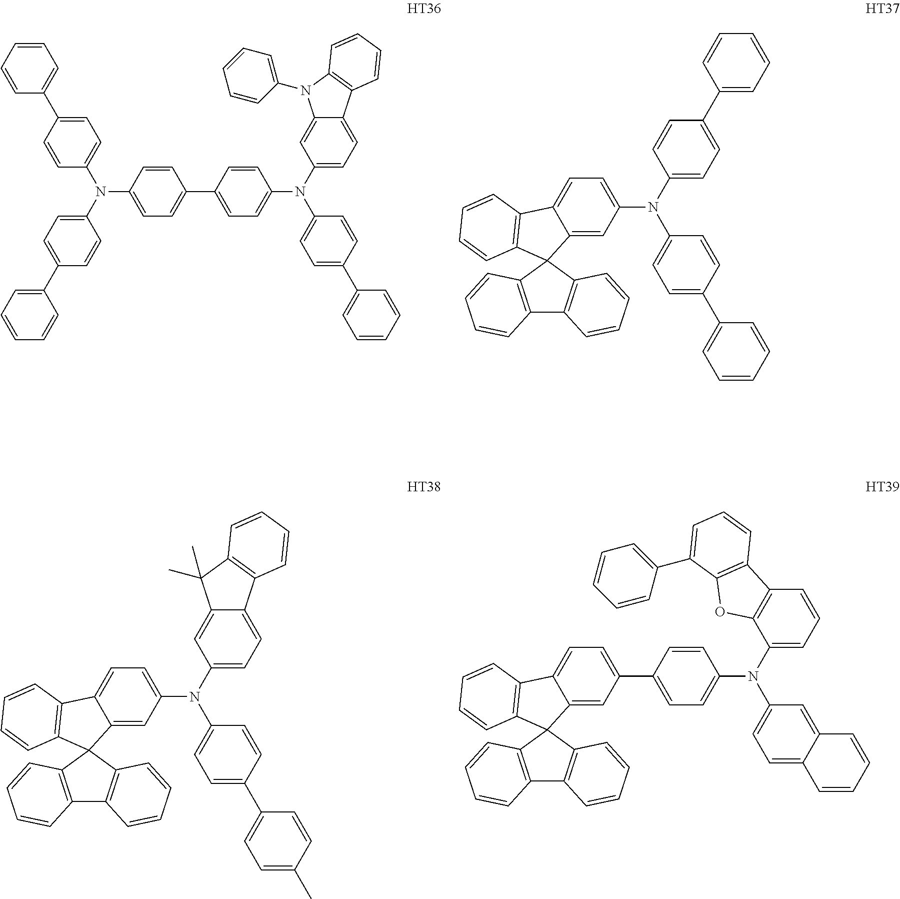 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

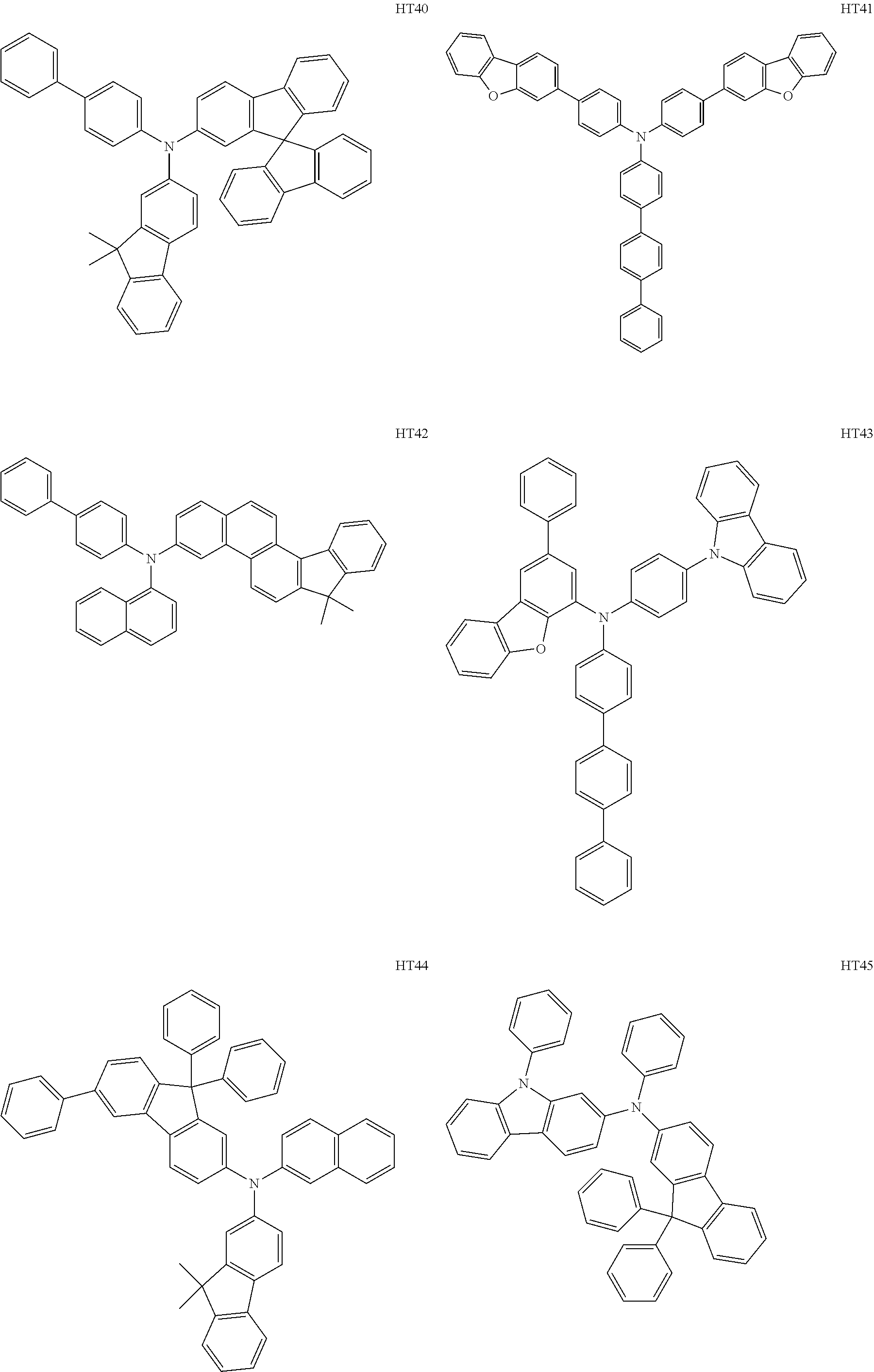

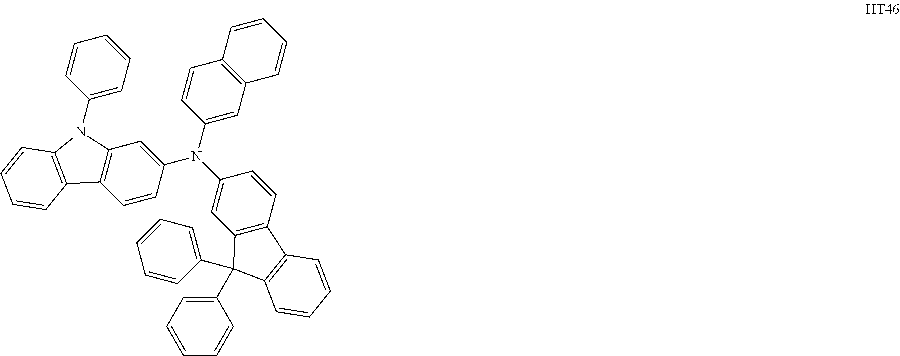 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

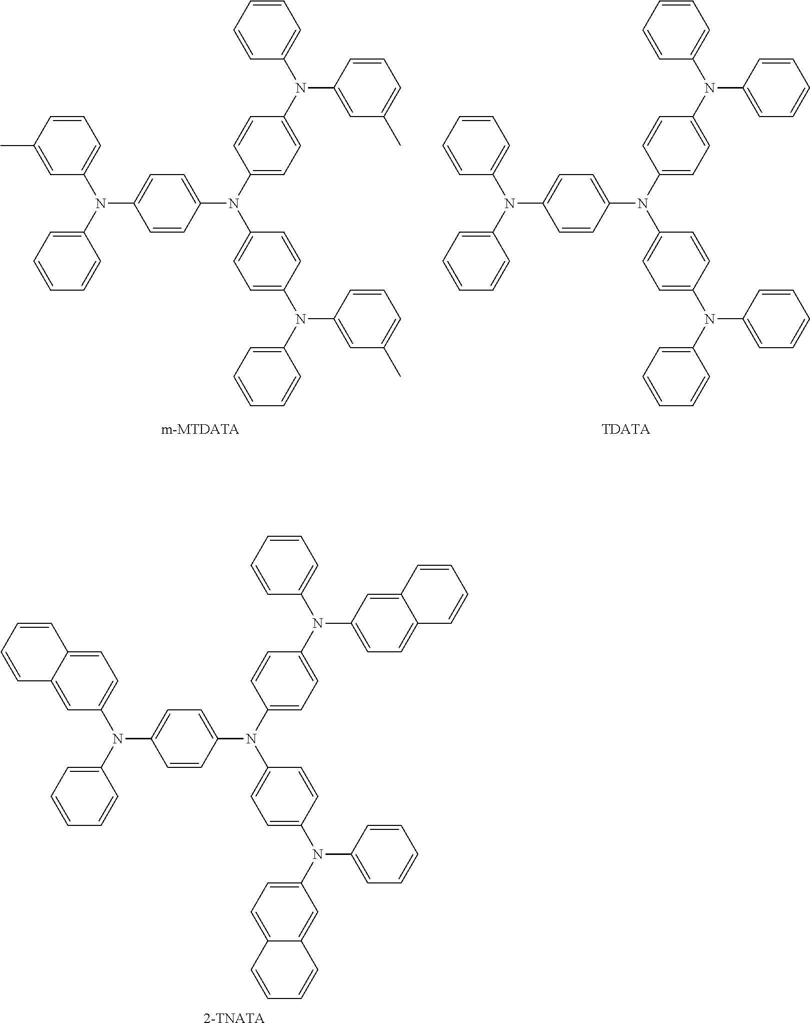

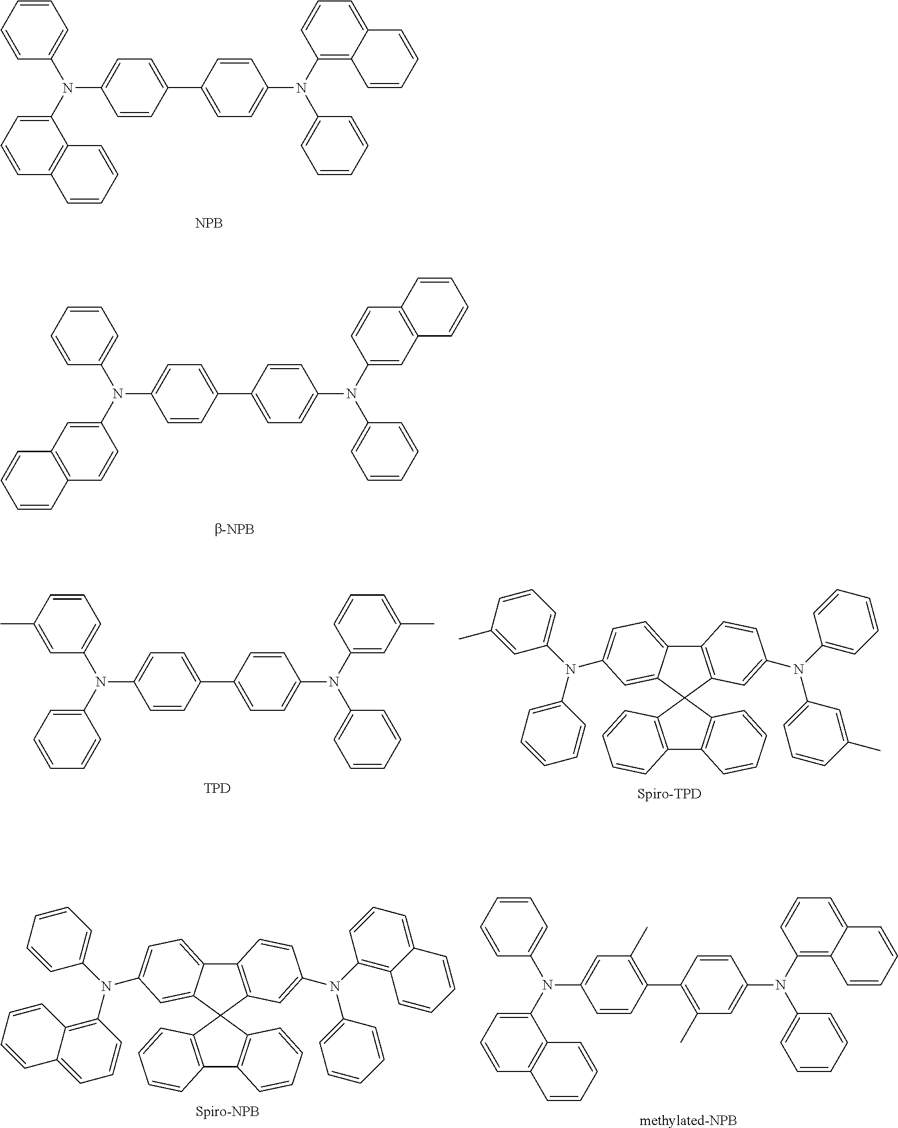

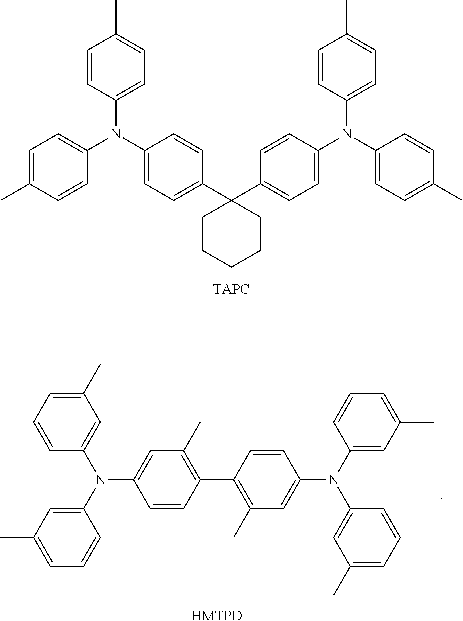

A thickness of the hole transport region 120 may be in a range of about 50 Å to about 10,000 Å, for example, about 100 Å to about 4,000 Å. When the hole transport region 120 includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region 120, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by the emission layer 130, and the electron blocking layer may block or reduce the flow (e.g., leakage) of electrons from the emission layer 130 to the hole transport region 120. Materials that may be included in the hole transport region 120 may be included in the emission auxiliary layer and the electron blocking layer.

p-Dopant

The hole transport region 120 may further include, in addition to the materials described above, a charge-generation material for improving conductive properties. The charge-generation material may be uniformly or non-uniformly dispersed in the hole transport region 120 (e.g., in the form of a single layer consisting of a charge-generation material).

The charge-generation material may be, for example, a p-dopant.

For example, a LUMO energy level of the p-dopant may be −3.5 eV or less.

In an embodiment, the p-dopant may include a quinone derivative, a cyano group-containing compound, a compound containing element EL1 and element EL2, or any combination thereof.

Examples of the quinone derivative may include TCNQ, F4-TCNQ, and/or the like.

Examples of the cyano group-containing compound may include HAT-CN, a compound represented by Formula 221, and/or the like:

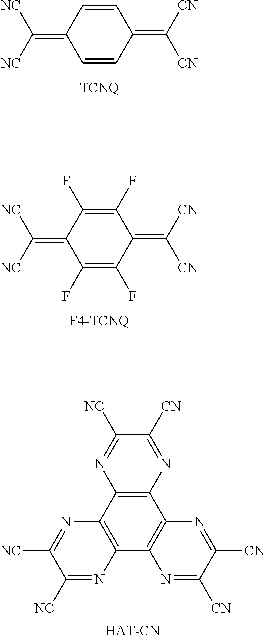

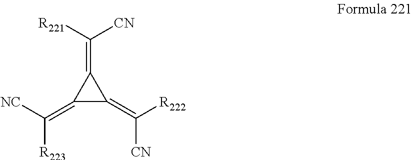

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or any combination thereof, and element EL2 may be a non-metal, a metalloid, or any combination thereof.

Examples of the metal may include: an alkali metal (e.g., lithium (Li), sodium (Na), potassium (K), rubidium (Rb), cesium (Cs), etc.); an alkaline earth metal (e.g., beryllium (Be), magnesium (Mg), calcium (Ca), strontium (Sr), barium (Ba), etc.); a transition metal (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (e.g., zinc (Zn), indium (In), tin (Sn), etc.); a lanthanide metal (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and/or the like.

Examples of the metalloid may include silicon (Si), antimony (Sb), tellurium (Te), and/or the like.

Examples of the non-metal may include oxygen (O), halogen (e.g., F, Cl, Br, I, etc.), and/or the like.

Examples of the compound containing element EL1 and element EL2 may include a metal oxide, a metal halide (e.g., a metal fluoride, a metal chloride, a metal bromide, a metal iodide, etc.), a metalloid halide (e.g., a metalloid fluoride, a metalloid chloride, a metalloid bromide, a metalloid iodide, etc.), a metal telluride, or any combination thereof.

Examples of the metal oxide may include a tungsten oxide (e.g., WO, W₂O₃, WO₂, WO₃, W₂O₅, etc.), a vanadium oxide (e.g., VO, V₂O₃, VO₂, V₂O₅, etc.), a molybdenum oxide (e.g., MoO, Mo₂O₃, MoO₂, MoO₃, Mo₂O₅, etc.), a rhenium oxide (e.g., ReO₃, etc.), and/or the like.

Examples of the metal halide may include an alkali metal halide, an alkaline earth metal halide, a transition metal halide, a post-transition metal halide, a lanthanide metal halide, and/or the like.

Examples of the alkali metal halide may include LiF, NaF, KF, RbF, CsF, LiCl, NaCl, KCl, RbCl, CsCl, LiBr, NaBr, KBr, RbBr, CsBr, Lil, NaI, KI, RbI, CsI, and/or the like.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, Bel₂, Mg₁₂, Cal₂, Srl₂, Bal₂, and/or the like.

Examples of the transition metal halide may include a titanium halide (e.g., TiF₄, TiCl₄, TiBr₄, TiI₄, etc.), a zirconium halide (e.g., ZrF₄, ZrCl₄, ZrBr₄, ZrI₄, etc.), a hafnium halide (e.g., HfF₄, HfCl₄, HfBr₄, HfI₄, etc.), a vanadium halide (e.g., VF₃, VCl₃, VBr₃, VI₃, etc.), a niobium halide (e.g., NbF₃, NbCl₃, NbBr₃, NbI₃, etc.), a tantalum halide (e.g., TaF₃, TaCl₃, TaBr₃, TaI₃, etc.), a chromium halide (e.g., CrF₃, CrO₃, CrBr₃, CrI₃, etc.), a molybdenum halide (e.g., MoF₃, MoCl₃, MoBr₃, MoI₃, etc.), a tungsten halide (e.g., WF₃, WCl₃, WBr₃, WI₃, etc.), a manganese halide (e.g., MnF₂, MnCl₂, MnBr₂, MnI₂, etc.), a technetium halide (e.g., TcF₂, TcCl₂, TcBr₂, TcI₂, etc.), a rhenium halide (e.g., ReF₂, ReCl₂, ReBr₂, ReI₂, etc.), an iron halide (e.g., FeF₂, FeCl₂, FeBr₂, FeI₂, etc.), a ruthenium halide (e.g., RuF₂, RuCl₂, RuBr₂, RuI₂, etc.), an osmium halide (e.g., OsF₂, OsCl₂, OsBr₂, OsI₂, etc.), a cobalt halide (e.g., CoF₂, COCl₂, CoBr₂, CoI₂, etc.), a rhodium halide (e.g., RhF₂, RhCl₂, RhBr₂, Rhl₂, etc.), an iridium halide (e.g., IrF₂, IrCl₂, IrBr₂, IrI₂, etc.), a nickel halide (e.g., NiF₂, NiCl₂, NiBr₂, NiI₂, etc.), a palladium halide (e.g., PdF₂, PdCl₂, PdBr₂, PdI₂, etc.), a platinum halide (e.g., PtF₂, PtCl₂, PtBr₂, PtI₂, etc.), a copper halide (e.g., CuF, CuCl, CuBr, CuI, etc.), a silver halide (e.g., AgF, AgCl, AgBr, AgI, etc.), a gold halide (e.g., AuF, AuCl, AuBr, AuI, etc.), and/or the like.

Examples of the post-transition metal halide may include a zinc halide (e.g., ZnF₂, ZnCl₂, ZnBr₂, ZnI₂, etc.), an indium halide (e.g., Ink₃, etc.), a tin halide (e.g., SnI₂, etc.), and/or the like.

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, Sm₁₃, and/or the like.

Examples of the metalloid halide may include antimony halide (e.g., SbCl₅, etc.) and/or the like.

Examples of the metal telluride may include an alkali metal telluride (e.g., Li₂Te, Na₂Te, K₂Te, Rb₂Te, Cs₂Te, etc.), an alkaline earth metal telluride (e.g., BeTe, MgTe, CaTe, SrTe, BaTe, etc.), a transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (e.g., ZnTe, etc.), a lanthanide metal telluride (e.g., LaTe, CeTe, PrTe, NdTe, PmTe, EuTe, GdTe, TbTe, DyTe, HoTe, ErTe, TmTe, YbTe, LuTe, etc.), and/or the like.

### Emission Layer 130

The light-emitting device 10 may include the emission layer 130 arranged on the hole transport region 120.

The emission layer 130 may further include, in addition to one or more suitable organic materials, a metal-containing compound, such as an organometallic compound, an inorganic material, such as a quantum dot, and/or the like.

In an embodiment, the emission layer 130 may include i) two or more emitting units sequentially stacked between the first electrode 110 and the second electrode 150, and ii) a charge generation layer arranged between the two or more emitting units. When the emission layer 130 includes the two or more emitting units and the charge generation layer as described above, the light-emitting device 10 may be a tandem light-emitting device.

When the light-emitting device 10 is a full-color light-emitting device, the emission layer 130 may be patterned into a red emission layer, a green emission layer, and/or a blue emission layer, according to a subpixel. In an embodiment, the emission layer 130 may have a stacked structure of two or more layers selected from a red emission layer, a green emission layer, and a blue emission layer, in which the two or more layers contact each other or are separated from each other to emit white light. In one or more embodiments, the emission layer 130 may include two or more materials selected from a red light-emitting material, a green light-emitting material, and a blue light-emitting material, in which the two or more materials are mixed with each other in a single layer to emit white light.

The emission layer 130 may include a host and a dopant. The dopant may include a phosphorescent dopant, a fluorescent dopant, or any combination thereof.

An amount of the dopant in the emission layer 130 may be in a range of about 0.01 part by weight to about 15 parts by weight based on 100 parts by weight of the host.

In an embodiment, the emission layer 130 may include a quantum dot.

In an embodiment, the emission layer 130 may include a delayed fluorescence material. The delayed fluorescence material may serve as a host or a dopant in the emission layer 130.

A thickness of the emission layer 130 may be in a range of about 100 Å to about 1,000 Å, for example, about 200 Å to about 600 Å. When the thickness of the emission layer 130 is within these ranges, excellent or suitable luminescence characteristics may be obtained without a substantial increase in driving voltage.

### Host

The host may include a compound represented by Formula 301:

[Ar₃₀₁]_{xb11}-[(L₃₀₁)_{xb1}-R₃₀₁]_{xb21} Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each independently be the same as defined herein in connection with Q₁.

For example, when xb11 in Formula 301 is 2 or more, two or more of Ar₃₀₁(s) may be linked to each other via a single bond.

In an embodiment, the host may include a compound represented by Formula 301-1, a compound represented by Formula 301-2, or any combination thereof:

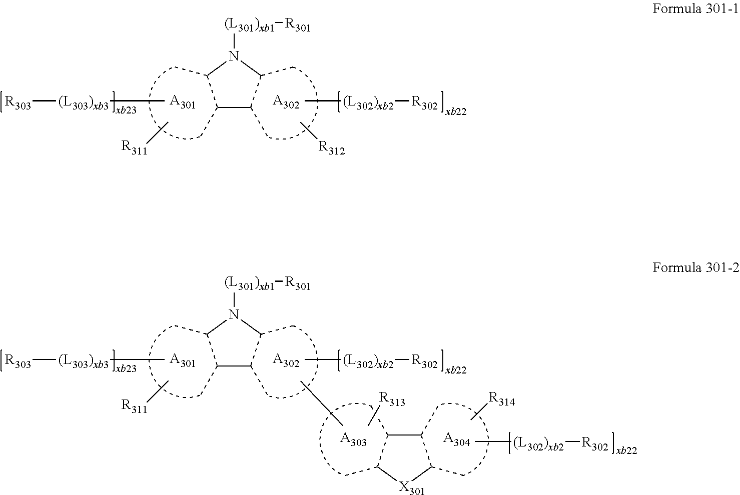

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as defined herein,
L₃₀₂ to L₃₀₄ may each independently be the same as defined herein in connection with L₃₀₁,
xb2 to xb4 may each independently be the same as defined herein in connection with xb1, and
R₃₀₂ to R₃₀₅ and R₃₁₁ to R₃₁₄ may each independently be the same as defined herein in connection with R₃₀₁.

In one or more embodiments, the host may include an alkaline earth metal complex, a post-transition metal complex, or any combination thereof. For example, the host may include a Be complex (e.g., Compound H55), an Mg complex, a Zn complex, or any combination thereof.

In one or more embodiments, the host may include one of Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di(carbazol-9-yl)benzene (mCP), 1,3,5-tri(carbazol-9-yl)benzene (TCP), or any combination thereof:

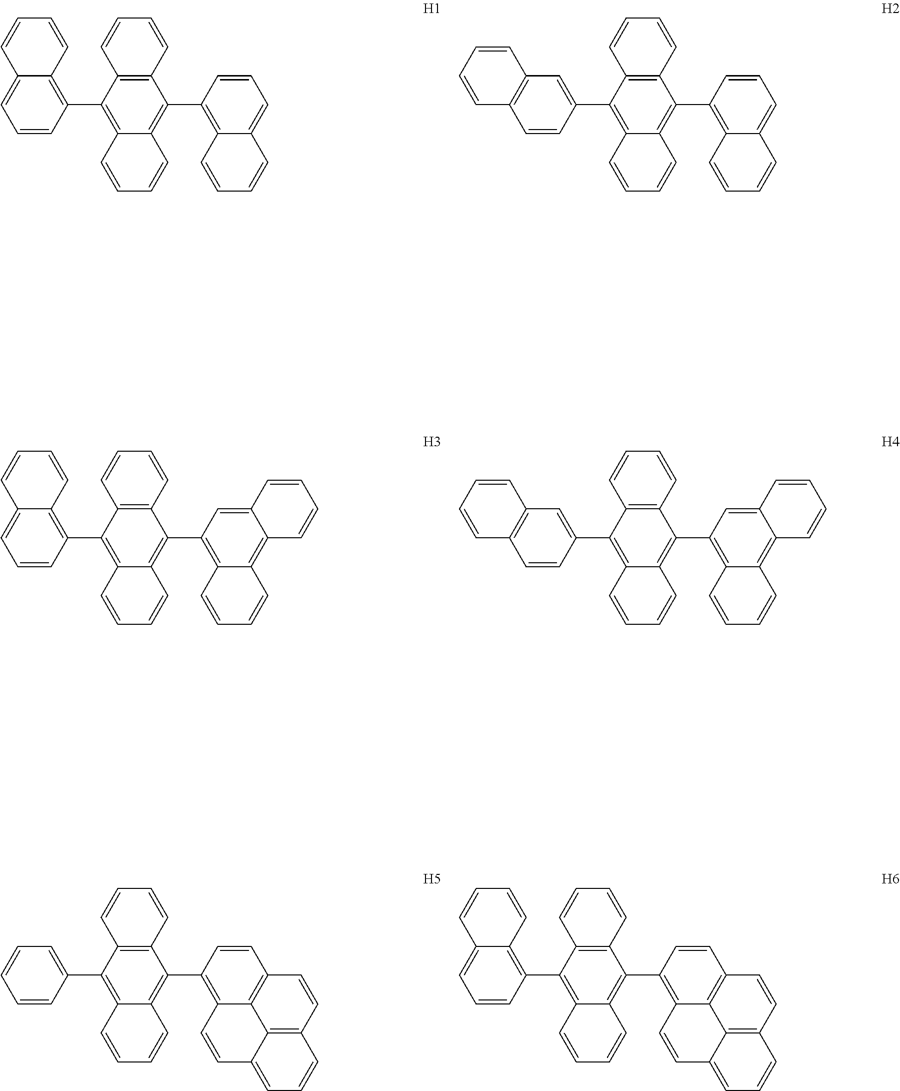

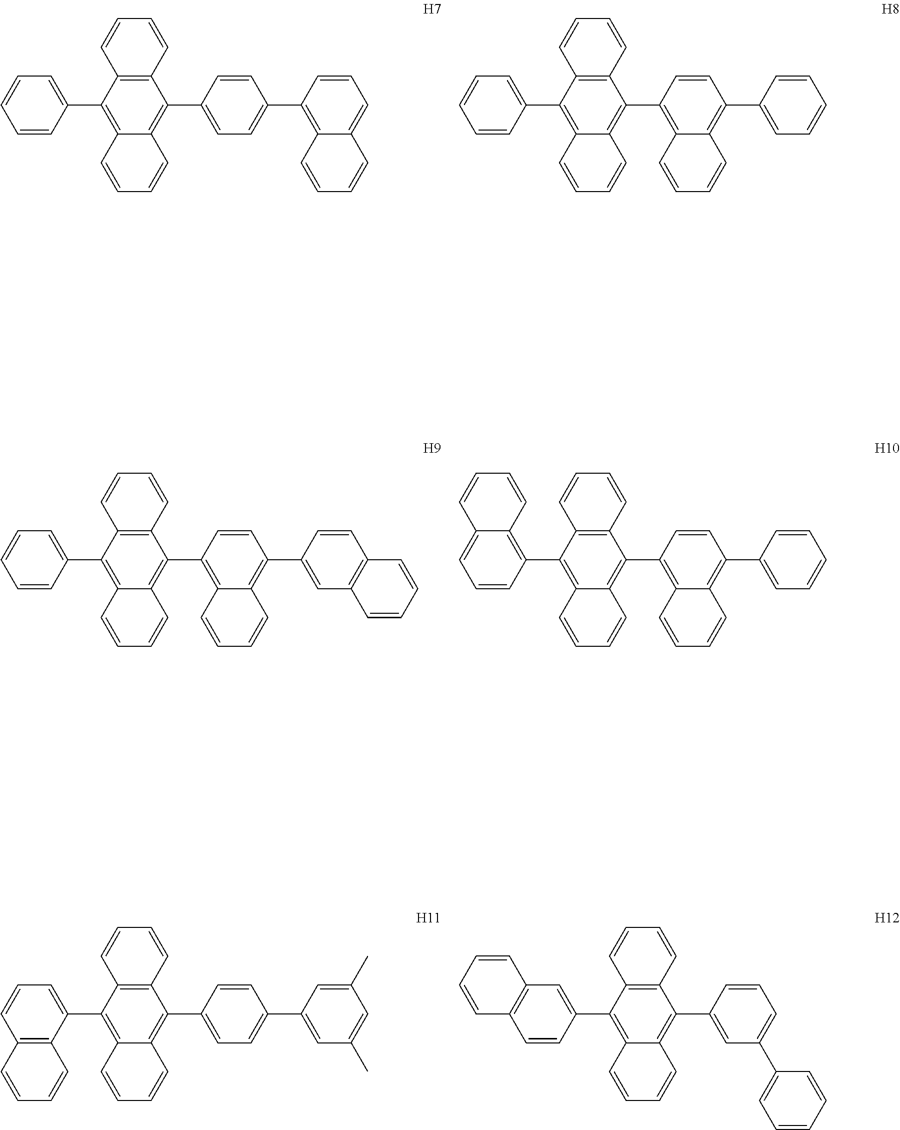

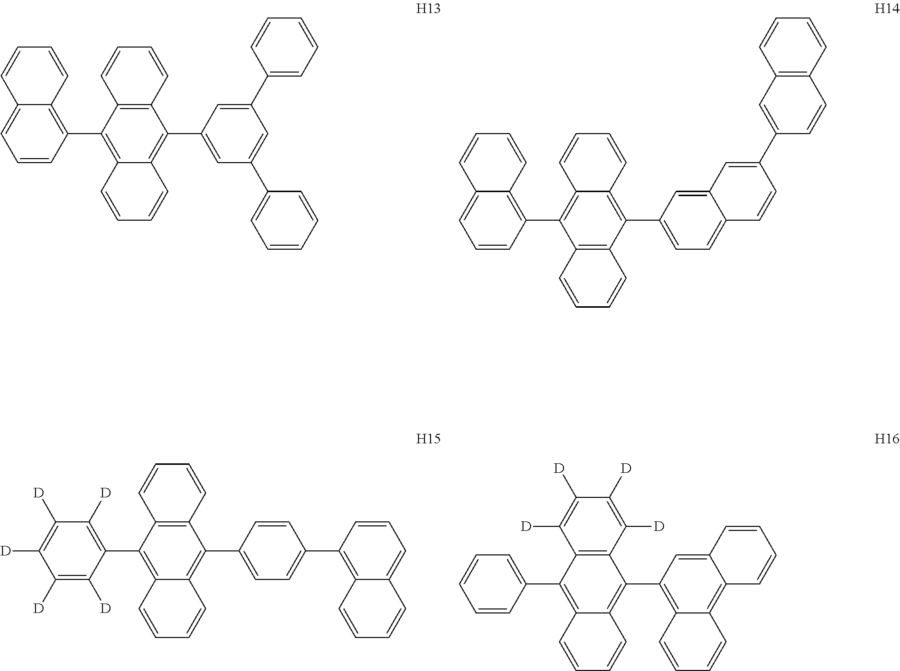

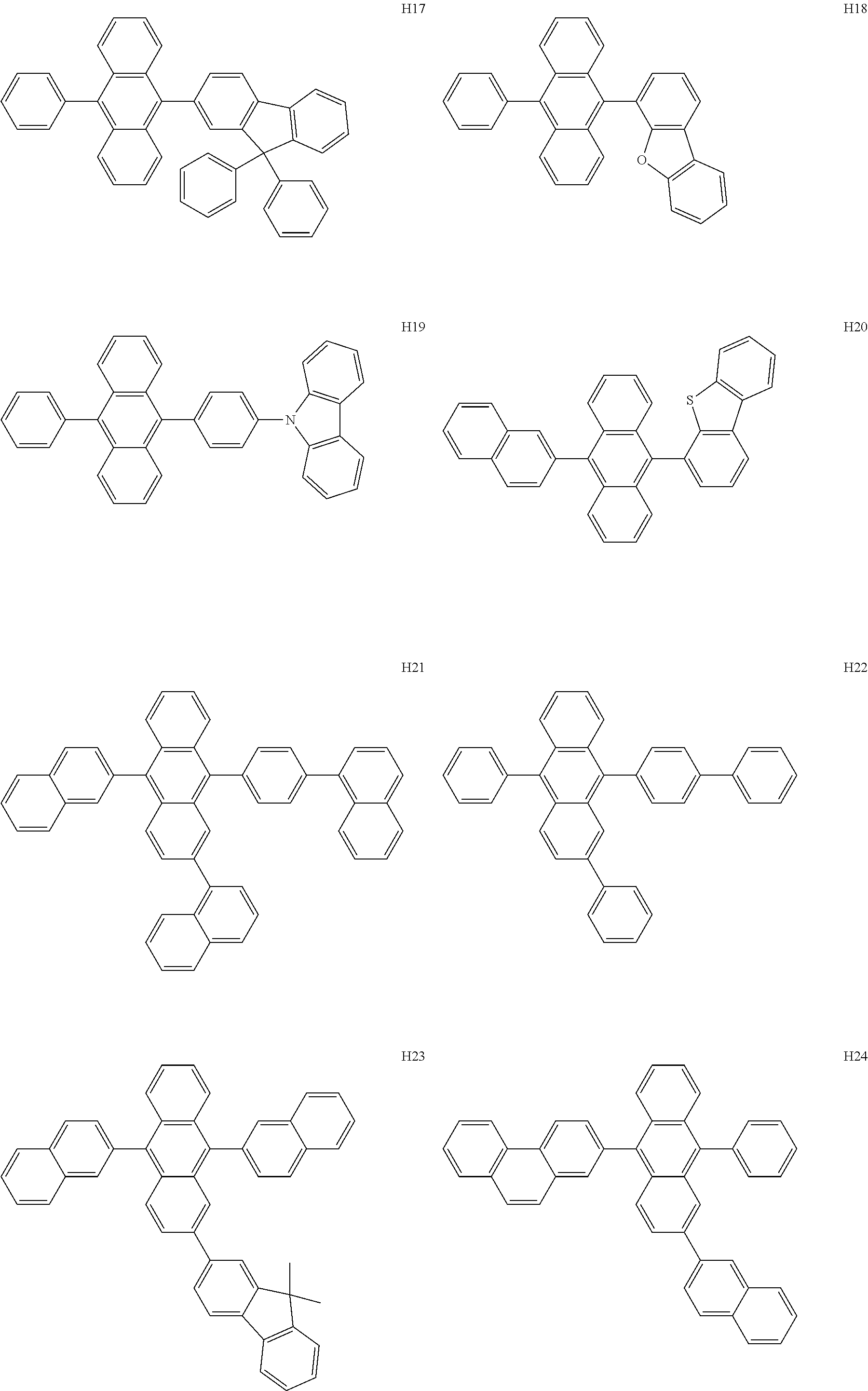

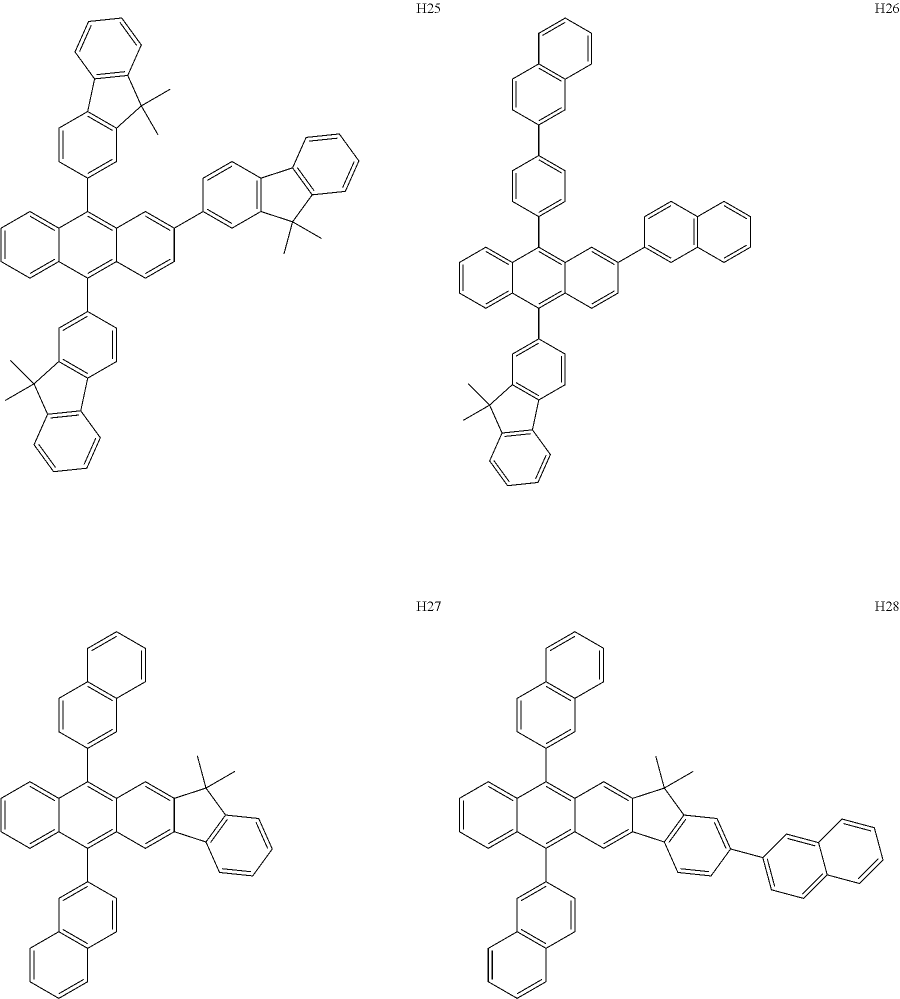

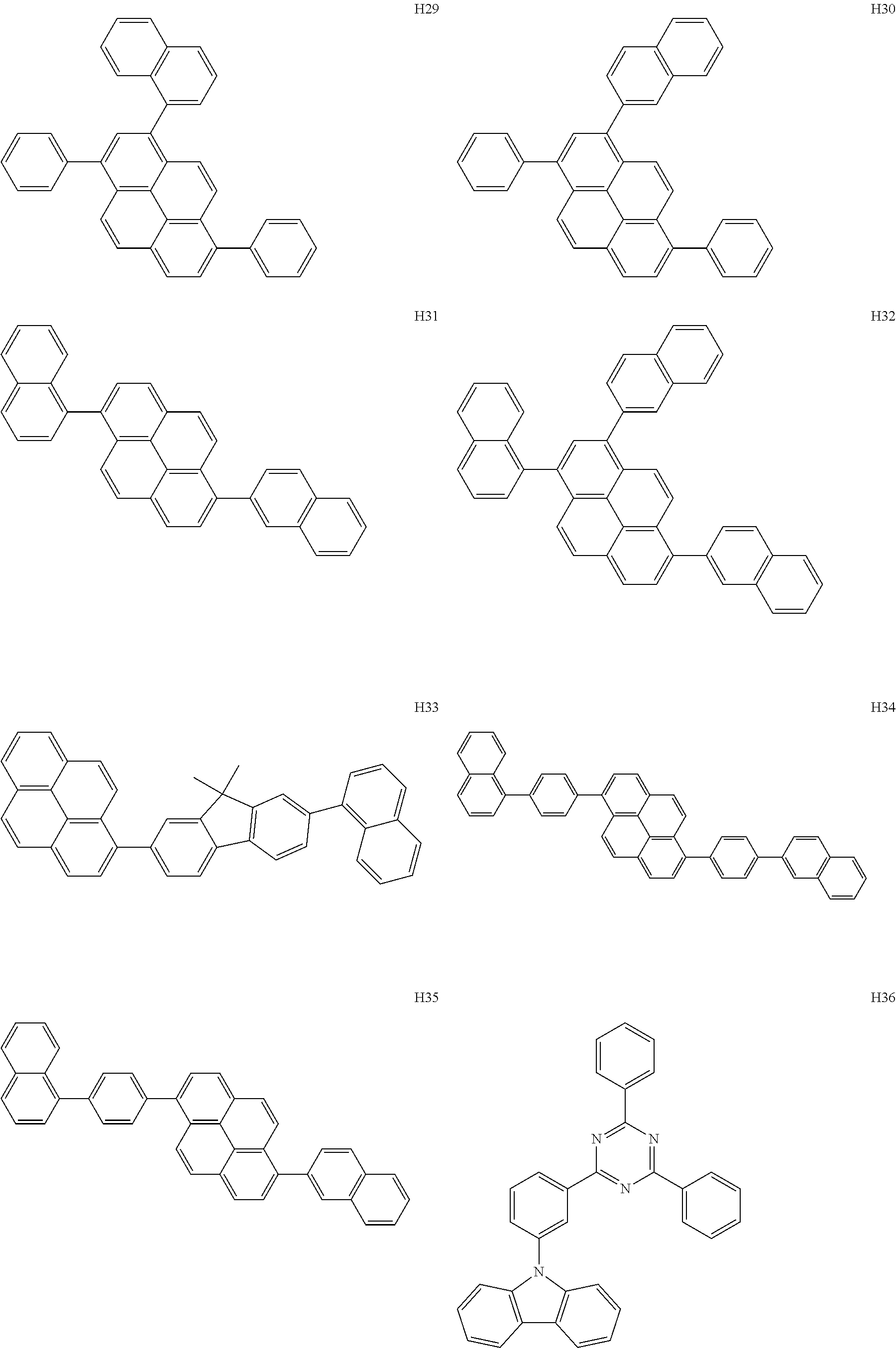

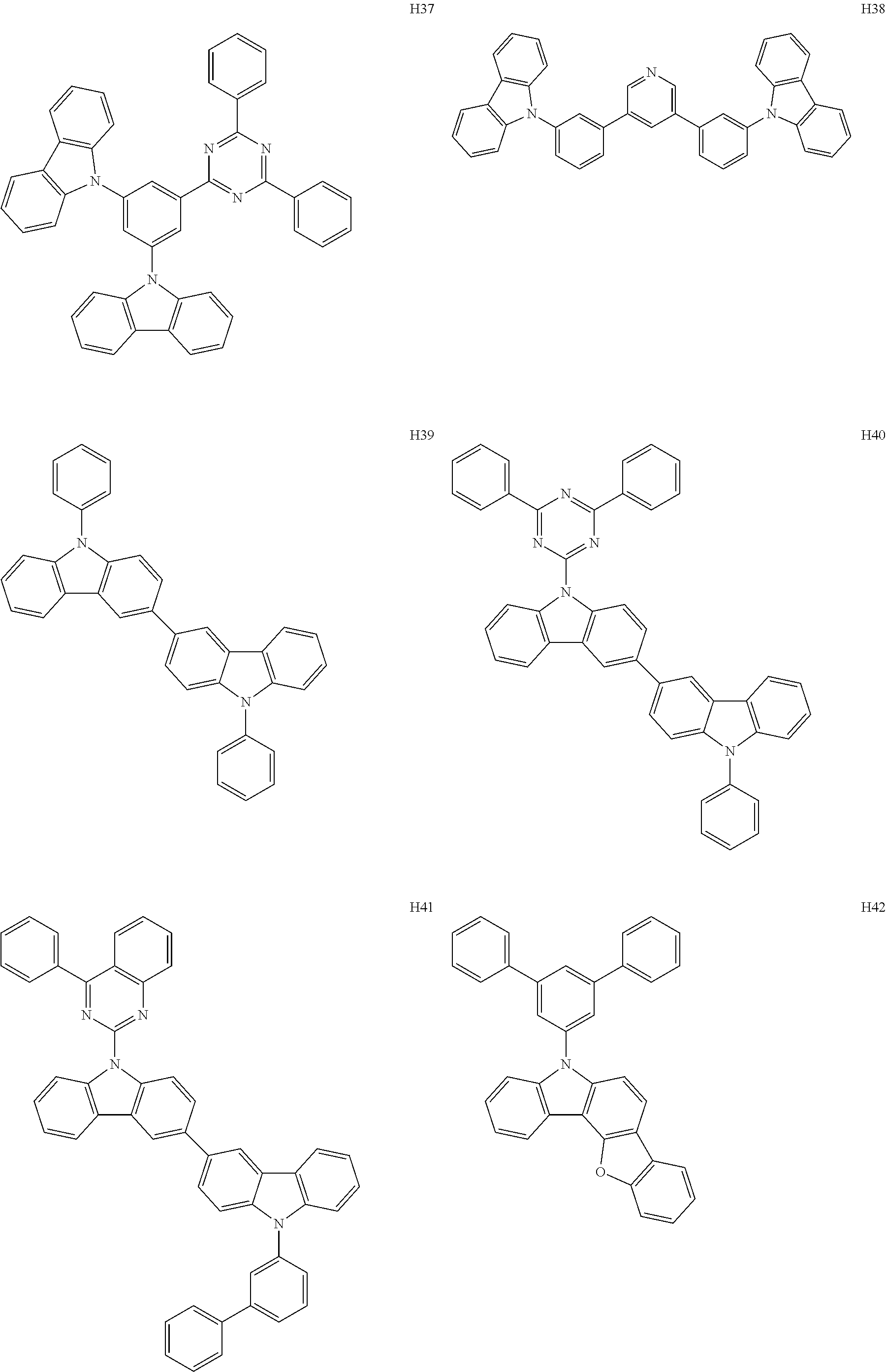 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

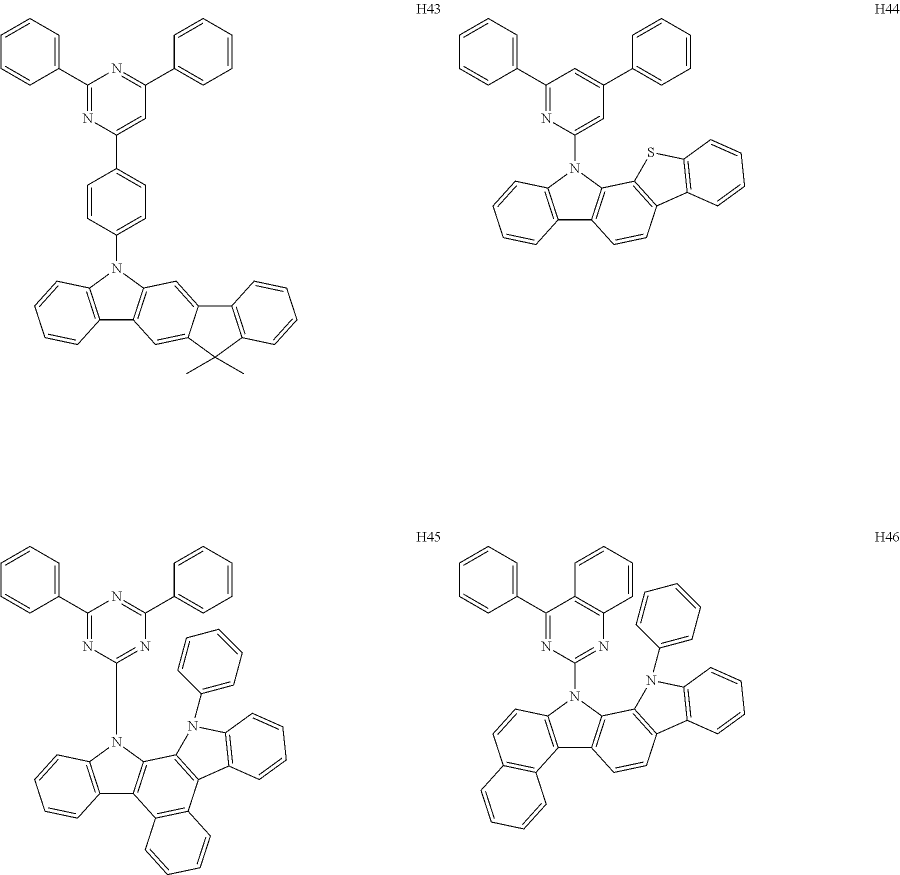 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

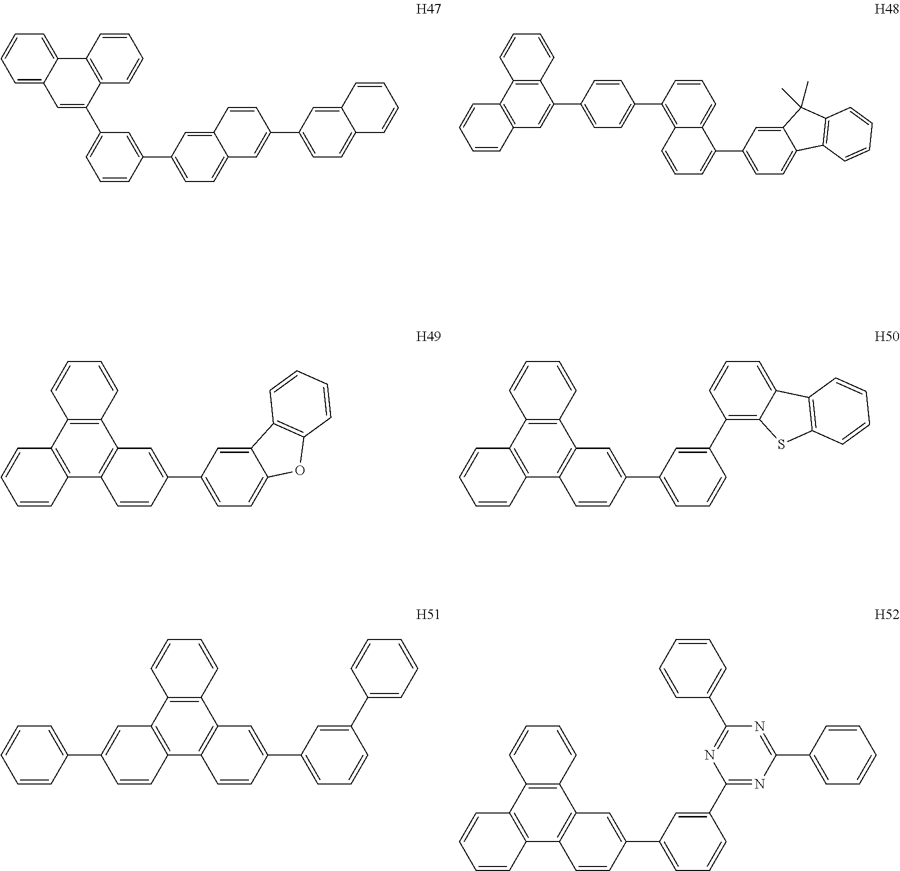

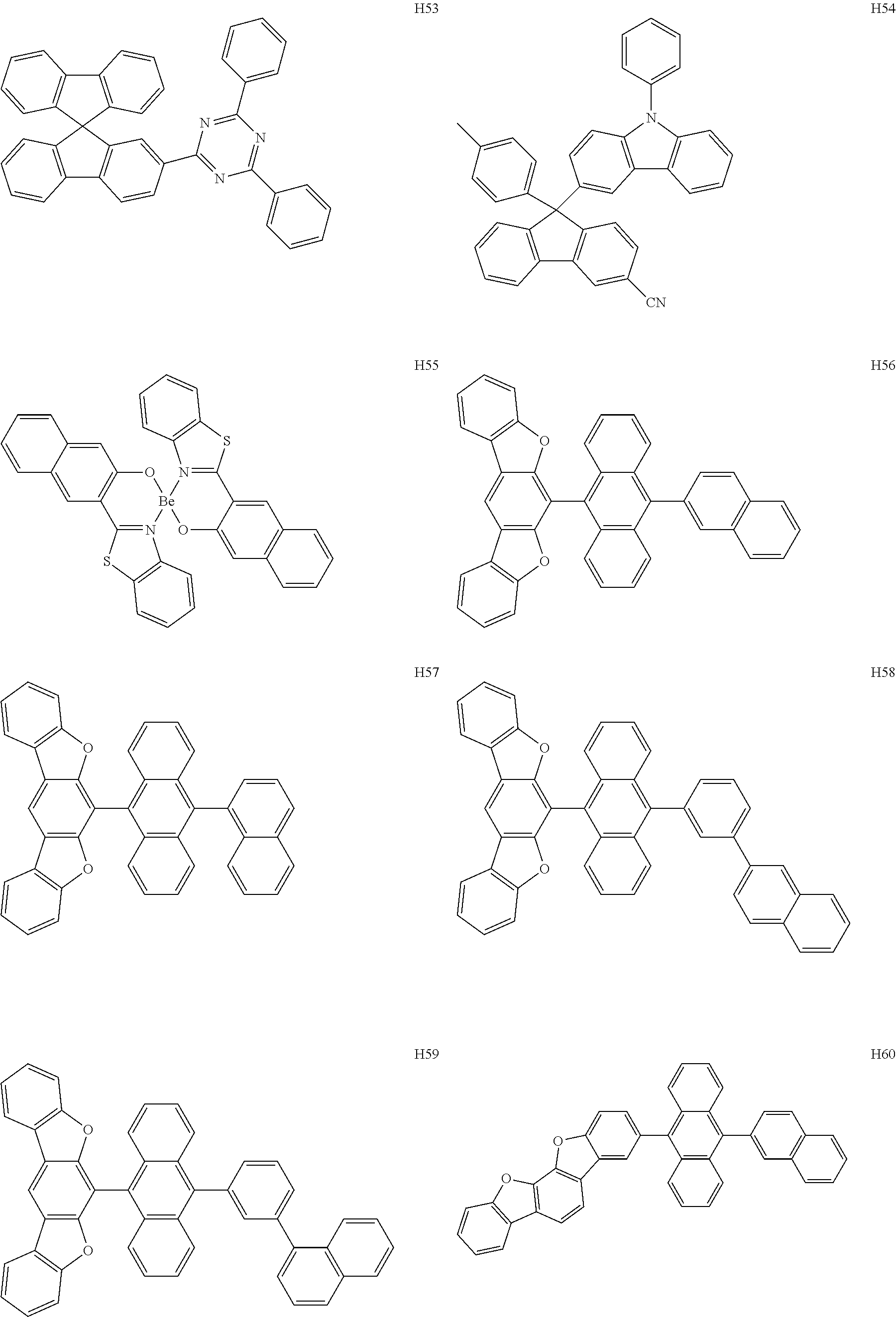

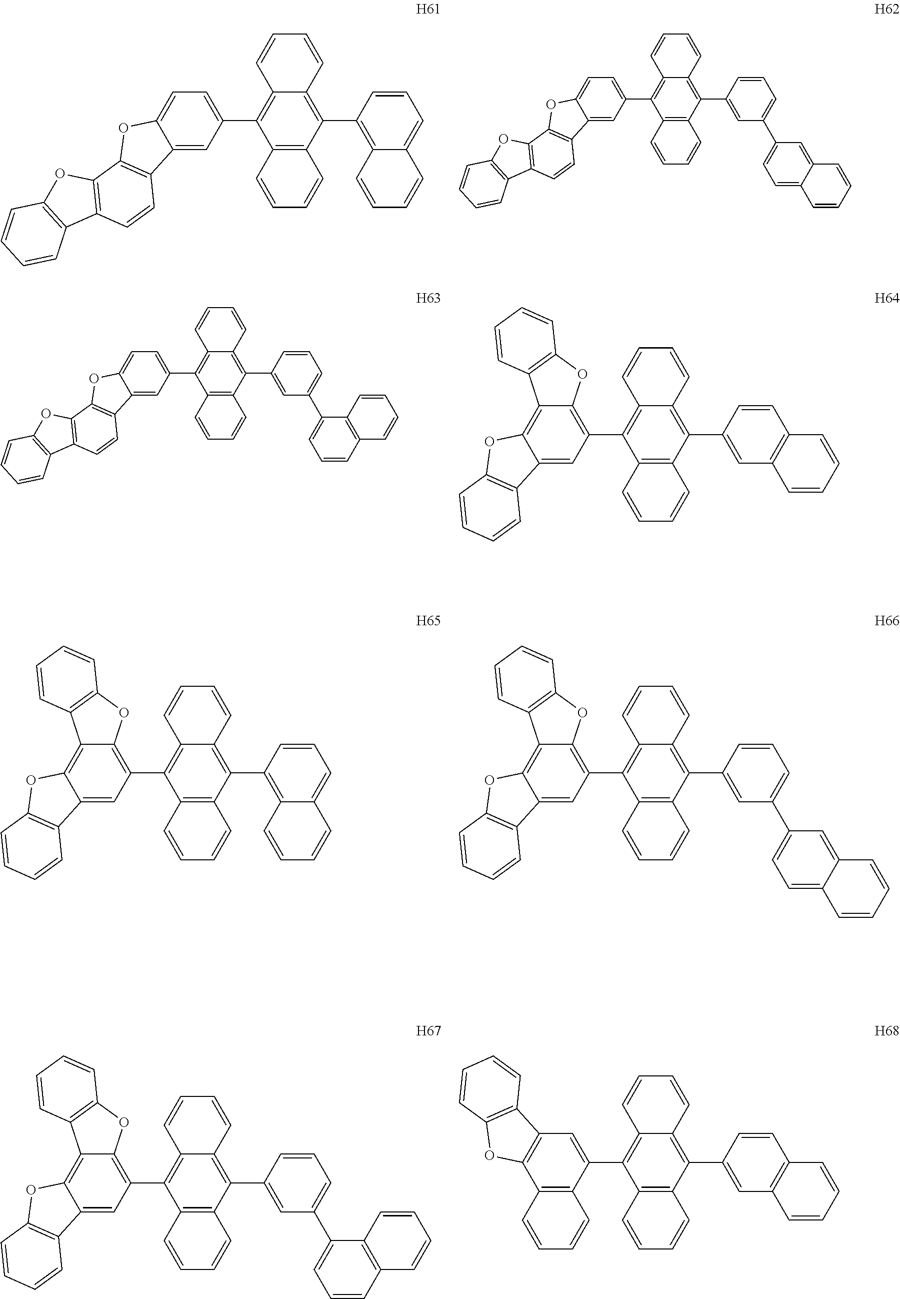

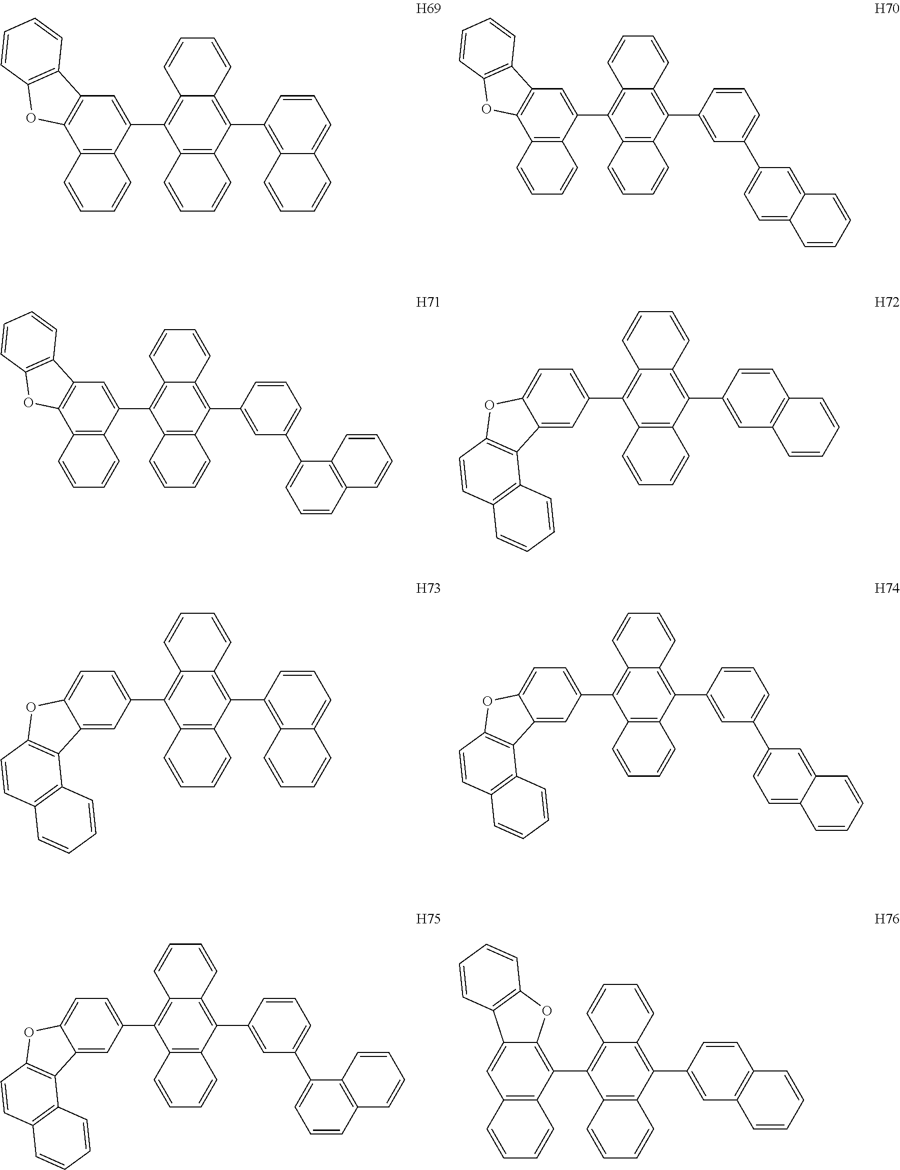

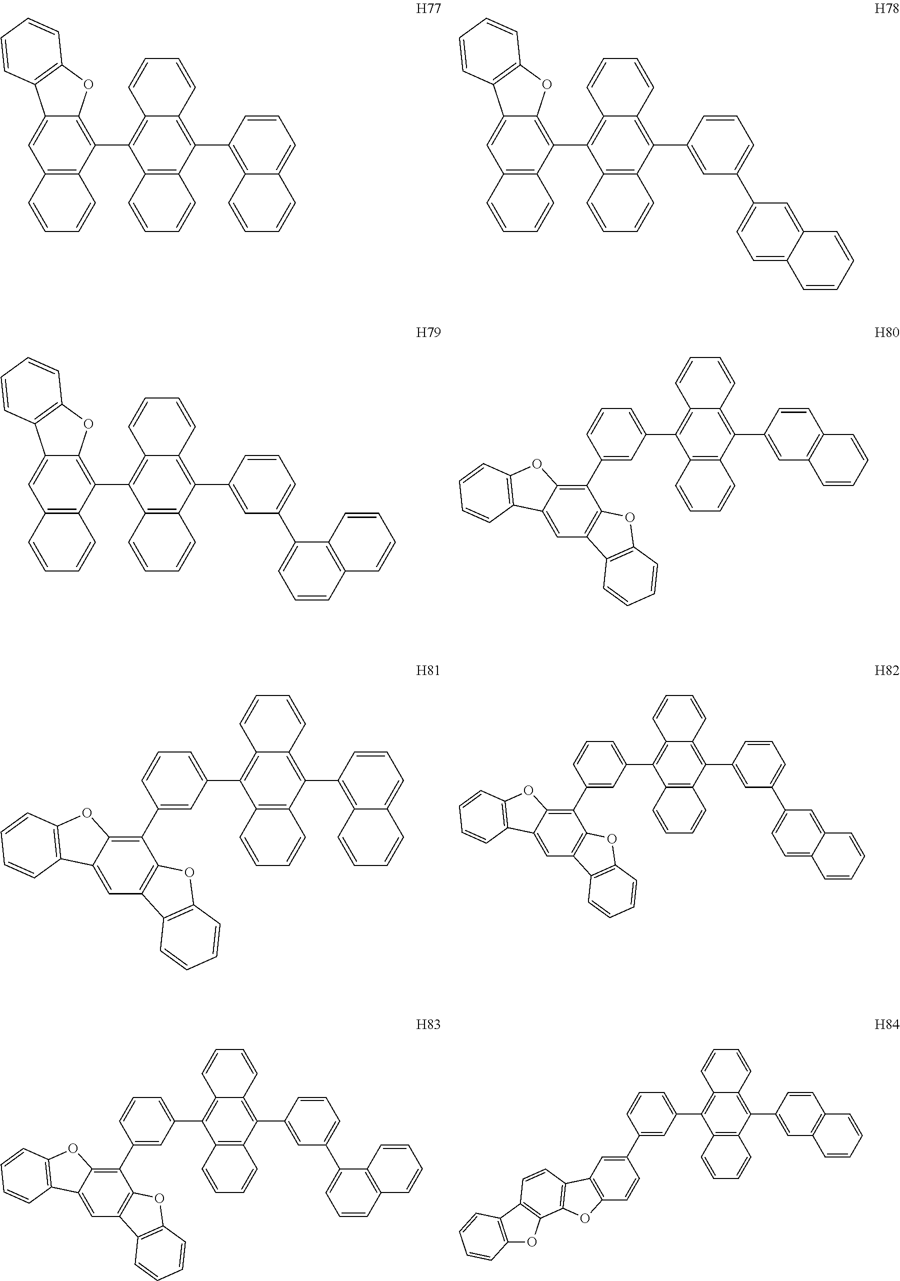

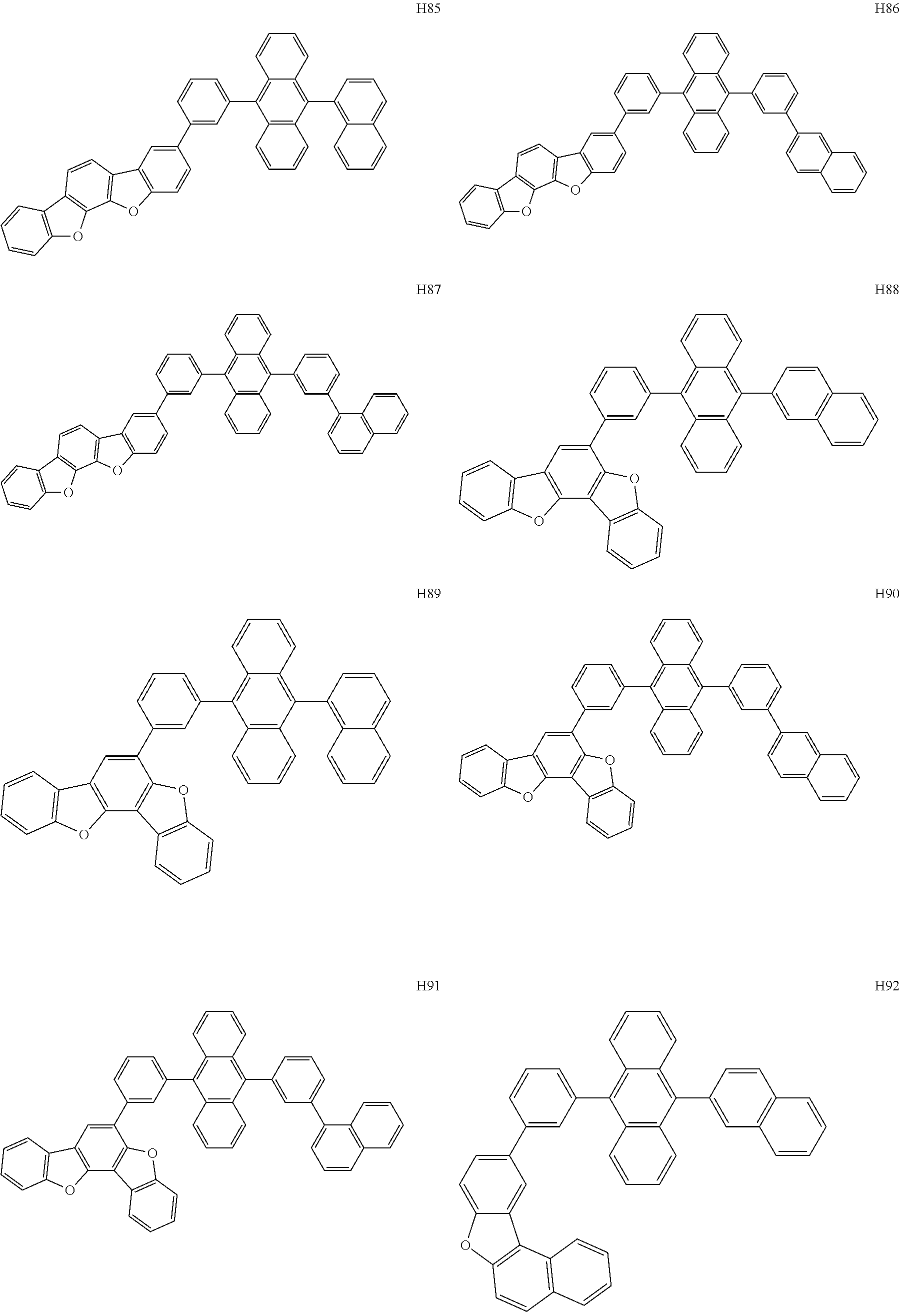

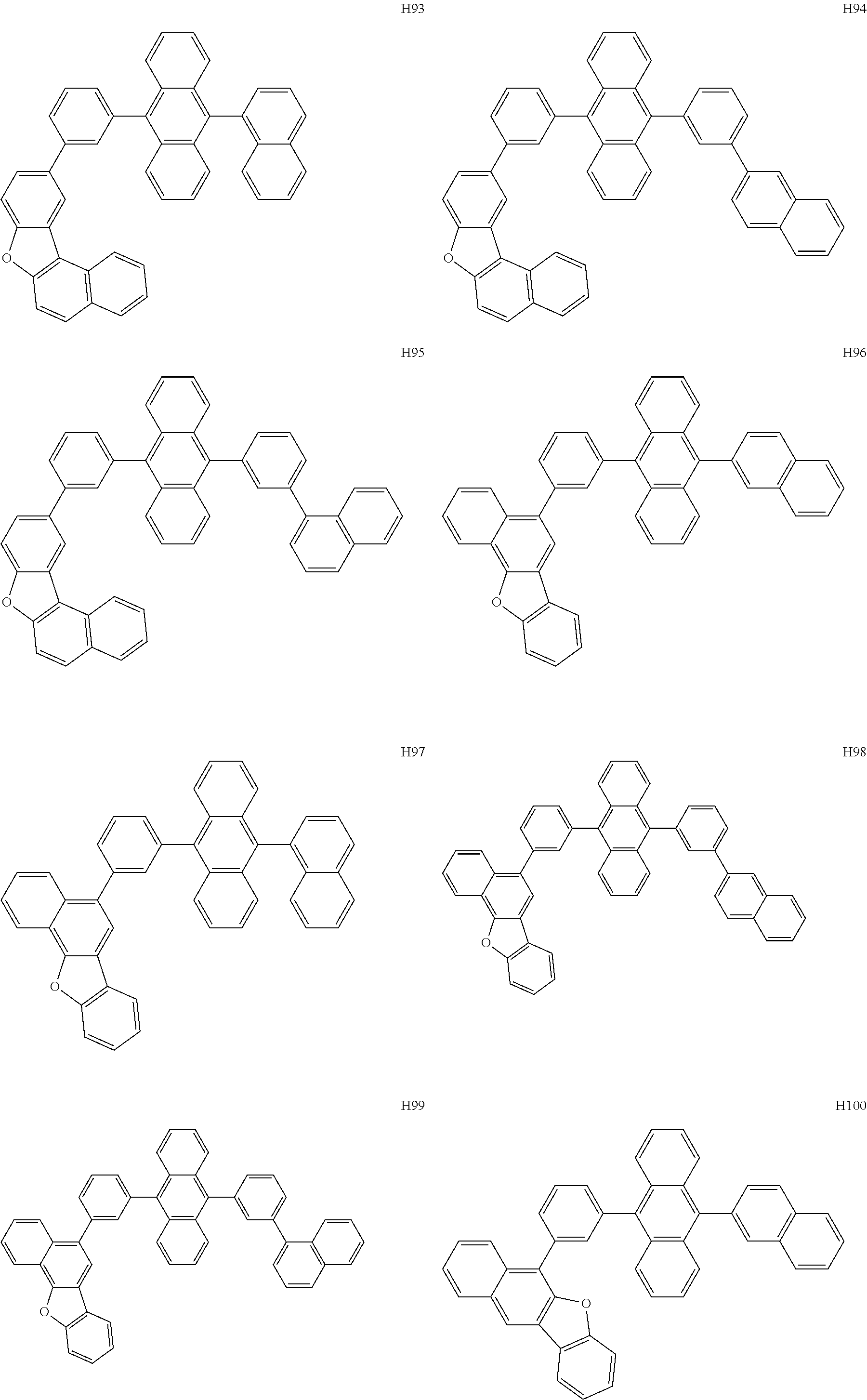

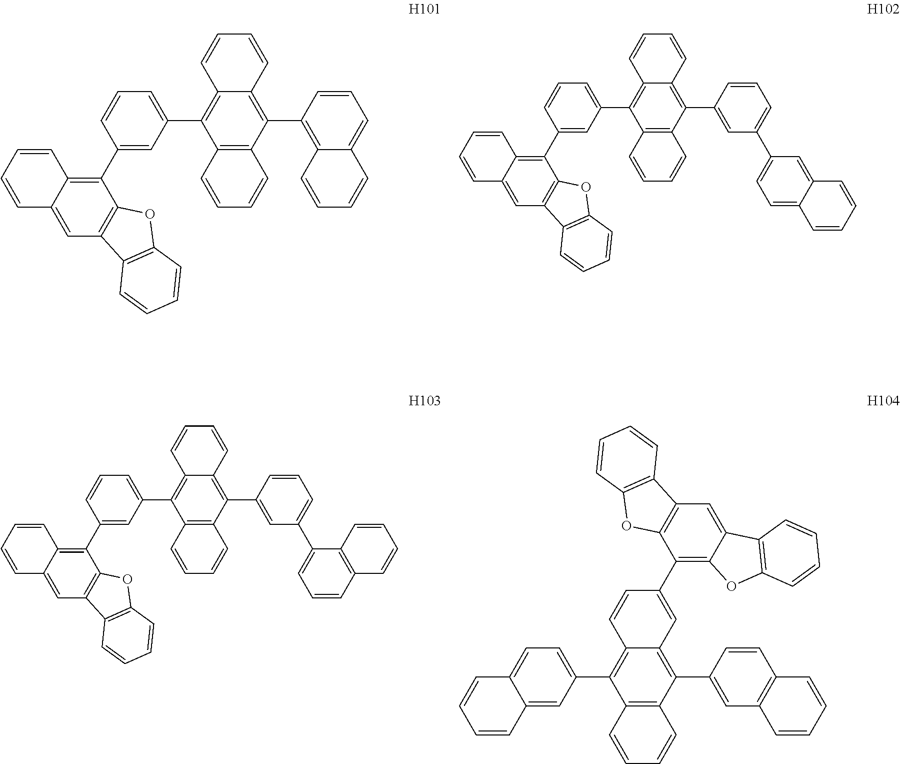 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

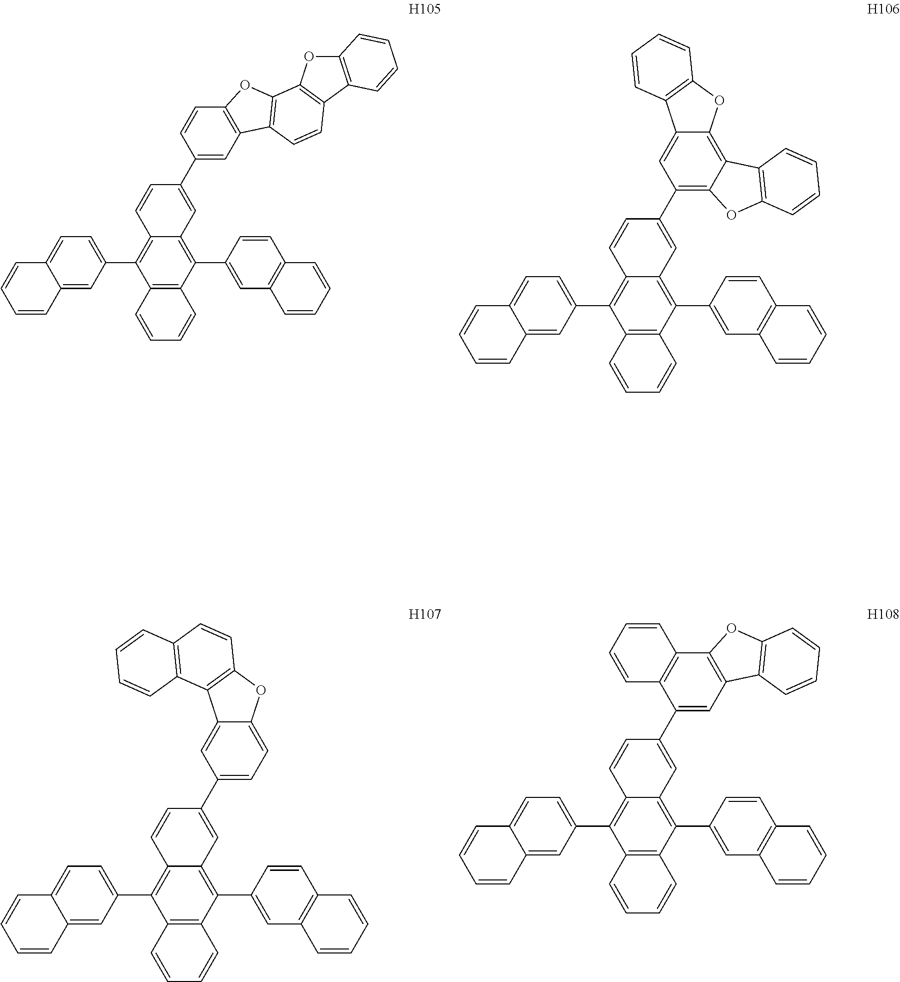 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

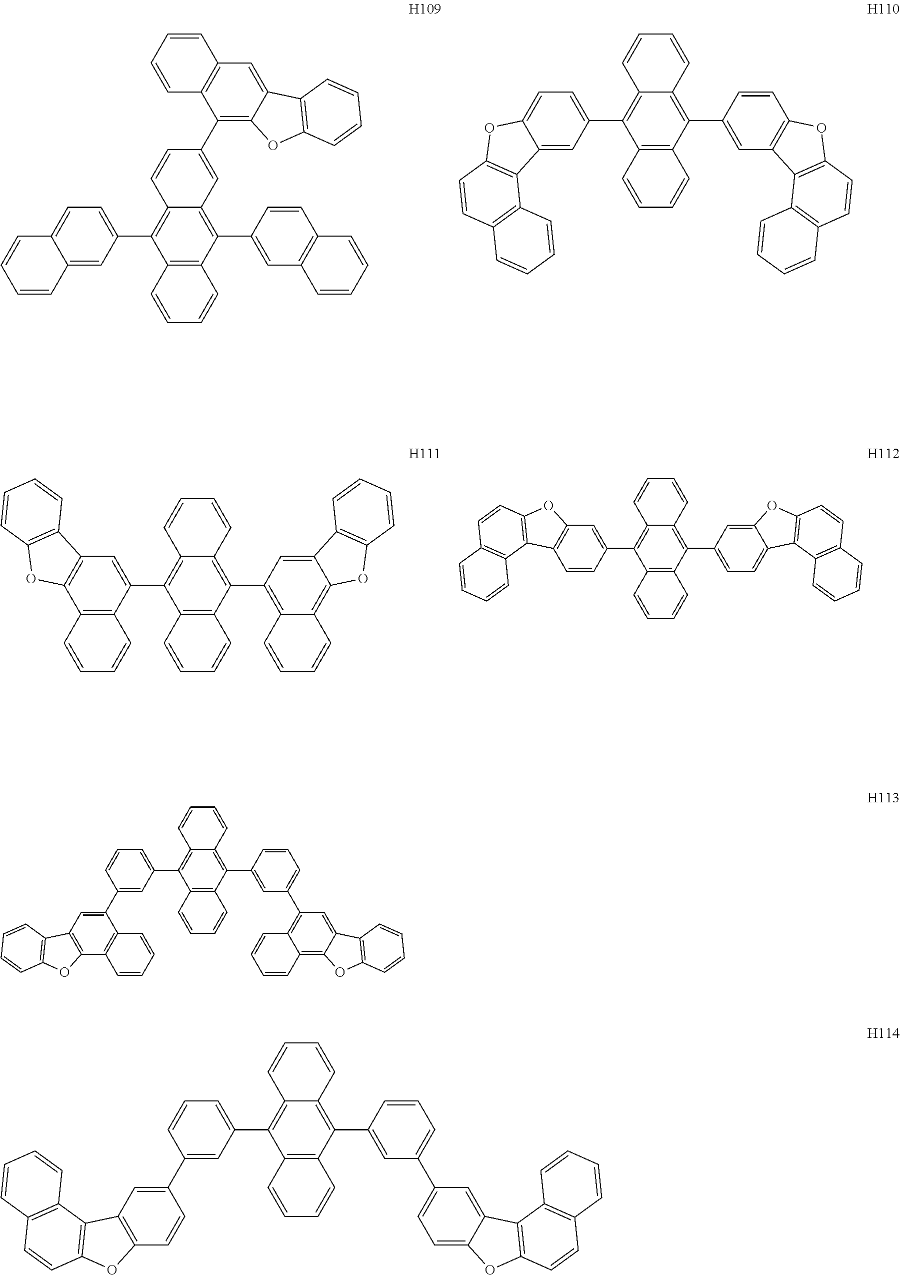

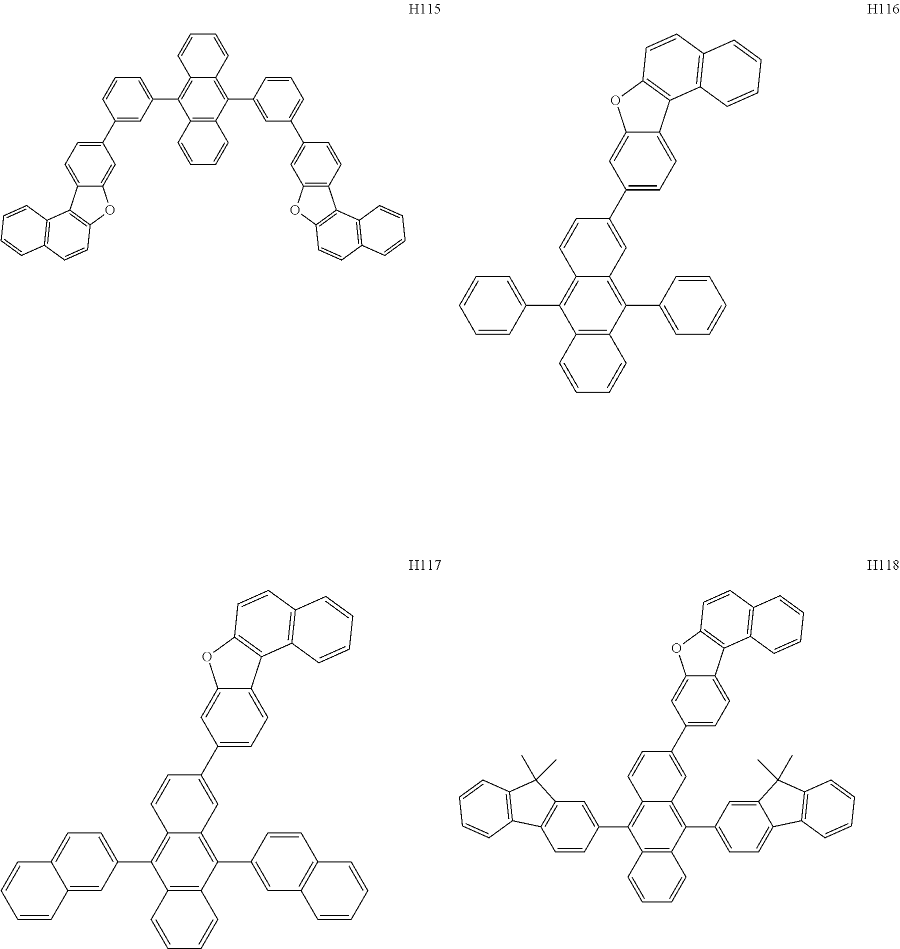 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

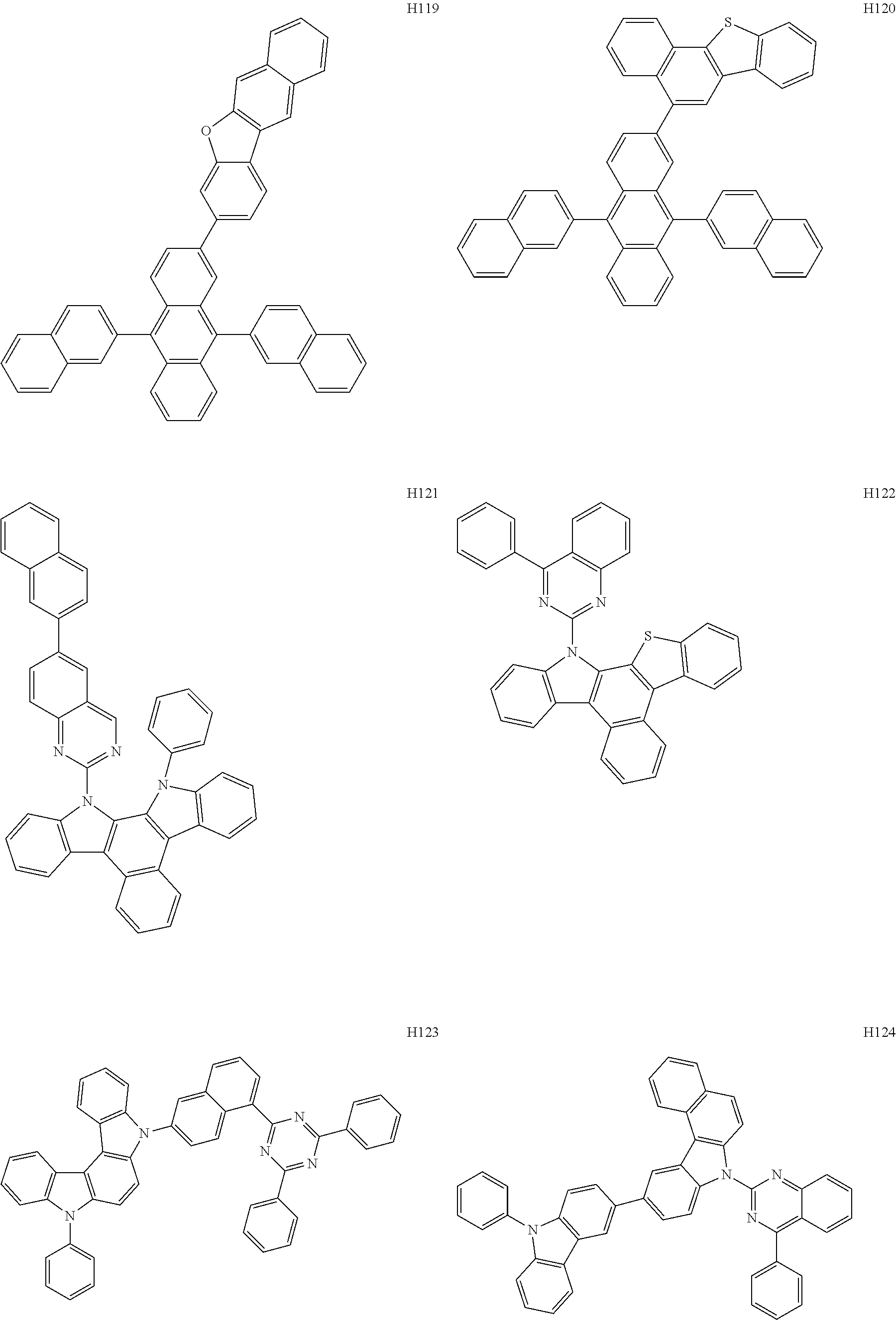

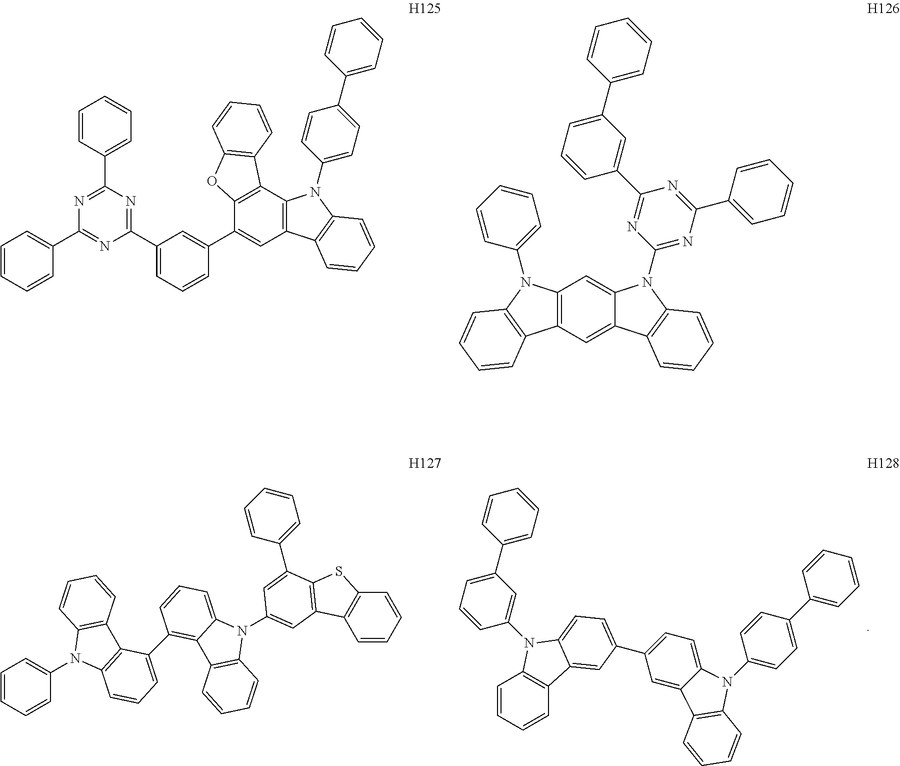 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

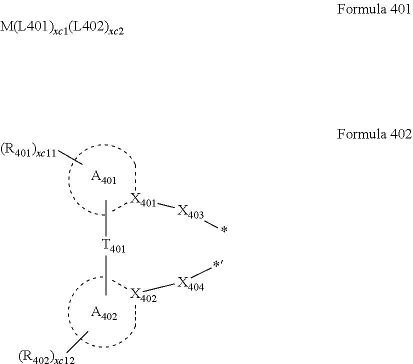

wherein, in Formulae 401 and 402,
M may be a transition metal (e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, when xc1 is 2 or more, two or more of L₄₀₁ (s) may be identical to or different from each other,
L₄₀₂ may be an organic ligand, and xc2 may be 0, 1, 2, 3, or 4, wherein, when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *═C═*′,
X₄₀₃ and X₄₀₄ may each independently be a chemical bond (e.g., a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each independently be the same as defined herein in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each independently be the same as defined herein in connection with Q₁,
xc11 and xc12 may each independently be an integer from 0 to 10, and
* and *′ in Formula 402 may each indicate a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In an embodiment, when xc1 in Formula 401 is 2 or more, two ring A₄₀₁(s) in two or more of L₄₀₁(s) may optionally be linked to each other via T₄₀₂, which is a linking group, and/or two ring A₄₀₂(s) may optionally be linked to each other via T₄₀₃, which is a linking group (see Compounds PD1 to PD4 and PD7). T₄₀₂ and T₄₀₃ may each independently be the same as defined herein in connection with T₄₀₁.

L₄₀₂ in Formula 401 may be an organic ligand. For example, L₄₀₂ may include a halogen group, a diketone group (e.g., an acetylacetonate group), a carboxylic acid group (e.g., a picolinate group), —C(═O), an isonitrile group, a —CN group, a phosphorus group (e.g., a phosphine group, a phosphite group, etc.), or any combination thereof.

The phosphorescent dopant may include, for example, at least one of Compounds PD1 to PD39 or any combination thereof:

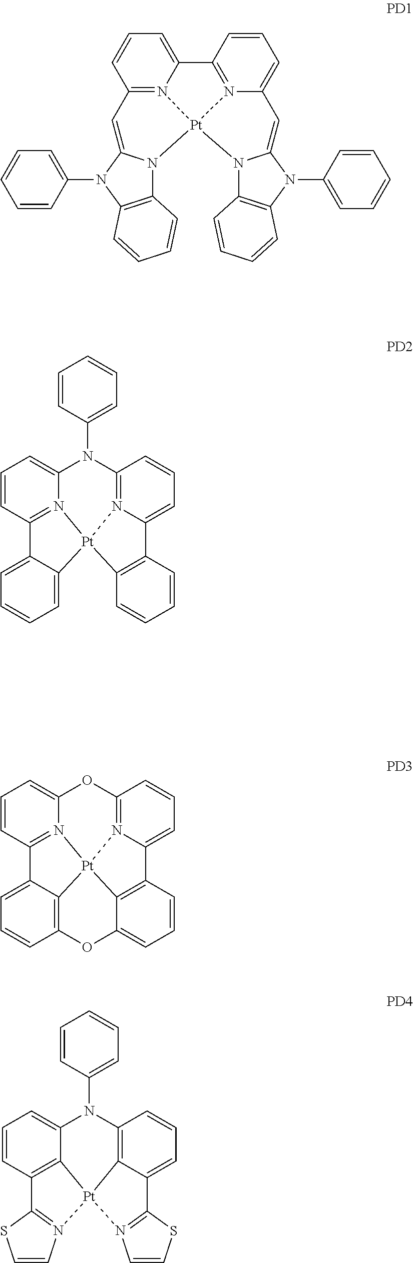

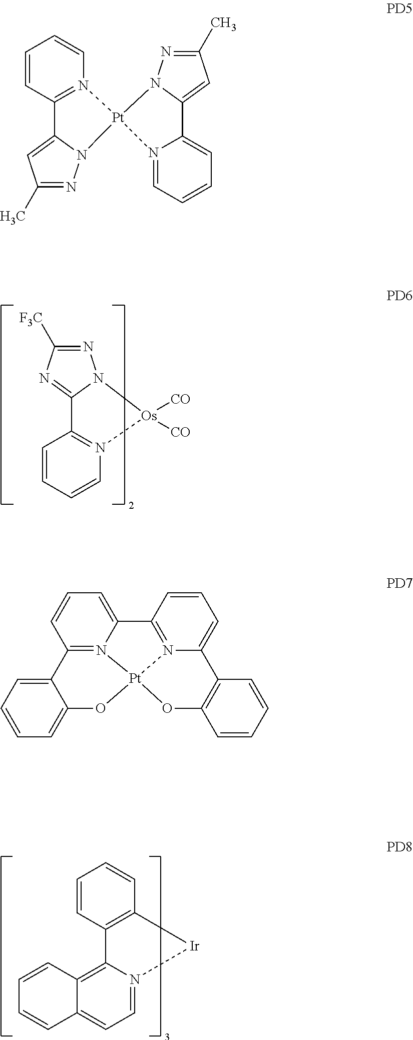

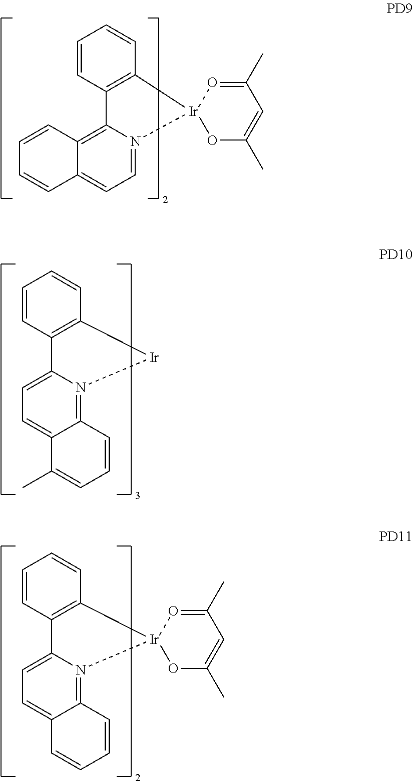

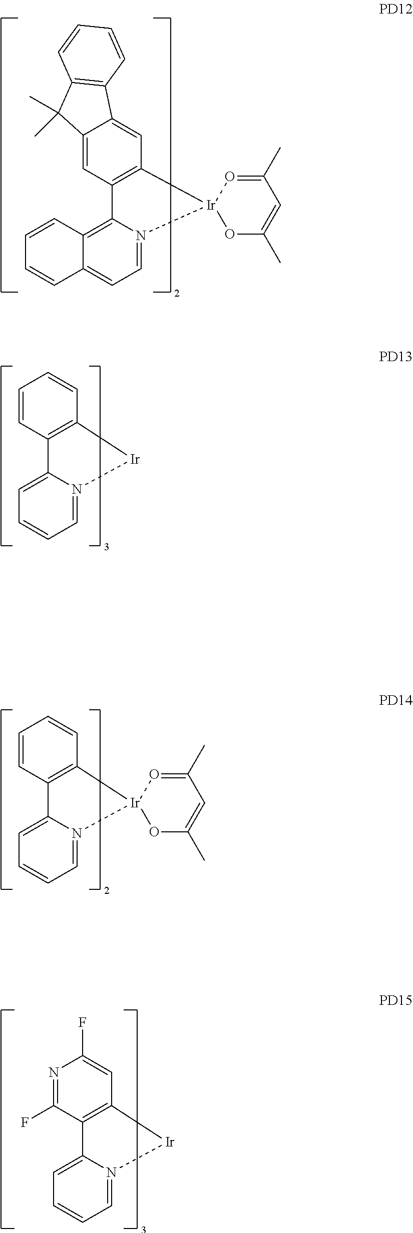

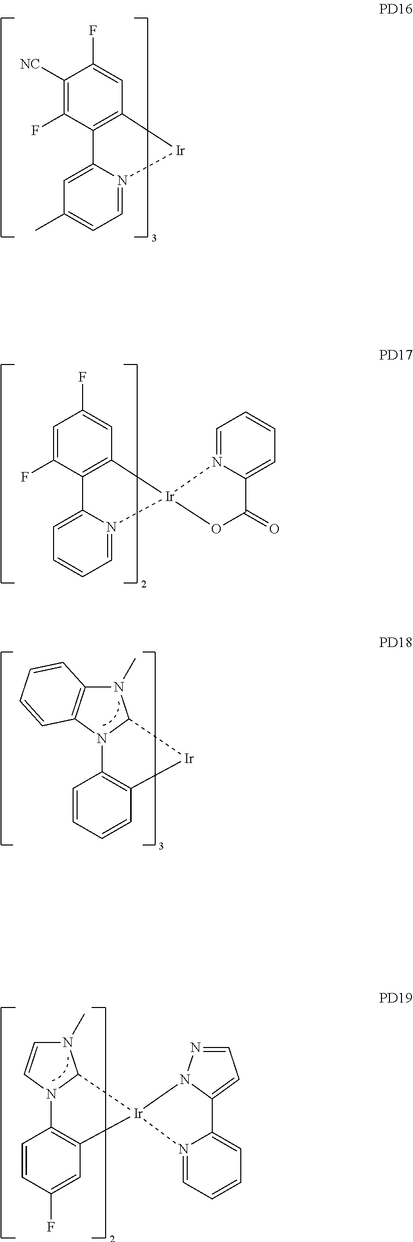

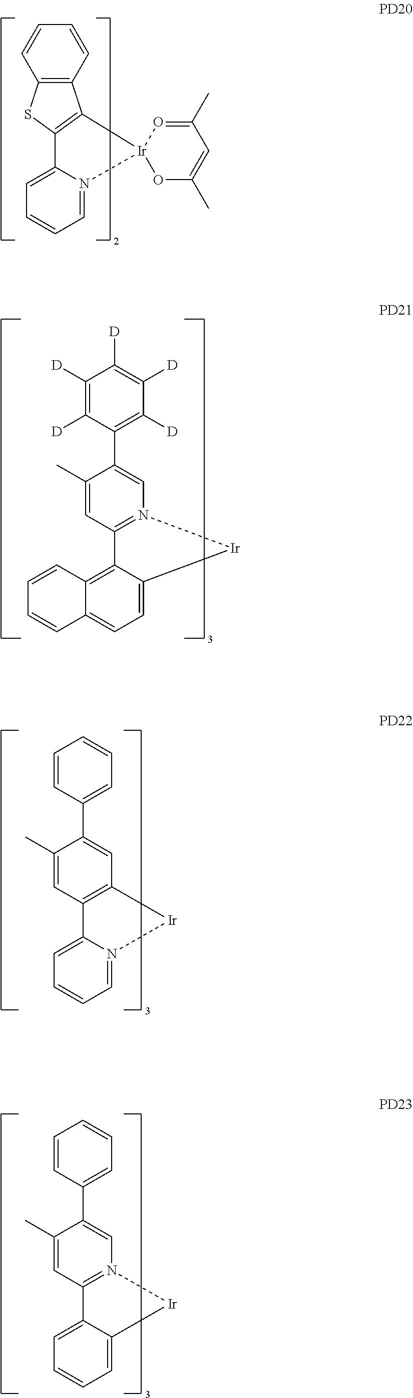

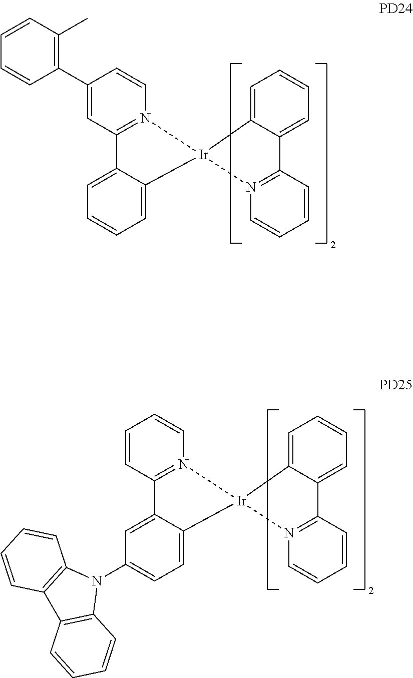

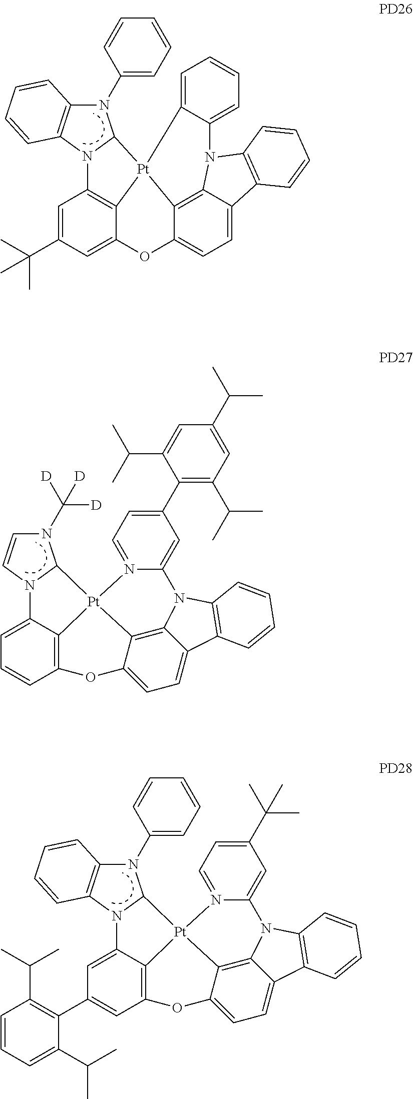

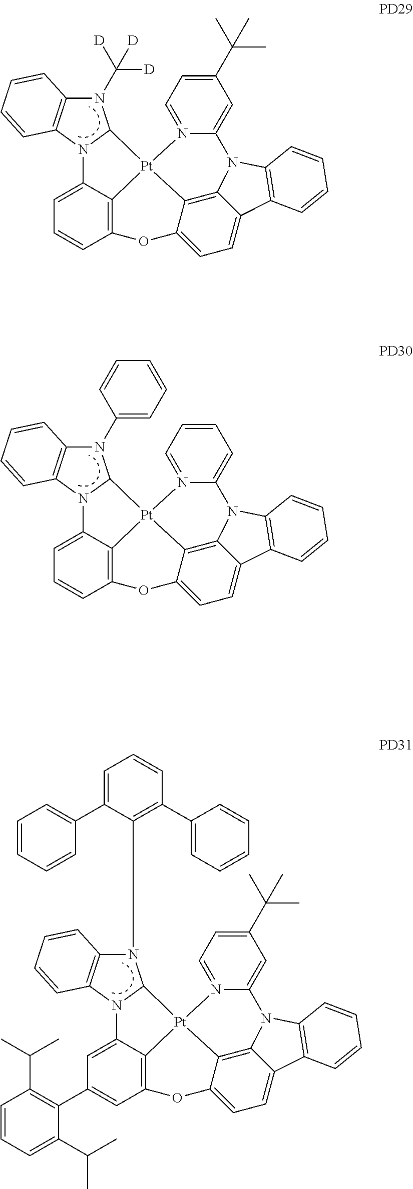

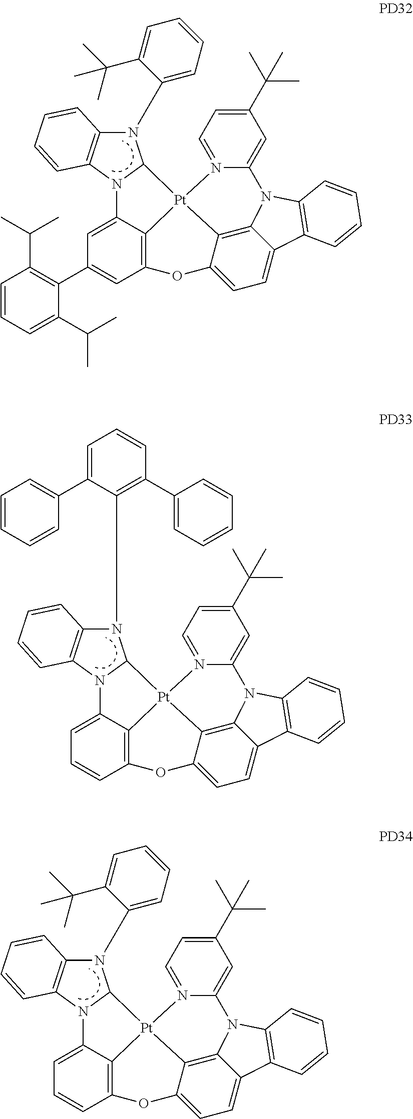

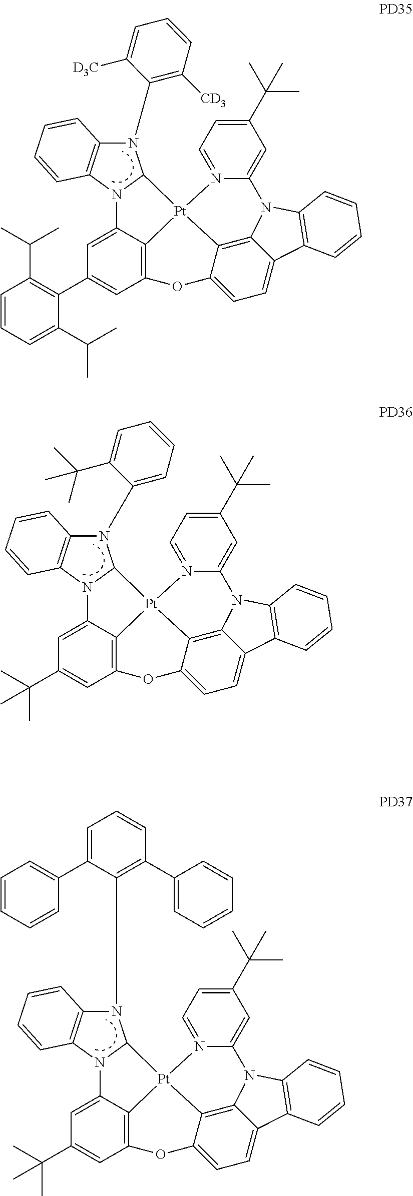

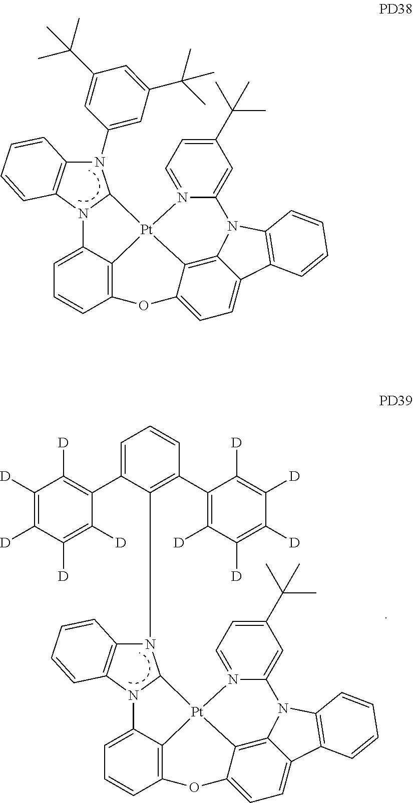

### Fluorescent Dopant

The fluorescent dopant may include an amine group-containing compound, a styryl group-containing compound, or any combination thereof.

For example, the fluorescent dopant may include a compound represented by Formula 501:

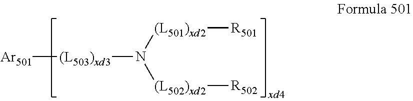

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, Ar₅₀₁ in Formula 501 may be a condensed cyclic group (e.g., an anthracene group, a chrysene group, a pyrene group, etc.) in which three or more monocyclic groups are condensed with each other.

In an embodiment, xd4 in Formula 501 may be 2.

For example, the fluorescent dopant may include at least one of Compounds FD1 to FD37, DPVBi, DPAVBi, or any combination thereof:

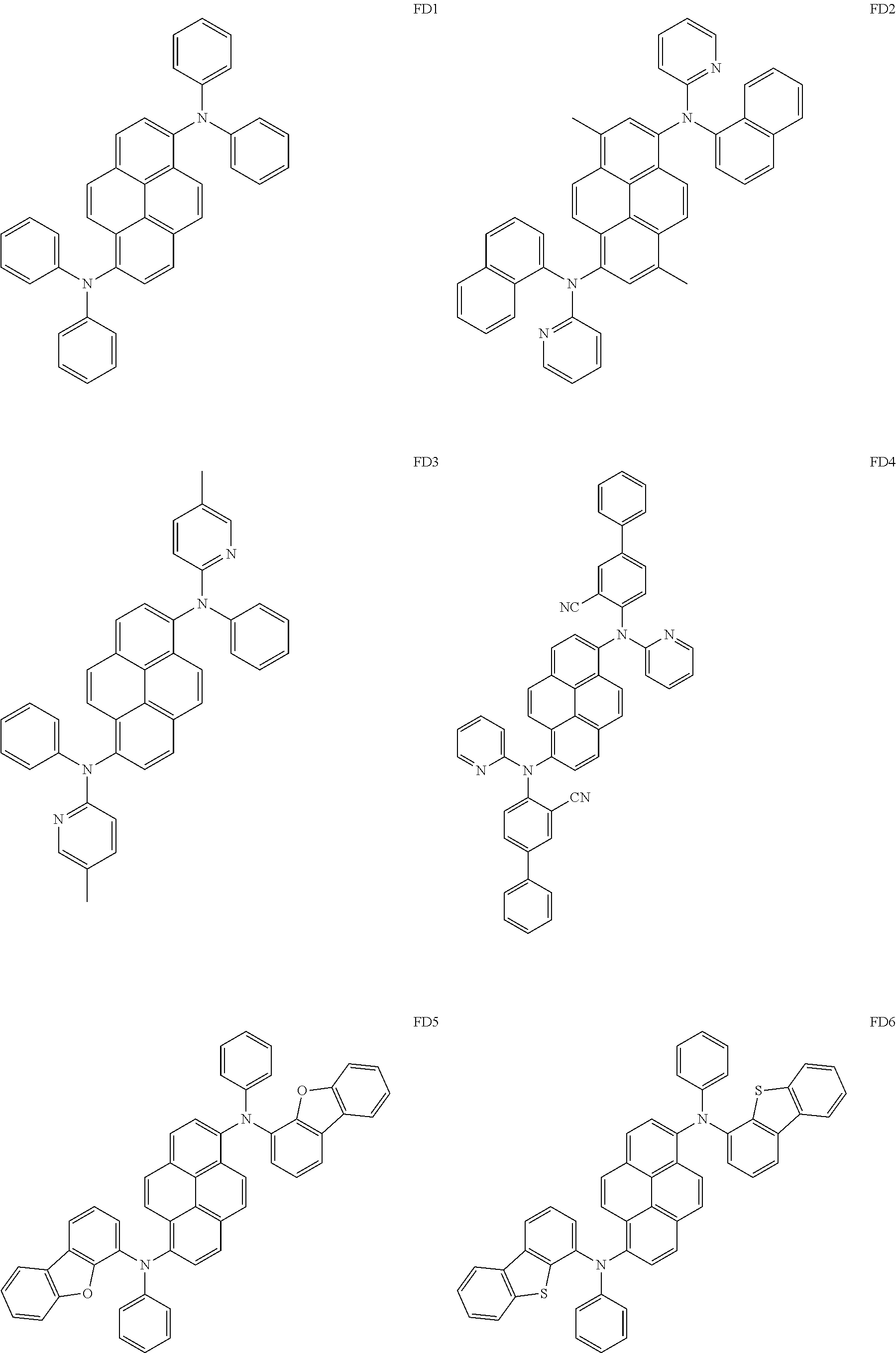

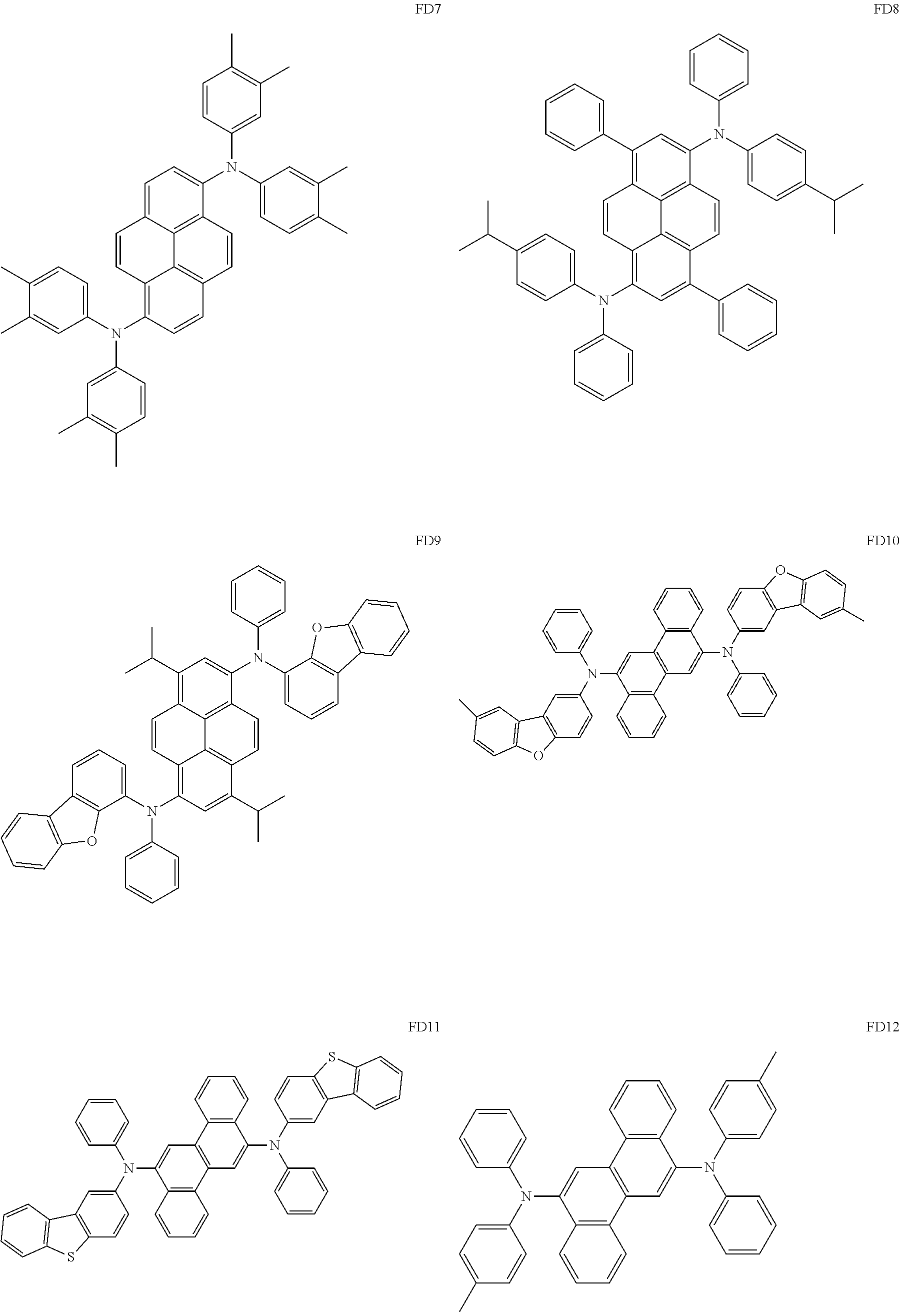

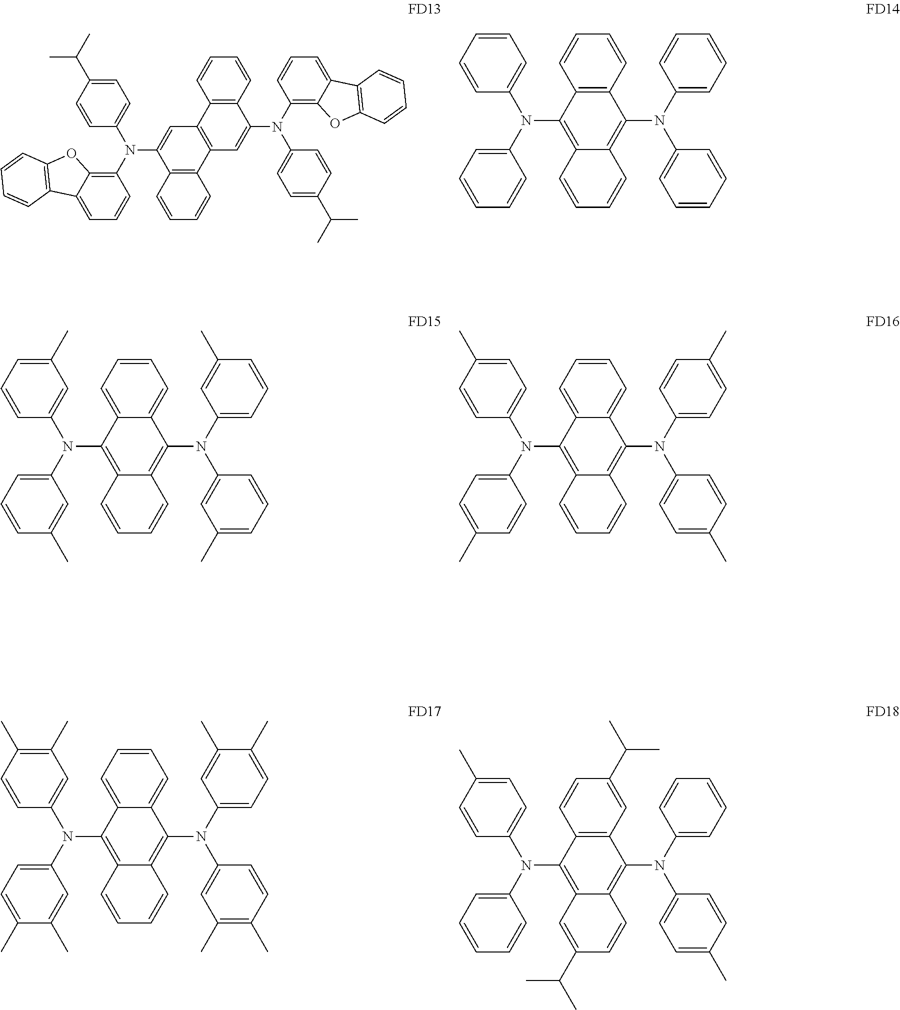 [SMILES: CC(C)c1ccc(N(c2cc3c4ccccc4c(N(c4ccc(C(C)C)cc4)c4cccc5c4oc4ccccc45)cc3c3ccccc23)c2cccc3c2oc2ccccc23)cc1 ; c1ccc(N(c2ccccc2)c2c3ccccc3c(N(c3ccccc3)c3ccccc3)c3ccccc23)cc1 ; Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

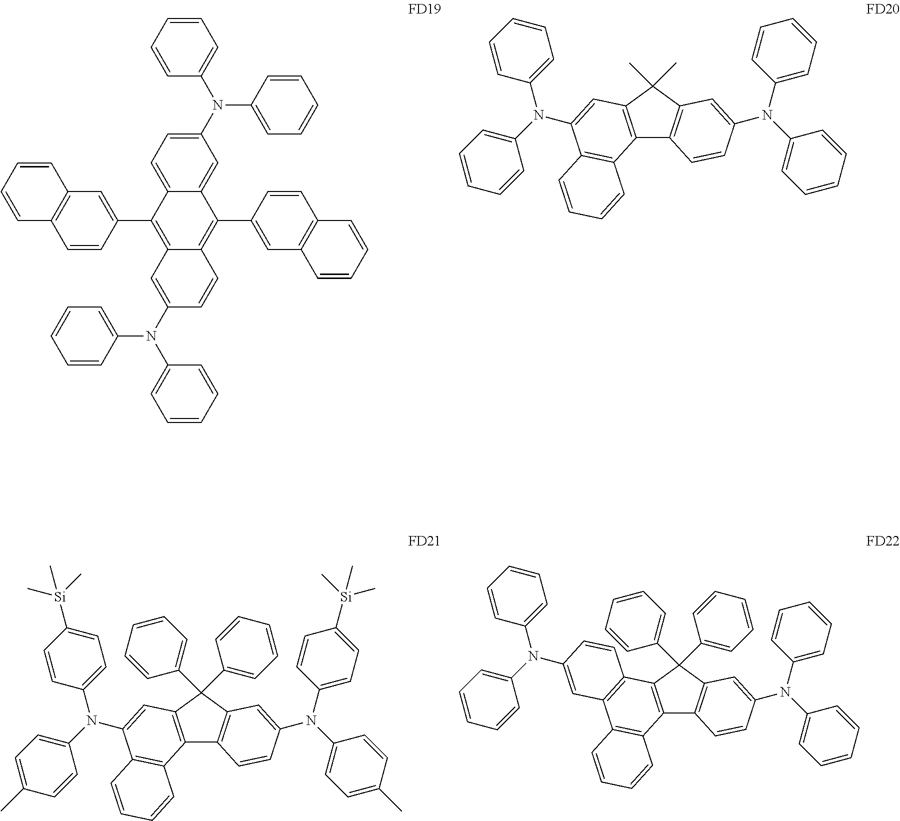

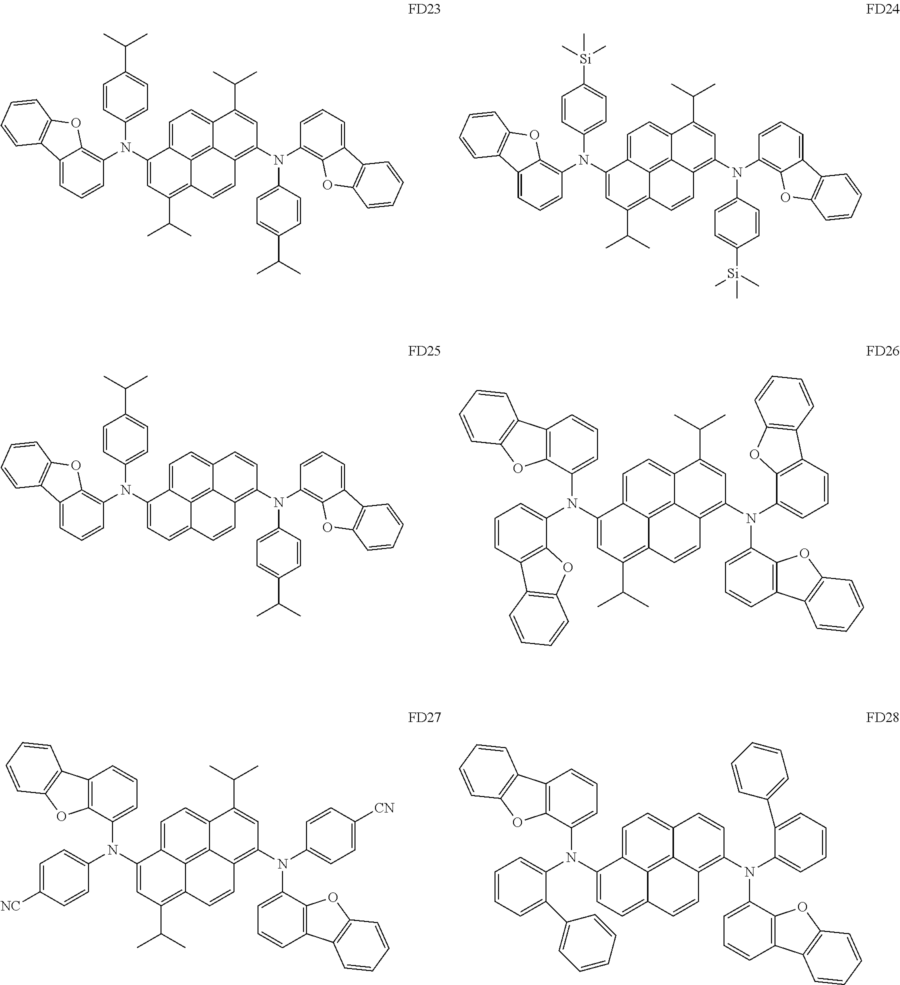

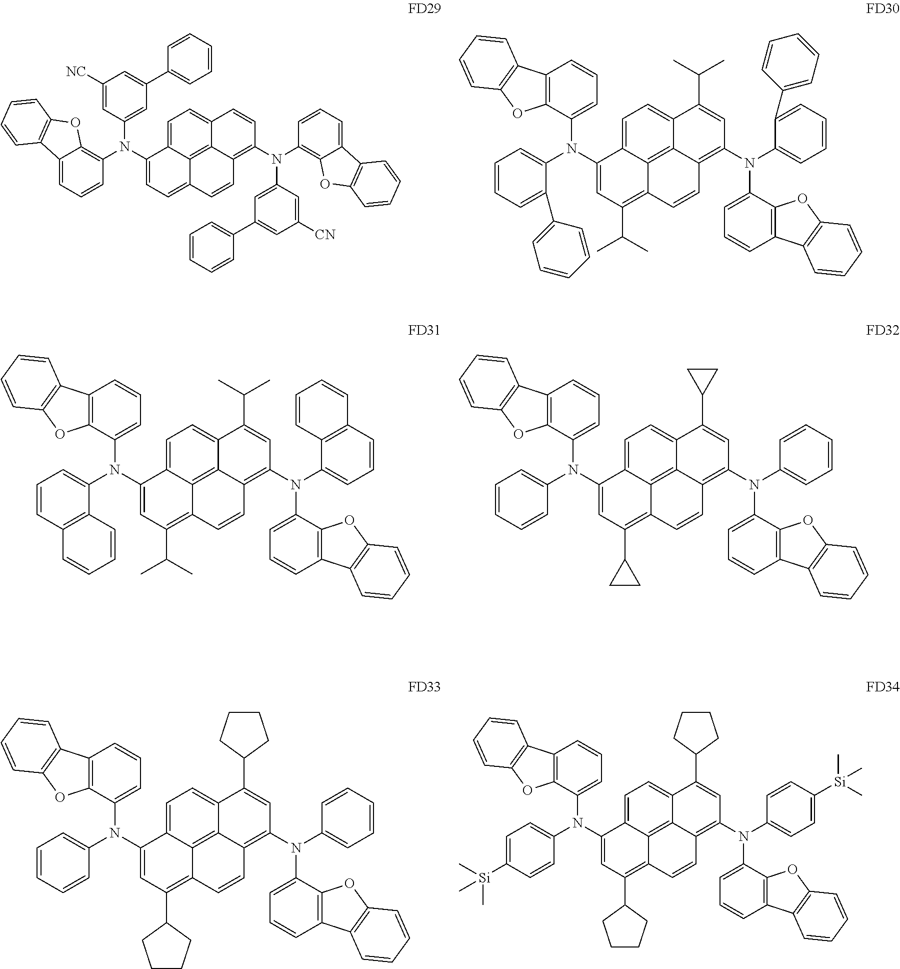

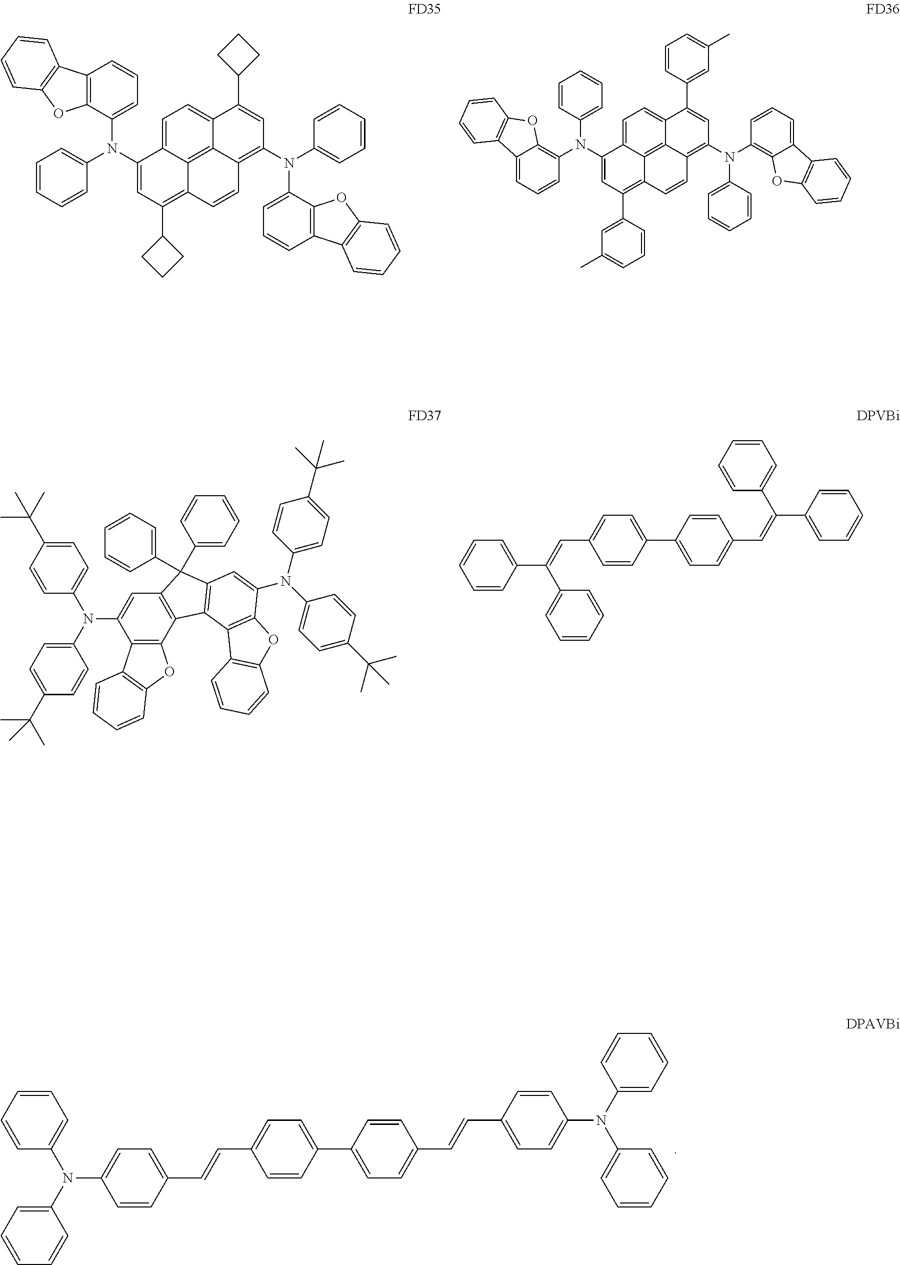

### Delayed Fluorescence Material

The emission layer 130 may include a delayed fluorescence material.

In the present specification, the delayed fluorescence material may be selected from compounds capable of emitting delayed fluorescent light based on a delayed fluorescence emission mechanism.

The delayed fluorescence material included in the emission layer 130 may serve as a host or a dopant depending on the type or kind of other materials included in the emission layer 130.

In an embodiment, a difference between a triplet energy level (eV) of the delayed fluorescence material and a singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

For example, the delayed fluorescence material may include i) a material including at least one electron donor (e.g., a π electron-rich C₃-C₆₀ cyclic group, such as a carbazole group, etc.) and at least one electron acceptor (e.g., a sulfoxide group, a cyano group, a π electron-deficient nitrogen-containing C₁-C₆₀ cyclic group, etc.), and/or ii) a material including a C₈-C₆₀ polycyclic group in which two or more cyclic groups are condensed together while sharing boron (B).

Examples of the delayed fluorescence material may include at least one of Compounds DF1 to DF14:

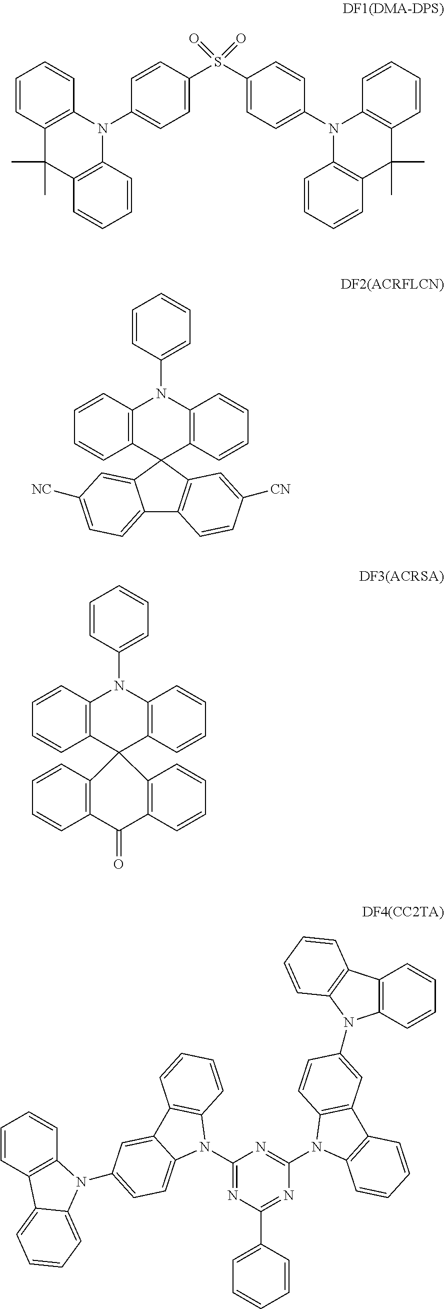

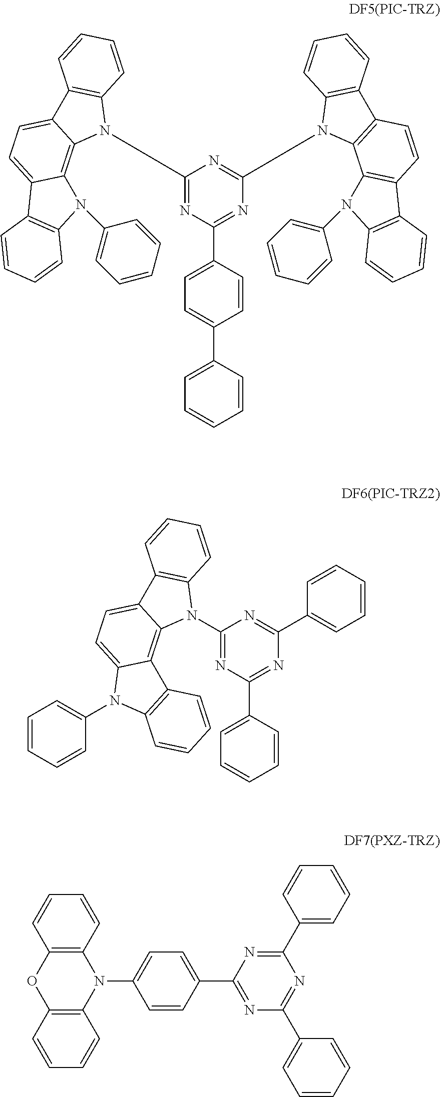 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

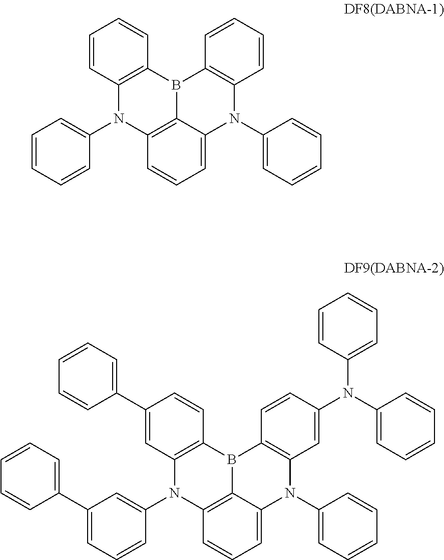 [SMILES: c1ccc(N2c3ccccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

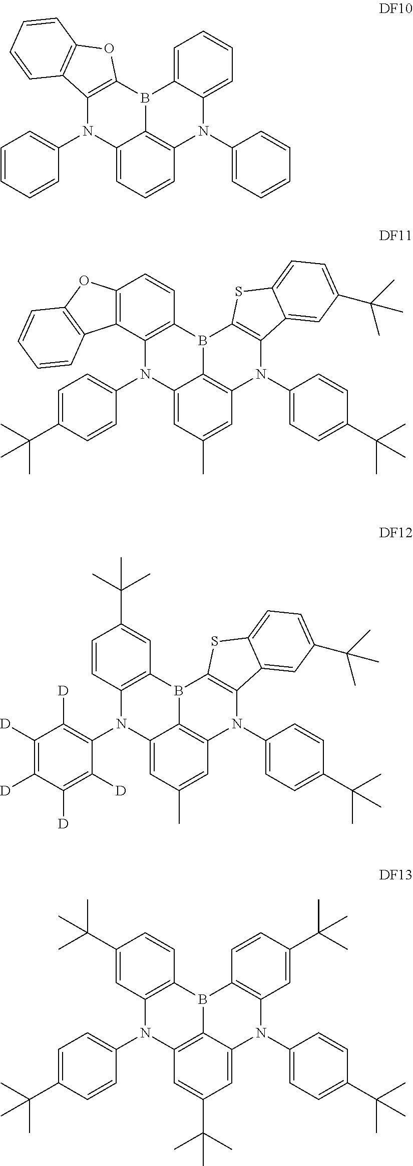

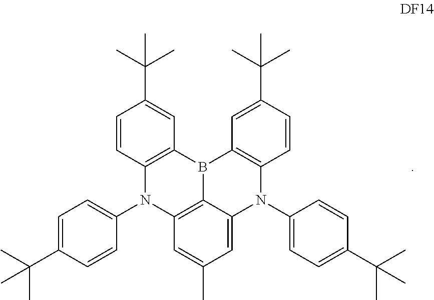 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

The emission layer 130 may include a quantum dot.

The term “quantum dot” as utilized herein refers to a crystal of a semiconductor compound, and may include any material capable of emitting light of one or more suitable emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled or selected through a process with a lower cost, and is easier than vapor deposition methods, such as metal organic chemical vapor deposition (MOCVD) and/or molecular beam epitaxy (MBE).

The quantum dot may include a Group II-VI semiconductor compound, a Group III-V semiconductor compound, a Group III-VI semiconductor compound, a Group I-III-VI semiconductor compound, a Group IV-VI semiconductor compound, a Group IV element or compound, or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, and/or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound, such as GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, and/or InAlPSb; or any combination thereof. In an embodiment, the Group III-V semiconductor compound may further include a Group II element. Examples of the Group III-V semiconductor compound further including a Group II element may include InZnP, InGaZnP, InAlZnP, and/or the like.

Examples of the Group III-VI semiconductor compound may include: a binary compound, such as GaS, GaSe, Ga₂Se₃, GaTe, InS, InSe, In₂S₃, In₂Se₃, and/or InTe; a ternary compound, such as InGaS₃ and/or InGaSes; or any combination thereof.

Examples of the Group I-III-VI semiconductor compound may include: a ternary compound, such as AgInS, AgInS₂, CuInS, CulnS₂, CuGaO₂, AgGaO₂, and/or AgAlO₂; a quaternary compound, such as AgInGaS and/or AgInGaS₂; or any combination thereof.

Examples of the Group IV-VI semiconductor compound may include: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or any combination thereof.

The Group IV element or compound may include: a single element, such as Si and/or Ge; a binary compound, such as SiC and/or SiGe; or any combination thereof.

Each element included in a multi-element compound, such as the binary compound, the ternary compound, and/or the quaternary compound, may be present at a substantially uniform concentration or non-substantially uniform concentration in a particle.

In an embodiment, the quantum dot may have a single structure in which the concentration of each element in the quantum dot is substantially uniform, or a core-shell dual structure. For example, a material included in the core and a material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. An interface between the core and the shell may have a concentration gradient in which the concentration of an element existing in the shell decreases toward the center of the core.

Examples of the shell of the quantum dot may include an oxide of metal, metalloid, or non-metal, a semiconductor compound, or any combination thereof.

Examples of the oxide of metal, metalloid, or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound, such as MgAl₂O₄, CoFe₂O₄, NiFe₂O₄, and/or CoMn₂O₄; or any combination thereof. Examples of the semiconductor compound may include, as described herein, a Group II-VI semiconductor compound, a Group III-V semiconductor compound, a Group III-VI semiconductor compound, a Group I-III-VI semiconductor compound, a Group IV-VI semiconductor compound, or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dot may be about 45 nm or less, for example, about 40 nm or less, or, about 30 nm or less, and within these ranges, color purity or color reproducibility may be improved. In some embodiments, because the light emitted through the quantum dot is emitted in all directions, the viewing angle of light may be improved.

In some embodiments, the quantum dot may be in the form of a spherical particle, a pyramidal particle, a multi-arm particle, a cubic nanoparticle, a nanotube particle, a nanowire particle, a nanofiber particle, or a nanoplate particle.

Because the energy band gap may be adjusted by controlling the size of the quantum dot, light having one or more suitable wavelength bands may be obtained from an emission layer including the quantum dot. Accordingly, by utilizing quantum dots of different sizes, a light-emitting device that emits light of one or more suitable wavelengths may be implemented. In one embodiment, the size of the quantum dot may be selected to emit red, green and/or blue light. In some embodiments, the size of the quantum dot may be configured to emit white light by combining light of one or more suitable colors.

### Photoactive Layer 135

The opto-electronic device 30 may include the photoactive layer 135 arranged on the hole transport region 120. The photoactive layer 135 may be arranged between the hole transport region 120 and the electron transport region 140. In an embodiment, the photoactive layer 135 may be arranged between the hole transport layer included in the hole transport region 120 and the buffer layer included in the electron transport region 140. In one or more embodiments, the photoactive layer 135 may be arranged between the emission auxiliary layer included in the hole transport region 120 and the buffer layer included in the electron transport region 140.

The photoactive layer 135 may include the organic compound and the electron accepting compound. In one embodiment, the organic compound and the electron accepting compound may be mixed and included in the photoactive layer 135. For example, the photoactive layer 135 may be a single layer including the organic compound and the electron accepting compound.

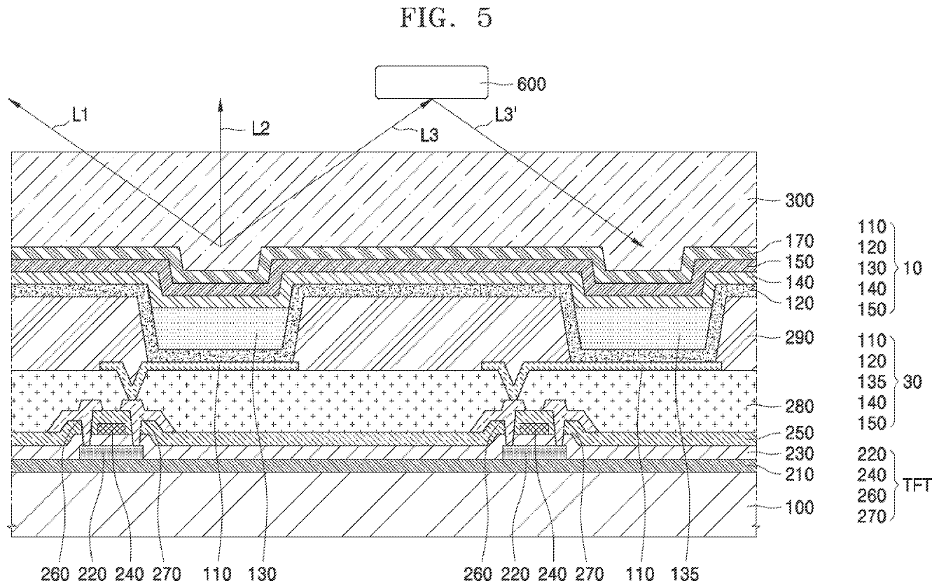

The photoactive layer 135 may be to absorb light incident to the electronic apparatus to form excitons. The excitons may generate holes and electrons. For example, the photoactive layer 135 may be to absorb light to generate an electrical signal. In one embodiment, the organic compound included in the photoactive layer 135 may serve as a donor for supplying electrons, and the electron accepting compound included in the photoactive layer 135 may serve as an acceptor for receiving electrons. Accordingly, the opto-electronic device 30 including the photoactive layer 135 may serve as a photosensor. For example, the opto-electronic device 30 may serve as a fingerprint recognition sensor, which will be described in more detail with reference to FIG. 5.

### Electron Transport Region 140

The electron transport region 140 may be arranged on the emission layer 130 of the light-emitting device 10 and on the photoactive layer 135 of the opto-electronic device 30. The electron transport region 140 may have i) a single-layered structure consisting of a single layer consisting of a single material, ii) a single-layered structure consisting of a single layer including a plurality of different materials, or iii) a multi-layered structure including a plurality of layers including different materials.

The electron transport region 140 may include a buffer layer, a hole blocking layer, an electron control layer, an electron transport layer, an electron injection layer, or any combination thereof.

For example, the electron transport region 140 may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein constituting layers of each structure are stacked sequentially from the emission layer 130 in the respective stated order.

The electron transport region 140 (e.g., the buffer layer, the hole blocking layer, the electron control layer, and/or the electron transport layer in the electron transport region 140) may include a metal-free compound including at least one π electron-deficient nitrogen-containing C₁-C₆₀ cyclic group.

For example, the electron transport region 140 may include a compound represented by Formula 601:

[Ar₆₀₁]ₓₑ₁₁-[(L₆₀₁)ₓₑ₁-R₆₀₁]ₓₑ₂₁ Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be the same as defined herein in connection with Q₁,
xe21 may be 1, 2, 3, 4, or 5,
at least one selected from the group consisting of Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In an embodiment, Ar₆₀₁ in Formula 601 may be a substituted or unsubstituted anthracene group.

In one or more embodiments, the electron transport region 140 may include a compound represented by Formula 601-1:

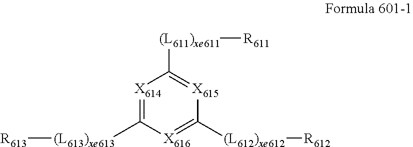

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ may each independently be the same as defined herein in connection with L₆₀₁,
xe611 to xe613 may each be the same as defined herein in connection with xe1,
R₆₁₁ to R₆₁₃ may each independently be the same as defined herein in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region 140 may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

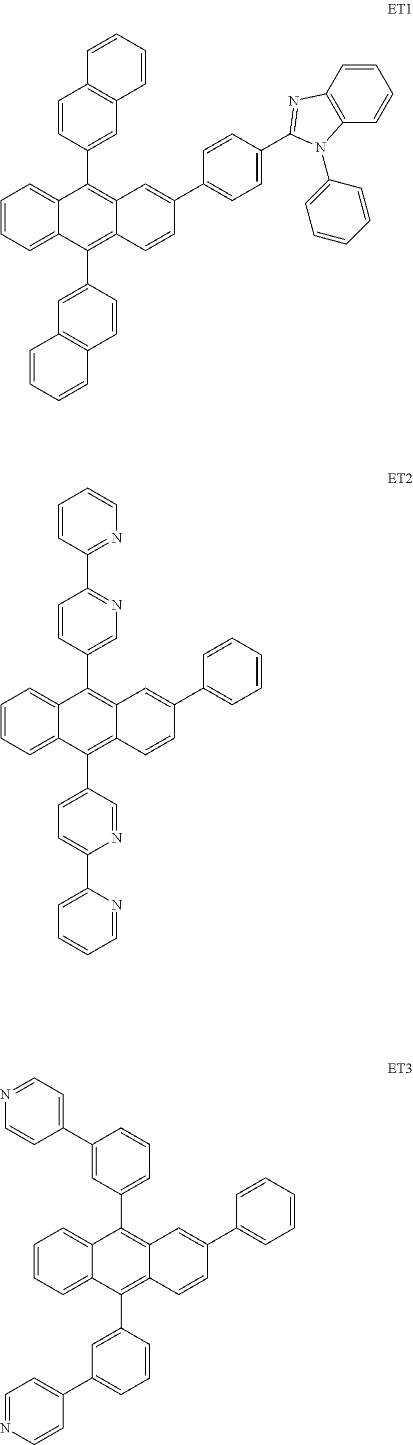 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

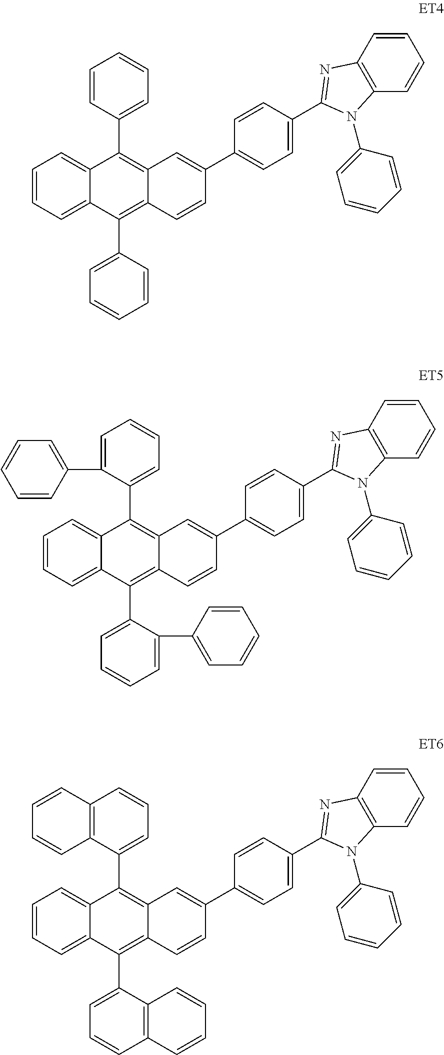 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

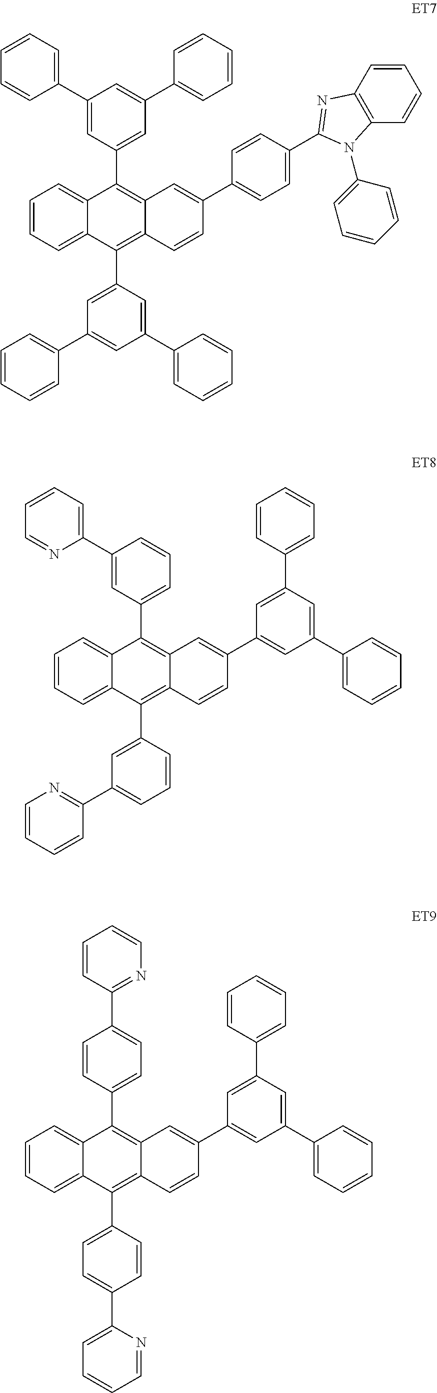 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

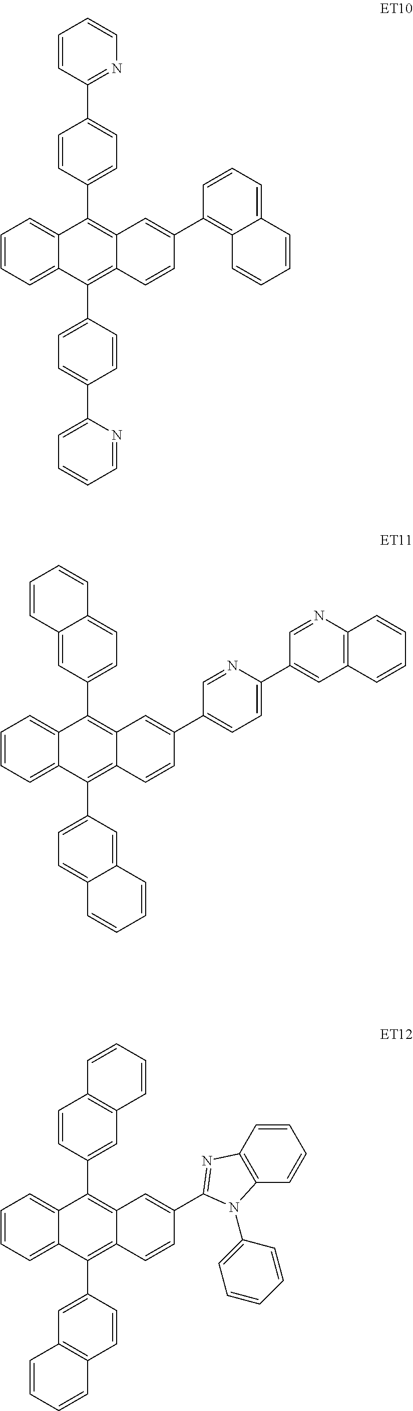 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

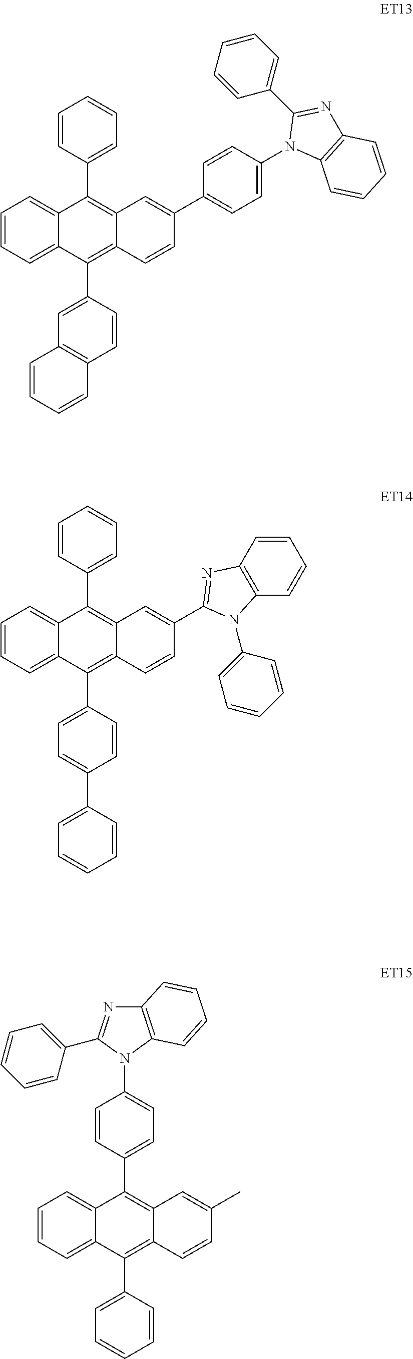 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

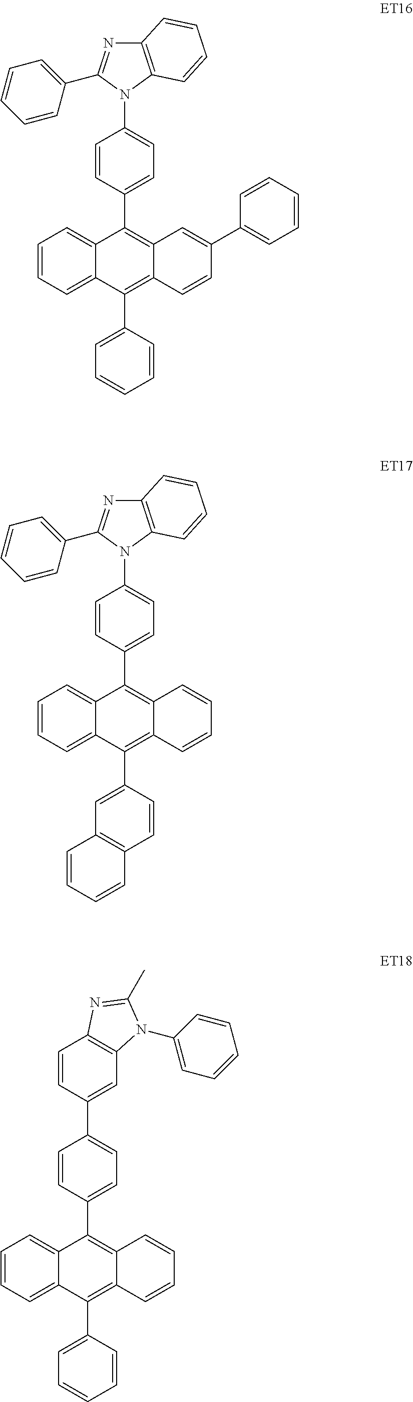 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

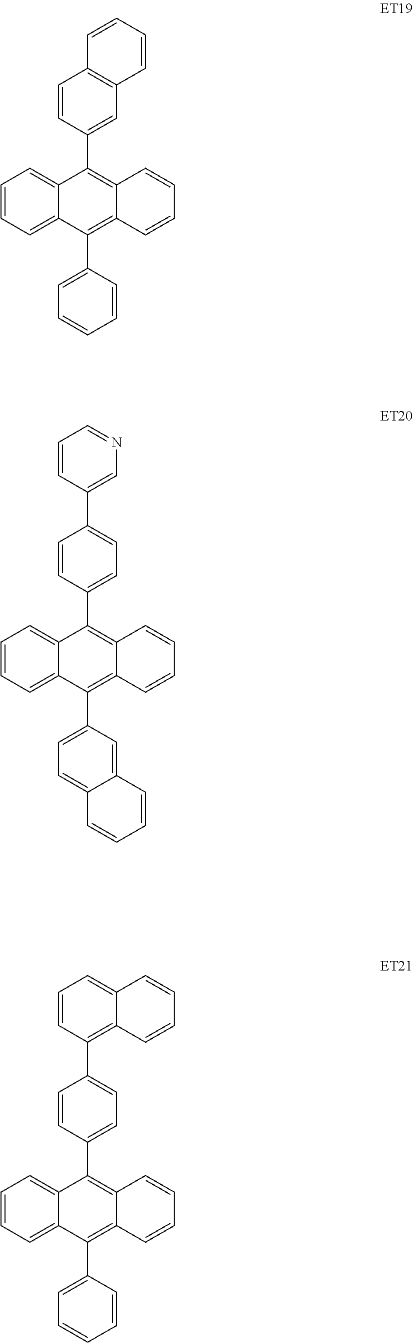

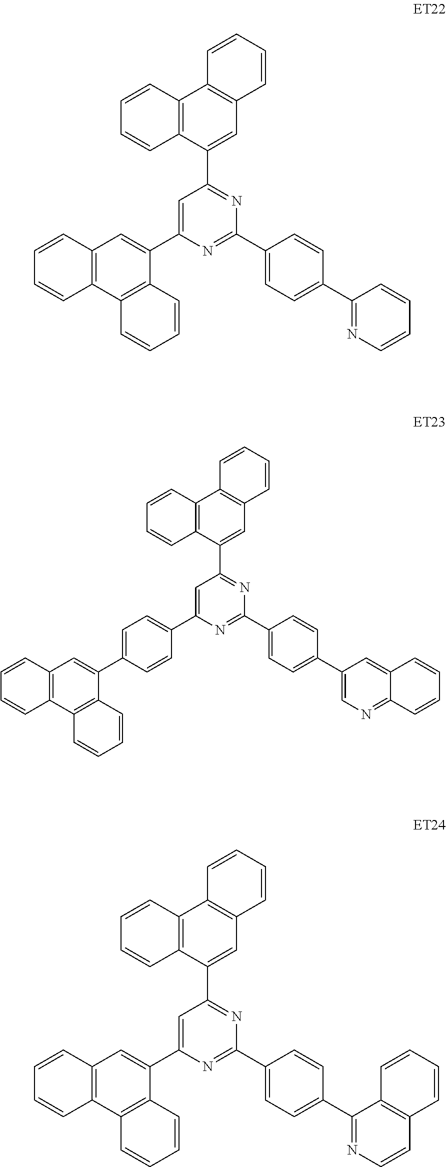 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

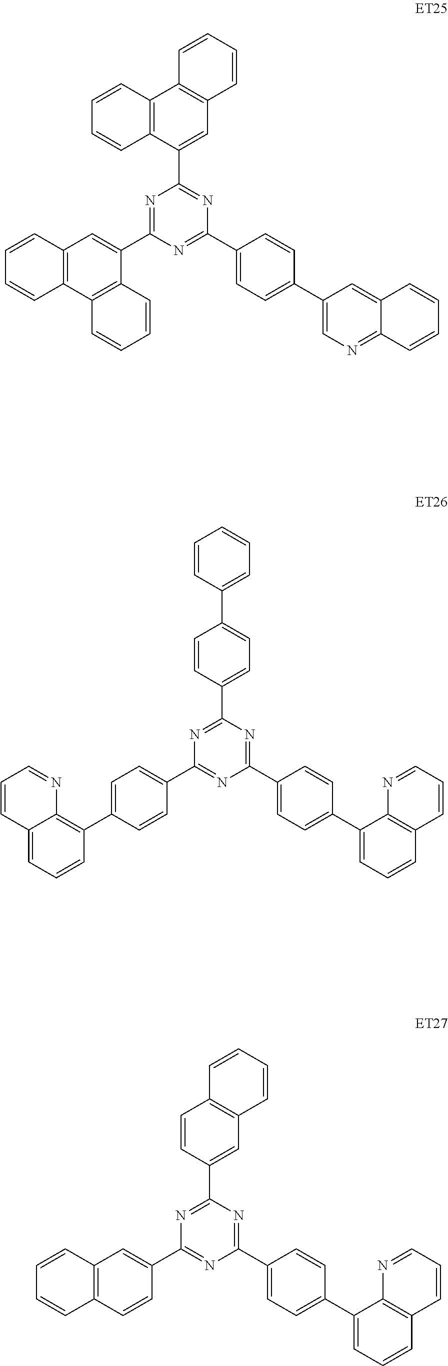 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

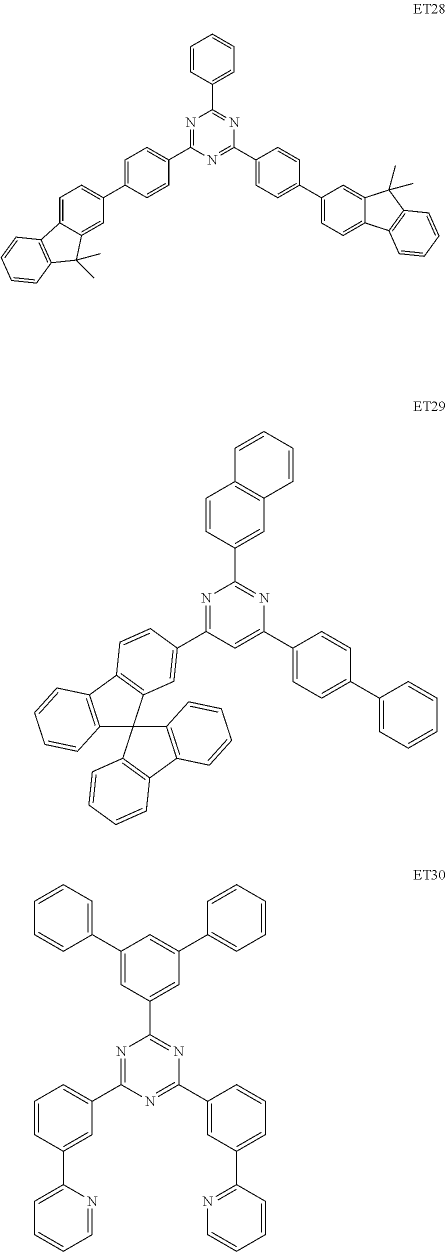 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

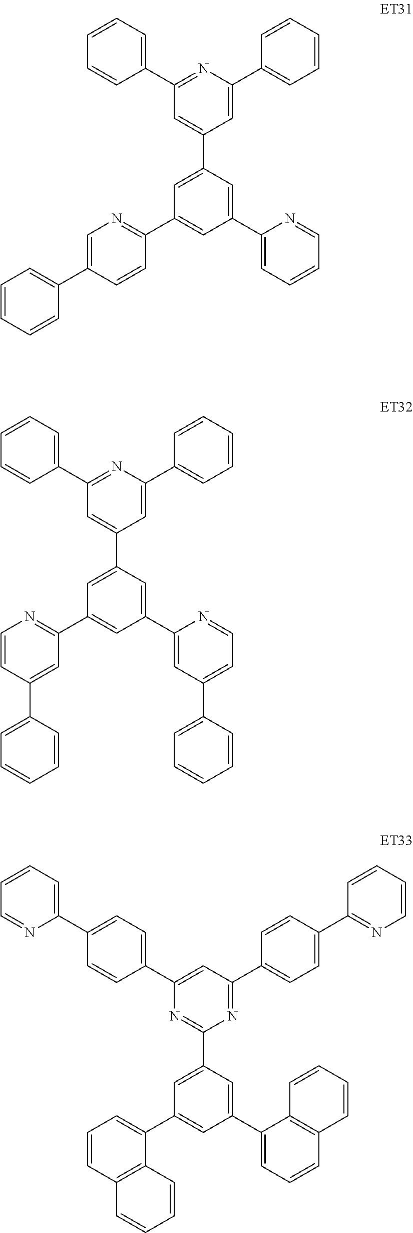 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

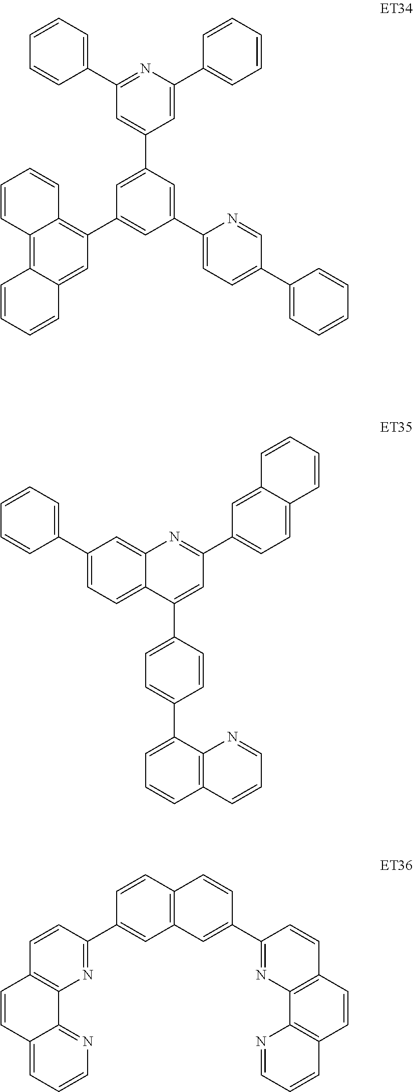 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

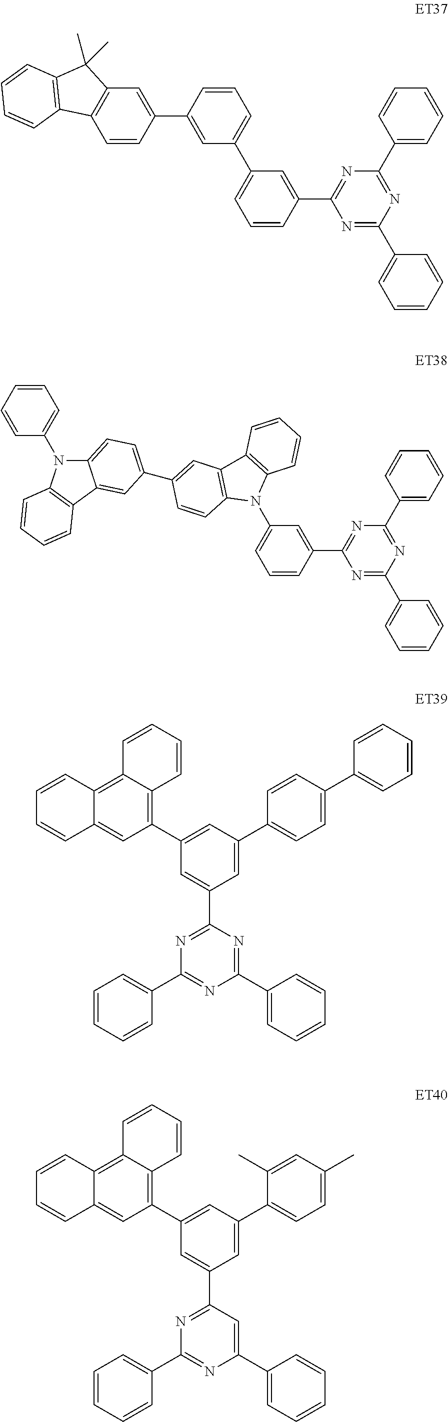 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

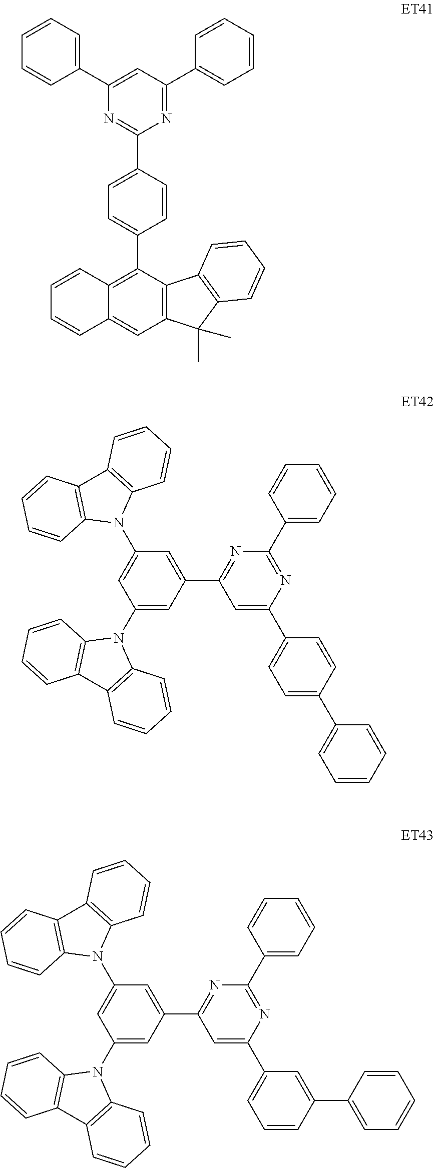 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

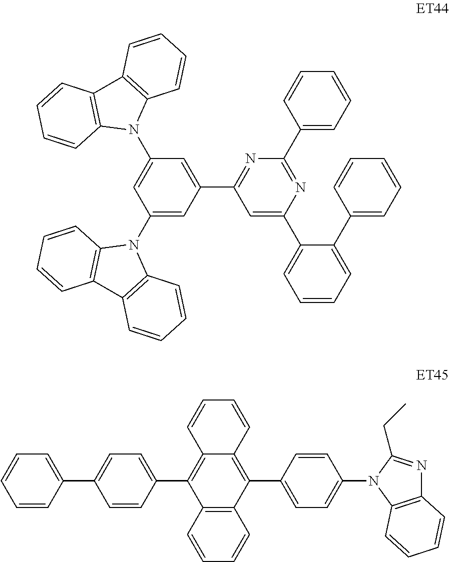 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

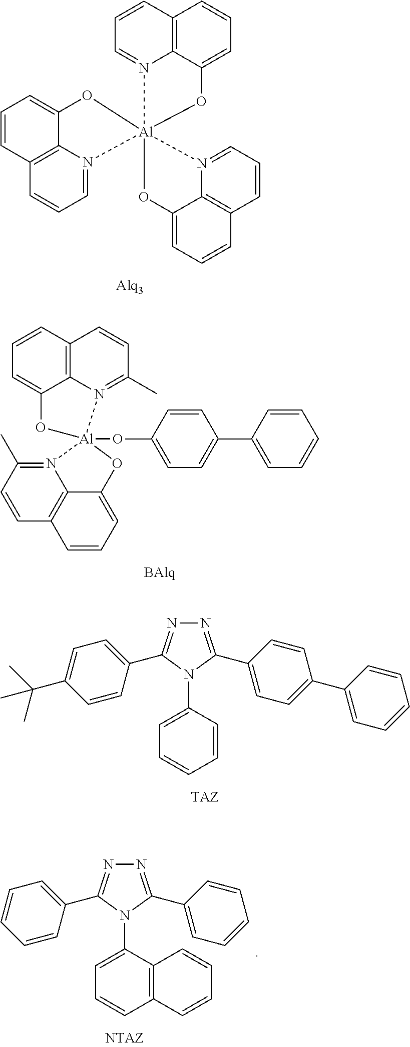

A thickness of the electron transport region 140 may be from about 100 Å to about 5,000 Å, for example, about 160 Å to about 4,000 Å. When the electron transport region 140 includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region 140 are within these ranges, satisfactory electron transporting characteristics may be obtained without a substantial increase in driving voltage.

The electron transport region 140 (e.g., the electron transport layer in the electron transport region 140) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, and/or a Cs ion, and a metal ion of the alkaline earth metal complex may be a Be ion, a Mg ion, a Ca ion, a Sr ion, and/or a Ba ion. A ligand coordinated with the metal ion of the alkali metal complex and/or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, for example, Compound ET-D1 (LiQ) or ET-D2:

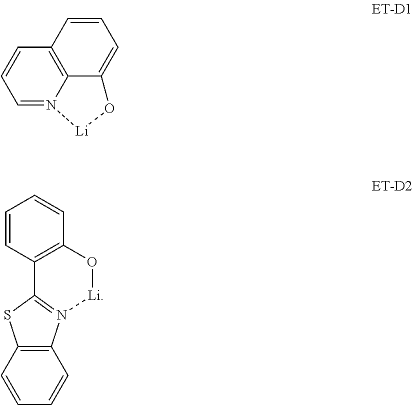

The electron transport region 140 may include an electron injection layer that facilitates injection of electrons from the second electrode 150. The electron injection layer may be in direct contact with the second electrode 150.

The electron injection layer may have i) a single-layered structure consisting of a single layer consisting of a single material, ii) a single-layered structure consisting of a single layer including a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof.

The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include one or more oxides, halides (e.g., fluorides, chlorides, bromides, iodides, etc.), and/or tellurides of the alkali metal, the alkaline earth metal, and the rare earth metal, or any combination thereof.

The alkali metal-containing compound may include an alkali metal oxide, such as Li₂O, Cs₂O, and/or K₂O, an alkali metal halide, such as LiF, NaF, CsF, KF, Lil, NaI, CsI, and/or KI, or any combination thereof. The alkaline earth metal-containing compound may include an alkaline earth metal compound (e.g., oxide), such as BaO, SrO, CaO, BaxSr₁₋ₓO (wherein x is a real number satisfying the condition of 0<x<1), and/or BaxCa₁₋ₓO (wherein x is a real number satisfying the condition of 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In an embodiment, the rare earth metal-containing compound may include a lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) one of ions of the alkali metal, the alkaline earth metal, and the rare earth metal and ii), as a ligand linked to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (e.g., a compound represented by Formula 601).

In an embodiment, the electron injection layer may include (e.g., consist of) i) an alkali metal-containing compound (e.g., an alkali metal halide), or ii) a) an alkali metal-containing compound (e.g., an alkali metal halide); and b) an alkali metal, an alkaline earth metal, a rare earth metal, or any combination thereof. For example, the electron injection layer may be a KI:Yb co-deposited layer, a RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within these ranges, satisfactory electron injection characteristics may be obtained without a substantial increase in driving voltage.

### Second Electrode 150

The second electrode 150 may be arranged on the electron transport region 140. The second electrode 150 may be a cathode, which is an electron injection electrode, and as the material for the second electrode 150, a metal, an alloy, an electrically conductive compound, or any combination thereof, each having a low work function, may be utilized.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be arranged outside the first electrode 110, and/or a second capping layer may be arranged outside the second electrode 150. In one embodiment, the light-emitting device 10 may have a structure in which the first capping layer, the first electrode 110, the emission layer 130, and the second electrode 150 are sequentially stacked in the stated order, a structure in which the first electrode 110, the emission layer 130, the second electrode 150, and the second capping layer are sequentially stacked in the stated order, or a structure in which the first capping layer, the first electrode 110, the emission layer 130, the second electrode 150, and the second capping layer are sequentially stacked in the stated order.

Light generated in the emission layer 130 of the light-emitting device 10 may be extracted (e.g., emitted) toward the outside through the first electrode 110, which is a semi-transmissive electrode or a transmissive electrode, and the first capping layer, or light generated in the emission layer 130 of the light-emitting device 10 may be extracted (e.g., emitted) toward the outside through the second electrode 150, which is a semi-transmissive electrode or a transmissive electrode, and the second capping layer.

The first capping layer and the second capping layer may increase external luminescence efficiency according to the principle of constructive interference. Accordingly, the light extraction efficiency of the light-emitting device 10 may be increased, so that the luminescence efficiency of the light-emitting device 10 may be improved.

Each of the first capping layer and the second capping layer may include a material having a refractive index of 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer or the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In an embodiment, at least one of the first capping layer or the second capping layer may each independently include an amine group-containing compound.

For example, at least one of the first capping layer or the second capping layer may each independently include a compound represented by Formula 201, a compound represented by Formula 202, or any combination thereof.

In one or more embodiments, at least one of the first capping layer or the second capping layer may each independently include one of Compounds HT28 to HT33, one of Compounds CP1 to CP6, β-NPB, or any combination thereof:

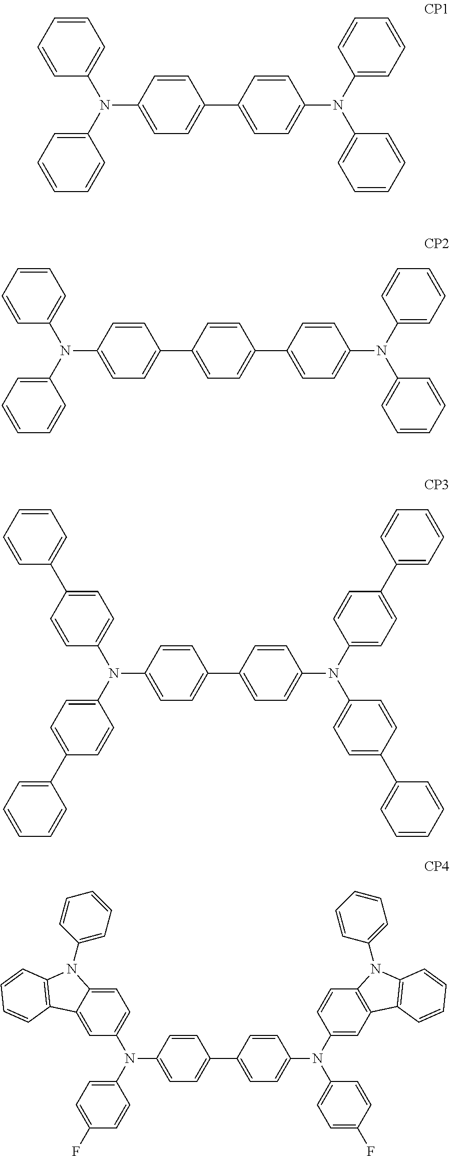 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

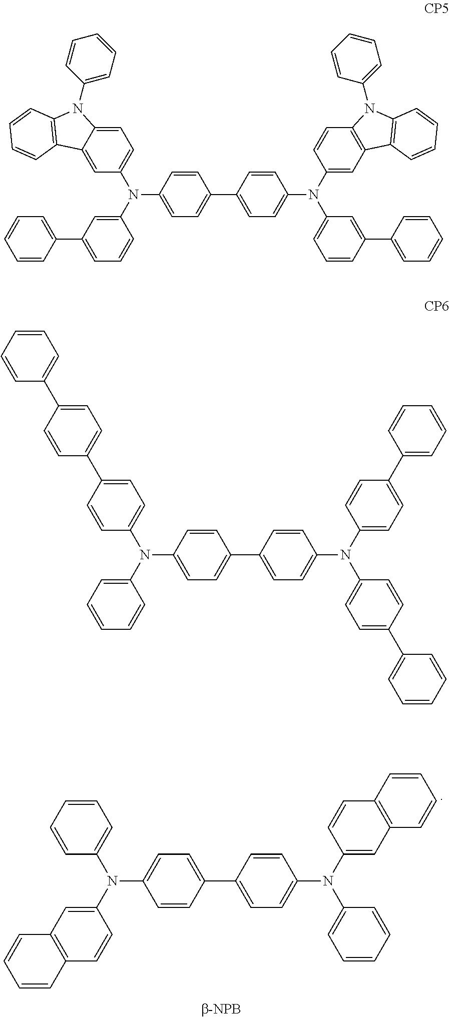 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

A film may be, for example, an optical member (or a light control member) (e.g., a color filter, a color conversion member, a capping layer, a light extraction efficiency improvement layer, a selective light absorption layer, a polarizing layer, a quantum dot-containing layer, etc.), a light blocking member (e.g., a light reflection layer, a light absorption layer, etc.), a protection member (e.g. an insulating layer, a dielectric layer, etc.), and/or the like.

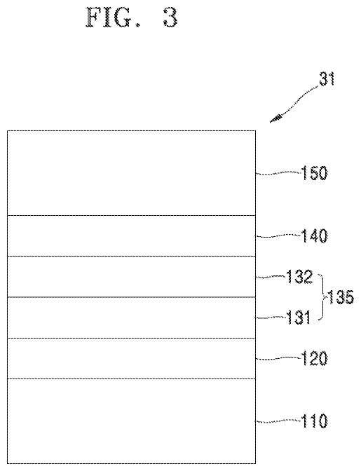

### Description for FIG. 3

FIG. 3 is a schematic view of an opto-electronic device 31 according to another embodiment.

The opto-electronic device 31 shown in FIG. 3 is the same as and/or similar to the opto-electronic device 30 shown in FIG. 1, except for the photoactive layer 135, and thus, descriptions of other components are omitted (e.g., not repeated).

The opto-electronic device 31 may include the photoactive layer 135 arranged between the hole transport region 120 and the electron transport region 140.

In an embodiment, the photoactive layer 135 may be arranged between the hole transport layer included in the hole transport region 120 and the buffer layer included in the electron transport region 140. In one or more embodiments, the photoactive layer 135 may be arranged between the emission auxiliary layer included in the hole transport region 120 and the buffer layer included in the electron transport region 140.

The photoactive layer 135 may include a first layer 131 adjacent to the hole transport region 120 and a second layer 132 adjacent to the electron transport region 140. In an embodiment, the first layer 131 may be in direct contact with the second layer 132.

In an embodiment, the first layer 131 may be in direct contact with the hole transport layer included in the hole transport region 120. In one or more embodiments, the first layer 131 may be in direct contact with the emission auxiliary layer arranged on the hole transport layer.

In an embodiment, the second layer 132 may be in direct contact with the buffer layer included in the electron transport region 140.

The first layer 131 may include the organic compound. For example, the first layer 131 may not include (e.g., may exclude) the electron accepting compound.

The second layer 132 may include the electron accepting compound. For example, the second layer 132 may not include (e.g., may exclude) the organic compound.

For example, the photoactive layer 135 may have a multi-layered structure divided into the first layer 131 including the organic compound and the second layer 132 including the electron accepting compound.

The photoactive layer 135 may be to absorb incident light to form excitons. The excitons may generate holes and electrons. For example, the photoactive layer 135 may be to absorb light to generate an electrical signal. In one embodiment, the organic compound included in the first layer 131 may serve as a donor for supplying electrons, and the electron accepting compound included in the second layer 132 may serve as an acceptor for receiving electrons. Accordingly, the opto-electronic device 31 including the photoactive layer 135 may serve as a photosensor. For example, the opto-electronic device 31 may serve as a fingerprint recognition sensor, which will be described with reference to FIG. 5.

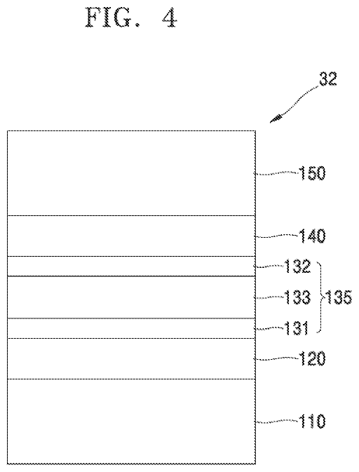

### Description of FIG. 4

FIG. 4 is a schematic view of an opto-electronic device 32 according to another embodiment.

The opto-electronic device 32 shown in FIG. 4 is the same as and/or similar to the opto-electronic device 31 shown in FIG. 3, except for the photoactive layer 135, and thus, descriptions of other components are omitted (e.g., not repeated).

The opto-electronic device 32 may include the photoactive layer 135 arranged between the hole transport region 120 and the electron transport region 140. In an embodiment, the photoactive layer 135 may be arranged between the hole transport layer included in the hole transport region 120 and the buffer layer included in the electron transport region 140. In one or more embodiments, the photoactive layer 135 may be arranged between the emission auxiliary layer included in the hole transport region 120 and the buffer layer included in the electron transport region 140.

The photoactive layer 135 may include the first layer 131 adjacent to the hole transport region 120, the second layer 132 adjacent to the electron transport region 140, and a third layer 133 arranged between the first layer 131 and the second layer 132. In an embodiment, the third layer 133 may be in direct contact with the first layer 131 and/or the second layer 132.

In an embodiment, the first layer 131 may be in direct contact with the hole transport layer included in the hole transport region 120. In one or more embodiments, the first layer 131 may be in direct contact with the emission auxiliary layer arranged on the hole transport layer.

In an embodiment, the second layer 132 may be in direct contact with the buffer layer included in the electron transport region 140.

The first layer 131 may include the organic compound. For example, the first layer 131 may not include (e.g., may exclude) the electron accepting compound.

The second layer 132 may include the electron accepting compound. For example, the second layer 132 may not include (e.g., may exclude) the organic compound.

The third layer 133 may include (e.g., both) the organic compound and the electron accepting compound. For example, the organic compound and the electron accepting compound may be mixed and included in the third layer 133.

For example, the photoactive layer 135 may have a multi-layered structure divided into the first layer 131 including (e.g., only) the organic compound, the third layer 133 including both (e.g., simultaneously) the organic compound and the electron accepting compound, and the second layer 132 including (e.g., only) the electron accepting compound.

The photoactive layer 135 may be to absorb incident light to form excitons. The excitons may generate holes and electrons. For example, the photoactive layer 135 may be to absorb light to generate an electrical signal. In one embodiment, the organic compound included in each of the first layer 131 and the third layer 133 may serve as a donor for supplying electrons, and the electron accepting compound included in each of the second layer 132 and the third layer 133 may serve as an acceptor for receiving electrons. Accordingly, the opto-electronic device 32 including the photoactive layer 135 may serve as a photosensor. For example, the opto-electronic device 32 may serve as a fingerprint recognition sensor, which will be described with reference to FIG. 5.

### Electronic Apparatus

The light-emitting device 10 and the opto-electronic device 30, 31, and/or 32 may be included in one or more suitable electronic apparatuses. For example, the electronic apparatus may be a light-emitting apparatus, an authentication apparatus, and/or the like.

The electronic apparatus (e.g., a light-emitting apparatus) may further include, in addition to the light-emitting device 10 and the opto-electronic device 30, 31, and/or 32, i) a color filter, ii) a color conversion layer, or iii) a color filter and a color conversion layer. The color filter and/or the color conversion layer may be arranged in at least one direction in which light emitted from the light-emitting device 10 travels. For example, the light emitted from the light-emitting device 10 may be blue light or white light. Details on the light-emitting device 10 may be as described above. In an embodiment, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the plurality of subpixel areas, and the color conversion layer may include a plurality of color conversion areas respectively corresponding to the plurality of subpixel areas.

A pixel defining layer may be arranged among the plurality of subpixel areas to define each of the subpixel areas.

The color filter may further include a plurality of color filter areas and light-shielding patterns arranged among the plurality of color filter areas, and the color conversion layer may further include a plurality of color conversion areas and light-shielding patterns arranged among the plurality of color conversion areas.

The plurality of color filter areas (or the plurality of color conversion areas) may include a first area emitting a first color light, a second area emitting a second color light, and/or a third area emitting a third color light, and the first color light, the second color light, and/or the third color light may have different maximum emission wavelengths. For example, the first color light may be red light, the second color light may be green light, and the third color light may be blue light. For example, the plurality of color filter areas (or the plurality of color conversion areas) may include quantum dots. In one embodiment, the first area may include a red quantum dot, the second area may include a green quantum dot, and the third area may not include (e.g., may exclude) a quantum dot. Details on the quantum dot may be as described herein. The first area, the second area, and/or the third area may each further include a scatterer.

For example, the light-emitting device 10 may be to emit a first light, the first area may be to absorb the first light to emit a first-first color light, the second area may be to absorb the first light to emit a second-first color light, and the third area may be to absorb the first light to emit a third-first color light. In this regard, the first-first color light, the second-first color light, and the third-first color light may have different maximum emission wavelengths. In one embodiment, the first light may be blue light, the first-first color light may be red light, the second-first color light may be green light, and the third-first color light may be blue light.

The electronic apparatus may further include a thin-film transistor, in addition to the opto-electronic device(s) 30, 31, and/or 32 and the light-emitting device 10 as described above. The thin-film transistor may include a source electrode, a drain electrode, and an active layer, and the source electrode or the drain electrode may be electrically connected to the first electrode 110 or the second electrode 150 of the light-emitting device 10.

The thin-film transistor may further include a gate electrode, a gate insulating film, and/or the like.

The active layer may include crystalline silicon, amorphous silicon, an organic semiconductor, an oxide semiconductor, and/or the like.

The electronic apparatus may further include a sealing portion for sealing the opto-electronic device 30, 31, and/or 32 and the light-emitting device 10. The sealing portion may be arranged between the color filter and/or the color conversion layer and the light-emitting device 10. The sealing portion may allow light from the light-emitting device 10 to be extracted (e.g., emitted) to the outside, and may concurrently (e.g., simultaneously) prevent or reduce ambient air and/or moisture from penetrating into the opto-electronic device(s) 30, 31, and/or 32 and the light-emitting device 10. The sealing portion may be a sealing substrate including a transparent glass substrate and/or a plastic substrate. The sealing portion may be a thin-film encapsulation layer including at least one layer of an organic layer and/or an inorganic layer. When the sealing portion is a thin-film encapsulation layer, the electronic apparatus may be flexible.

Various suitable functional layers may be additionally arranged on the sealing portion, in addition to the color filter and/or the color conversion layer, according to the usage of the electronic apparatus. Examples of the functional layer may include a touch screen layer, a polarizing layer, and/or the like. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (e.g., fingertips, pupils, etc.).

The authentication apparatus may further include, in addition to the opto-electronic device(s) 30, 31, and/or 32 and the light-emitting device 10 as described above, a biometric information collector.

The electronic apparatus may be applied to one or more suitable displays, light sources, lighting, personal computers (e.g., a mobile personal computer), mobile phones, digital cameras, electronic organizers, electronic dictionaries, electronic game machines, medical instruments (e.g., electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (e.g., meters for a vehicle, an aircraft, and a vessel), projectors, and/or the like.

### Electronic Device

The opto-electronic device(s) 30, 31, and/or 32 may be included in one or more suitable electronic devices.

For example, the electronic device including the opto-electronic device 30, 31, and/or 32 may be at least one of flat panel displays, curved displays, computer monitors, medical monitors, televisions, billboards, indoor or outdoor lightings, signal lights, head-up displays, fully or partially transparent displays, flexible displays, rollable displays, foldable displays, stretchable displays, laser printers, telephones, mobile phones, tablets, phablets, personal digital assistants (PDAs), wearable devices, laptop computers, digital cameras, camcorders, viewfinders, microdisplays, three-dimensional (3D) displays, virtual or augmented-reality displays, vehicles, video walls including multiple displays tiled together, theater or stadium screens, phototherapy devices, and signboards.

Because the opto-electronic device(s) 30, 31, and/or 32 has excellent or suitable photoelectric characteristics, the electronic device including the opto-electronic device(s) 30, 31, and/or 32 may serve as an optical sensor, such as a fingerprint recognition sensor.

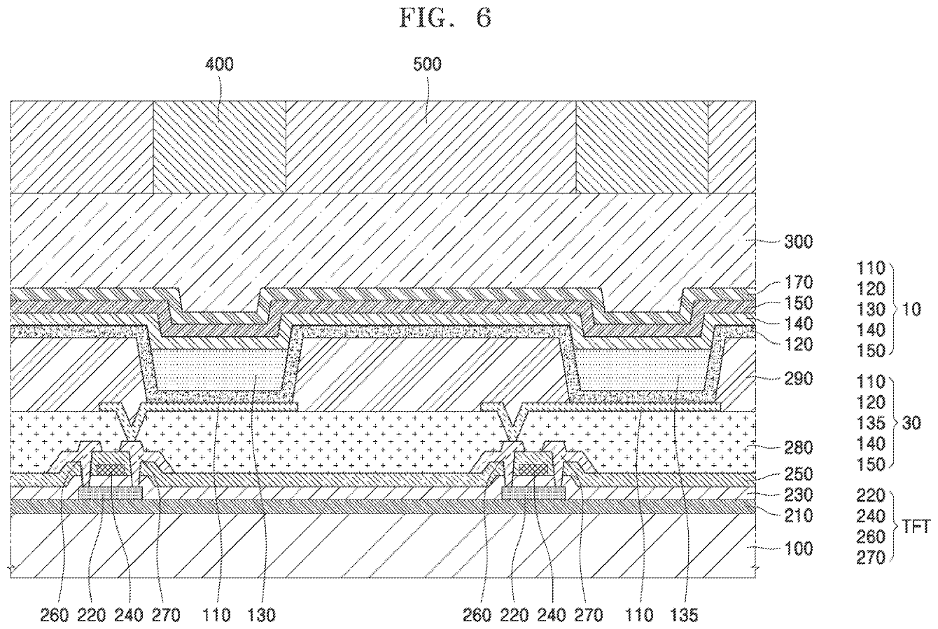

### Description of FIGS. 5 and 6

FIG. 5 is a cross-sectional view of an electronic apparatus according to an embodiment.

The electronic apparatus of FIG. 5 may include a substrate 100, a thin-film transistor TFT, the light-emitting device 10, the opto-electronic device 30, and an encapsulation portion 300. The opto-electronic device 30 of FIG. 5 may be the opto-electronic device 30 described above with reference to FIG. 1, but is not limited thereto. For example, the opto-electronic device 30 of FIG. 5 may be the opto-electronic device 31 of FIG. 3 and/or the opto-electronic device 32 of FIG. 4.

The substrate 100 may be a flexible substrate, a glass substrate, and/or a metal substrate. A buffer layer 210 may be arranged on the substrate 100. The buffer layer 210 may prevent or reduce penetration of impurities through the substrate 100 and may provide a flat surface on the substrate 100.

The thin-film transistor TFT may be arranged on the buffer layer 210. The thin-film transistor TFT may include an active layer 220, a gate electrode 240, a source electrode 260, and a drain electrode 270.

The active layer 220 may include an inorganic semiconductor, such as silicon and/or polysilicon, an organic semiconductor, or an oxide semiconductor, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be arranged on the interlayer insulating film 250. The interlayer insulating film 250 and the gate insulating film 230 may be formed to expose the source region and the drain region of the active layer 220, and the source electrode 260 and the drain electrode 270 may be in contact with the exposed portions of the source region and the drain region of the active layer 220.

The light-emitting device 10 and the opto-electronic device 30 may be arranged on the thin-film transistor TFT.

The thin-film transistor TFT electrically connected to the light-emitting device 10 may be to transmit an electrical signal for driving the light-emitting device 10. The thin-film transistor TFT electrically connected to the opto-electronic device 30 may be to transmit an electrical signal generated by the opto-electronic device 30. The thin-film transistor TFT may be covered and protected by a passivation layer 280. The passivation layer 280 may include an inorganic insulating film, an organic insulating film, or any combination thereof. The light-emitting device 10 and the opto-electronic device 30 may be provided on the passivation layer 280.

The light-emitting device 10 may include the first electrode 110, the hole transport region 120, the emission layer 130, the electron transport region 140, and the second electrode 150. The opto-electronic device 30 may include the first electrode 110, the hole transport region 120, the photoactive layer 135, the electron transport region 140, and the second electrode 150.

The first electrode 110 may be arranged on the passivation layer 280. The passivation layer 280 may expose certain regions of the source electrode 260 and the drain electrode 270 without completely covering the source electrode 260 and the drain electrode 270, and the first electrode 110 may be connected to the exposed regions of the source electrode 260 and the drain electrode 270.

A pixel defining layer 290 including an insulating material may be arranged on the first electrode 110. The pixel defining layer 290 may expose a certain region of the first electrode 110. The pixel defining layer 290 may be a polyimide or polyacrylic organic film.

The hole transport region 120 may be arranged on the pixel defining layer 290. The hole transport region 120 included in the light-emitting device 10 and the hole transport region 120 included in the opto-electronic device 30 may be integrally formed as a single body. The hole transport region 120 included in the light-emitting device 10 and the hole transport region 120 included in the opto-electronic device 30 may be arranged on the pixel defining layer 290, may be connected to each other, may include substantially the same material, and may be formed substantially at the same time.

Each of the emission layer 130 and the photoactive layer 135 may be arranged on the hole transport region 120. Each of the emission layer 130 and the photoactive layer 135 may overlap the certain region of the first electrode 110 that is exposed by the pixel defining layer 290.

The electron transport region 140 may be arranged on the emission layer 130 and the photoactive layer 135. The electron transport region 140 included in the light-emitting device 10 and the electron transport region 140 included in the opto-electronic device 30 may be integrally formed as a single body. The electron transport region 140 included in the light-emitting device 10 and the electron transport region 140 included in the opto-electronic device 30 may be arranged on the pixel defining layer 290, may be connected to each other, may include substantially the same material, and may be formed substantially at the same time.

The second electrode 150 may be arranged on the electron transport region 140. The second electrode 150 included in the light-emitting device 10 and the second electrode 150 included in the opto-electronic device 30 may be integrally formed as a single body. The second electrode 150 included in the light-emitting device 10 and the second electrode 150 included in the opto-electronic device 30 may be arranged on the pixel defining layer 290, may be connected to each other, may include substantially the same material, and may be formed substantially at the same time.

A capping layer 170 may be additionally formed on the second electrode 150. The capping layer 170 may be formed to cover the second electrode 150.

The encapsulation portion 300 may be arranged on the capping layer 170. The encapsulation portion 300 may be arranged on the light-emitting device 10 and the opto-electronic device 30 to protect the light-emitting device 10 and the opto-electronic device 30 from moisture or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (e.g., polymethyl methacrylate, polyacrylic acid, etc.), an epoxy-based resin (e.g., aliphatic glycidyl ether (AGE), etc.), or any combination thereof; or a combination of the inorganic film and the organic film.

The light-emitting device 10 may be to emit light L1, L2, and L3. For example, the light L1, L2, and L3 may each be green light.

The light L3 from among the lights L1, L2, and L3 that have been emitted may be incident on an object 600 outside the electronic apparatus. For example, the object 600 may be a finger of a user of the electronic apparatus. A light L3′ reflected by the object 600 may be incident on the opto-electronic device 30.

The photoactive layer 135 may be to absorb the incident light L₃′ to form excitons. The excitons may generate holes and electrons. For example, the photoactive layer 135 may be to absorb light to generate an electrical signal. In one embodiment, the organic compound included in the photoactive layer 135 may serve as a donor for supplying electrons, and the electron accepting compound included in the photoactive layer 135 may serve as an acceptor for receiving electrons. For example, the opto-electronic device 30 may detect energy of the light L₃′ and convert the detected energy into an electrical signal. Accordingly, the opto-electronic device 30 may recognize the object 600 that has come into contact with (or approached) the electronic apparatus. Accordingly, the opto-electronic device 30 including the photoactive layer 135 may serve as an optical sensor (e.g., a fingerprint recognition sensor).

FIG. 6 is a cross-sectional view of an electronic apparatus according to another embodiment.

The electronic apparatus of FIG. 6 is the same as the electronic apparatus of FIG. 5, except that a light shielding pattern 500 and a functional region 400 are additionally arranged on the encapsulation portion 300. The functional region 400 may be i) a color filter area, ii) a color conversion area, or iii) a combination of the color filter area and the color conversion area. In an embodiment, the light-emitting device included in the electronic apparatus of FIG. 6 may be a tandem light-emitting device.

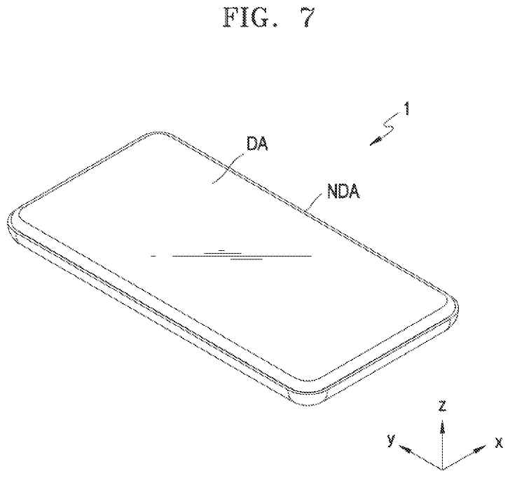

### Description of FIG. 7

FIG. 7 is a schematic perspective view of an electronic device 1 including an opto-electronic device according to an embodiment. The electronic device 1 may be an apparatus that displays a moving image and/or a still image, and may include a portable electronic device, such as a mobile phone, a smart phone, a tablet personal computer (PC), a mobile communication terminal, an electronic notebook, an electronic book, a portable multimedia player (PMP), a navigation device, and/or an ultra mobile PC (UMPC), as well as one or more suitable products, such as a television, a laptop, a monitor, a billboard, and/or an internet of things (IOT) device, and/or a part thereof. In some embodiments, the electronic device 1 may be a wearable device, such as a smart watch, a watch phone, a glasses-type or kind display, and/or a head mounted display (HMD), and/or a part thereof. However, embodiments of the present disclosure are not limited thereto. For example, the electronic device 1 may be an instrument panel of a vehicle, a center information display (CID) arranged on a center fascia or a dashboard of a vehicle, a room mirror display that replaces a side mirror of a vehicle, an entertainment display for a rear seat of a vehicle or a display arranged on a rear surface of a front seat, a head up display (HUD) installed at a front of a vehicle or projected on a front window glass, and/or a computer generated hologram augmented reality head up display (CGH AR HUD). FIG. 7 shows a case in which the electronic device 1 is a smart phone, for convenience of description.

The electronic device 1 may include a display area DA and a non-display area NDA outside the display area DA. The electronic device 1 may implement an image through an array of a plurality of pixels that are two-dimensionally arranged in the display area DA.

The non-display area NDA is an area in which an image is not displayed, and may be around (e.g., entirely surround) the display area DA. A driver for providing electrical signals and/or power to display elements arranged in the display area DA may be arranged in the non-display area NDA. A pad, which is an area to which an electronic element and/or a printed circuit board may be electrically connected, may be arranged in the non-display area NDA.

The electronic device 1 may have different lengths in the x-axis direction and in the y-axis direction. For example, as shown in FIG. 7, the length in the x-axis direction may be shorter than the length in the y-axis direction. In one or more embodiments, the length in the x-axis direction may be the same as the length in the y-axis direction. In one or more embodiments, the length in the x-axis direction may be longer than the length in the y-axis direction.

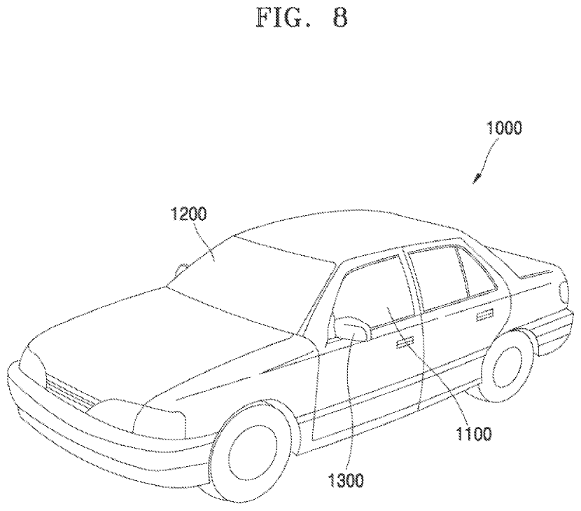

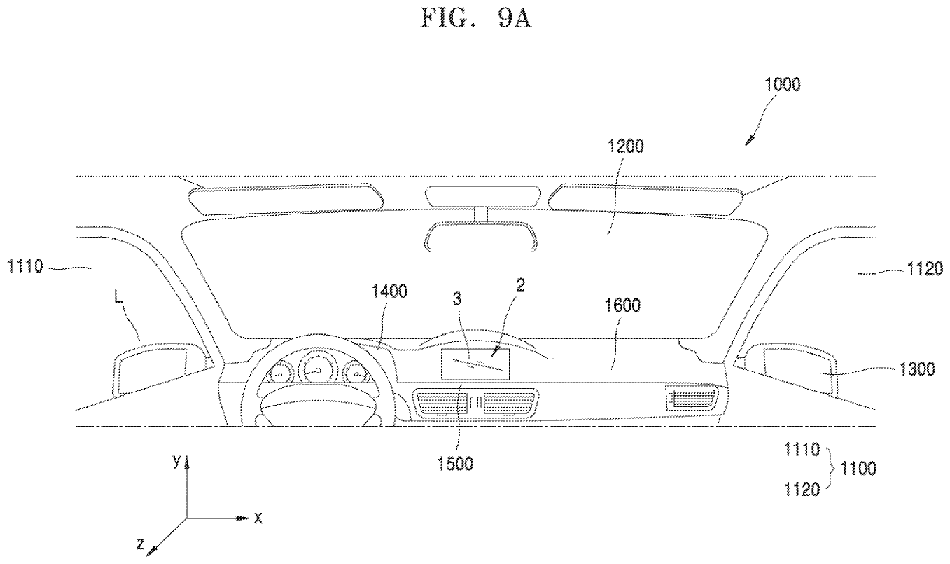

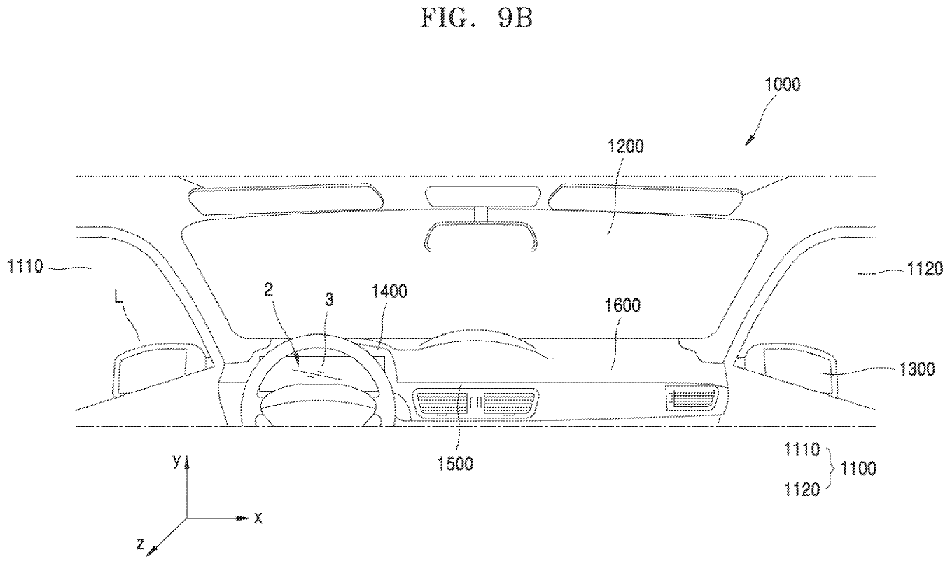

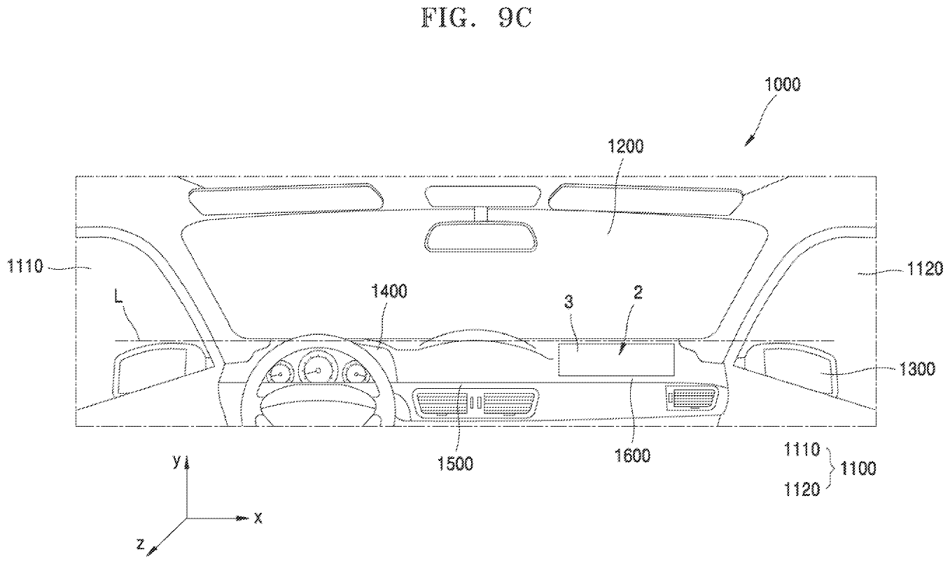

### Description of FIGS. 8 and 9A-9C

FIG. 8 is a schematic view of the exterior of a vehicle 1000 as an electronic device including an opto-electronic device according to an embodiment. FIGS. 9A-9C are schematic views of the interior of the vehicle 1000 according to one or more suitable embodiments.

Referring to FIGS. 8, 9A, 9B, and 9C, the vehicle 1000 may refer to one or more suitable apparatuses for moving a subject to be transported, such as a human, an object, and/or an animal, from a departure point to a destination. The vehicle 1000 may include a vehicle traveling on a road and/or a track, a vessel moving over the sea and/or a river, an airplane flying in the sky by utilizing the action of air, and/or the like.

The vehicle 1000 may travel on a road and/or a track. The vehicle 1000 may move in a certain direction according to rotation of at least one wheel. For example, the vehicle 1000 may include a three-wheeled or four-wheeled vehicle, a construction machine, a two-wheeled vehicle, a prime mover apparatus, a bicycle, and/or a train running on a track.

The vehicle 1000 may include a body having an interior and an exterior, and a chassis in which mechanical apparatuses necessary for driving are installed as the remaining parts except for the body. The exterior of the body may include a front panel, a bonnet, a roof panel, a rear panel, a trunk, and/or a pillar provided at a boundary between doors. The chassis of the vehicle 1000 may include a power generating apparatus, a power transmitting apparatus, a driving apparatus, a steering apparatus, a braking apparatus, a suspension apparatus, a transmission apparatus, a fuel apparatus, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on the side of the vehicle 1000. In an embodiment, the side window glass 1100 may be installed on a door of the vehicle 1000. A plurality of side window glasses 1100 may be provided and may face each other. In an embodiment, the side window glass 1100 may include a first side window glass 1110 and a second side window glass 1120. In an embodiment, the first side window glass 1110 may be arranged adjacent to the cluster 1400. The second side window glass 1120 may be arranged adjacent to the passenger seat dashboard 1600.

In an embodiment, the side window glasses 1100 may be apart from each other in the x-direction or the −x-direction. For example, the first side window glass 1110 and the second side window glass 1120 may be apart from each other in the x direction or the −x direction. In other words, an imaginary straight line L connecting the side window glasses 1100 to each other may extend in the x direction or the −x direction. For example, the imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a view of the rear of the vehicle 1000. The side mirror 1300 may be installed on the exterior of the body. In an embodiment, a plurality of side mirrors 1300 may be provided. One of the plurality of side mirrors 1300 may be arranged outside the first side window glass 1110. Another one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge, a turn indicator, a high beam indicator, a warning lamp, a seat belt warning lamp, an odometer, a hodometer, an automatic transmission selection lever indicator, a door open warning lamp, an engine oil warning lamp, and/or a low fuel warning lamp.

The center fascia 1500 may include a control panel on which a plurality of buttons for adjusting an audio apparatus, an air conditioning apparatus, and a heater of a seat are arranged. The center fascia 1500 may be arranged on one side of the cluster 1400.

The passenger seat dashboard 1600 may be apart from the cluster 1400 with the center fascia 1500 therebetween. In an embodiment, the cluster 1400 may be arranged to correspond to a driver seat, and the passenger seat dashboard 1600 may be arranged to correspond to a passenger seat. In an embodiment, the cluster 1400 may be adjacent to the first side window glass 1110, and the passenger seat dashboard 1600 may be adjacent to the second side window glass 1120.

In an embodiment, the display apparatus 2 may include a display panel 3, and the display panel 3 may display an image. The display apparatus 2 may be arranged inside the vehicle 1000. In an embodiment, the display apparatus 2 may be arranged between the side window glasses 1100 facing each other. The display apparatus 2 may be arranged on at least one of the cluster 1400, the center fascia 1500, or the passenger seat dashboard 1600.

The display apparatus 2 may include an organic light-emitting display apparatus, an inorganic electroluminescent (EL) display apparatus, a quantum dot display apparatus, and/or the like. Hereinafter, as the display apparatus 2 according to an embodiment, an organic light-emitting display apparatus including the light-emitting device according to an embodiment will be described as an example, but one or more suitable types (kinds) of display apparatuses as described above may be utilized in embodiments.

Referring to FIG. 9A, the display apparatus 2 may be arranged on the center fascia 1500. In an embodiment, the display apparatus 2 may display navigation information. In an embodiment, the display apparatus 2 may display audio, video, or information regarding vehicle settings.

Referring to FIG. 9B, the display apparatus 2 may be arranged on the cluster 1400. In this case, the cluster 1400 may display driving information and/or the like through the display apparatus 2. For example, the cluster 1400 may be digitally implemented. The digital cluster 1400 may display vehicle information and driving information as images. For example, a needle and a gauge of a tachometer and one or more suitable warning lamp icons may be displayed by digital signals.

Referring to FIG. 9C, the display apparatus 2 may be arranged on the passenger seat dashboard 1600. The display apparatus 2 may be embedded in the passenger seat dashboard 1600 or arranged on the passenger seat dashboard 1600. In an embodiment, the display apparatus 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information that is different from information displayed on the cluster 1400 and/or information displayed on the center fascia 1500.

### Manufacturing Method

Respective layers included in the hole transport region 120, the emission layer 130, the photoactive layer 135, and respective layers included in the electron transport region 140 may be formed in a certain region by utilizing one or more suitable methods selected from vacuum deposition, spin coating, casting, Langmuir-Blodgett (LB) deposition, ink-jet printing, laser-printing, laser-induced thermal imaging, and/or the like.

When respective layers included in the hole transport region 120, the emission layer 130, the photoactive layer 135, and respective layers included in the electron transport region 140 are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed. Because the organic compound, in which a first moiety represented by Formula 1 and a second moiety represented by Formula 2 are bonded to each other, has excellent or suitable heat resistance characteristics, when the first layer 131 included in the photoactive layer 135 is formed utilizing the organic compound by vacuum deposition, the first layer 131 may be easily formed without defects.

### Definition of Terms

The term “C₃-C₆₀ carbocyclic group” as used herein refers to a cyclic group consisting of only carbon atoms as ring-forming atoms and having 3 to 60 carbon atoms, and the term “C₁-C₆₀ heterocyclic group” as used herein refers to a cyclic group that has, in addition to 1 to 60 carbon atoms, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the number of ring-forming atoms of the C₁-C₆₀ heterocyclic group may be 3 to 61.

The term “cyclic group” as used herein may include the C₃-C₆₀ carbocyclic group and the C₁-C₆₀ heterocyclic group.

The term “π electron-rich C₃-C₆₀ cyclic group” as used herein refers to a cyclic group that has 3 to 60 carbon atoms and does not include *—N═*′ as a ring-forming moiety, and the term “π electron-deficient nitrogen-containing C₁-C₆₀ cyclic group” as used herein refers to a heterocyclic group that has 1 to 60 carbon atoms and includes *—N═*′ as a ring-forming moiety.

For example,
the C₃-C₆₀ carbocyclic group may be i) a group T1 or ii) a condensed cyclic group in which two or more groups T1 are condensed with each other (e.g., the C₃-C₆₀ carbocyclic group may be a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a group T2, ii) a condensed cyclic group in which two or more groups T2 are condensed with each other, or iii) a condensed cyclic group in which at least one group T2 and at least one group T1 are condensed with each other (e.g., the C₁-C₆₀ heterocyclic group may be a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) a group T1, ii) a condensed cyclic group in which two or more groups T1 are condensed with each other, iii) a group T3, iv) a condensed cyclic group in which two or more groups T3 are condensed with each other, or v) a condensed cyclic group in which at least one group T3 and at least one group T1 are condensed with each other (e.g., the π electron-rich C₃-C₆₀ cyclic group may be the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.), and
the π electron-deficient nitrogen-containing C₁-C₆₀ cyclic group may be i) a group T4, ii) a condensed cyclic group in which two or more groups T4 are condensed with each other, iii) a condensed cyclic group in which at least one group T4 and at least one group T1 are condensed with each other, iv) a condensed cyclic group in which at least one group T4 and at least one group T3 are condensed with each other, or v) a condensed cyclic group in which at least one group T4, at least one group T1, and at least one group T3 are condensed with each other (e.g., the π electron-deficient nitrogen-containing C₁-C₆₀ cyclic group may be a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
wherein the group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
the group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the group T3 may be a furan group, a thiophene group, a 1H-pyrrole group, a silole group, or a borole group, and
the group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group,” “C₃-C₆₀ carbocyclic group,” “C₁-C₆₀ heterocyclic group,” “π electron-rich C₃-C₆₀ cyclic group,” or “π electron-deficient nitrogen-containing C₁-C₆₀ cyclic group” as used herein each refers to a group condensed with any cyclic group, a monovalent group, or a polyvalent group (e.g., a divalent group, a trivalent group, a tetravalent group, etc.), depending on the structure of a formula in connection with which the terms are used. For example, the “benzene group” may be a benzo group, a phenyl group, a phenylene group, and/or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁ cycloalkyl group, a C₁-C₁₀ heterocycloalkyl group, a C₃-C₁ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and examples of the divalent C₃-C₆₀ carbocyclic group and the divalent C₁-C₆₀ heterocyclic group may include a C₃-C₁ cycloalkylene group, a C₁-C₁₀ heterocycloalkylene group, a C₃-C₁ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has 1 to 60 carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and/or the like. The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle and/or at a terminal end (e.g., the terminus) of the C₂-C₆₀ alkyl group, and examples thereof may include an ethenyl group, a propenyl group, a butenyl group, and/or the like. The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle and/or at a terminal end (e.g., the terminus) of the C₂-C₆₀ alkyl group, and examples thereof may include an ethynyl group, a propynyl group, and/or the like. The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof may include a methoxy group, an ethoxy group, an isopropyloxy group, and/or the like.

The term “C₃-C₁₀ cycloalkyl group” as used herein refers to a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or a bicyclo[2.2.1]heptyl group), a bicyclo[1.1.1]pentyl group, a bicyclo[2.1.1]hexyl group, a bicyclo[2.2.2]octyl group, and/or the like. The term “C₃-C₁₀ cycloalkylene group” as used herein refers to a divalent group having substantially the same structure as the C₃-C₁ cycloalkyl group.

The term “C₁-C₁₀ heterocycloalkyl group” as used herein refers to a monovalent cyclic group that has, in addition to 1 to 10 carbon atoms, at least one heteroatom as a ring-forming atom, and examples thereof may include a 1,2,3,4-oxatriazolidinyl group, a tetrahydrofuranyl group, a tetrahydrothiophenyl group, and/or the like. The term “C₁-C₁₀ heterocycloalkylene group” as used herein refers to a divalent group having substantially the same structure as the C₁-C₁₀ heterocycloalkyl group.

The term “C₃-C₁ cycloalkenyl group” as used herein refers to a monovalent cyclic group that has 3 to 10 carbon atoms and at least one carbon-carbon double bond in the ring thereof and no aromaticity, and examples thereof may include a cyclopentenyl group, a cyclohexenyl group, a cycloheptenyl group, and/or the like. The term “C₃-C₁₀ cycloalkenylene group” as used herein refers to a divalent group having substantially the same structure as the C₃-C₁₀ cycloalkenyl group.

The term “C₁-Cia heterocycloalkenyl group” as used herein refers to a monovalent cyclic group that has, in addition to 1 to 10 carbon atoms, at least one heteroatom as a ring-forming atom, and at least one double bond in the ring thereof. Examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and/or the like. The term “C₁-C₁₀ heterocycloalkenylene group” as used herein refers to a divalent group having substantially the same structure as the C₁-Cia heterocycloalkenyl group.

The term “C₆-C₆₀ aryl group” as used herein refers to a monovalent group having a carbocyclic aromatic system of 6 to 60 carbon atoms, and the term “C₆-C₆a arylene group” as used herein refers to a divalent group having a carbocyclic aromatic system of 6 to 60 carbon atoms. Examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, a fluorenyl group, and/or the like. When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having a heterocyclic aromatic system that includes, in addition to 1 to 60 carbon atoms, at least one heteroatom, as ring-forming atoms, and the term “C₁-C₆₀ heteroarylene group” as used herein refers to a divalent group having a heterocyclic aromatic system that includes, in addition to 1 to 60 carbon atoms, at least one heteroatom, as ring-forming atoms. Examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, a carbazolyl group, a dibenzofuranyl group, a dibenzothiofuranyl group, and/or the like. When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group that has two or more rings condensed with each other, only carbon atoms (e.g., 8 to 60 carbon atoms) as ring-forming atoms, and no aromaticity in its entire molecular structure (e.g., is not aromatic when considered as a whole). Examples of the monovalent non-aromatic condensed polycyclic group may include an indenyl group, a fluorenyl group, a spiro-bifluorenyl group, a benzofluorenyl group, an indenophenanthrenyl group, an indenoanthracenyl group, an adamantyl group, and/or the like. The term polyvalent (e.g., divalent) non-aromatic condensed polycyclic group” as used herein refers to a polyvalent (e.g., divalent) group having substantially the same structure as the monovalent non-aromatic condensed polycyclic group.

The term “monovalent non-aromatic condensed heteropolycyclic group” as used herein refers to a monovalent group that has two or more rings condensed with each other, at least one heteroatom in addition to carbon atoms (e.g., 1 to 60 carbon atoms), as a ring-forming atom, and no aromaticity in its entire molecular structure (e.g., is not aromatic when considered as a whole). Examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphtho silolyl group, a benzofurodibenzofuranyl group, a benzofurodibenzothiophenyl group, a benzothienodibenzothiophenyl group, an azaadamantyl group, and/or the like. The term “polyvalent (e.g., divalent) non-aromatic condensed heteropolycyclic group” as utilized herein refers to a polyvalent (e.g., divalent) group having substantially the same structure as the monovalent non-aromatic condensed heteropolycyclic group.

The term “C₆-C₆₀ aryloxy group” as used herein refers to a monovalent group represented by —OA₁₀₂ (wherein A₁₀₂ is the C₆-C₆₀ aryl group), and the term “C₆-C₆₀ arylthio group” as used herein refers to a monovalent group represented by —SA₁₀₃ (wherein A₁₀₃ is the C₆-C₆₀ aryl group).

The term “C₇-C₆₀ arylalkyl group” as used herein refers to a monovalent group represented by -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as used herein refers to a monovalent group represented by -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃ and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as used herein refers to any atom other than a carbon atom. Examples of the heteroatom may include O, S, N, P, Si, B, Ge, Se, or any combination thereof.

The term “third-row transition metal” as used herein may include hafnium (Hf), tantalum (Ta), tungsten (W), rhenium (Re), osmium (Os), iridium (Ir), platinum (Pt), gold (Au), and/or the like.

The term “Ph” as used herein refers to a phenyl group, the term “Me” as used herein refers to a methyl group, the term “Et” as used herein refers to an ethyl group, the term “tert-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group.” In other words, the “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula or moiety.

The x-axis, y-axis, and z-axis as used herein are not limited to three axes in an orthogonal coordinate system, and may be interpreted in a broad sense including these axes. For example, the x-axis, y-axis, and z-axis may be orthogonal to each other, but the x-axis, y-axis, and z-axis may also refer to different directions that are not orthogonal to each other.

Hereinafter, compounds according to embodiments and light-emitting devices according to embodiments will be described in more detail with reference to Synthesis Examples and Examples. The wording “B was utilized instead of A” used in describing Synthesis Examples refers to that an identical molar equivalent of B was utilized in place of A.

### Synthesis Example 1 (Synthesis of Compound 3)

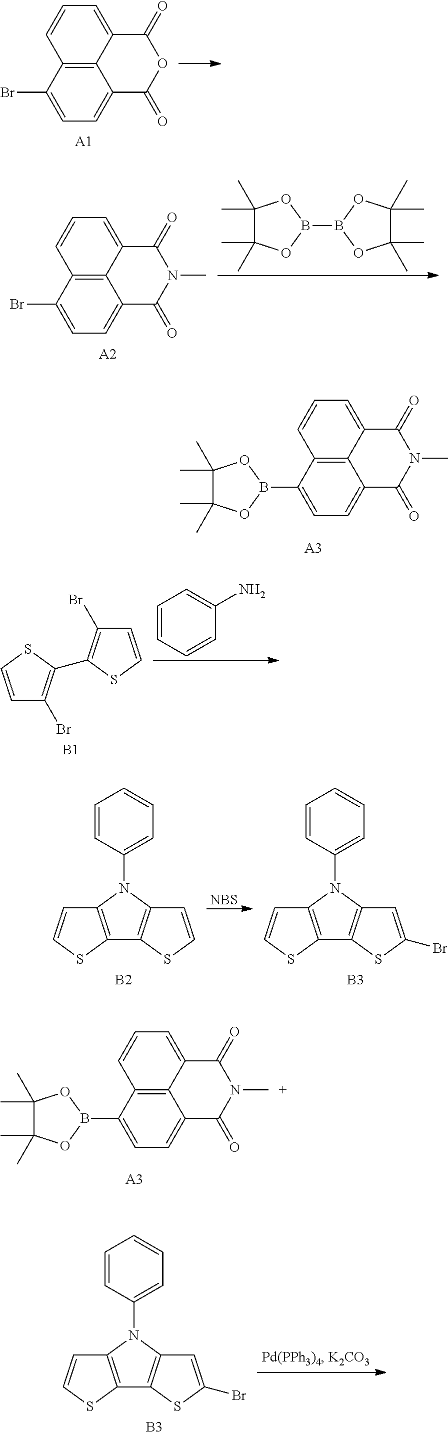

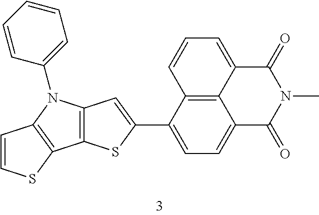 [SMILES: CN1C(=O)c2cccc3c(-c4cc5c(s4)c4sccc4n5-c4ccccc4)ccc(c23)C1=O]

### Synthesis of Intermediate A2

3.00 g (10.83 mmol) of Intermediate A1 was dissolved in 10 mL of 40% aqueous methylamine, and then, the mixture was stirred at 50° C. for 6 hours. After cooling the reaction solution to room temperature, the resulting solid was filtered, and then, the obtained solid was extracted three times utilizing 50 mL of water and 50 mL of dichloromethane. The obtained organic layer was dried utilizing MgSO₄, and the residue obtained by evaporating the solvent was separated and purified by silica gel column chromatography to obtain 2.83 g (yield of 90%) of Intermediate A2 (6-bromo-2-methyl-1H-benzo[de]isoquinoline-1,3(2H)-dione). Intermediate A2 thus produced was identified by ¹H NMR (CDCl₃, 500 MHz) and MS/FAB.

(500 MHz, CDCl₃): δ 8.54 (m, 1H), 8.48 (m, 1H), 8.29 (d, 1H), 8.24 (d, 1H), 7.98 (m, 1H), 3.05 (s, 3H)

C₁₃H8BrNO₂: calc. 290.12, found 288.97.

### Synthesis of Intermediate A3

2.00 g (6.89 mmol) of Intermediate A2, 2.63 g (10.34 mmol, 1.5 eq) of bis(pinacolato)diborane, 0.30 g (0.34 mmol, 0.05 eq) of PdCl₂(dppf)₂, and 2.03 g (20.68 mmol, 3 eq) of CH₃COOK were dissolved in 60 mL of dimethyl sulfoxide (DMSO), and then, the mixture was stirred at 80° C. for 12 hours. After cooling the reaction solution to room temperature, an extraction process was performed thereon three times utilizing 50 mL of water and 50 mL of dichloromethane. The obtained organic layer was dried utilizing MgSO₄, and the residue obtained by evaporating the solvent was separated and purified by silica gel column chromatography to obtain 1.63 g (yield of 70%) of Intermediate A3. Intermediate A3 (2-methyl-6-(4,4,5,5-tetramethyl-1,3,2-dioxaborolan-2-yl)-1H-benzo[de]isoquinoline-1,3(2H)-dione) thus produced was identified by ¹H NMR (CDCl₃, 500 MHz) and MS/FAB.

(500 MHz, CDCl₃): δ 8.50 (m, 1H), 8.49 (d, 1H), 8.37 (m, 1H), 7.87 (m, 1H), 7.86 (d, 1H), 3.05 (s, 3H), 1.20 (s, 12H)

C₁₉H₂₀BNO₄: calc. 337.18, found 337.15.

### Synthesis of Intermediate B2

In a nitrogen atmosphere, 0.85 g (0.93 mmol, 0.1 eq) of Pd₂(dba)₃ and 0.27 g (0.93 mmol, 0.1 eq) of [(t-Bu)₃PH]BF₄ were added to 80 mL of toluene. Successively, 2.67 g (27.8 mmol, 3 eq) of NaOC(CH₃)₃ and 3.00 g (9.26 mmol) of 3,3′-dibromo-2,2′-bithiophene were added thereto. After 5 minutes, 1.12 g (12.04 mmol, 1.3 eq) of aniline was added thereto, and the mixture was stirred at 120° C. for 6 hours. After cooling the reaction solution to room temperature, an extraction process was performed thereon three times utilizing 50 mL of water and 50 mL of diethyl ether. The obtained organic layer was dried utilizing MgSO₄, and the residue obtained by evaporating the solvent was separated and purified by silica gel column chromatography to obtain 1.30 g (yield of 55%) of Intermediate B2 (4-phenyl-4H-dithieno[3,2-b:2′,3′-d]pyrrole). Intermediate B2 thus produced was identified by ¹H NMR (CDCl₃, 500 MHz) and MS/FAB.

(500 MHz, CDCl₃): δ 7.62 (m, 2H), 7.58 (m, 1H), 7.50 (m, 2H), 7.31 (d, 2H), 7.12 (d, 2H)

C₁₄H₉NS₂, calc. 255.35, found 255.02.

### Synthesis of Intermediate B3

1.00 g (3.92 mmol, 1 eq) of Intermediate B2 was dissolved in 90 mL of dochloromethane, and then, 0.77 g (4.31 mmol, 1.1 eq) of N-bromosuccinimide was stirred at room temperature for 6 hours. 60 mL of water was added to the reaction solution, and an extraction process was performed thereon three times utilizing 60 mL of dichloromethane. The obtained organic layer was dried utilizing MgSO₄, and the residue obtained by evaporating the solvent was separated and purified by silica gel column chromatography to obtain 1.08 g (yield of 80%) of Intermediate B3 (2-bromo-4-phenyl-4H-dithieno[3,2-b:2′,3′-d]pyrrole). Intermediate B3 thus produced was identified by ¹H NMR (CDCl₃, 500 MHz) and MS/FAB.

(500 MHz, CDCl₃): δ 7.62 (m, 2H), 7.58 (m, 1H), 7.50 (m, 2H), 7.31 (d, 1H), 7.12 (d, 1H), 6.91 (s, 1H)

C₁₄H₈BrNS₂: calc. 334.25, found 332.93.

### Synthesis of Compound 3

1.01 g (3.00 mmol, 1 eq) of Intermediate A3, 1.0 g (3.00 mmol, 1 eq) of Intermediate B3, 0.70 g (0.6 mmol, 0.2 eq) of Pd(PPh₃)₄, and 1.03 g (17.92 mmol, 2.5 eq) of K₂CO₃ were dissolved in 60 mL of a mixed solution of toluene/H₂O (volume ratio of 2/1), and then, the mixture was stirred at 100° C. for 12 hours. After cooling the reaction solution to room temperature, 60 mL of water was added thereto, and an extraction process was performed thereon three times utilizing 60 mL of dichloromethane. The obtained organic layer was dried utilizing MgSO₄, and the residue obtained by evaporating the solvent was separated and purified by silica gel column chromatography to obtain 0.97 g (yield of 70%) of Compound 3 (2-methyl-6-(4-phenyl-4H-dithieno[3,2-b:2′,3′-d]pyrrol-2-yl)-1H-benzo[de]isoquinoline-1,3(2H)-dione). Compound 3 thus produced was identified by ¹H NMR (CDCl₃, 500 MHz) and MS/FAB.

(500 MHz, CDCl₃): δ 9.22 (m, 1H), 8.68 (d, 1H), 8.50 (m, 1H), 8.23 (d, 1H), 7.87 (m, 1H), 7.62 (m, 2H), 7.58 (m, 1H), 7.50 (m, 2H), 7.31 (d, 1H), 7.12 (d, 1H), 7.10 (s, 1H) 3.05 (s, 3H)

C₂₇H₁₆N₂O₂S₂: calc. 464.56, found 464.07.

### Synthesis Example 2 (Synthesis of Compound 6)

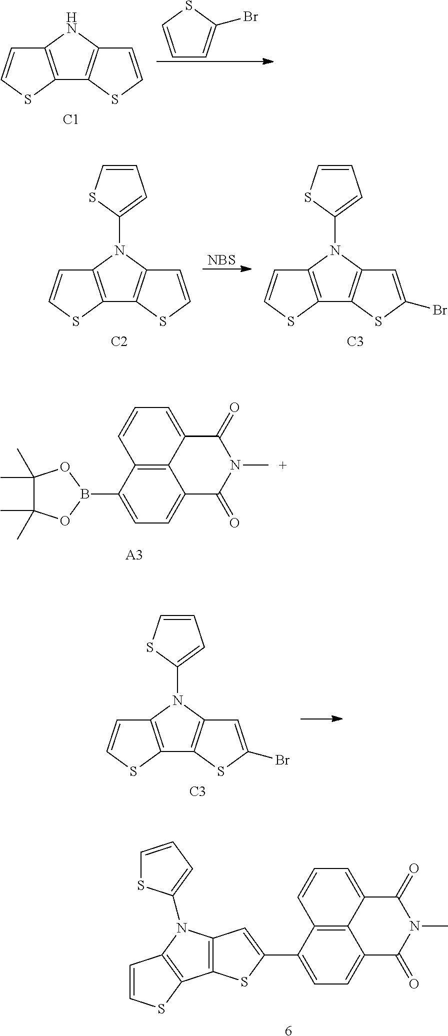

### Synthesis of Intermediate C2

1.00 g (5.58 mmol, 1 eq) of Intermediate C1 (4H-dithieno[3,2-b:2′,3′-d]pyrrole), 0.64 g (3.35 mmol, 0.6 eq) of copper(I) Iodide, 1.27 g (11.16 mmol, 2 eq) of trans-1,2-cyclohexanediamine, and 11.84 g (55.78 mmol, 10 eq) of tripotassium phosphate were dissolved in 100 mL of dioxane. 1.82 g (38.9 mmol, 2 eq) of 2-bromothiophene was added thereto, and the mixture was heated under reflux for 30 hours. After cooling the reaction solution to room temperature, 60 mL of water was added thereto, and an extraction process was performed thereon three times utilizing 60 mL of dichloromethane. The obtained organic layer was dried utilizing MgSO₄, and the residue obtained by evaporating the solvent was separated and purified by silica gel column chromatography to obtain 1.02 g (yield of 70%) of Intermediate C2 (4-(thiophen-2-yl)-4H-dithieno[3,2-b:2′,3′-d]pyrrole). Intermediate C2 thus produced was identified by ¹H NMR (CDCl₃, 500 MHz) and MS/FAB.

(500 MHz, CDCl₃): 7.54 (m, 1H), 7.31 (d, 2H), 7.12 (d, 2H), 7.10 (m, 1H), 6.99 (m, 1H)

C₁₂H₇NS₃: calc. 261.38, found 260.97.

### Synthesis of Intermediate C3

Intermediate C3 (2-bromo-4-(thiophen-2-yl)-4H-dithieno[3,2-b:2′,3′-d]pyrrole) was synthesized in substantially the same manner as utilized to synthesize Intermediate B3, except that Intermediate C2 was utilized instead of Intermediate B2 in the synthesis of Intermediate B3 in Synthesis Example 1. Intermediate C3 thus produced was identified by ¹H NMR (CDCl₃, 500 MHz) and MS/FAB.

(500 MHz, CDCl₃): 7.54 (m, 1H), 7.31 (d, 1H), 7.12 (d, 1H), 7.10 (m, 1H), 6.99 (m, 1H), 6.91 (s, 1H)

C₁₂H₆BrNS₃: calc. 340.27, found 340.88.

### Synthesis of Compound 6

Compound 6 (2-methyl-6-(4-(thiophen-2-yl)-4H-dithieno[3,2-b:2′,3′-d]pyrrol-2-yl)-1H-benzo[de]isoquinoline-1,3(2H)-dione) was synthesized in substantially the same manner as utilized to synthesize Compound 3, except that Intermediate C3 was utilized instead of Intermediate B3 in the synthesis of Compound 3 in Synthesis Example 1. Compound 6 thus produced was identified by ¹H NMR (CDCl₃, 500 MHz) and MS/FAB.

(500 MHz, CDCl₃): 9.22 (m, 1H), 8.68 (d, 1H), 8.50 (m, 1H), 8.23 (d, 1H), 7.87 (m, 1H), 7.54 (m, 1H), 7.31 (d, 1H), 7.12 (d, 1H), 7.10 (m, 2H), 6.99 (m, 1H), 3.05 (s, 3H)

C₂₅H₁₄N₂O₂S₃: calc. 470.58, found 470.02.

### Synthesis Example 3 (Synthesis of Compound 14)

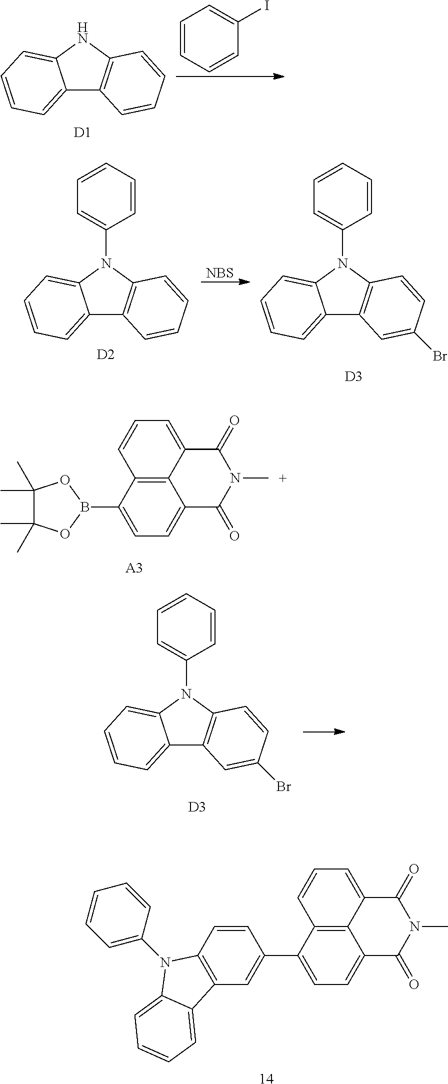

### Synthesis of Intermediate D2

Intermediate D2 (9-phenyl-9H-carbazole) was synthesized in substantially the same manner as utilized to synthesize Intermediate C2, except that, in the synthesis of Intermediate C2 in Synthesis Example 2, Intermediate D1 (9H-carbazole) was utilized instead of Intermediate C1, and iodobenzene was utilized instead of 2-bromothiophene. Intermediate D2 thus produced was identified by ¹H NMR (CDCl₃, 500 MHz) and MS/FAB.

(500 MHz, CDCl₃): 8.55 (m, 2H), 7.94 (m, 2H), 7.62 (m, 2H), 7.58 (m, 1H), 7.50 (m, 2H), 7.35 (m, 2H), 7.16 (m, 2H)

C₁₈H₁₃N: calc. 243.31, found 243.10.

### Synthesis of Intermediate D3

Intermediate D3 (3-bromo-9-phenyl-9H-carbazole) was synthesized in substantially the same manner as utilized to synthesize Intermediate B3, except that Intermediate D2 was utilized instead of Intermediate B2 in the synthesis of Intermediate B3 in Synthesis Example 1. Intermediate D3 thus produced was identified by ¹H NMR (CDCl₃, 500 MHz) and MS/FAB.

(500 MHz, CDCl₃): 8.55 (m, 1H), 8.05 (s, 1H), 7.94 (m, 1H), 7.62 (m, 2H), 7.58 (m, 1H), 7.50 (m, 2H), 7.47 (d, 1H), 7.42 (d, 1H), 7.35 (m, 1H), 7.16 (m, 1H)

C₁₈H₁₂BrN, calc. 322.21, found 321.02.

### Synthesis of Compound 14

Compound 14 (2-methyl-6-(9-phenyl-9H-carbazol-3-yl)-1H-benzo[de]isoquinoline-1,3(2H)-dione) was synthesized in substantially the same manner as utilized to synthesize Compound 3, except that Intermediate D3 was utilized instead of Intermediate B3 in the synthesis of Compound 3 in Synthesis Example 1. Compound 14 thus produced was identified by ¹H NMR (CDCl₃, 500 MHz) and MS/FAB.

(500 MHz, CDCl₃): 9.20 (m, 1H), 8.55 (m, 1H), 8.45 (d, 1H), 8.37 (m, 1H), 7.99 (d, 1H), 7.94 (m, 1H), 7.89 (s, 1H), 7.80 (m, 1H), 7.78 (d, 1H), 7.77 (d, 1H), 7.62 (m, 2H), 7.58 (m, 1H), 7.50 (m, 2H), 7.35 (m, 1H), 7.16 (m, 1H), 3.05 (s, 3H)

C₃₁H₂₀N₂O₂, calc. 452.51, found 452.15.

### Synthesis Example 4 (Synthesis of Compound 13)

Compound 13 (6-(9,9-dimethyl-10-phenyl-9,10-dihydroacridin-2-yl)-2-methyl-1H-benzo[de]isoquinoline-1,3(2H)-dione) was synthesized in substantially the same manner as utilized to synthesize Compound 14, except that 9,9-dimethyl-9,10-dihydroacridine was utilized instead of Intermediate D1 in the synthesis of Compound 14 in Synthesis Example 3. Compound 13 thus produced was identified by ¹H NMR (CDCl₃, 500 MHz) and MS/FAB.

(500 MHz, CDCl₃): 9.20 (m, 1H), 8.45 (d, 1H), 8.37 (m, 1H), 7.80 (m, 1H), 7.78 (d, 1H), 7.76 (s, 1H), 7.45 (d, 1H), 7.36 (d, 1H), 7.24 (m, 2H), 7.19 (m, 1H), 7.17 (m, 1H), 7.14 (m, 1H), 7.08 (m, 2H), 7.00 (m, 1H), 6.95 (m, 1H), 3.05 (s, 3H), 1.69 (s, 6H)

C₃₂H₂₂N₂O₂S: calc. 494.59, found 494.20.

### Synthesis Example 5 (Synthesis of Compound 37)

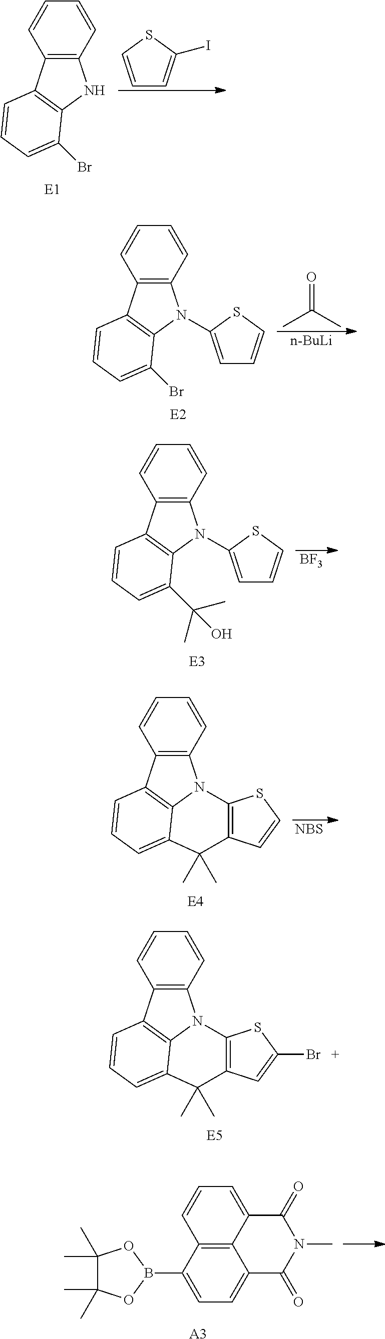

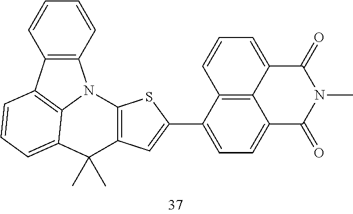 [SMILES: CN1C(=O)c2cccc3c(-c4cc5c(s4)-n4c6ccccc6c6cccc(c64)C5(C)C)ccc(c23)C1=O]

### Synthesis of Intermediate E2

Intermediate E2 (1-bromo-9-(thiophen-2-yl)-9H-carbazole) was synthesized in substantially the same manner as utilized to synthesize Intermediate C2, except that, in the synthesis of Intermediate C2 in Synthesis Example 2, Intermediate E1 (1-bromo-9H-carbazole) was utilized instead of Intermediate C1, and 2-iodothiophene was utilized instead of 2-bromothiophene. Intermediate E2 thus produced was identified by ¹H NMR (CDCl₃, 500 MHz) and MS/FAB.

(500 MHz, CDCl₃): 8.55 (m, 1H), 8.13 (m, 1H), 7.94 (m, 1H), 7.54 (m, 1H), 7.42 (m, 1H), 7.35 (m, 1H), 7.18 (d, 1H), 7.16 (m, 1H), 7.10 (m, 1H), 6.99 (m, 1H),
C₁₆H₁₀BrNS: calc. 328.23, found 328.97.

### Synthesis of Intermediate E3

3.0 g (9.14 mmol) of Intermediate E2 was dissolved in 60 mL of dehydrated diethyl ether. A solution of 2.5 M (5.5 mL, 13.71 mmol, 1.5 eq) of n-BuLi in hexane was added dropwise thereto at −50° C., and the mixture was stirred at room temperature for 1 hour. 0.58 g (10.97 mmol, 1.2 eq) of dehydrated acetone was added thereto at −50° C., and the mixture was stirred at room temperature for 2 hours. The organic layer extracted from dehydrated diethyl ether was washed utilizing an aqueous sodium chloride solution, and then dried by adding MgSO₄. 1.83 g (yield of 65%) of Intermediate E3 (2-(9-(thiophen-2-yl)-9H-carbazol-1-yl)propan-2-ol) was obtained, and the next step was performed immediately without a separate purification process. Intermediate E3 thus produced was identified by MS/FAB.

C₁₉H₁₇NOS, calc. 307.41, found 307.10.

### Synthesis of Intermediate E4

2.0 g (6.51 mmol) of Intermediate E3 was dissolved in 50 mL of dichloromethane. 2.31 g (16.26 mmol, 2.5 eq) of a boron trifluoride-ethyl ether complex was added dropwise thereto at 0° C., and the mixture was stirred for 2 hours. The organic layer extracted from dichloromethane was washed utilizing an aqueous sodium chloride solution, and then dried by adding MgSO₄. The obtained product was separated and purified by silica gel column chromatography to obtain 4.09 g (yield of 85%) of Intermediate E4 (4,4-dimethyl-4H-thieno[3′,2′:5,6]pyrido[3,2,1-jk]carbazole). Intermediate E4 thus produced was identified by ¹H NMR (CDCl₃, 500 MHz) and MS/FAB.

(500 MHz, CDCl₃): 8.55 (m, 1H), 8.09 (m, 1H), 7.94 (m, 1H), 7.53 (d, 1H), 7.45 (m, 1H), 7.35 (m, 1H), 7.16 (m, 1H), 7.07 (m, 1H), 7.02 (d, 1H), 1.69 (s, 6H)

C₁₉H₁₅NS: calc. 289.40, found 289.09.

### Synthesis of Intermediate E5

Intermediate E5 (2-bromo-4,4-dimethyl-4H-thieno[3′,2′:5,6]pyrido[3,2,1-jk]carbazole) was synthesized in substantially the same manner as utilized to synthesize Intermediate B3, except that Intermediate E4 was utilized instead of Intermediate B2 in the synthesis of Intermediate B3 in Synthesis Example 1. Intermediate E5 thus produced was identified by ¹H NMR (CDCl₃, 500 MHz) and MS/FAB.

(500 MHz, CDCl₃): 8.55 (m, 1H), 8.09 (m, 1H), 7.94 (m, 1H), 7.45 (m, 1H), 7.35 (m, 1H), 7.16 (m, 1H), 7.07 (m, 1H), 7.00 (s, 1H), 1.69 (s, 6H)

C₁₉H₁₄BrNS: calc. 368.29, found 369.00.

### Synthesis of Compound 37

Compound 37 was synthesized in substantially the same manner as utilized to synthesize Compound 3, except that Intermediate E5 was utilized instead of Intermediate B3 in the synthesis of Compound 3 in Synthesis Example 1. Compound 37 thus produced was identified by ¹H NMR (CDCl₃, 500 MHz) and MS/FAB.

(500 MHz, CDCl₃): 9.22 (m, 1H), 8.68 (m, 1H), 8.55 (m, 1H), 8.50 (d, 1H), 8.23 (d, 1H), 8.09 (m, 1H), 7.94 (m, 1H), 7.87 (m, 1H), 7.45 (m, 1H), 7.35 (m, 1H), 7.16 (m, 1H), 7.07 (m, 1H), 7.05 (s, 1H), 3.05 (s, 3H), 1.69 (s, 6H)

C₃₂H₂₂N₂O₂S: calc. 498.60, found 498.14.

### Synthesis Example 6 (Synthesis of Compound 42)

Compound 42 (2-methyl-6-(4,4,8,8-tetramethyl-4H,8H-thieno[3′,2′:5,6]pyrido[3,2,1-de]acridin-2-yl)-1H-benzo[de]isoquinoline-1,3(2H)-dione) was synthesized in substantially the same manner as utilized to synthesize Compound 37, except that 9,9-dimethyl-9,10-dihydroacridine was utilized instead of Intermediate E1 in the synthesis of Intermediate E2 in Synthesis Example 5. Compound 42 thus produced was identified by ¹H NMR (CDCl₃, 500 MHz) and MS/FAB.

(500 MHz, CDCl₃): 9.22 (m, 1H), 8.68 (m, 1H), 8.50 (d, 1H), 8.23 (d, 1H), 7.87 (m, 1H), 7.14 (m, 1H), 7.19 (m, 1H), 7.17 (m, 2H), 7.04 (m, 2H), 6.95 (m, 1H), 6.60 (s, 1H), 3.05 (s, 3H), 1.69 (s, 12H),
C₃₅H₂₈N₂O₂S: calc. 540.68, found 540.19.

### Synthesis Example 7 (Synthesis of Compound 55)

Compound 55 (6-(8,8-dimethyl-8H-indolo[3,2,1-de]acridin-10-yl)-2-methyl-1H-benzo[de]isoquinoline-1,3(2H)-dione) was synthesized in substantially the same manner as utilized to synthesize Compound 37, except that iodobenzene was utilized instead of 2-iodothiophene in the synthesis of Intermediate E2 in Synthesis Example 5. Compound 55 thus produced was identified by ¹H NMR (CDCl₃, 500 MHz) and MS/FAB.

(500 MHz, CDCl₃): 8.55 (m, 1H), 8.09 (m, 1H), 7.94 (m, 2H), 7.91 (d, 1H), 7.81 (d, 1H), 7.45 (m, 1H), 7.35 (m, 1H), 7.16 (m, 1H), 7.07 (s, 1H), 1.69 (s, 6H), 9.22 (m, 1H), 8.45 (d, 1H), 8.37 (m, 1H), 7.80 (m, 1H), 7.78 (d, 1H), 3.05 (s, 3H)

C₃₄H₂₄N₂O₂: calc. 492.58, found 492.18.

### Synthesis Example 8 (Synthesis of Compound 56)

Compound 56 (2-methyl-6-(5,5,9,9-tetramethyl-5,9-dihydroquinolino[3,2,1-de]acridin-3-yl)-1H-benzo[de]isoquinoline-1,3(2H)-dione) was synthesized in substantially the same manner as utilized to synthesize Compound 37, except that, in the synthesis of Intermediate E2 in Synthesis Example 5, 9,9-dimethyl-9,10-dihydroacridine was utilized instead of Intermediate D1, and iodobenzene was utilized instead of 2-iodothiophene. Compound 56 thus produced was identified by ¹H NMR (CDCl₃, 500 MHz) and MS/FAB.

(500 MHz, CDCl₃): 9.22 (m, 1H), 8.45 (d, 1H), 8.37 (m, 1H), 7.80 (m, 1H), 7.78 (d, 1H), 7.76 (s, 1H), 7.45 (d, 1H), 7.36 (d, 1H), 7.19 (m, 1H), 7.17 (m, 1H), 7.17 (m, 1H), 7.14 (m, 1H), 7.04 (m, 2H), 6.95 (m, 1H), 1.69 (s, 12H), 3.05 (s, 3H)

C₃₇H₃₀N₂O₂: calc. 534.66, found 534.23.

### Synthesis Example 9 (Synthesis of Compound 73)

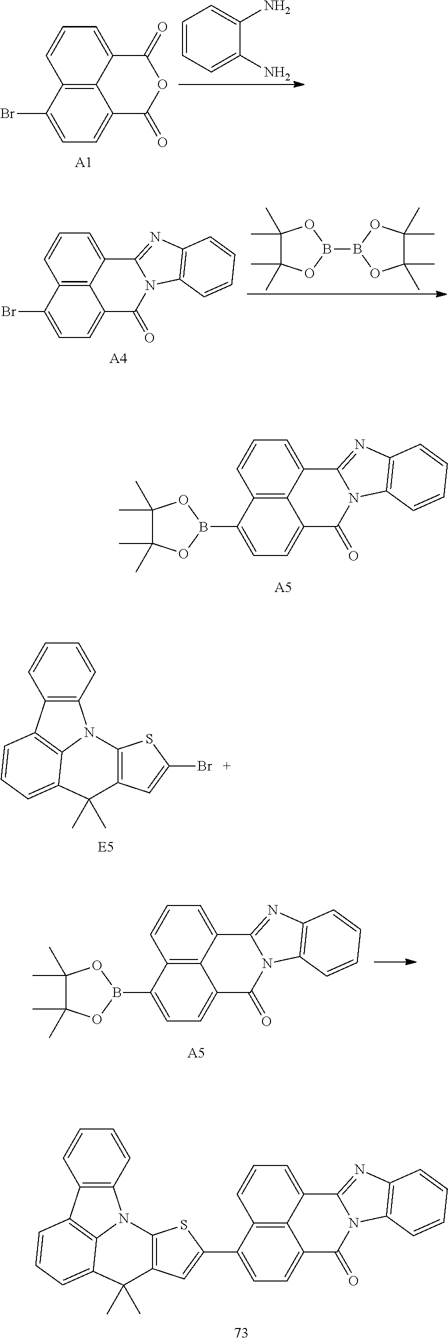

### Synthesis of Intermediate A4

Intermediate A4 (4-bromo-7H-benzo[de]benzo[4,5]imidazo[2,1-a]isoquinolin-7-one) was synthesized in substantially the same manner as utilized to synthesize Intermediate A2, except that benzene-1,2-diamine was utilized instead of methylamine in the synthesis of Intermediate A2 in Synthesis Example 1. Intermediate A4 thus produced was identified by ¹H NMR (CDCl₃, 500 MHz) and MS/FAB.

(500 MHz, CDCl₃): 8.38 (m, 1H), 8.31 (m, 1H), 8.23 (d, 1H), 8.17 (m, 1H), 8.07 (d, 1H), 7.57 (m, 1H), 7.54 (m, 1H), 7.24 (m, 1H), 7.21 (m, 1H)

C₁₈H₉BrN₂O: calc. 349.19, found 347.99.

### Synthesis of Intermediate A5

Intermediate A5 (4-(4,4,5,5-tetramethyl-1,3,2-dioxaborolan-2-yl)-7H-benzo[de]benzo[4,5]imidazo[2,1-a]isoquinolin-7-one) was synthesized in substantially the same manner as utilized to synthesize Intermediate A3, except that Intermediate A₄ was utilized instead of Intermediate A2 in the synthesis of Intermediate A3 in Synthesis Example 1. Intermediate A5 thus produced was identified by ¹H NMR (CDCl₃, 500 MHz) and MS/FAB.

(500 MHz, CDCl₃): 8.32 (d, 1H), 8.27 (m, 2H), 8.06 (m, 1H), 7.80 (d, 1H), 7.57 (m, 1H), 7.54 (m, 1H), 7.24 (m, 1H), 7.21 (m, 1H), 1.20 (s, 12H)

C₂₄H₂₁BN₂O₃: calc. 396.25, found 396.16.

### Synthesis of Compound 73

Compound 73 was synthesized in substantially the same manner as utilized to synthesize Compound 37, except that Intermediate A5 was utilized instead of Intermediate A3 in the synthesis of Compound 37 in Synthesis Example 5. Compound 73 thus produced was identified by ¹H NMR (CDCl₃, 500 MHz) and MS/FAB.

(500 MHz, CDCl₃): 9.12 (m, 1H), 8.55 (m, 1H), 8.51 (d, 1H), 8.27 (m, 1H), 8.17 (d, 1H), 8.09 (m, 1H), 8.06 (m, 1H), 7.94 (m, 1H), 7.57 (m, 1H), 7.54 (m, 1H), 7.45 (m, 1H), 7.35 (m, 1H), 7.24 (m, 1H), 7.21 (m, 1H), 7.16 (m, 1H), 7.07 (m, 1H), 7.05 (s, 1H), 1.69 (s, 6H)

C₃₇H₂₃N3OS: calc. 557.67, found 557.16.

### Example 1

A substrate on which an ITO anode was deposited was prepared to have a size of 50 mm×50 mm×0.7 mm. The substrate was ultrasonically cleaned utilizing isopropyl alcohol and pure water, each for 5 minutes. Subsequently, ultraviolet rays were irradiated onto the substrate for 30 minutes, and the substrate was exposed to ozone to be cleaned. The cleaned substrate was loaded onto a vacuum deposition apparatus. A hole injection layer having a thickness of 600 Å was formed on the ITO substrate by vacuum deposition. A hole transport layer vacuum-deposited to a thickness of 300 Å on the hole injection layer. An auxiliary layer was formed to a thickness of 300 Å on the hole transport layer. Compound 3 and Compound N16 were co-deposited on the auxiliary layer to form a photoactive layer having a thickness of 500 Å. An electron transport layer was formed to a thickness of 300 Å on the photoactive layer by deposition. An electron injection layer was formed to a thickness of 10 Å on the electron transport layer by deposition. Subsequently, Al was vacuum-deposited to a thickness of 3,000 Å to form a LiF/Al cathode, thereby completing the manufacture of an opto-electronic device.

### Examples 2 to 9 and Comparative Examples 1 and 2

Opto-electronic devices were manufactured in substantially the same manner as in Example 1, except that compounds shown in Table 1 were each utilized instead of Compound 3 in forming the photoactive layer.

### Evaluation Example 1

The HOMO and LUMO of each of the compounds of Examples and Comparative Examples were measured, and the results are shown in Table 1.

By utilizing cyclic voltammetry (CV) (electrolyte: 0.1 M Bu₄NPF₆, solvent: dimethylforamide (DMF), electrode: 3-electrode system (working electrode: GC, reference electrode: Ag/AgCl, auxiliary electrode: Pt)), the potential (V)-current (A) graph of each compound was obtained. The HOMO and LUMO values of each compound were calculated from the oxidation onset and reduction onset of the graph.

The maximum absorption wavelength and intensity were measured utilizing UV-vis spectroscopy.

TABLE 1

| | | | | | Maximum | |
|---|---|---|---|---|---|---|
| | | Molec- | | | absorption | |
| | Com- | ular | HOMO | LUMO | wavelength | Inten- |
| | pound | weight | (eV) | (eV) | (nm) | sity |
| Comparative | A | 745.29 | −5.98 | −2.60 | 489.7 | 0.261 |
| Example 1 | | | | | | |
| Comparative | B | 496.6 | −5.46 | −2.54 | 419.4 | 0.185 |
| Example 2 | | | | | | |
| Example 1 | 3 | 464.1 | −5.61 | −2.67 | 479.4 | 0.400 |
| Example 2 | 6 | 470.0 | −5.65 | −2.68 | 476.0 | 0.398 |
| Example 3 | 14 | 452.2 | −5.82 | −2.48 | 425.2 | 0.228 |
| Example 4 | 13 | 494.2 | −5.38 | −2.48 | 494.3 | 0.299 |
| Example 5 | 37 | 498.1 | −5.47 | −2.68 | 513.1 | 0.345 |
| Example 6 | 42 | 540.19 | −5.34 | −2.62 | 533.9 | 0.320 |
| Example 7 | 55 | 492.57 | −5.65 | −2.62 | 465.9 | 0.372 |
| Example 8 | 56 | 534.65 | −5.43 | −2.54 | 493.4 | 0.356 |
| Example 9 | 73 | 557.67 | −5.47 | −2.76 | 532.9 | 0.377 |

From Table 1, it was confirmed that i) the compound utilized in each of Examples 1 to 9 had a smaller molecular weight than the compound utilized in each of Comparative Examples 1 and 2, and/or ii) the opto-electronic device of each of Examples 1 to 9 had a higher intensity than the opto-electronic devices of Comparative Examples 1 and 2. Accordingly, it was confirmed that the opto-electronic device of each of Examples 1 to 9 had, as compared with the opto-electronic devices of Comparative Examples 1 and 2, i) excellent or suitable heat resistance characteristics and/or ii) excellent or suitable light absorption efficiency for green light (or light having a wavelength similar to that of green light).

### Evaluation Example 2

Light was irradiated to the opto-electronic devices manufactured utilizing the compounds of Examples 1 to 9 and Comparative Examples 1 and 2, and the external quantum efficiency according to the wavelength of each device was measured. The external quantum efficiency refers to the ratio of electrical energy generated from energy of the irradiated light.

The maximum external quantum efficiency (EQE) refers to the maximum value from among external quantum efficiencies according to wavelengths. The Δ^{max EQE} refers to the wavelength of the maximum external quantum efficiency (EQE). The maximum external quantum efficiency (EQE) and A^{max EQE} are shown in Table 2.

TABLE 2

| | Compound | EQE (%) | λ^{max EQE} (nm) |
|---|---|---|---|
| Comparative Example 1 | A | 15.7 | 510 |
| Comparative Example 2 | B | 11.1 | 439 |
| Example 1 | 3 | 48.0 | 499 |
| Example 2 | 6 | 47.8 | 496 |
| Example 3 | 14 | 27.4 | 445 |
| Example 4 | 13 | 35.9 | 514 |
| Example 5 | 37 | 41.4 | 533 |
| Example 6 | 42 | 38.4 | 554 |
| Example 7 | 55 | 44.6 | 486 |
| Example 8 | 56 | 42.7 | 513 |
| Example 9 | 73 | 45.2 | 553 |

From Table 2, it was confirmed that the opto-electronic device of each of Examples 1 to 9 had excellent or suitable photoelectric characteristics compared to the opto-electronic devices of Comparative Examples 1 and 2.

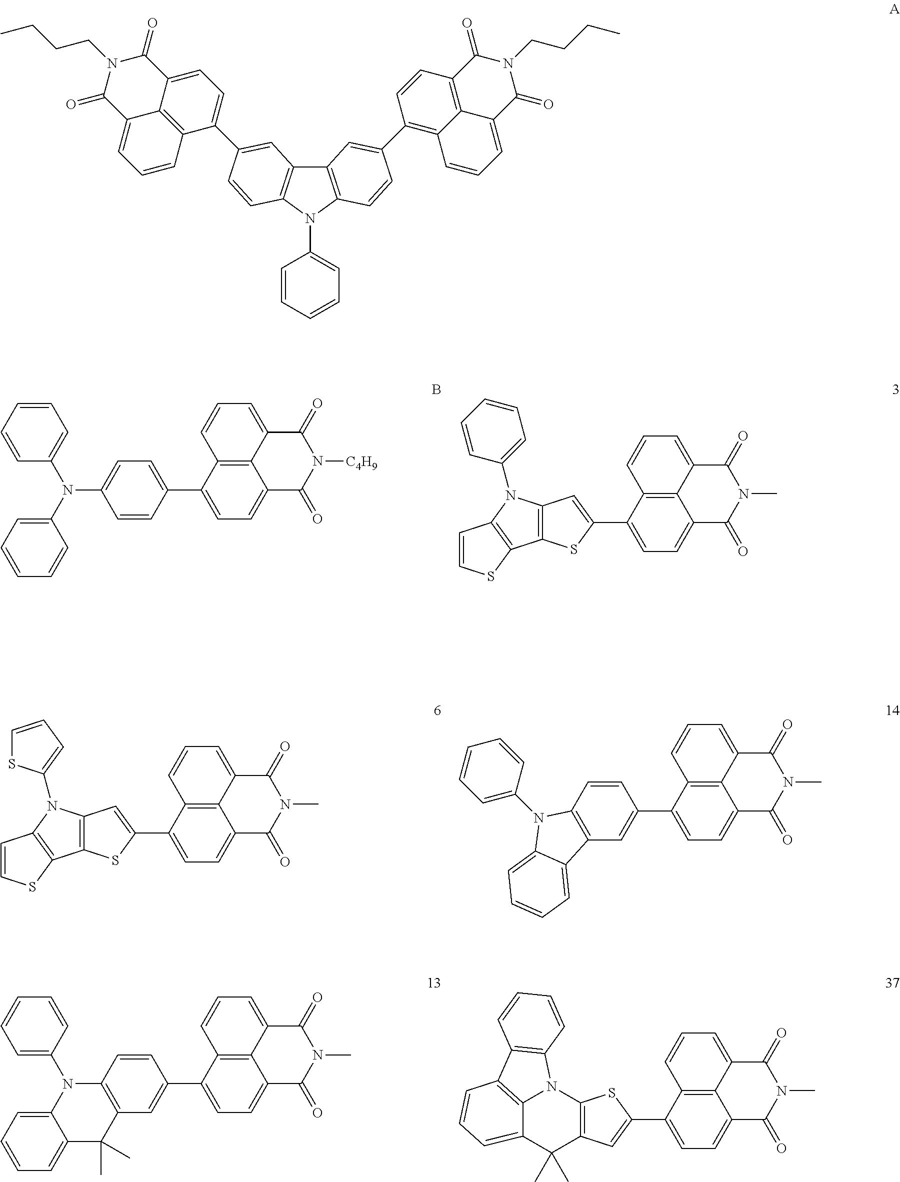

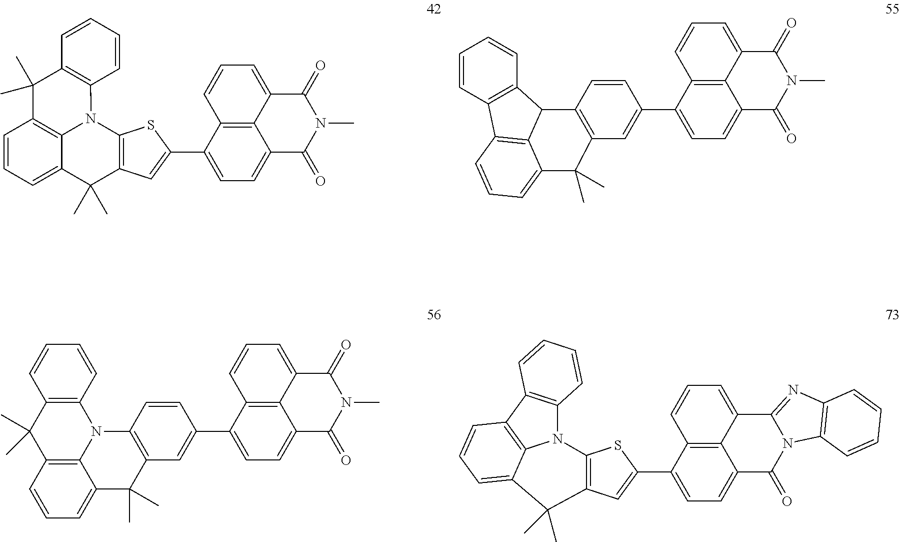

According to the one or more embodiments, the organic compound represented by Formula 1 may have high heat resistance characteristics. An opto-electronic device including the organic compound may have high light absorption efficiency. An electronic apparatus including the opto-electronic device and an electronic device utilizing the electronic apparatus may have improved quality.

The use of “may” when describing embodiments of the inventive concept refers to “one or more embodiments of the inventive concept.”

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

In the present disclosure, when particles are spherical, “diameter” indicates a particle diameter or an average particle diameter, and when the particles are non-spherical, the “diameter” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic apparatus and/or any other relevant devices or components according to embodiments of the present invention described herein may be implemented utilizing any suitable hardware, firmware (e.g. an application-specific integrated circuit), software, or a combination of software, firmware, and hardware. For example, the various components of the apparatus may be formed on one integrated circuit (IC) chip or on separate IC chips. Further, the various components of the apparatus may be implemented on a flexible printed circuit film, a tape carrier package (TCP), a printed circuit board (PCB), or formed on one substrate. Further, the various components of the apparatus may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. An opto-electronic device comprising:
a first electrode;
a second electrode facing the first electrode;
a photoactive layer between the first electrode and the second electrode; and
an organic compound represented by Formula 1: 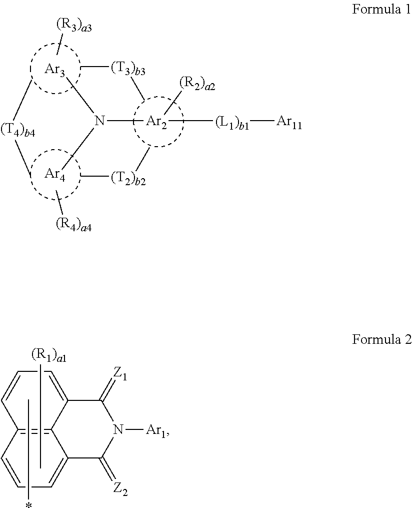
wherein, in Formula 1, Ar₁₁ is a group represented by Formula 2,
in Formulae 1 and 2,
Ar₁ is a C₁-C₆₀ alkyl group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
Z₁ and Z₂ are each independently O, S, or N, wherein, i) when Z₁ is N, Z₁ and Ar₁ are bonded to each other, and ii) when Z₂ is N, Z₂ and Ar₁ are bonded to each other,
L₁ is a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b1 is an integer from 0 to 2,
when b1 is 2, two L₁(s) are identical to or different from each other,
Ar₂ to Ar₄ are each independently a C₃-C₆₀ carbocyclic group or a C₁-C₆₀ heterocyclic group,
T₂ is a single bond, *—C(T₂₁)(T₂₂)-*′, *—Si(T₂₁)(T₂₂)-*′, *-0*′, *—S—*′, or —N(T₂₁)-*′,
T₃ is a single bond, *—C(T₃₁)(T₃₂)-*′, *—Si(T₃₁)(T₃₂)-*′, *-0*′, *—S—*′, or —N(T₃₁)-*′,
T₄ is a single bond, *—C(T₄₁)(T₄₂)-*′, *—Si(T₄₁)(T₄₂)-*′, *-0*′, *—S—*′, or —N(T₄₁)-*′,
b2 to b4 are each independently an integer from 0 to 2, wherein, i) when b2 is 0, *-(T₂)_{b2}-*′ is not present, ii) when b3 is 0, *-(T₃)_{b3}-*′ is not present, iii) when b4 is 0, *-(T₄)_{b4}-*′ is not present, iv) when b2 is 2, two T₂(s) are identical to or different from each other, v) when b3 is 2, two T₃(s) are identical to or different from each other, and vi) when b4 is 2, two T₄(s) are identical to or different from each other,
at least one of b2 to b4 is 1 or 2,
no group other than Ar₁₁ in Formula 1 is represented by Formula 2,
R₁ to R₅, T₂₁, T₂₂, T₃₁, T₃₂, T₄₁, and T₄₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1 to a5 are each independently an integer from 0 to 5,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

2. The opto-electronic device of claim 1, wherein
the opto-electronic device further comprises a hole transport region between the first electrode and the photoactive layer and an electron transport region between the photoactive layer and the second electrode, and
the photoactive layer comprises the organic compound.

3. The opto-electronic device of claim 2, wherein
the photoactive layer comprises a first layer adjacent to the hole transport region and a second layer adjacent to the electron transport region, and
the first layer comprises the organic compound.

4. The opto-electronic device of claim 3, wherein the photoactive layer further comprises a third layer between the first layer and the second layer, and
the third layer comprises the organic compound.

5. An electronic apparatus comprising the opto-electronic device of claim 1.

6. The electronic apparatus of claim 5, further comprising:
a thin-film transistor electrically connected to the first electrode;
an emission layer between the first electrode and the second electrode and not overlapping the photoactive layer; and
a color filter, a color conversion layer, a touch screen layer, and/or a polarizing layer.

7. An electronic device comprising the electronic apparatus of claim 5,
wherein the electronic device is at least one selected from the group consisting of flat panel displays, curved displays, computer monitors, medical monitors, televisions, billboards, indoor or outdoor lightings, signal lights, head-up displays, fully or partially transparent displays, flexible displays, rollable displays, foldable displays, stretchable displays, laser printers, telephones, mobile phones, tablets, phablets, personal digital assistants (PDAs), wearable devices, laptop computers, digital cameras, camcorders, viewfinders, microdisplays, three-dimensional (3D) displays, virtual or augmented-reality displays, vehicles, video walls comprising multiple displays tiled together, theater or stadium screens, phototherapy devices, signboards, and combinations thereof.

8. An organic compound represented by Formula 1: 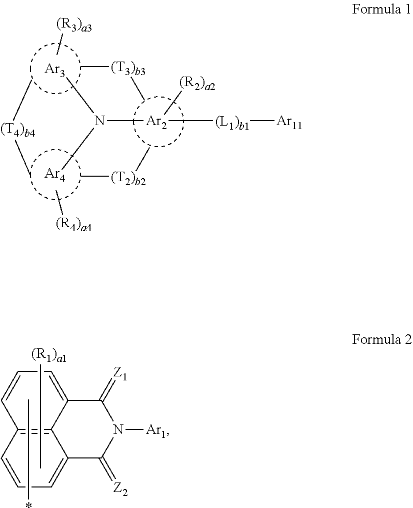
wherein, in Formula 1, Ar₁₁ is a group represented by Formula 2,
in Formulae 1 and 2,
Ar₁ is a C₁-C₆₀ alkyl group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
Z₁ and Z₂ are each independently O, S, or N, wherein, i) when Z₁ is N, Z₁ and Ar₁ are linked to each other, and ii) when Z₂ is N, Z₂ and Ar₁ are linked to each other,
L₁ is a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b1 is an integer from 0 to 2,
when b1 is 2, two L₁(s) are identical to or different from each other,
Ar₂ to Ar₄ are each independently a C₃-C₆₀ carbocyclic group or a C₁-C₆₀ heterocyclic group,
T₂ is a single bond, *—C(T₂₁)(T₂₂)-*′, *—Si(T₂₁)(T₂₂)-*′, *-0*′, *—S—*′, or —N(T₂₁)-*′,
T₂₁ and T₂₂ are optionally linked to each other via a single bond, a double bond, or *—O—*′,
T₃ is a single bond, *—C(T₃₁)(T₃₂)-*′, *—Si(T₃₁)(T₃₂)-*′, *-0*′, *—S—*′, or —N(T₃₁)-*′,
T₃₁ and T₃₂ are optionally linked to each other via a single bond, a double bond, or *—O—*′,
T₄ is a single bond, *—C(T₄₁)(T₄₂)-*′, *—Si(T₄₁)(T₄₂)-*′, *-0*′, *—S—*′, or —N(T₄₁)-*′,
T₄₁ and T₄₂ are optionally linked to each other via a single bond, a double bond, or *—O—*′,
b2 to b4 are each independently an integer from 0 to 2, wherein, i) when b2 is 0, *-(T₂)_{b2}-*′ is not present, ii) when b3 is 0, *-(T₃)_{b3}-*′ is not present, iii) when b4 is 0, *-(T₄)_{b4}-*′ is not present, iv) when b2 is 2, two T₂(s) are identical to or different from each other, v) when b3 is 2, two T₃(s) are identical to or different from each other, and vi) when b4 is 2, two T₄(s) are identical to or different from each other,
at least one of b2 to b4 is 1 or 2,
no group other than Ar₁₁ in Formula 1 is represented by Formula 2,
R₁ to R₅, T₂₁, T₂₂, T₃₁, T₃₂, T₄₁, and T₄₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1 to a5 are each independently an integer from 0 to 5,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

9. The organic compound of claim 8, wherein Formula 2 is represented by Formula 2a, 2b, or 2c:

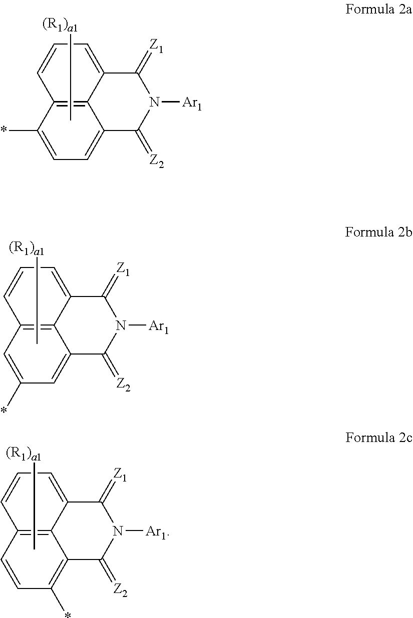

10. The organic compound of claim 8, wherein
i) b2 is 1, b3 is 0, and b4 is 0;
ii) b2 is 0, b3 is 1, and b4 is 0; or
iii) b2 is 1, b3 is 0, and b4 is 1.

11. The organic compound of claim 8, wherein Ar₁ is:
a methyl group, an ethyl group, an n-propyl group, an isopropyl group, a cyclohexyl group, a phenyl group, a naphthyl group, or a thiophenyl group; or
a cyclohexyl group, a phenyl group, a naphthyl group, or a thiophenyl group, each substituted with at least one selected from the group consisting of —F, —Cl, and a cyano group.

12. The organic compound of claim 8, wherein, Z₁ is N, and a ring formed by bonding between Z₁ and Ar₁ is a 5-membered ring.

13. The organic compound of claim 8, wherein, Z₁ is N, and Formula 2 is represented by Formula 2-1: 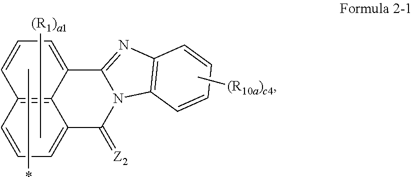
wherein, in Formula 2-1,
Z₂ is O or S,
a1 is an integer from 0 to 5,
c4 is an integer from 0 to 4, and
R₁ and R₁₀ₐ are the same as respectively defined in Formulae 1 and 2.

14. The organic compound of claim 8, wherein the organic compound represented by Formula 1 is represented by one of Formulae 3-1 to 3-3: 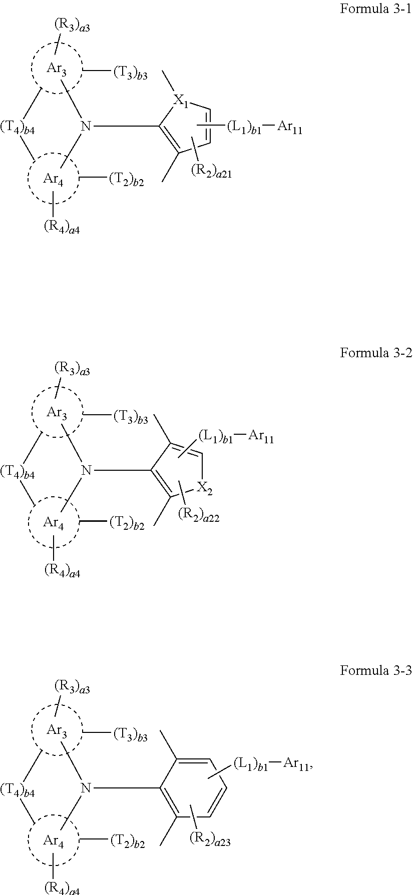
wherein, in Formulae 3-1 to 3-3,
X₁ and X₂ are each independently O, S, Se, Te, C(Q₄₁)(Q₄₂), N(Q₄₁), B(Q₄₁), Si(Q₄₁)(Q₄₂), or Ge(Q₄₁)(Q₄₂),
when X₁ is O, S, Se, or Te, b3 is 0,
a21 and a22 are each independently an integer from 0 to 2,
a23 is an integer from 0 to 4, and
Q₄₁ and Q₄₂ are each independently the same as defined in connection with Q₁ in Formulae 1 and 2.

15. The organic compound of claim 8, wherein L₁ is a single bond.

16. The organic compound of claim 8, wherein a molecular weight of the organic compound is about 1,000 or less.

17. The organic compound of claim 8, wherein at least one of Ar₂ to Ar₄ comprises a thiophene group.

18. The organic compound of claim 8, wherein Ar₃ and Ar₄ are each independently a benzene group, a naphthalene group, a thiophene group, a pyrrole group, a furan group, an indole group, a benzothiophene group, a benzofuran group, a quinoline group, or an isoquinoline group.

19. The organic compound of claim 8, wherein
i) T₂ is *—C(T₂₁)(T₂₂)-*′ or *—Si(T₂₁)(T₂₂)-*′, and T₂₁ and T₂₂ are linked to each other via a single bond;
ii) T₃ is *—C(T₃₁)(T₃₂)-*′ or *—Si(T₃₁)(T₃₂)-*′, and T₃₁ and T₃₂ are linked to each other via a single bond; or
iii) T₄ is *—C(T₄₁)(T₄₂)-*′ or *—Si(T₄₁)(T₄₂)-*′, and T₄₁ and T₄₂ are linked to each other via a single bond.

20. The organic compound of claim 8, wherein the organic compound is one of Compounds 1 to 96: 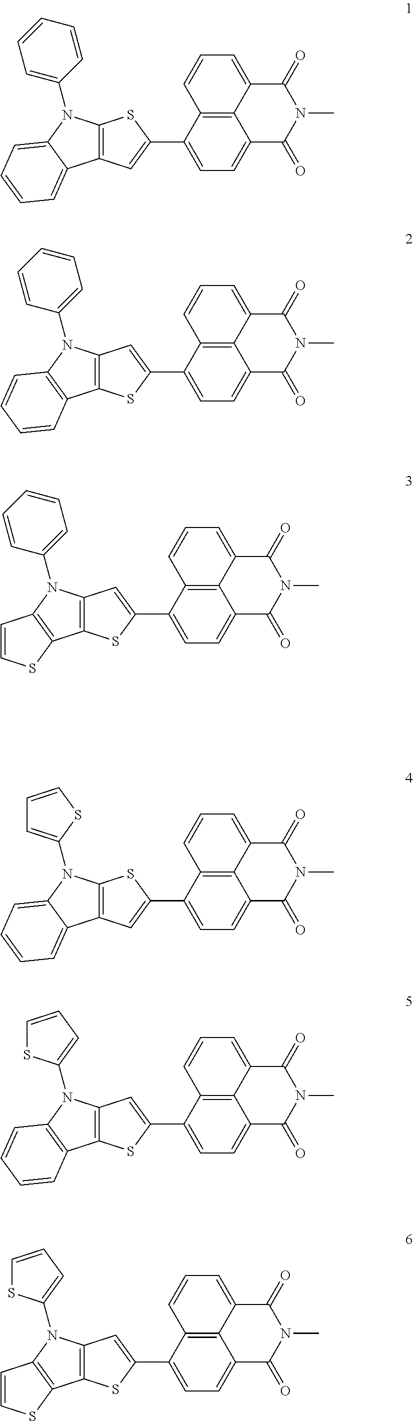 [SMILES: CN1C(=O)c2cccc3c(-c4cc5c6ccccc6n(-c6ccccc6)c5s4)ccc(c23)C1=O ; CN1C(=O)c2cccc3c(-c4cc5c(s4)c4ccccc4n5-c4ccccc4)ccc(c23)C1=O ; CN1C(=O)c2cccc3c(-c4cc5c(s4)c4sccc4n5-c4ccccc4)ccc(c23)C1=O ; CN1C(=O)c2cccc3c(-c4cc5c6ccccc6n(-c6cccs6)c5s4)ccc(c23)C1=O ; CN1C(=O)c2cccc3c(-c4cc5c(s4)c4ccccc4n5-c4cccs4)ccc(c23)C1=O ; CN1C(=O)c2cccc3c(-c4cc5c(s4)c4sccc4n5-c4cccs4)ccc(c23)C1=O] 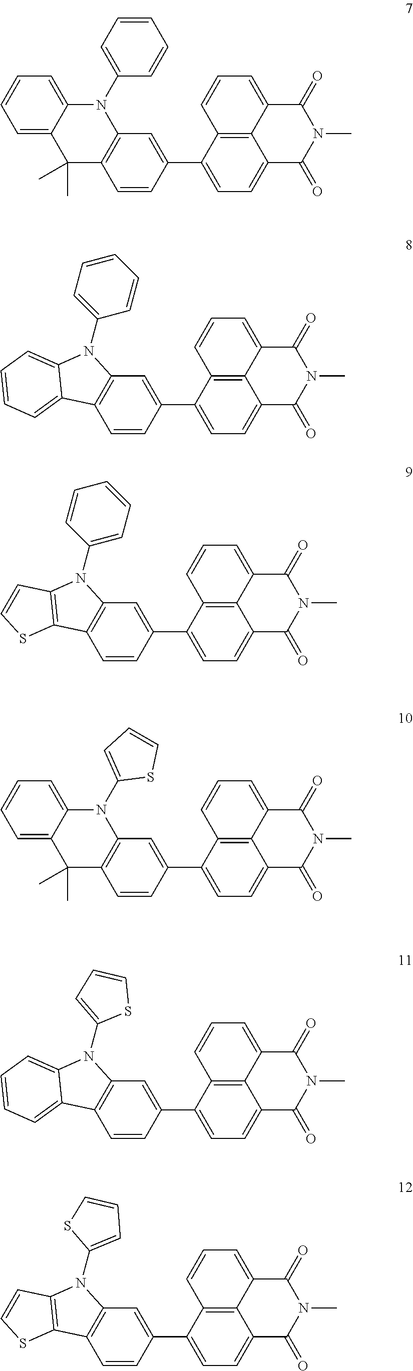 [SMILES: CN1C(=O)c2cccc3c(-c4ccc5c(c4)N(c4ccccc4)c4ccccc4C5(C)C)ccc(c23)C1=O ; CN1C(=O)c2cccc3c(-c4ccc5c6ccccc6n(-c6ccccc6)c5c4)ccc(c23)C1=O ; CN1C(=O)c2cccc3c(-c4ccc5c6sccc6n(-c6ccccc6)c5c4)ccc(c23)C1=O ; CN1C(=O)c2cccc3c(-c4ccc5c(c4)N(c4cccs4)c4ccccc4C5(C)C)ccc(c23)C1=O ; CN1C(=O)c2cccc3c(-c4ccc5c6ccccc6n(-c6cccs6)c5c4)ccc(c23)C1=O ; CN1C(=O)c2cccc3c(-c4ccc5c6sccc6n(-c6cccs6)c5c4)ccc(c23)C1=O] 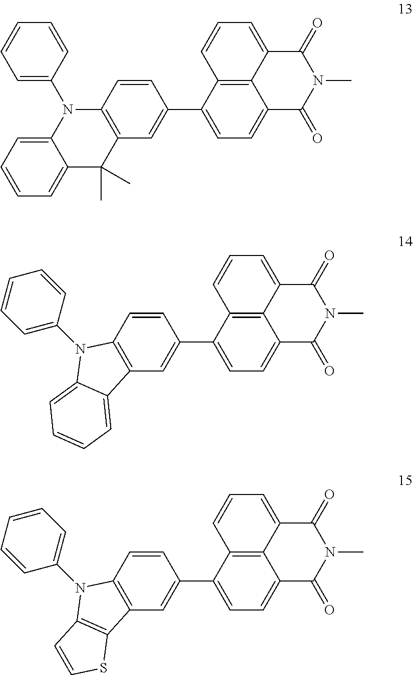 [SMILES: CN1C(=O)c2cccc3c(-c4ccc5c(c4)C(C)(C)c4ccccc4N5c4ccccc4)ccc(c23)C1=O ; CN1C(=O)c2cccc3c(-c4ccc5c(c4)c4ccccc4n5-c4ccccc4)ccc(c23)C1=O ; CN1C(=O)c2cccc3c(-c4ccc5c(c4)c4sccc4n5-c4ccccc4)ccc(c23)C1=O] 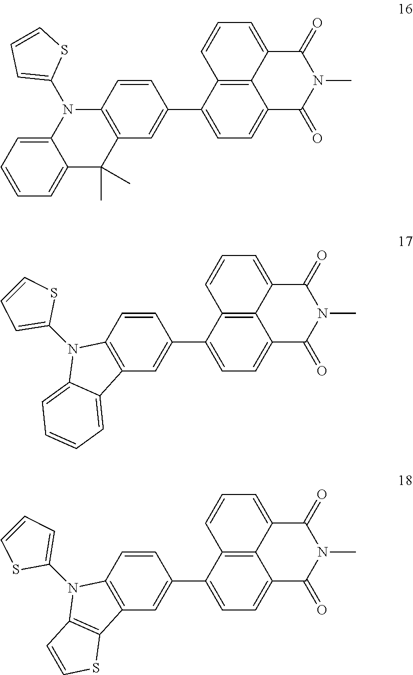 [SMILES: CN1C(=O)c2cccc3c(-c4ccc5c(c4)C(C)(C)c4ccccc4N5c4cccs4)ccc(c23)C1=O ; CN1C(=O)c2cccc3c(-c4ccc5c(c4)c4ccccc4n5-c4cccs4)ccc(c23)C1=O ; CN1C(=O)c2cccc3c(-c4ccc5c(c4)c4sccc4n5-c4cccs4)ccc(c23)C1=O] 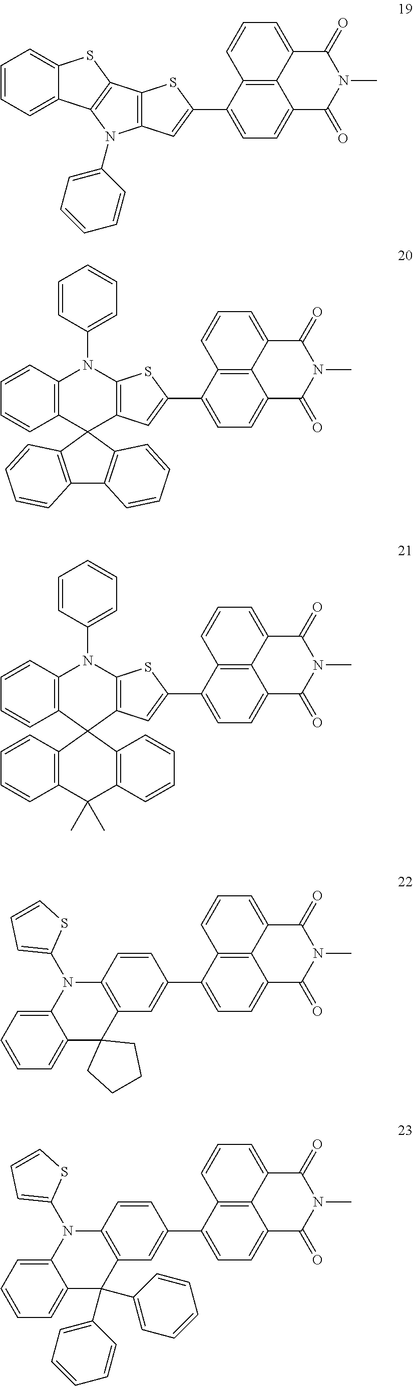 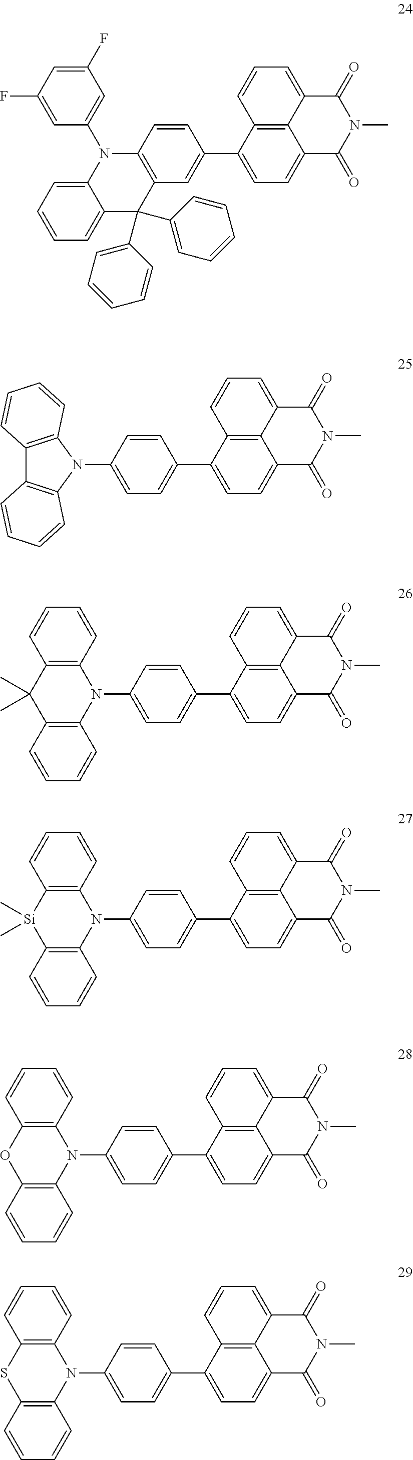 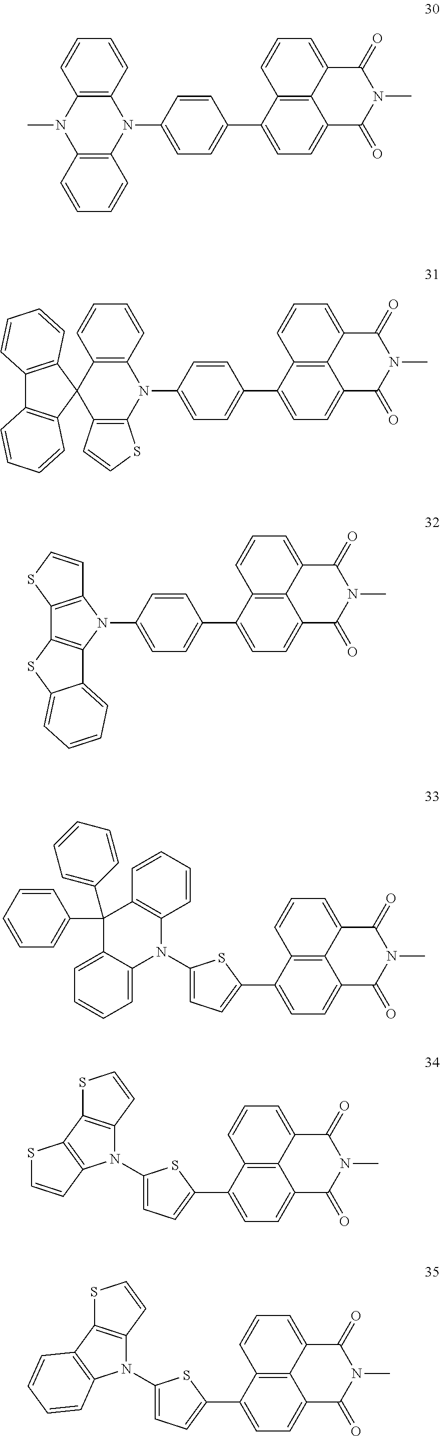 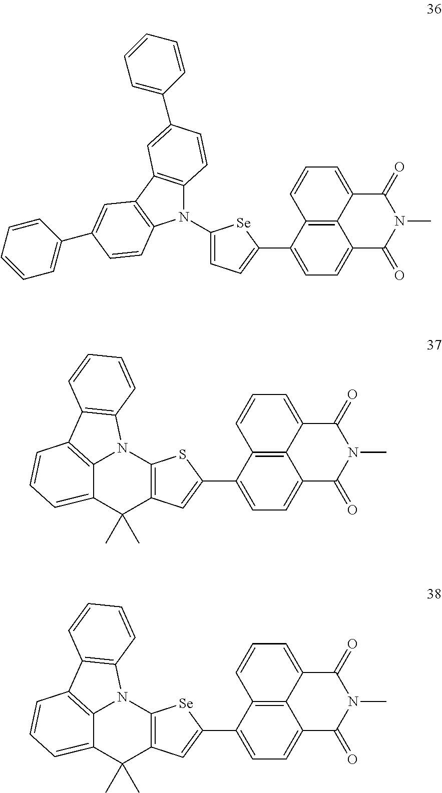 [SMILES: CN1C(=O)c2cccc3c(-c4ccc(-n5c6ccc(-c7ccccc7)cc6c6cc(-c7ccccc7)ccc65)[se]4)ccc(c23)C1=O ; CN1C(=O)c2cccc3c(-c4cc5c(s4)-n4c6ccccc6c6cccc(c64)C5(C)C)ccc(c23)C1=O ; CN1C(=O)c2cccc3c(-c4cc5c([se]4)-n4c6ccccc6c6cccc(c64)C5(C)C)ccc(c23)C1=O] 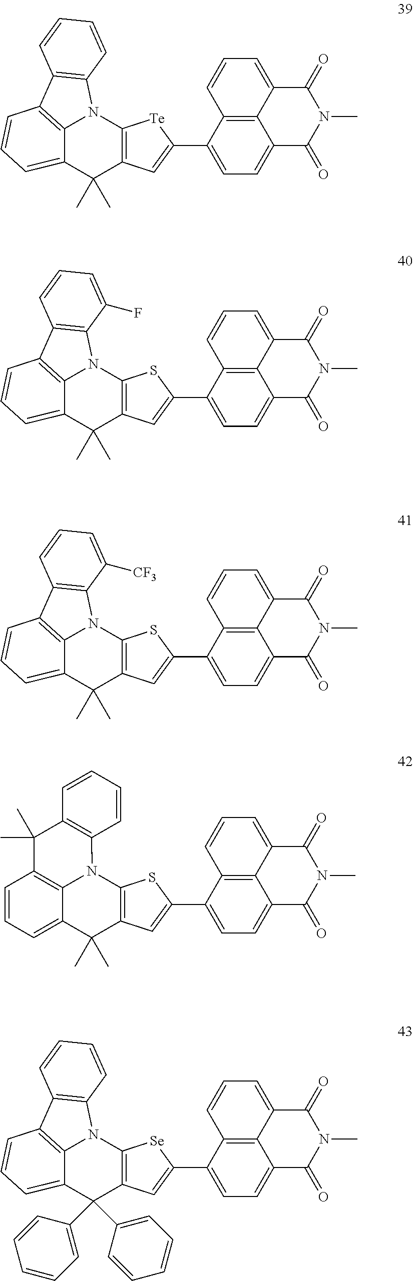 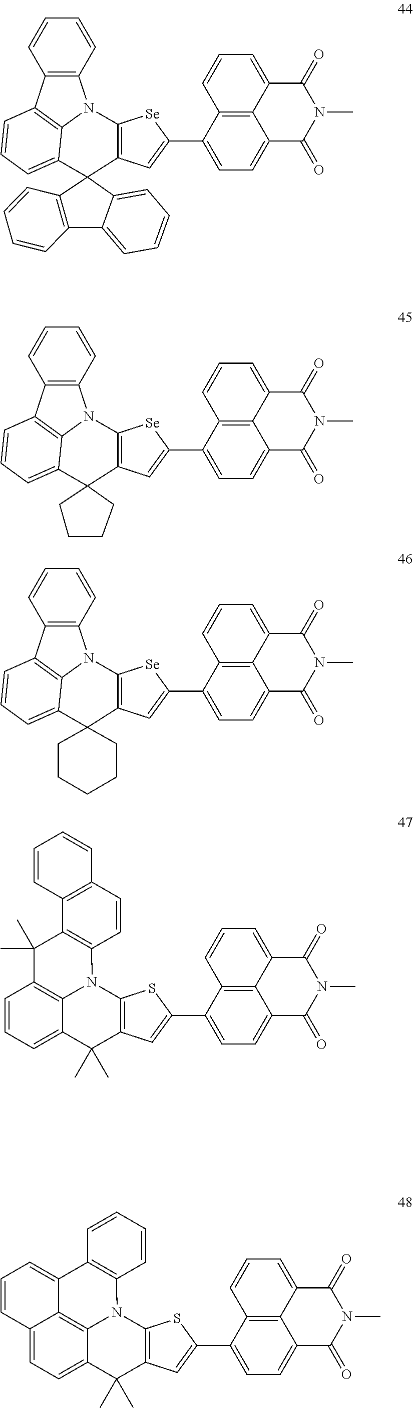 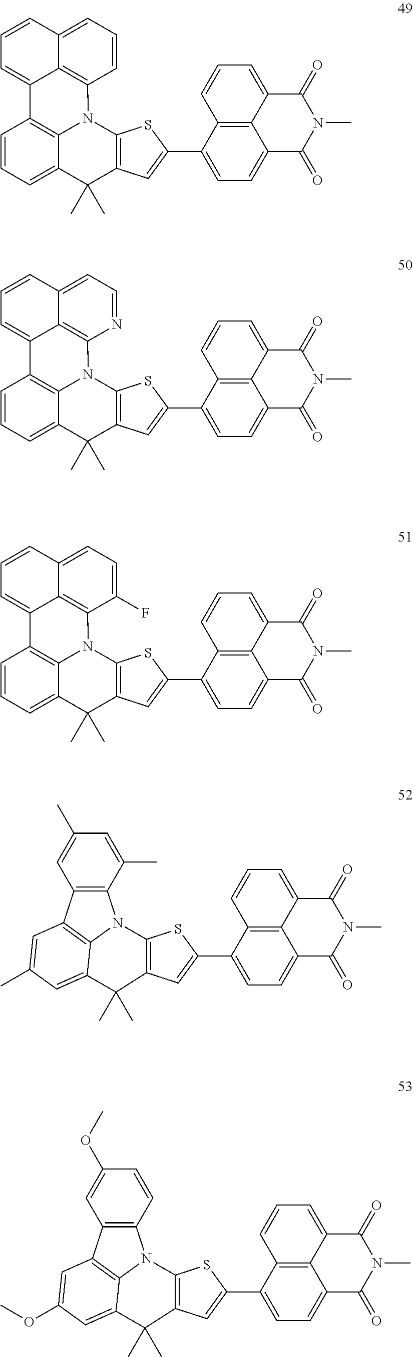 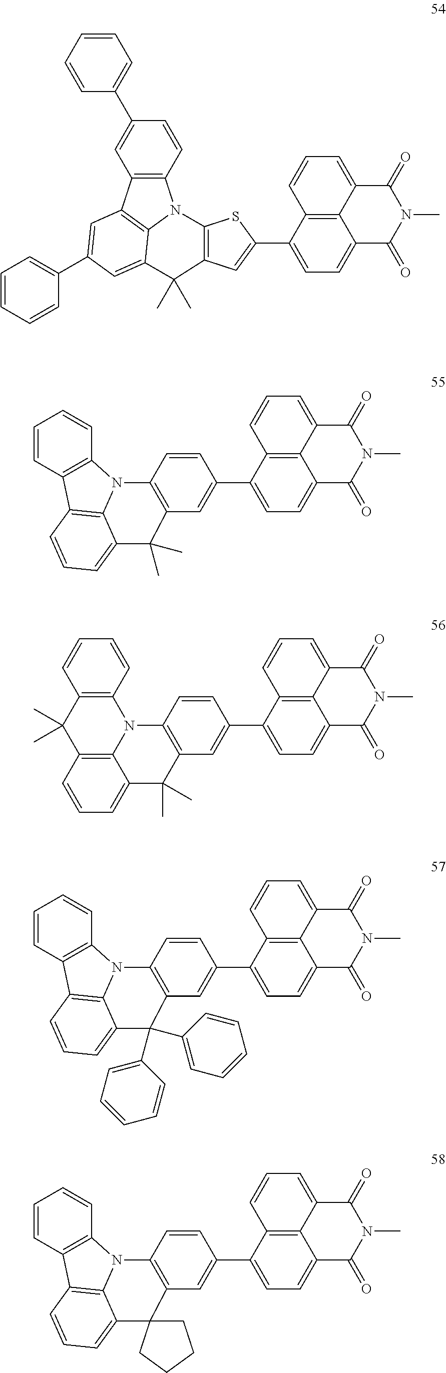 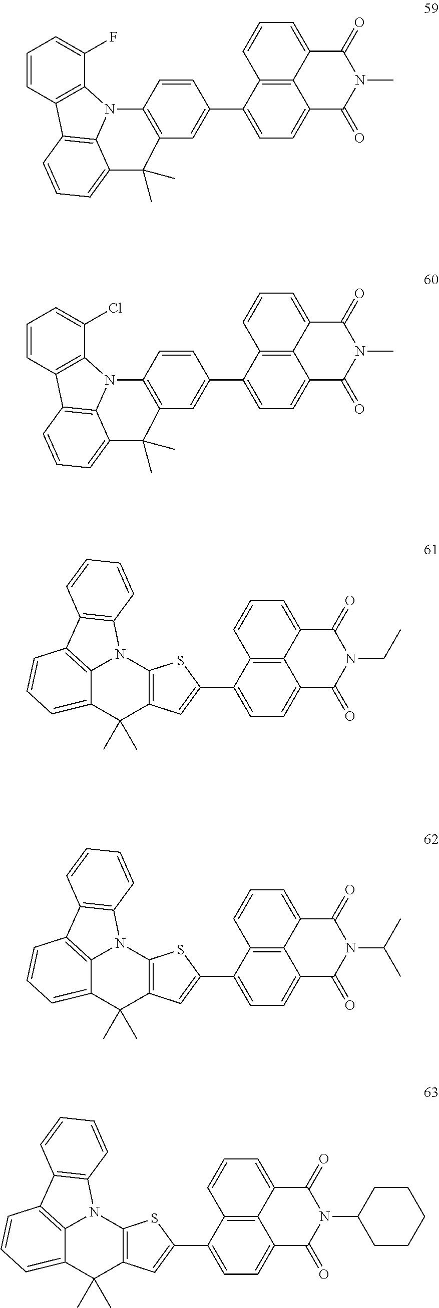 [SMILES: CN1C(=O)c2cccc3c(-c4ccc5c(c4)C(C)(C)c4cccc6c7cccc(F)c7n-5c46)ccc(c23)C1=O ; CN1C(=O)c2cccc3c(-c4ccc5c(c4)C(C)(C)c4cccc6c7cccc(Cl)c7n-5c46)ccc(c23)C1=O ; CCN1C(=O)c2cccc3c(-c4cc5c(s4)-n4c6ccccc6c6cccc(c64)C5(C)C)ccc(c23)C1=O ; CC(C)N1C(=O)c2cccc3c(-c4cc5c(s4)-n4c6ccccc6c6cccc(c64)C5(C)C)ccc(c23)C1=O ; CC1(C)c2cc(-c3ccc4c5c(cccc35)C(=O)N(C3CCCCC3)C4=O)sc2-n2c3ccccc3c3cccc1c32] 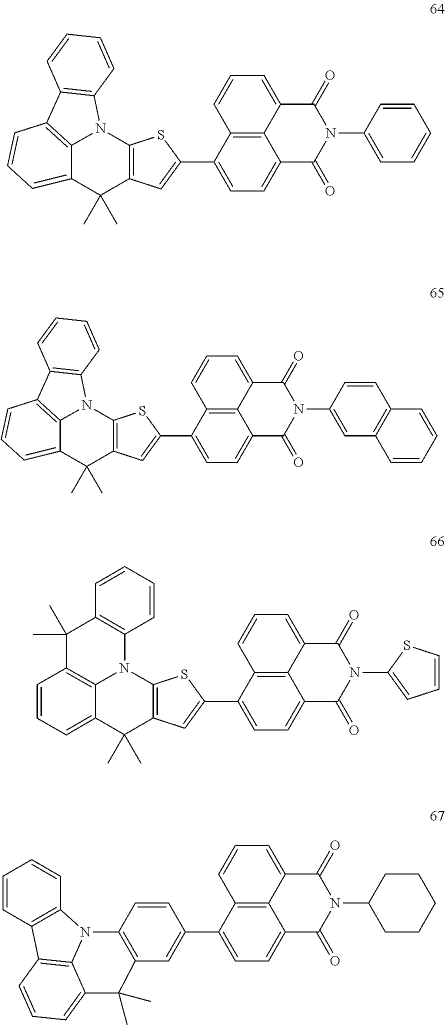 [SMILES: CC1(C)c2cc(-c3ccc4c5c(cccc35)C(=O)N(c3ccccc3)C4=O)sc2-n2c3ccccc3c3cccc1c32 ; CC1(C)c2cc(-c3ccc4c5c(cccc35)C(=O)N(c3ccc5ccccc5c3)C4=O)sc2-n2c3ccccc3c3cccc1c32 ; CC1(C)c2ccccc2N2c3sc(-c4ccc5c6c(cccc46)C(=O)N(c4cccs4)C5=O)cc3C(C)(C)c3cccc1c32 ; CC1(C)c2cc(-c3ccc4c5c(cccc35)C(=O)N(C3CCCCC3)C4=O)ccc2-n2c3ccccc3c3cccc1c32] 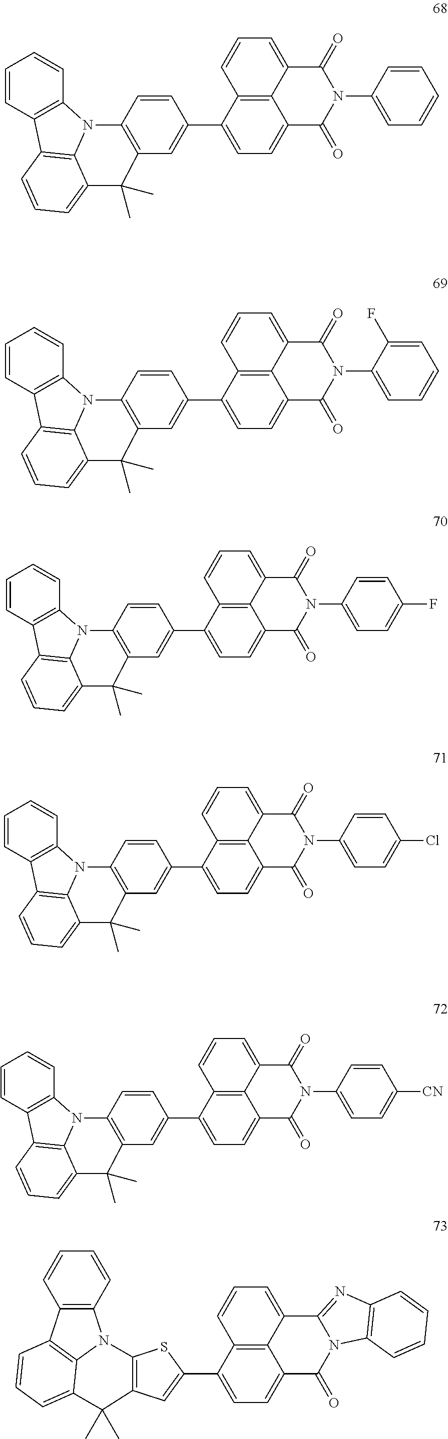 [SMILES: CC1(C)c2cc(-c3ccc4c5c(cccc35)C(=O)N(c3ccccc3)C4=O)ccc2-n2c3ccccc3c3cccc1c32 ; CC1(C)c2cc(-c3ccc4c5c(cccc35)C(=O)N(c3ccccc3F)C4=O)ccc2-n2c3ccccc3c3cccc1c32 ; CC1(C)c2cc(-c3ccc4c5c(cccc35)C(=O)N(c3ccc(F)cc3)C4=O)ccc2-n2c3ccccc3c3cccc1c32 ; CC1(C)c2cc(-c3ccc4c5c(cccc35)C(=O)N(c3ccc(Cl)cc3)C4=O)ccc2-n2c3ccccc3c3cccc1c32 ; CC1(C)c2cc(-c3ccc4c5c(cccc35)C(=O)N(c3ccc(C#N)cc3)C4=O)ccc2-n2c3ccccc3c3cccc1c32 ; CC1(C)c2cc(-c3ccc4c(=O)n5c6ccccc6nc5c5cccc3c45)sc2-n2c3ccccc3c3cccc1c32] 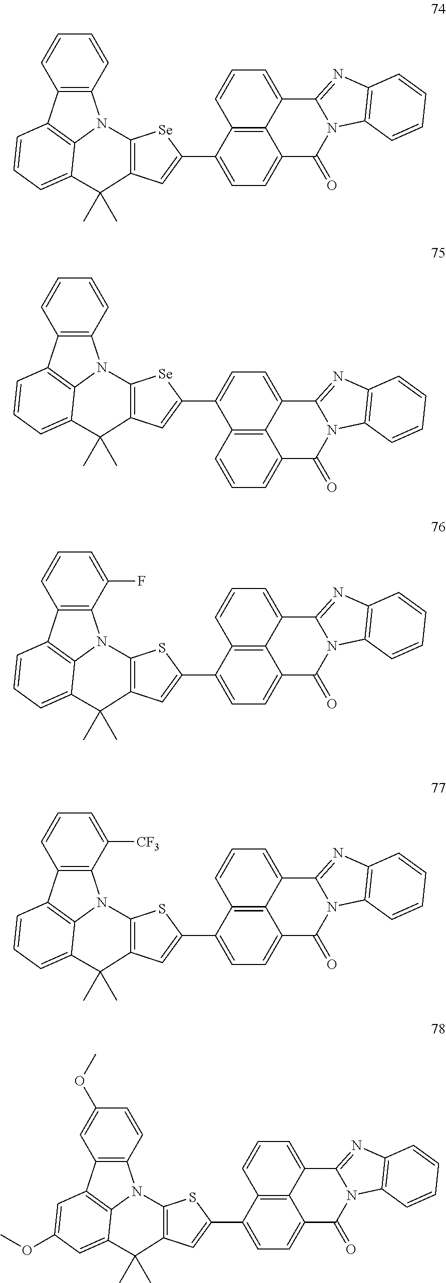 [SMILES: CC1(C)c2cc(-c3ccc4c(=O)n5c6ccccc6nc5c5cccc3c45)[se]c2-n2c3ccccc3c3cccc1c32 ; CC1(C)c2cc(-c3ccc4c5c3cccc5c(=O)n3c5ccccc5nc43)[se]c2-n2c3ccccc3c3cccc1c32 ; CC1(C)c2cc(-c3ccc4c(=O)n5c6ccccc6nc5c5cccc3c45)sc2-n2c3c(F)cccc3c3cccc1c32 ; CC1(C)c2cc(-c3ccc4c(=O)n5c6ccccc6nc5c5cccc3c45)sc2-n2c3c(C(F)(F)F)cccc3c3cccc1c32 ; COc1ccc2c(c1)c1cc(OC)cc3c1n2-c1sc(-c2ccc4c(=O)n5c6ccccc6nc5c5cccc2c45)cc1C3(C)C] 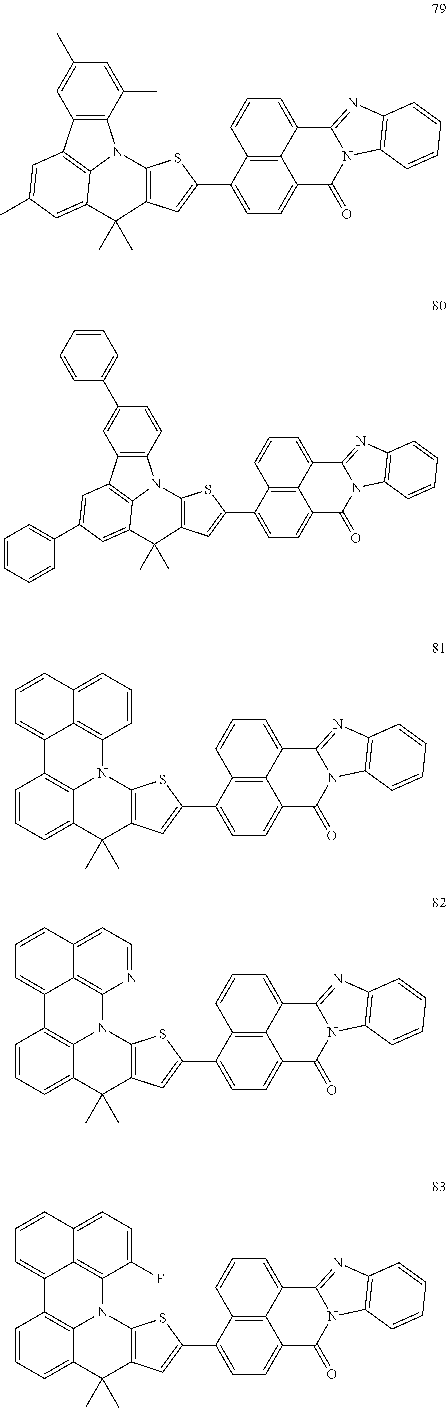 [SMILES: Cc1cc(C)c2c(c1)c1cc(C)cc3c1n2-c1sc(-c2ccc4c(=O)n5c6ccccc6nc5c5cccc2c45)cc1C3(C)C ; CC1(C)c2cc(-c3ccc4c(=O)n5c6ccccc6nc5c5cccc3c45)sc2-n2c3ccc(-c4ccccc4)cc3c3cc(-c4ccccc4)cc1c32 ; CC1(C)c2cc(-c3ccc4c(=O)n5c6ccccc6nc5c5cccc3c45)sc2N2c3c(cccc31)-c1cccc3cccc2c13 ; CC1(C)c2cc(-c3ccc4c(=O)n5c6ccccc6nc5c5cccc3c45)sc2N2c3c(cccc31)-c1cccc3ccnc2c13 ; CC1(C)c2cc(-c3ccc4c(=O)n5c6ccccc6nc5c5cccc3c45)sc2N2c3c(cccc31)-c1cccc3ccc(F)c2c13] 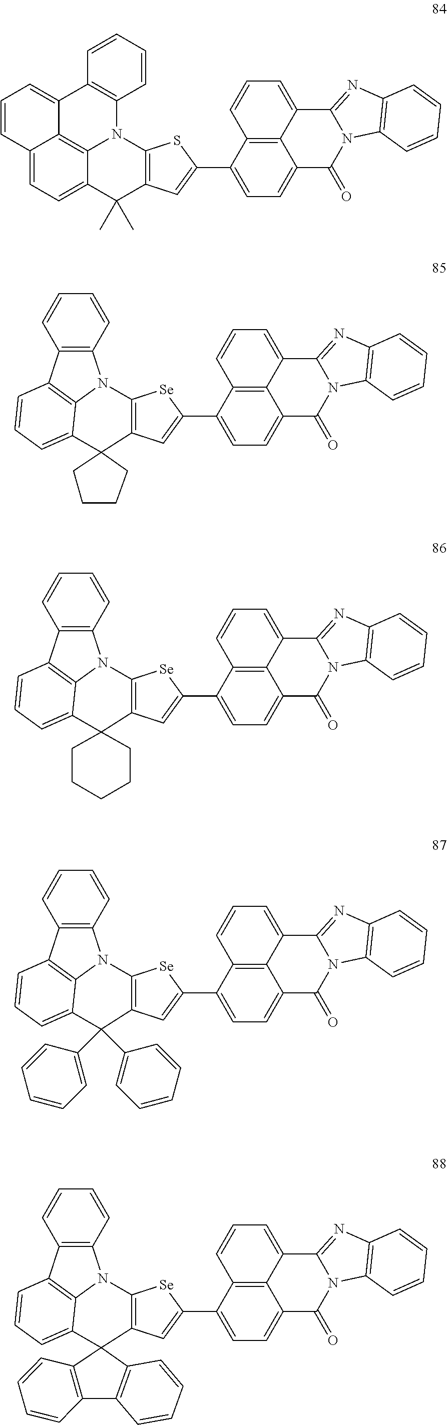 [SMILES: CC1(C)c2cc(-c3ccc4c(=O)n5c6ccccc6nc5c5cccc3c45)sc2N2c3ccccc3-c3cccc4ccc1c2c34 ; O=c1c2ccc(-c3cc4c([se]3)-n3c5ccccc5c5cccc(c53)C43CCCC3)c3cccc(c32)c2nc3ccccc3n12 ; O=c1c2ccc(-c3cc4c([se]3)-n3c5ccccc5c5cccc(c53)C43CCCCC3)c3cccc(c32)c2nc3ccccc3n12 ; O=c1c2ccc(-c3cc4c([se]3)-n3c5ccccc5c5cccc(c53)C4(c3ccccc3)c3ccccc3)c3cccc(c32)c2nc3ccccc3n12 ; O=c1c2ccc(-c3cc4c([se]3)-n3c5ccccc5c5cccc(c53)C43c4ccccc4-c4ccccc43)c3cccc(c32)c2nc3ccccc3n12] 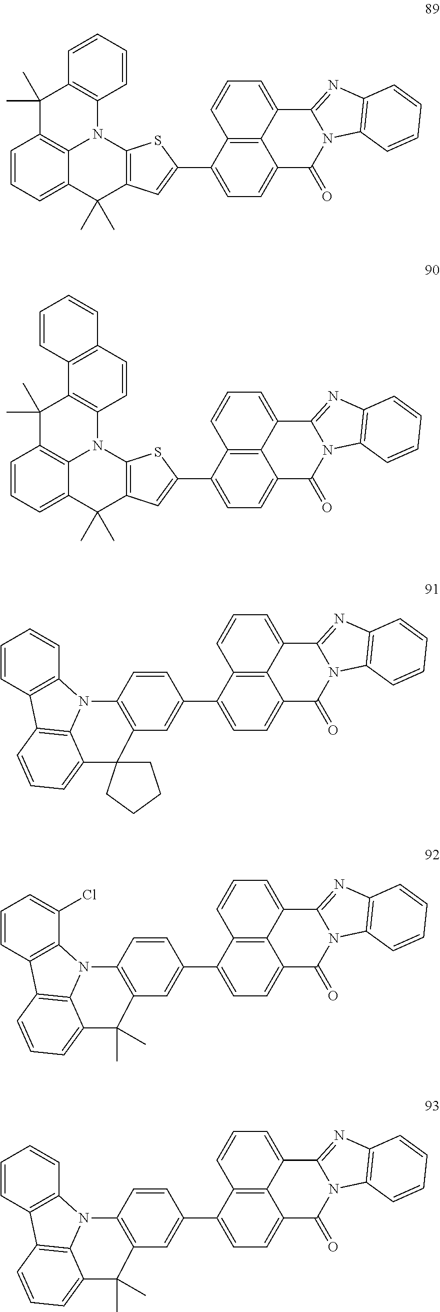 [SMILES: CC1(C)c2ccccc2N2c3sc(-c4ccc5c(=O)n6c7ccccc7nc6c6cccc4c56)cc3C(C)(C)c3cccc1c32 ; CC1(C)c2cc(-c3ccc4c(=O)n5c6ccccc6nc5c5cccc3c45)sc2N2c3ccc4ccccc4c3C(C)(C)c3cccc1c32 ; O=c1c2ccc(-c3ccc4c(c3)C3(CCCC3)c3cccc5c6ccccc6n-4c35)c3cccc(c32)c2nc3ccccc3n12 ; CC1(C)c2cc(-c3ccc4c(=O)n5c6ccccc6nc5c5cccc3c45)ccc2-n2c3c(Cl)cccc3c3cccc1c32 ; CC1(C)c2cc(-c3ccc4c(=O)n5c6ccccc6nc5c5cccc3c45)ccc2-n2c3ccccc3c3cccc1c32] 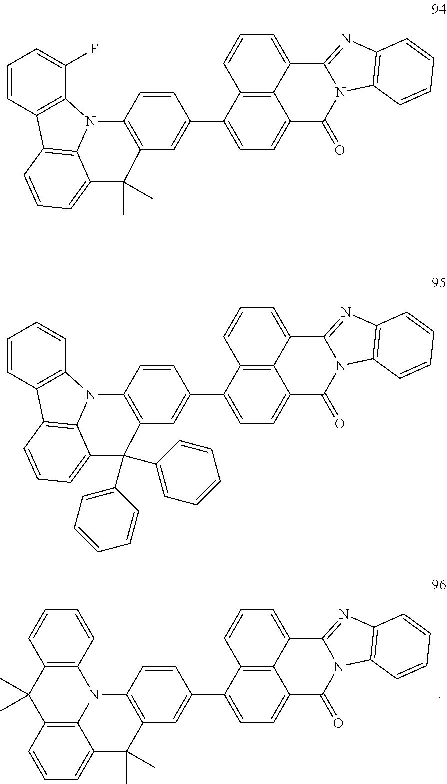 [SMILES: CC1(C)c2cc(-c3ccc4c(=O)n5c6ccccc6nc5c5cccc3c45)ccc2-n2c3c(F)cccc3c3cccc1c32 ; O=c1c2ccc(-c3ccc4c(c3)C(c3ccccc3)(c3ccccc3)c3cccc5c6ccccc6n-4c35)c3cccc(c32)c2nc3ccccc3n12 ; CC1(C)c2ccccc2N2c3ccc(-c4ccc5c(=O)n6c7ccccc7nc6c6cccc4c56)cc3C(C)(C)c3cccc1c32]
